(19) United States
(12) Patent Application Publication
(10) Pub. No.: US 2011/0301122 A1
(43) Pub. Date: Dec. 8, 2011

(54) HETEROAROMATIC COMPOUNDS FOR USE AS HIF INHIBITORS

(75) Inventors: Michael Härter, Leverkusen (DE); Hartmut Beck, Koln (DE); Peter Ellinghaus, Melle (DE); Kerstin Berhörster, Essen (DE); Susanne Greschat, Wagenfeld (DE); Karl-Heinz Thierauch, Berlin (DE); Frank Süssmeier, Munchen (DE)

(73) Assignee: BAYER SCHERING PHARMA AKTIENGESELLSCHAFT, Berlin (DE)

(21) Appl. No.: 13/129,409

(22) PCT Filed: Oct. 31, 2009
(86) PCT No.: PCT/EP09/07807

§ 371 Date: Aug. 17, 2011

(30) Foreign Application Priority Data
Nov. 14, 2008 (DE) 10 2008 057 364.7
Sep. 11, 2009 (DE) 10 2009 041 241.7

(51) Int. Cl. A61K 31/695; C07F 7/10; C07D 413/04; A61K 31/4439; A61K 31/4245; A61K 31/454; A61K 31/444; A61K 31/422; A61K 31/4545; A61P 9/10; A61P 9/00; A61P 9/06; A61P 9/12; A61P 35/00; A61P 17/06; A61P 27/02; A61P 19/02; A61P 7/00; C07D 413/14

(52) U.S. Cl. 514/63; 546/269.1; 546/14; 548/131; 546/209; 546/256; 548/235; 546/194; 514/340; 514/364; 514/326; 514/333; 514/374; 514/318

(57) ABSTRACT

The present application relates to novel substituted aryl compounds, processes for their preparation, their use for treatment and/or prevention of diseases and their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

The present application relates to novel substituted aryl compounds, processes for their preparation, their use for treatment and/or prevention of diseases and their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

Cancer diseases are the consequence of uncontrolled cell growth of the most diverse tissue. In many cases the new cells penetrate into existing tissue (invasive growth), or they metastase into remote organs. Cancer diseases occur in the most diverse organs and often have tissue-specific courses of the disease. The term cancer as a generic term therefore describes a large group of defined diseases of various organs, tissue and cell types.

In the year 2002 4.4 million people worldwide were diagnosed with tumour diseases of the breast, intestine, ovaries, lung or prostate. In the same year, approx. 2.5 million deaths were assumed to be a consequence of these diseases (Globocan 2002 Report). In the USA alone, for the year 2005 over 1.25 million new cases and over 500,000 deaths were predicted from cancer diseases. The majority of these new cases concern cancer diseases of the intestine (˜100,000), lung (˜170,000), breast (˜210,000) and prostate (˜230,000). A further increase in cancer diseases of approx. 15% over the next 10 years is assumed (American Cancer Society, Cancer Facts and Figures 2005).

Tumours in early stages can possibly be removed by surgical and radiotherapy measures. Metastased tumours as a rule can only be treated palliatively by chemotherapeutics. The aim here is to achieve the optimum combination of an improvement in the quality of life and prolonging of life.

Chemotherapies are often composed of combinations of cytotoxic medicaments. The majority of these substances have as their action mechanism bonding to tubulin, or they are compounds which interact with the formation and processing of nucleic acids. More recently these also include enzyme inhibitors, which interfere with epigenetic DNA modification or cell cycle progression (e.g. histone deacetylase inhibitors, aurora kinase inhibitors). Since such therapies are toxic, more recently the focus has increasingly been on targeted therapies in which specific processes in the cell are blocked without there being a high toxic load. These include in particular inhibitors of kinases which inhibit the phosphorylation of receptors and signal transmission molecules. An example of these is imatinib, which is employed very successfully for treatment of chronic myeloid leukaemia (CML) and gastrointestinal stromal tumours (GIST). Further examples are substances which block EGFR kinase and HER2, such as erlotinib, and VEGFR kinase inhibitors, such as sorafenib and sunitinib, which are employed on kidney cell carcinomas, liver carcinomas and advanced stages of GIST.

The life expectancy of colorectal carcinoma patients has been successfully prolonged with an antibody directed against VEGF. Bevacizumab inhibits growth of blood vessels, which obstructs rapid expansion of tumours since this requires connection to the blood vessel system for a continuously functioning supply and disposal.

One stimulus of angiogenesis is hypoxia, which occurs again and again with solid tumours since the blood supply is inadequate because of the unregulated growth. If there is a lack of oxygen, cells switch their metabolism from oxidative phosphorylation to glycolysis so that the ATP level in the cell is stabilized. This process is controlled by a transcription factor, which is regulated upwards depending on the oxygen content in the cell. This transcription factor, called “hypoxia-induced factor” (HIF) is normally removed posttranslationally by rapid degradation and prevented from transportation into the cell nucleus. This is effected by hydroxylation of two proline units in the oxygen degradable domain (ODD) and an asparagine unit in the vicinity of the C terminus by the enzymes prolyl dehydrogenase and FIH (“factor inhibiting HIF”). After the modification of the proline units, HIF can be degraded with mediation by the Hippel-Lindau protein (part of a ubiquitin-E3-ligase complex) via the proteasome apparatus (Maxwell, Wiesener et al, 1999). In the event of oxygen deficiency, the degradation does not take place and the protein is regulated upwards and leads to transcription or blockade of the transcription of numerous (more than 100) other proteins (Semenza and Wang, 1992; Wang and Semenza, 1995).

The transcription factor HIF is formed by the regulated α-subunit and a constitutively present β-subunit (ARNT, aryl hydrocarbon receptor nuclear translocator). There are three different species of the α-subunit, 1α, 2α and 3α, the last being rather to be assumed as a suppressor (Makino, Cao et al, 2001) The HIF subunits are bHLH (basic helix loop helix) proteins, which dimerize via their HLH and PAS (Per-Arnt-Sim) domain, which starts their transactivation activity (Jiang, Rue et al., 1996).

In the most important tumour entities, overexpression of the HIF1α protein is correlated with increasing density of blood vessels and enhanced VEGF expression (Hirota and Semenza, 2006). At the same time glucose metabolism is changed to glycolysis, and the Krebs cycle is reduced in favour of the production of cell units. This also implies a change in fat metabolism. Such changes appear to guarantee the survival of the tumours. On the other hand, if the activity of HIF is now inhibited, the development of tumours could consequently be suppressed. This has already been observed in various experimental models (Chen, Zhao et al., 2003; Stoeltzing, McCarty et al., 2004; Li, Lin et al., 2005; Mizukami, Jo et al., 2005; Li, Shi et al., 2006). Specific inhibitors of the metabolism controlled by HIF should therefore be suitable as tumour therapeutics.

The object of the present invention was therefore to provide novel compounds which act as inhibitors of the transactivating action of the transcription factor HIF and can be employed as such for treatment and/or prevention of diseases, in particular of hyperproliferative and angiogenic diseases, such as cancer diseases.

Substituted multicyclic heteroaryl compounds with pyrrole, pyrazole and/or oxadiazole partial structures and the use of these compounds for treatment of diverse diseases are described in numerous forms in the patent literature, thus inter alia in EP 0 908 456-A1, WO 97/36881-A1, WO 01/12627-A1, WO 01/85723-A1, WO 02/100826-A2, WO 2004/014370-A2, WO 2004/014881-A2, WO 2004/014902-A2, WO 2004/035566-A1, WO 2004/058176-A2, WO 2004/089303-A2, WO 2004/089308-A2, WO 2005/070925-A1, WO 2006/114313-A1, WO 2007/002559-A1, WO 2007/034279-A2, WO 2008/004096-A1, WO 2008/024390-A2 and WO 2008/114157-A1. WO 2005/030121-A2 and WO 2007/065010-A2 claim the use of certain pyrazole derivatives for inhibition of the expression of HIF and HIF-regulated genes in tumour cells. WO 2008/141731-A2 describes heteroaryl-substituted N-benzylpyrazoles as inhibitors of the HIF regulation pathway for treatment of cancer diseases. Heteroaryl-substituted 5-(1H-pyrazol-3-yl)-1,2,4-oxadiazoles as cannabinoid receptor modulators for treatment of diverse diseases are disclosed in US 2008/0255211-A1. Further diaryl-substituted isoxazole and 1,2,4-oxadiazole derivatives are described in WO 2009/029632-A1 as inhibitors of monoamine oxidase B for treatment of psychiatric diseases.

The present invention provides compounds of the general formula (I)

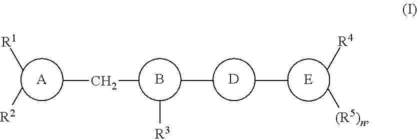

in which
either (a)
   the ring

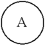

represents a pyridyl ring
and
the ring

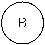

with the substituent R³ represents a heteroaryl ring of the formula

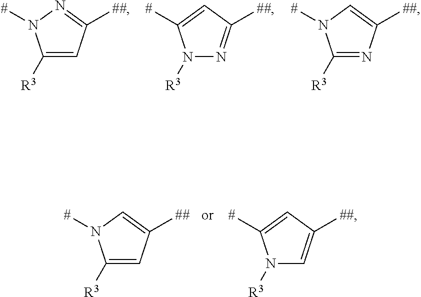

wherein
# designates the linkage point with the adjacent CH₂ group
and
## designates the linkage point with the ring

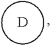

or (b)
   the ring

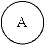

represents a phenyl ring
and
the ring

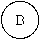

with the substituent R³ represents a heteroaryl ring of the formula

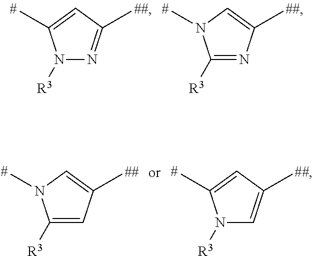

wherein
# designates the linkage point with the adjacent CH₂ group
and
## designates the linkage point with the ring

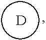

the ring

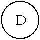

represents a heteroaryl ring of the formula

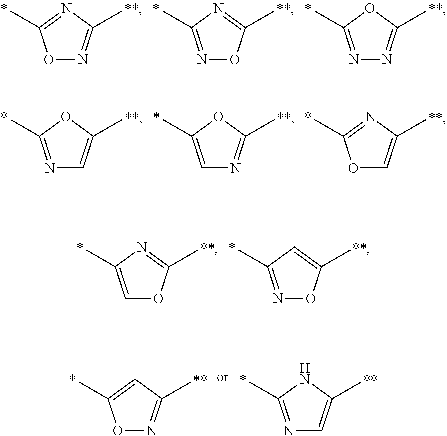

wherein
   * designates the linkage point with the ring

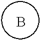

and
   ** designates the linkage point with the ring

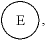

the ring

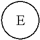

represents a phenyl or pyridyl ring,
R¹ represents hydrogen or a substituent chosen from the series halogen, cyano, (C₁-C₆)-alkyl, (C₂-C₆)-alkenyl, (C₂-C₆)-alkynyl, (C₃-C₆)-cycloalkyl, oxetanyl, tetrahydrofuranyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷, —NR⁶R⁸, —N(R⁶)—C(═O)—R⁷ and —N(R⁶)—S(═O)₂—R⁷,
   wherein (C₁-C₆)-alkyl, (C₂-C₆)-alkenyl and (C₂-C₆)-alkynyl in their turn can be substituted up to three times by fluorine and up to two times in an identical or different manner by a radical chosen from the series hydroxyl, (C₁-C₄)-alkoxy, trifluoromethoxy, tri-(C₁-C₄)-alkylsilyl, (C₁-C₄)-alkoxycarbonyl and (C₃-C₆)-cycloalkyl
   and
   oxetanyl, tetrahydrofuranyl, tetrahydropyranyl and the cycloalkyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
   and wherein
   R⁶ and R⁷ independently of each other denote hydrogen, (C₁-C₆)-alkyl or (C₃-C₆)-cycloalkyl,
      wherein (C₁-C₆)-alkyl can be substituted up to three times by fluorine and up to two times in an identical or different manner by a radical chosen from the series hydroxyl, (C₁-C₄)-alkoxy, trifluoromethoxy, (C₁-C₄)-alkoxycarbonyl and (C₃-C₆)-cycloalkyl
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
   and
   R⁸ denotes hydrogen, amino, (C₁-C₆)-alkyl, (C₃-C₆)-cycloalkyl or 5- or 6-membered heteroaryl,
      wherein (C₁-C₆)-alkyl can be substituted up to three times by fluorine and up to two times in an identical or different manner by a radical chosen from the series hydroxyl, (C₁-C₄)-alkoxy, trifluoromethoxy, (C₁-C₄)-alkoxycarbonyl, (C₃-C₆)-cycloalkyl, oxetanyl, tetrahydrofuranyl, tetrahydropyranyl and 5- or 6-membered heteroaryl
      and wherein
      oxetanyl, tetrahydrofuranyl, tetrahydropyranyl and the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and (C₁-C₄)-alkoxycarbonyl,
      and
      the heteroaryl groups mentioned can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
R² represents hydrogen or a substituent chosen from the series fluorine, chlorine, cyano, methyl, trifluoromethyl, hydroxyl, methoxy and trifluoromethoxy,
R³ represents methyl, ethyl or trifluoromethyl,
R⁴ represents hydrogen or a substituent chosen from the series halogen, cyano, pentafluorothio, (C₁-C₆)-alkyl, tri-(C₁-C₄)-alkylsilyl, —OR⁹, —NR⁹R¹⁰, —N(R⁹)—C(═O)—R¹⁰, —N(R⁹)—C(═O)—OR¹⁰, —N(R⁹)—S(═O)₂—R¹⁰, —C(═O)—OR⁹, —C(═O)—NR⁹R¹⁰, —SR⁹, —S(═O)—R⁹, —S(═O)₂—R⁹, —S(═O)₂—NR⁹R¹⁰, —S(═O)(═NH)—R⁹, —S(═O)(═NCH₃)—R⁹, (C₃-C₆)-cycloalkyl, 4- to 6-membered heterocyclyl and 5- or 6-membered heteroaryl,
   wherein (C₁-C₆)-alkyl in its turn can be substituted up to three times by fluorine and up to two times in an identical or different manner by a radical chosen from the series —OR⁹, —NR⁹R¹⁰, —N(R⁹)—C(═O)—R¹⁰, —N(R⁹)—C(═O)—OR¹⁰, —C(═O)—OR⁹, —C(═O)—NR⁹R¹⁰, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkyl-carbonyl, (C₁-C₄)-alkoxycarbonyl, aminocarbonyl, mono-(C₁-C₄)-alkylaminocarbonyl and di-(C₁-C₄)-alkylaminocarbonyl
   and
   the heteroaryl groups mentioned in their turn can be substituted up to two times in an identical or different manner by a radical chosen from the series fluorine, chlorine, cyano, (C₁-C₄)-alkyl and (C₁-C₄)-alkoxy
      wherein the (C₁-C₄)-alkyl substituents mentioned herein and the (C₁-C₄)-alkoxy substituents mentioned herein in their turn can be substituted by hydroxyl, (C₁-C₄)-alkoxy, trifluoromethoxy, (C₁-C₄)-alkylcarbonyloxy, (C₁-C₄)-alkoxycarbonyl, aminocarbonyl, mono-(C₁-C₄)-alkylaminocarbonyl or di-(C₁-C₄)-alkylaminocarbonyl or up to three times by fluorine,
   and wherein
   R⁹ and R¹⁰ independently of each other for each individual occurrence denote hydrogen, (C₁-C₆)-alkyl, (C₃-C₆)-cycloalkyl or 4- to 6-membered heterocyclyl,
      wherein (C₁-C₆)-alkyl can be substituted up to three times by fluorine and up to two times in an identical or different manner by a radical chosen from the series hydroxyl, (C₁-C₄)-alkoxy, trifluoromethoxy, amino, mono-(C₁-C₄)-alkylamino, di-(C₁-C₄)-alkylamino, (C₁-C₄)-alkoxycarbonyl, (C₃-C₆)-cycloalkyl and 4- to 6-membered heterocyclyl
      and
      the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
   or
   R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
R⁵ represents a substituent chosen from the series fluorine, chlorine, cyano, methyl, trifluoromethyl and hydroxyl
and
n represents the number 0, 1 or 2,
   wherein in the case where the substituent R⁵ occurs twice, its meanings can be identical or different,
and their salts, solvates and solvates of the salts.

An alternative embodiment within the subject matter of the invention described above comprises compounds of the formula (I) in which
R¹ represents hydrogen or a substituent chosen from the series halogen, cyano, (C₁-C₆)-alkyl, (C₂-C₆)-alkenyl, (C₂-C₆)-alkynyl, (C₃-C₆)-cycloalkyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷, —NR⁶R⁸, —N(R⁶)—C(═O)—R⁷ and —N(R⁶)—S(═O)₂—R⁷,
   wherein (C₁-C₆)-alkyl, (C₂-C₆)-alkenyl and (C₂-C₆)-alkynyl in their turn can be substituted up to three times by fluorine and up to two times in an identical or different manner by a radical chosen from the series hydroxyl, (C₁-C₄)-alkoxy, trifluoromethoxy, tri-(C₁-C₄)-alkylsilyl, (C₁-C₄)-alkoxycarbonyl and (C₃-C₆)-cycloalkyl
   and
   the cycloalkyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
   and wherein
   R⁶ and R⁷ have the meanings given above
   and
   R⁸ denotes hydrogen, (C₁-C₆)-alkyl, (C₃-C₆)-cycloalkyl or 5- or 6-membered heteroaryl,
      wherein (C₁-C₆)-alkyl can be substituted up to three times by fluorine and up to two times in an identical or different manner by a radical chosen from the series hydroxyl, (C₁-C₄)-alkoxy, trifluoromethoxy, (C₁-C₄)-alkoxycarbonyl, (C₃-C₆)-cycloalkyl, oxetanyl, tetrahydrofuranyl, tetrahydropyranyl and 5- or 6-membered heteroaryl
      and wherein
      oxetanyl, tetrahydrofuranyl, tetrahydropyranyl and the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and (C₁-C₄)-alkoxycarbonyl,
      and
      the heteroaryl groups mentioned can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
and
R⁴ represents hydrogen or a substituent chosen from the series halogen, cyano, pentafluorothio, (C₁-C₆)-alkyl, tri-(C₁-C₄)-alkylsilyl, —OR⁹, —NR⁹R¹⁰, —N(R⁹)—C(═O)—R¹⁰, —N(R⁹)—C(═O)—OR¹⁰, —N(R⁹)—S(═O)₂—R¹⁰, —C(═O)—OR⁹, —C(═O)—NR⁹R¹⁰, —SR⁹, —S(═O)—R⁹, —S(═O)₂—R⁹, —S(═O)₂—NR⁹R¹⁰, (C₃-C₆)-cycloalkyl, 4- to 6-membered heterocyclyl and 5- or 6-membered heteroaryl,
   wherein (C₁-C₆)-alkyl in its turn can be substituted up to three times by fluorine and up to two times in an identical or different manner by a radical chosen from the series —OR⁹, —NR⁹R¹⁰, —N(R⁹)—C(═O)—R¹⁰, —N(R⁹)—C(═O)—OR¹⁰, —C(═O)—OR⁹, —C(═O)—NR⁹R¹⁰, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkylcarbonyl and (C₁-C₄)-alkoxycarbonyl
   and
   the heteroaryl groups mentioned in their turn can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
   and wherein R⁹ and R¹⁰ have the meanings given above
and their salts, solvates and solvates of the salts.

Compounds according to the invention are the compounds of the formula (I) and their salts, solvates and solvates of the salts, the compounds included in the formula (I) of the formulae mentioned in the following and their salts, solvates and solvates of the salts, and the compounds included in the formula (I) and mentioned in the following as embodiment examples and their salts, solvates and solvates of the salts, where the compounds included in the formula (I) and mentioned in the following are not already salts, solvates and solvates of the salts.

The compounds according to the invention can exist in stereoisomeric forms (enantiomers, diastereomers), depending on their structure. The invention therefore includes the enantiomers or diastereomers and their particular mixtures. The stereoisomerically uniform constituents can be isolated from such mixtures of enantiomers and/or diastereomers in a known manner; chromatography processes are preferably used for this, in particular HPLC chromatography on an achiral or chiral phase.

Where the compounds according to the invention can occur in tautomeric forms, the present invention includes all the tautomeric forms.

Preferred salts in the context of the present invention are physiologically acceptable salts of the compounds according to the invention. Salts which are not themselves suitable for pharmaceutical uses but can be used, for example, for isolation or purification of the compounds according to the invention are also included.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds according to the invention which form a complex in the solid or liquid state by coordination with solvent molecules. Hydrates are a specific form of solvates, in which the coordination takes place with water. Hydrates are preferred solvates in the context of the present invention.

The N-oxides of pyridyl rings and tertiary cyclic amine groupings contained in compounds according to the invention are similarly included in the present invention.

The present invention moreover also includes prodrugs of the compounds according to the invention. The term “prodrugs” here designates compounds which themselves can be biologically active or inactive, but are converted (for example metabolically or hydrolytically) into compounds according to the invention during their dwell time in the body.

In the context of the present invention, the substituents have the following meaning, unless specified otherwise:

(C₁-C₆)-Alkyl and (C₁-C₄)-alkyl in the context of the invention represent a straight-chain or branched alkyl radical having 1 to 6 or, respectively, 1 to 4 carbon atoms. A straight-chain or branched alkyl radical having 1 to 4 carbon atoms is preferred. There may be mentioned by way of example and preferably: methyl, ethyl, n-propyl, isopropyl, n-butyl, iso-butyl, sec-butyl, tert-butyl, n-pentyl, 2-pentyl, 3-pentyl, neopentyl, n-hexyl, 2-hexyl and 3-hexyl.

(C₂-C₆)-Alkenyl and (C₂-C₄)-alkenyl in the context of the invention represent a straight-chain or branched alkenyl radical having 2 to 6 or, respectively, 2 to 4 carbon atoms and a double bond. A straight-chain or branched alkenyl radical having 2 to 4 carbon atoms is preferred. There may be mentioned by way of example and preferably: vinyl, allyl, n-prop-1-en-1-yl, isopropenyl, 2-methyl-2-propen-1-yl, n-but-1-en-1-yl, n-but-2-en-1-yl and n-but-3-en-1-yl.

(C₂-C₆)-Alkynyl and (C₂-C₄)-alkynyl in the context of the invention represent a straight-chain or branched alkynyl radical having 2 to 6 or, respectively, 2 to 4 carbon atoms and a triple bond. A straight-chain or branched alkynyl radical having 2 to 4 carbon atoms is preferred. There may be mentioned by way of example and preferably: ethynyl, n-prop-1-yn-1-yl, n-prop-2-yn-1-yl, n-but-1-yn-1-yl, n-but-2-yn-1-yl and n-but-3-yn-1-yl.

(C₁-C₄)-Alkylcarbonyl in the context of the invention represents a straight-chain or branched alkyl radical having 1 to 4 carbon atoms which is linked via a carbonyl group [—C(═O)—]. There may be mentioned by way of example and preferably: acetyl, propionyl, n-butyryl, iso-butyryl, n-pentanoyl and pivaloyl.

Tri-(C₁-C₄)-alkylsilyl in the context of the invention represents a silyl group with three identical or different straight-chain or branched alkyl substituents, each of which contains 1 to 4 carbon atoms. There may be mentioned by way of example and preferably: trimethylsilyl, tert-butyl-dimethylsilyl and triisopropylsilyl.

(C₁-C₄)-Alkoxy in the context of the invention represents a straight-chain or branched alkoxy radical having 1 to 4 carbon atoms. There may be mentioned by way of example and preferably: methoxy, ethoxy, n-propoxy, isopropoxy, n-butoxy, iso-butoxy, sec-butoxy and tert-butoxy.

(C₁-C₄)-Alkoxycarbonyl in the context of the invention represents a straight-chain or branched alkoxy radical having 1 to 4 carbon atoms which is linked via a carbonyl group [—C(═O)—]. There may be mentioned by way of example and preferably: methoxycarbonyl, ethoxycarbonyl, n-propoxycarbonyl, isopropoxycarbonyl, n-butoxycarbonyl and tert-butoxycarbonyl.

Mono-(C₁-C₄)-alkylamino in the context of the invention represents an amino group with a straight-chain or branched alkyl substituent which contains 1 to 4 carbon atoms. There may be mentioned by way of example and preferably: methylamino, ethylamino, n-propylamino, isopropylamino, n-butylamino and tert-butylamino.

Di-(C₁-C₄)-alkylamino in the context of the invention represents an amino group with two identical or different straight-chain or branched alkyl substituents which each contain 1 to 4 carbon atoms. There may be mentioned by way of example and preferably: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino and N-tert-butyl-N-methylamino.

Mono- or di-(C₁-C₄)-alkylaminocarbonyl in the context of the invention represents an amino group which is linked via a carbonyl group [—C(═O)—] and which has a straight-chain or branched or, respectively, two identical or different straight-chain or branched alkyl substituents having in each case 1 to 4 carbon atoms. There may be mentioned by way of example and preferably: methylaminocarbonyl, ethylaminocarbonyl, n-propylaminocarbonyl, isopropylaminocarbonyl, n-butylaminocarbonyl, tert-butylaminocarbonyl, N,N-dimethylaminocarbonyl, N,N-diethylamino-carbonyl, N-ethyl-N-methylaminocarbonyl, N-methyl-N-n-propylaminocarbonyl, N-isopropyl-N-methylaminocarbonyl, N,N-diisopropylaminocarbonyl, N-n-butyl-N-methylaminocarbonyl and N-tert-butyl-N-methylaminocarbonyl.

(C₁-C₄)-Alkylcarbonylamino in the context of the invention represents an amino group with a straight-chain or branched alkylcarbonyl substituent which contains 1 to 4 carbon atoms in the alkyl radical and is linked to the N atom via the carbonyl group. There may be mentioned by way of example and preferably: acetylamino, propionylamino, n-butyrylamino, iso-butyrylamino, n-pentanoylamino and pivaloylamino.

(C₁-C₄)-Alkylcarbonyloxy in the context of the invention represents an oxy radical with a straight-chain or branched alkylcarbonyl substituent which contains 1 to 4 carbon atoms in the alkyl radical and is linked to the O atom via the carbonyl group. There may be mentioned by way of example and preferably: acetoxy, propionoxy, n-butyroxy, iso-butyroxy, n-pentanoyloxy and pivaloyloxy.

(C₁-C₄)-Alkoxycarbonylamino in the context of the invention represents an amino group with a straight-chain or branched alkoxycarbonyl substituent which contains 1 to 4 carbon atoms in the alkoxy radical and is linked to the N atom via the carbonyl group. There may be mentioned by way of example and preferably: methoxycarbonylamino, ethoxycarbonylamino, n-propoxycarbonyl-amino, isopropoxycarbonylamino, n-butoxycarbonylamino and tert-butoxycarbonylamino.

(C₃-C₆)-Cycloalkyl in the context of the invention represents a monocyclic, saturated cycloalkyl group having 3 to 6 ring carbon atoms. There may be mentioned by way of example and preferably: cyclopropyl, cyclobutyl, cyclopentyl and cyclohexyl.

4- to 6-membered heterocyclyl in the context of the invention represents a monocyclic, saturated heterocycle with 4 to 6 ring atoms in total, which contains one or two ring hetero atoms from the series N, O, S and/or S(O)₂ and is linked via a ring carbon atom or optionally via a ring nitrogen atom. 4- to 6-membered heterocyclyl with one or two ring hetero atoms from the series N, O and/or S is preferred. There may be mentioned by way of example: azetidinyl, oxetanyl, thietanyl, pyrrolidinyl, pyrazolidinyl, tetrahydrofuranyl, thiolanyl, 1,1-dioxidothiolanyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, morpholinyl, thiomorpholinyl and 1,1-dioxidothiomorpholinyl. Azetidinyl, oxetanyl, pyrrolidinyl, tetrahydrofuranyl, piperidinyl, piperazinyl, tetrahydropyranyl, morpholinyl and thiomorpholinyl are preferred.

5- or 6-membered heteroaryl in the context of the invention represents an aromatic heterocyclic radical (heteroaromatic) having 5 or, respectively, 6 ring atoms in total which contains up to three identical or different ring hetero atoms from the series N, O and/or S and is linked via a ring carbon atom or optionally via a ring nitrogen atom. There may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. 5- or 6-membered heteroaryl radicals having up to two ring hetero atoms from the series N, O and/or S, such as, for example, furyl, pyrrolyl, thienyl, thiazolyl, oxazolyl, isothiazolyl, isoxazolyl, pyrazolyl, imidazolyl, pyridyl, pyrimidinyl, pyridazinyl and pyrazinyl, are preferred.

Halogen in the context of the invention includes fluorine, chlorine, bromine and iodine. Chlorine, fluorine or bromine are preferred, and fluorine or chlorine are particularly preferred.

An oxo substituent in the context of the invention represents an oxygen atom, which is bonded to a carbon atom via a double bond.

If radicals in the compounds according to the invention are substituted, the radicals can be mono- or polysubstituted, unless specified otherwise. In the context of the present invention, for all the radicals which occur several times, the meaning thereof is independent of each other. Substitution by one or by two or three identical or different substituents is preferred. Substitution by one or by two identical or different substituents is particularly preferred.

The present invention provides in particular those compounds of the general formula (I) in which the ring

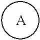

represents a phenyl or pyridyl ring and the adjacent groups R¹ and CH₂ are bonded
to ring carbon atoms

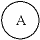

in 1, 3 or 1,4 relation to one another
and
the ring

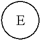

with the substituents R⁴ and R⁵ represents a phenyl ring of the formula

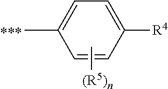

wherein
*** designates the linkage point with the ring

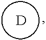

and their salts, solvates and solvates of the salts.

Compounds of the formula (I) which are preferred in the context of the present invention are those in which
either (a)
   the ring

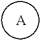

represents a pyridyl ring and the adjacent groups R¹ and CH₂ are bonded to ring carbon atoms of this pyridyl ring in 1, 3 or 1,4 relation to one another
and
the ring

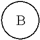

with the substituent R³ represents a heteroaryl ring of the formula

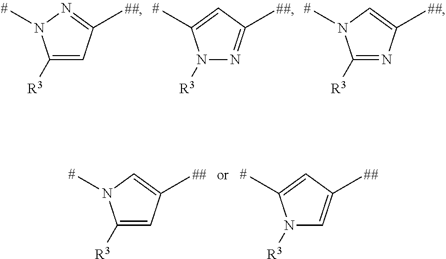

wherein
# designates the linkage point with the adjacent CH₂ group
and
## designates the linkage point with the ring

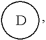

or (b)
   the ring

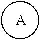

represents a phenyl ring and the adjacent groups R¹ and CH₂ are bonded to this phenyl ring in 1, 3 or 1,4 relation to one another,
and
the ring

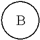

with the substituent R³ represents a heteroaryl ring of the formula

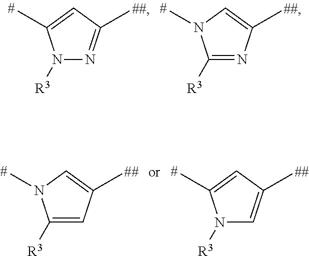

wherein
# designates the linkage point with the adjacent CH₂ group
and
## designates the linkage point with the ring

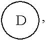

the ring

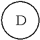

represents a heteroaryl ring of the formula

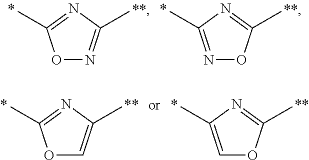

wherein
   * designates the linkage point with the ring

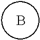

and
** designates the linkage point with the ring

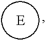

the ring

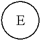

with the substituents R⁴ and R⁵ represents a phenyl ring of the formula

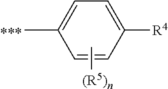

wherein
*** designates the linkage point with the ring

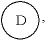

R¹ represents hydrogen or a substituent chosen from the series fluorine, chlorine, bromine, cyano, (C₁-C₄)-alkyl, (C₂-C₄)-alkynyl, (C₃-C₆)-cycloalkyl, oxetanyl, tetrahydrofuranyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
   wherein (C₁-C₄)-alkyl and (C₂-C₄)-alkynyl in their turn can be substituted by a radical chosen from the series hydroxyl, (C₁-C₄)-alkoxy, trifluoromethoxy, trimethylsilyl, (C₁-C₄)-alkoxycarbonyl and (C₃-C₆)-cycloalkyl and up to three times by fluorine
   and
   oxetanyl, tetrahydrofuranyl, tetrahydropyranyl and the cycloalkyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
   and wherein
   R⁶ and R⁷ independently of each other denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   and
   R⁸ denotes hydrogen, amino, (C₁-C₆)-alkyl, (C₃-C₆)-cycloalkyl or 5- or 6-membered heteroaryl,
      wherein (C₁-C₆)-alkyl can be substituted by a radical chosen from the series hydroxyl, (C₁-C₄)-alkoxy, trifluoromethoxy, (C₁-C₄)-alkoxycarbonyl, (C₃-C₆)-cycloalkyl, tetrahydrofuranyl, tetrahydropyranyl and 5- or 6-membered heteroaryl and up to three times by fluorine
      and wherein
      tetrahydrofuranyl, tetrahydropyranyl and the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and (C₁-C₄)-alkoxycarbonyl,
      and
      the heteroaryl groups mentioned can be substituted up to three times in an identical or different manner by a radical chosen from the series (C₁-C₄)-alkyl, trifluoromethyl, (C₁-C₄)-alkoxy and trifluoromethoxy
R² represents hydrogen or a substituent chosen from the series fluorine, chlorine, methyl, trifluoromethyl, methoxy and trifluoromethoxy,
R³ represents methyl, ethyl or trifluoromethyl,
R⁴ represents a substituent chosen from the series chlorine, cyano, pentafluorothio, (C₁-C₆)-alkyl, tri-(C₁-C₄)-alkylsilyl, —OR⁹, —NR⁹R¹⁰, —SR⁹, —S(═O)—R⁹, —S(═O)₂—R⁹, —S(═O)(═NH)—R⁹, —S(═O)(═NCH₃)—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —N(R⁹)—C(═O)—R¹⁰, —C(═O)—NR⁹R¹⁰, (C₃-C₆)-cycloalkyl, 4- to 6-membered heterocyclyl and 5- or 6-membered heteroaryl and up to three times by fluorine
   and wherein
   the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, oxo, (C₁-C₄)-alkylcarbonyl, mono-(C₁-C₄)-alkylaminocarbonyl and di-(C₁-C₄)-alkylaminocarbonyl
   and
   the heteroaryl groups mentioned in their turn can be substituted up to two times in an identical or different manner by a radical chosen from the series fluorine, chlorine, cyano, (C₁-C₄)-alkyl and (C₁-C₄)-alkoxy
      wherein the (C₁-C₄)-alkyl substituents mentioned herein and the (C₁-C₄)-alkoxy substituents mentioned herein in their turn can be substituted by hydroxyl, (C₁-C₄)-alkoxy, trifluoromethoxy, (C₁-C₄)-alkoxycarbonyl, mono-(C₁-C₄)-alkyl-aminocarbonyl or di-(C₁-C₄)-alkylaminocarbonyl or up to three times by fluorine,
   and wherein
   R⁹ and R¹⁰ independently of each other for each individual occurrence denote hydrogen, (C₁-C₄)-alkyl, (C₃-C₆)-cycloalkyl or 4- to 6-membered heterocyclyl,
      wherein (C₁-C₄)-alkyl can be substituted by a radical chosen from the series hydroxyl, (C₁-C₄)-alkoxy, trifluoromethoxy, (C₃-C₆)-cycloalkyl and 4- to 6-membered heterocyclyl and up to three times by fluorine
      and
      the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
and
n represents the number 0 or 1,
and their salts, solvates and solvates of the salts.

An alternative embodiment within the embodiment described last comprises compounds of the formula (I) in which
R¹ represents hydrogen or a substituent chosen from the series fluorine, chlorine, bromine, cyano, (C₁-C₄)-alkyl, (C₂-C₄)-alkynyl, (C₃-C₆)-cycloalkyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
   wherein (C₁-C₄)-alkyl and (C₂-C₄)-alkynyl in their turn can be substituted by a radical chosen from the series hydroxyl, (C₁-C₄)-alkoxy, trifluoromethoxy, trimethylsilyl, (C₁-C₄)-alkoxycarbonyl and (C₃-C₆)-cycloalkyl and up to three times by fluorine
   and
   the cycloalkyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
   and wherein
   R⁶ and R⁷ have the meanings given in the embodiment last described,
   and
   R⁸ denotes hydrogen, (C₁-C₆)-alkyl, (C₃-C₆)-cycloalkyl or 5- or 6-membered heteroaryl,
      wherein (C₁-C₆)-alkyl can be substituted by a radical chosen from the series hydroxyl, (C₁-C₄)-alkoxy, trifluoromethoxy, (C₁-C₄)-alkoxycarbonyl, (C₃-C₆)-cycloalkyl, tetrahydrofuranyl, tetrahydropyranyl and 5- or 6-membered heteroaryl and up to three times by fluorine
      and wherein
      tetrahydrofuranyl, tetrahydropyranyl and the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and (C₁-C₄)-alkoxycarbonyl,
      and
      the heteroaryl groups mentioned can be substituted up to three times in an identical or different manner by a radical chosen from the series (C₁-C₄)-alkyl, trifluoromethyl, (C₁-C₄)-alkoxy and trifluoromethoxy
and
R⁴ represents a substituent chosen from the series chlorine, cyano, pentafluorothio, (C₁-C₆)-alkyl, tri-(C₁-C₄)-alkylsilyl, —OR⁹, —NR⁹R¹⁰, —SR⁹, —S(═O)—R⁹, —S(═O)₂—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —N(R⁹)—C(═O)—R¹⁰, —C(═O)—NR⁹R¹⁰, (C₃-C₆)-cycloalkyl, 4- to 6-membered heterocyclyl and 5- or 6-membered heteroaryl and up to three times by fluorine
   and wherein
   the cycloalkyl and heterocyclyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl groups mentioned in their turn can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
   and wherein R⁹ and R¹⁰ have the meanings given in the embodiment last described,
and their salts, solvates and solvates of the salts.

Compounds of the formula (I) which are particularly preferred in the context of the present invention are those in which
the ring

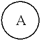

with the substituents R¹ and R² represents a pyridyl ring of the formula

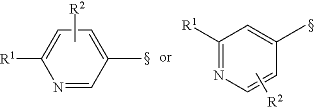

wherein
§ designates the linkage point with the adjacent CH₂ group,
the ring

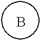

with the substituent R³ represents a heteroaryl ring of the formula

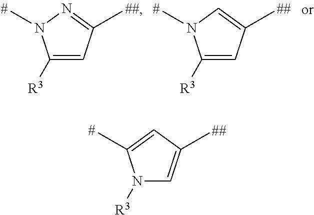

wherein
designates the linkage point with the adjacent CH₂ group
and
## designates the linkage point with the ring

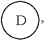

the ring

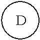

represents a heteroaryl ring of the formula

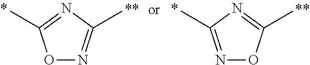

wherein
* designates the linkage point with the ring

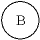

and
** designates the linkage point with the ring

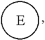

the ring

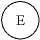

with the substituents R⁴ and R⁵ represents a phenyl ring of the formula

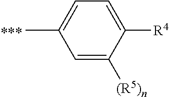

wherein
*** designates the linkage point with the ring

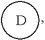

R¹ represents hydrogen or a substituent chosen from the series chlorine, cyano, (C₁-C₄)-alkyl, (C₂-C₄)-alkynyl, cyclopropyl, cyclobutyl, oxetanyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
   wherein (C₁-C₄)-alkyl and (C₂-C₄)-alkynyl in their turn can be substituted by a radical chosen from the series hydroxyl, methoxy, ethoxy, trifluoromethoxy, cyclopropyl and cyclobutyl and up to three times by fluorine
   and
   oxetanyl and tetrahydropyranyl in their turn can be substituted by methyl, ethyl, hydroxyl, methoxy or ethoxy
   and
   the cyclopropyl and cyclobutyl groups mentioned in their turn can be substituted up to two times in an identical or different manner by a radical chosen from the series fluorine, methyl, ethyl and trifluoromethyl,
   and wherein
   R⁶ and R⁷ independently of each other denote hydrogen, (C₁-C₄)-alkyl or (C₃-C₆)-cycloalkyl,
      wherein (C₁-C₄)-alkyl can be substituted by a radical chosen from the series hydroxyl, methoxy, ethoxy, trifluoromethoxy, cyclopropyl and cyclobutyl and up to three times by fluorine
   and
   R⁸ denotes hydrogen, (C₁-C₄)-alkyl, (C₃-C₆)-cycloalkyl or 5- or 6-membered heteroaryl,
      wherein (C₁-C₄)-alkyl can be substituted by a radical chosen from the series hydroxyl, methoxy, ethoxy, trifluoromethoxy, (C₃-C₆)-cycloalkyl, tetrahydrofuranyl, tetrahydropyranyl and 5- or 6-membered heteroaryl and up to three times by fluorine
      and wherein
      tetrahydrofuranyl, tetrahydropyranyl and the cycloalkyl groups mentioned can be substituted up to two times in an identical or different manner by a radical chosen from the series fluorine, methyl, ethyl, trifluoromethyl, hydroxyl, methoxy and ethoxy
      and
      the heteroaryl groups mentioned can be substituted up to three times in an identical or different manner by a radical chosen from the series methyl, ethyl and trifluoromethyl
R² represents hydrogen or a substituent chosen from the series fluorine, chlorine, methyl and methoxy,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—R⁹, —S(═O)(═NCH₃)—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, (C₁-C₄)-alkoxy and oxo,
      wherein the (C₁-C₄)-alkyl substituent mentioned and the (C₁-C₄)-alkoxy substituent in their turn can be substituted by hydroxyl, methoxy, trifluoromethoxy, ethoxy, methoxycarbonyl, ethoxycarbonyl, tert-butoxycarbonyl, methylaminocarbonyl or dimethylaminocarbonyl or up to three times by fluorine,
   and wherein
   R⁹ and R¹⁰ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
   or
   R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
and their salts, solvates and solvates of the salts.

An alternative embodiment within the embodiment described last comprises compounds of the formula (I) in which
R¹ represents hydrogen or a substituent chosen from the series chlorine, cyano, (C₁-C₄)-alkyl, (C₂-C₄)-alkynyl, cyclopropyl, cyclobutyl —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
   wherein (C₁-C₄)-alkyl and (C₂-C₄)-alkynyl in their turn can be substituted by a radical chosen from the series hydroxyl, methoxy, ethoxy, trifluoromethoxy, cyclopropyl and cyclobutyl and up to three times by fluorine
   and
   the cyclopropyl and cyclobutyl groups mentioned in their turn can be substituted up to two times in an identical or different manner by a radical chosen from the series fluorine, methyl, ethyl and trifluoromethyl,
   and wherein R⁶, R⁷ and R⁸ have the meanings given in the embodiment last described,
and
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁹ and R¹⁰ have the meanings given in the embodiment last described,
and their salts, solvates and solvates of the salts.

Compounds of the formula (I) which are also particularly preferred are those in which
the ring

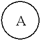

with the substituents R¹ and R² represents a phenyl ring of the formula

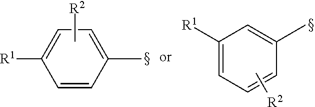

wherein
§ designates the linkage point with the adjacent CH₂ group,
the ring

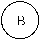

with the substituent R³ represents a heteroaryl ring of the formula

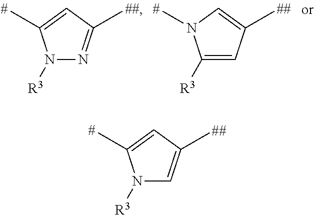

wherein
# designates the linkage point with the adjacent CH₂ group
and
## designates the linkage point with the ring

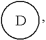

the ring

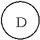

represents a heteroaryl ring of the formula

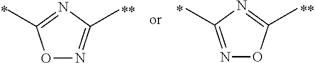

wherein
* designates the linkage point with the ring

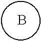

and
** designates the linkage point with the ring

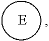

the ring

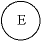

with the substituents R⁴ and R⁵ represents a phenyl ring of the formula

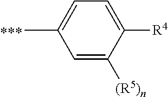

wherein
*** designates the linkage point with the ring

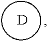

R¹ represents hydrogen or a substituent chosen from the series chlorine, cyano, (C₁-C₄)-alkyl, (C₂-C₄)-alkynyl, cyclopropyl, cyclobutyl, oxetanyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
   wherein (C₁-C₄)-alkyl and (C₂-C₄)-alkynyl in their turn can be substituted by a radical chosen from the series hydroxyl, methoxy, ethoxy, trifluoromethoxy, cyclopropyl and cyclobutyl and up to three times by fluorine
   and
   oxetanyl and tetrahydropyranyl in their turn can be substituted by methyl, ethyl, hydroxyl, methoxy or ethoxy
   and
   the cyclopropyl and cyclobutyl groups mentioned in their turn can be substituted up to two times in an identical or different manner by a radical chosen from the series fluorine, methyl, ethyl and trifluoromethyl,
   and wherein
   R⁶ and R⁷ independently of each other denote hydrogen, (C₁-C₄)-alkyl or (C₃-C₆)-cycloalkyl,
      wherein (C₁-C₄)-alkyl can be substituted by a radical chosen from the series hydroxyl, methoxy, ethoxy, trifluoromethoxy, cyclopropyl and cyclobutyl and up to three times by fluorine
   and
   R⁸ denotes hydrogen, (C₁-C₄)-alkyl, (C₃-C₆)-cycloalkyl or 5- or 6-membered heteroaryl,
      wherein (C₁-C₄)-alkyl can be substituted by a radical chosen from the series hydroxyl, methoxy, ethoxy, trifluoromethoxy, (C₃-C₆)-cycloalkyl, tetrahydrofuranyl, tetrahydropyranyl and 5- or 6-membered heteroaryl and up to three times by fluorine
      and wherein
      tetrahydrofuranyl, tetrahydropyranyl and the cycloalkyl groups mentioned can be substituted up to two times in an identical or different manner by a radical chosen from the series fluorine, methyl, ethyl, trifluoromethyl, hydroxyl, methoxy and ethoxy
      and
      the heteroaryl groups mentioned can be substituted up to three times in an identical or different manner by a radical chosen from the series methyl, ethyl and trifluoromethyl
R² represents hydrogen or a substituent chosen from the series fluorine, chlorine, methyl and methoxy,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—R⁹, —S(═O)(═NCH₃)—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, (C₁-C₄)-alkoxy and oxo,
      wherein the (C₁-C₄)-alkyl substituent mentioned and the (C₁-C₄)-alkoxy substituent in their turn can be substituted by hydroxyl, methoxy, trifluoromethoxy, ethoxy, methoxycarbonyl, ethoxycarbonyl, tert-butoxycarbonyl, methylaminocarbonyl or dimethylaminocarbonyl or up to three times by fluorine,
   and wherein
   R⁹ and R¹⁰ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
   or
   R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
and their salts, solvates and solvates of the salts.

An alternative embodiment within the embodiment described last comprises compounds of the formula (I) in which
R¹ represents hydrogen or a substituent chosen from the series chlorine, cyano, (C₁-C₄)-alkyl, (C₂-C₄)-alkynyl, cyclopropyl, cyclobutyl —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
   wherein (C₁-C₄)-alkyl and (C₂-C₄)-alkynyl in their turn can be substituted by a radical chosen from the series hydroxyl, methoxy, ethoxy, trifluoromethoxy, cyclopropyl and cyclobutyl and up to three times by fluorine
   and
   the cyclopropyl and cyclobutyl groups mentioned in their turn can be substituted up to two times in an identical or different manner by a radical chosen from the series fluorine, methyl, ethyl and trifluoromethyl,
   and wherein R⁶, R⁷ and R⁸ have the meanings given in the embodiment last described,
and
   R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁹ and R¹⁰ have the meanings given in the embodiment last described,
and their salts, solvates and solvates of the salts.

Compounds of the formula (I) which are very particularly preferred in the context of the present invention are those in which
the ring

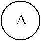

with the substituents R¹ and R² represents a pyridyl ring of the formula

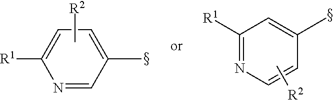

wherein
§ designates the linkage point with the adjacent CH₂ group,
the ring

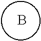

with the substituent R³ represents a heteroaryl ring of the formula

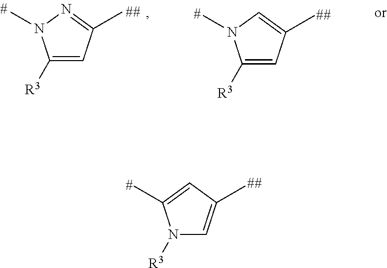

wherein
# designates the linkage point with the adjacent CH₂ group
and
## designates the linkage point with the ring

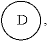

the ring

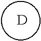

represents a heteroaryl ring of the formula

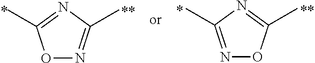

wherein
* designates the linkage point with the ring

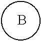

and
** designates the linkage point with the ring

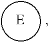

the ring

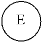

with the substituents R⁴ and R⁵ represents a phenyl ring of the formula

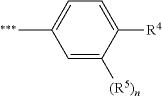

wherein
*** designates the linkage point with the ring

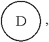

R¹ represents methyl or the group —NR⁶R⁸, wherein
   R⁶ denotes hydrogen, methyl, ethyl or cyclopropyl,
   and
   R⁸ denotes (C₁-C₄)-alkyl or (C₃-C₆)-cycloalkyl,
      wherein (C₁-C₄)-alkyl can be substituted by a radical chosen from the series hydroxyl, methoxy, ethoxy, (C₃-C₆)-cycloalkyl, tetrahydrofuranyl, tetrahydropyranyl and 5- or 6-membered heteroaryl and up to three times by fluorine
      and wherein
      tetrahydrofuranyl, tetrahydropyranyl and the cycloalkyl groups mentioned can be substituted up to two times in an identical or different manner by a radical chosen from the series fluorine, methyl, ethyl, trifluoromethyl, hydroxyl, methoxy and ethoxy
      and
      the heteroaryl group mentioned can be substituted up to three times in an identical or different manner by a radical chosen from the series methyl, ethyl and trifluoromethyl
R² represents hydrogen,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—CH₃, —S(═O)(═NH)—CF₃, —S(═O)(═NCH₃)—CH₃, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁹ and R¹⁹ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
   or
   R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
and their salts, solvates and solvates of the salts.

An alternative embodiment within the embodiment described last comprises compounds of the formula (I) in which
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁹ and R¹⁰ have the meanings given in the embodiment last described,
and their salts, solvates and solvates of the salts.

Compounds of the formula (I) which are also very particularly preferred are those in which
the ring

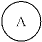

with the substituents R¹ and R² represents a phenyl ring of the formula

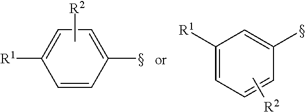

wherein
§ designates the linkage point with the adjacent CH₂ group,
the ring

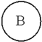

with the substituent R³ represents a heteroaryl ring of the formula

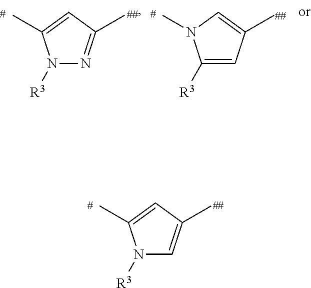

wherein
# designates the linkage point with the adjacent CH₂ group
and
## designates the linkage point with the ring

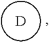

the ring

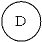

represents a heteroaryl ring of the formula

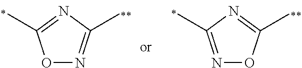

wherein
* designates the linkage point with the ring

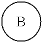

and
** designates the linkage point with the ring

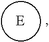

the ring

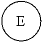

with the substituents R⁴ and R⁵ represents a phenyl ring of the formula

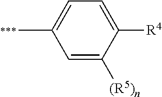

wherein
*** designates the linkage point with the ring

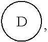

R¹ represents chlorine, cyano, methyl, ethyl, isopropyl, cyclopropyl, cyclobutyl, methoxy, ethoxy, methylsulphonyl, ethylsulphonyl, isopropylsulphonyl or the group —C(═O)—NR⁶R⁷, wherein
   R⁶ and R⁷ independently of each other denote hydrogen, (C₁-C₄)-alkyl or (C₃-C₆)-cycloalkyl,
      wherein (C₁-C₄)-alkyl can be substituted by a radical chosen from the series hydroxyl, methoxy, ethoxy, cyclopropyl and cyclobutyl and up to three times by fluorine
R² represents hydrogen,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—CH₃, —S(═O)(═NH)—CF₃, —S(═O)(═NCH₃)—CH₃, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁹ and R¹⁰ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
   or
   R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
and their salts, solvates and solvates of the salts.

An alternative embodiment within the embodiment described last comprises compounds of the formula (I) in which
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, (C₃-C₆)-cycloalkyl and 4- to 6-membered heterocyclyl,
   wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁹ and R¹⁰ have the meanings given in the embodiment last described,
and their salts, solvates and solvates of the salts.

The radical definitions given in detail in the particular combinations or preferred combinations of radicals are also replaced as desired by radical definitions of other combinations, independently of the particular combinations of radicals given.

Combinations of two or more of the abovementioned preferred ranges are very particularly preferred.

The compounds according to the invention can be prepared in many ways. The main methods which are called process A, B and C in the following and can be carried out in various variants were used here in particular.

Process A (with variants A.1 and A.2; see equations 1 and 2) is characterized in that compounds of the formula (IV), in which B, D, E, R³, R⁴, R⁵ and n have the meanings described above and in which the hydrogen atom shown is bonded to a nitrogen atom of the ring B, are reacted with compounds of the formula (II) or (III), in which A, R¹ and R² have the meanings described above and in which Y¹ quite generally represents an atom or a group from which or with the aid of which the substituent R¹ can optionally be built up or introduced, and in which X represents a leaving group. Examples of Y¹ are chlorine, bromine, iodine, cyano, nitro, hydroxyl, formyl, carboxyl and alkoxycarbonyl; examples of X are chlorine, bromine, iodine, methanesulphonate (mesylate), trifluoromethanesulphonate (triflate) and 4-methylbenzenesulphonate (tosylate).

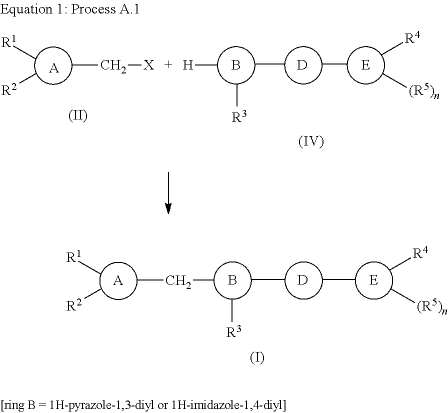

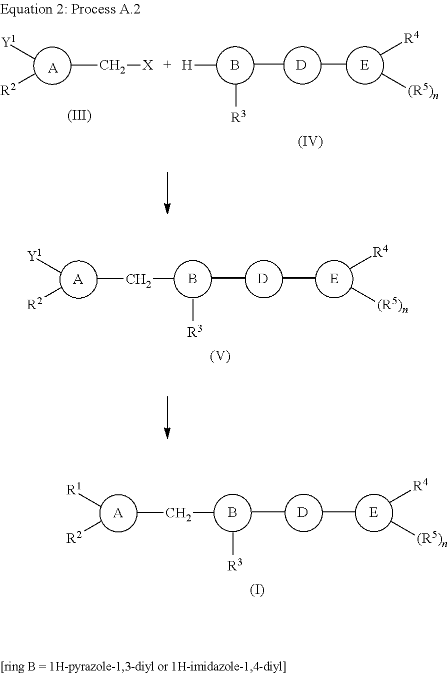

In processes A.1 and A.2 shown in equation 1 and 2, the first reaction step is a substitution reaction in which the leaving group X, which can represent, for example, chlorine, bromine, methanesulphonate (mesylate) or 4-methylbenzenesulphonate (tosylate), is exchanged for the N′ nitrogen atom of the ring B (ring B=1H-pyrazole-1,3-diyl or 1H-imidazole-1,4-diyl). This reaction is preferably carried out using a base, such as potassium tert-butylate or sodium hydride, in solvents, such as tetrahydrofuran or toluene, at temperatures between 0° C. and the boiling point of the solvent.

In the conversion of compounds of the formula (V) into the products of the formula (I) shown in equation 2 (process A.2), various chemical transformations are used, which are familiar to the person skilled in the art and some of which are described by way of example below. In the case where the substituent R² is not chemically inert towards the transformation of Y¹ to R¹, temporary protective groups can be used in R². There may be mentioned as an example protection of a hydroxyl group as a silyl ether and subsequent splitting off of the silyl group with the aid of fluoride reagents, such as tetrabutylammonium fluoride or potassium fluoride. Such protective group operations are described in literature and known to the person skilled in the art.

In process B (equations 3 and 4), the ring D is built up, the ring D representing a 1,2,4-oxadiazole here. Process B is also used in various modifications (variants B.1 and B.2). Process variant B.2 is similar to process variant A.2 with respect to the part reactions relating to the conversion of the radical Y¹ into the substituent R¹. Only variant B.1 is therefore to be described in more detail in the following (equation 3). Compounds of the formula (VI), in which A, B, R¹, R² and R³ have the meanings described above, are reacted here with hydroxyamidines of the formula (VIII), in which E, R⁴, R⁵ and n have the meanings given above, to give the oxadiazole derivatives of the formula (I-A).

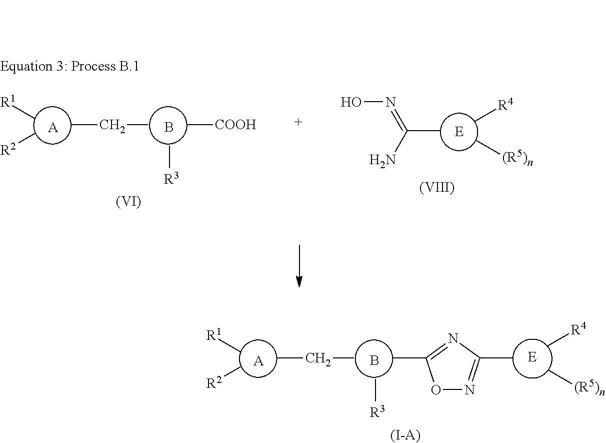

The reaction of the compounds of the formula (VI) with the compounds of the formula (VIII) is carried out in the presence of coupling reagents, such as, for example, 1H-benzotriazol-1-ol and N-[3-(dimethylamino)propyl]-N′-ethylcarbodiimide hydrochloride, and in the presence of tertiary amine bases, such as, for example, triethylamine, and in suitable solvents, such as, for example, N,N-dimethylformamide. The reaction partners are first reacted with one another at room temperature for some time, before the mixture is then heated to temperatures in the range of from +80° C. to +140° C. Alternatively, the compounds of the formula (VI) can first be converted into the corresponding carboxylic acid chlorides. Chlorinating reagents, such as, for example, oxalyl chloride or thionyl chloride, in inert solvents, such as, for example, methylene chloride or chloroform, are employed for this. The reaction is preferably carried out at room temperature and in the presence of a catalytic amount of N,N-dimethylformamide. The acid chloride obtained in this way is then reacted with the compounds of the formula (VIII). The primary condensation product of this reaction is then heated to temperatures in the range of from +80° C. to +140° C. in inert solvents, such as, for example, dimethylsulphoxide or N,N-dimethylformamide, and gives the target compound of the formula (I-A) in this way.

In process variant B.2, instead of compounds of the formula (VI), carboxylic acids of the formula (VII), in which A, B, R², R³ and Y¹ have the meanings described above, are employed.

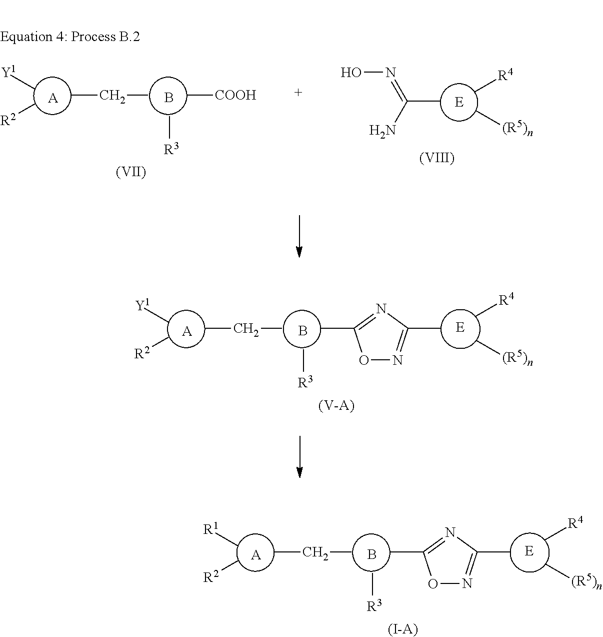

If the ring D represents a 1,3-oxazole, process C can be used. Compounds of the formula (VI) are reacted here with compounds of the formula (IX) to give intermediates of the formula (X), which are in turn cyclized to give intermediates of the formula (XI), which are finally oxidized to give the products of the formula (I-B). A, B, E, R¹, R², R³, R⁴, R⁵ and n in each case have the meanings given above.

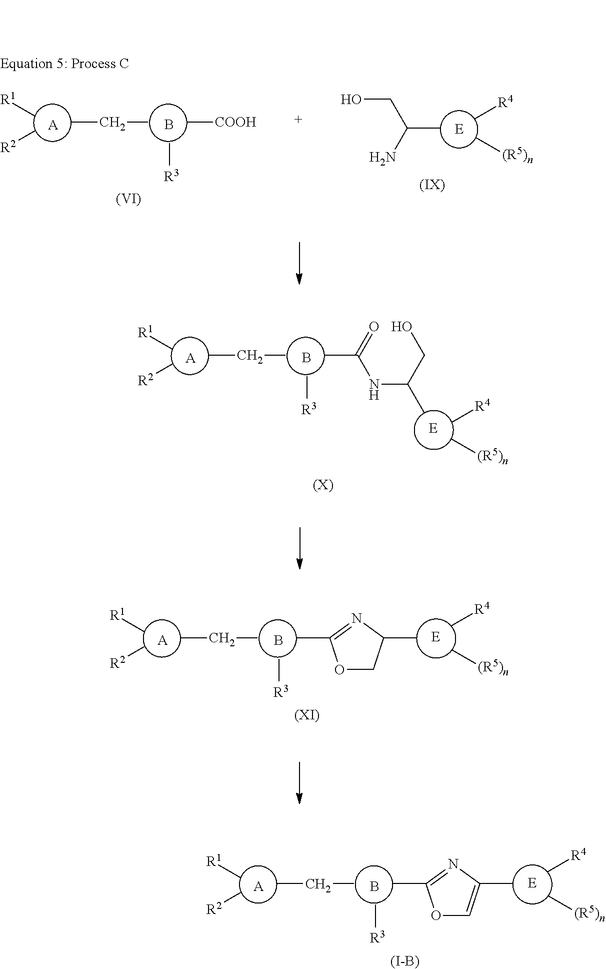

The compounds of the formula (VI) are reacted with the amino alcohols of the formula (IX) in the presence of coupling reagents, such as, for example, O-(7-azabenzotriazol-1-yl)-N,N,N′,N′-tetramethyluronium hexafluorophosphate, to give the intermediates of the formula (X). The reaction is carried out at room temperature in the presence of tertiary amine bases, such as, for example, triethylamine, in polar aprotic solvents, such as, for example, N,N-dimethylformamide. Subsequent cyclization to give the compounds of the formula (XI) is achieved with the aid of a cyclizing reagent, such as, for example and preferably, with Burgess reagent (carbomethoxysulphamoyl-triethylammonium hydroxide). The reaction is carried out in suitable solvents, such as, for example, tetrahydrofuran, at the boiling point of the solvent. The final oxidation to give the 1,3-oxazole derivatives of the formula (I-B) can be carried out with various oxidizing agents; oxidation with activated manganese dioxide in tetrahydrofuran at the boiling point of the solvent is preferred.

In the following, two processes are described by way of example (see equations 6 and 7), in which the intermediates of the formula (V) (cf. equations 2 and 4) are reacted to give target compounds of the formula (I). Further reactions of this type are described in the experimental part and require no further description here, since they do not have the character of a general process but rather of a specific conversion of functional groups. In the two cases described below, the ring A is a pyridine ring which carries the radical Y¹ in the direct neighbourhood of the pyridine nitrogen atom.

If the substituent R¹ in the target compounds of the formula (I) represents the group —NR⁶R⁸, wherein R⁶ and R⁸ have the meanings described above, and Y¹ represents chlorine, bromine or iodine, the intermediates of the formula (V) are reacted with amines of the formula (XII) (see equation 6). The addition of a tertiary amine as an auxiliary base, such as, for example, N,N-diisopropylethylamine, may possibly be of advantage here. The reaction preferably takes place in solvents, such as diethylene glycol dimethyl ether or N-methylpyrrolidinone, or the compounds of the formula (XII), employed in excess, themselves serve as solvents. The reaction is carried out at elevated temperature, preferably in a temperature range of between +80° C. and +200° C. Reactions in the upper region of the temperature interval mentioned are preferably carried out in closed pressure vessels in a microwave apparatus.

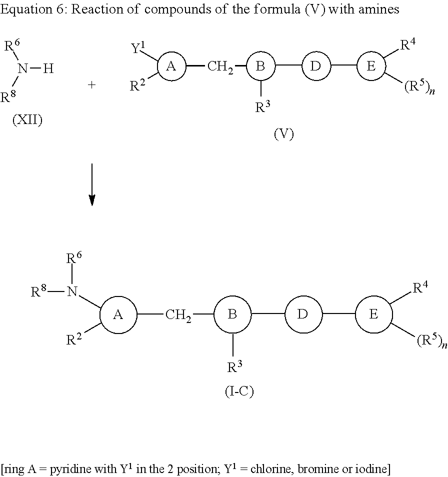

If the substituent R¹ in the target compounds of the formula (I) represents an optionally substituted alkynyl or alkyl group and Y¹ represents chlorine, bromine or iodine, the intermediates of the formula (V) can be reacted, for example, with propargyl alcohol (XIII) to give products of the formula (I-D) (see equation 7). The reaction is preferably carried out at room temperature in an aprotic solvent, such as tetrahydrofuran, in the presence of an amine base, such as triethylamine, and a palladium catalyst, such as, for example, tetrakis(triphenylphosphine)palladium(0), and of copper(I) iodide (variant of so-called “Sonogashira coupling”). A reduction of the alkyne bond which optionally follows to give products of the formula (I-E) is carried out with hydrogen, preferably under normal pressure or also under an increased pressure of up to approx. 100 bar, in the presence of a metal catalyst, preferably based on platinum, palladium or nickel; there may be mentioned by way of example platinum(IV) oxide, palladium on active charcoal and Raney nickel.

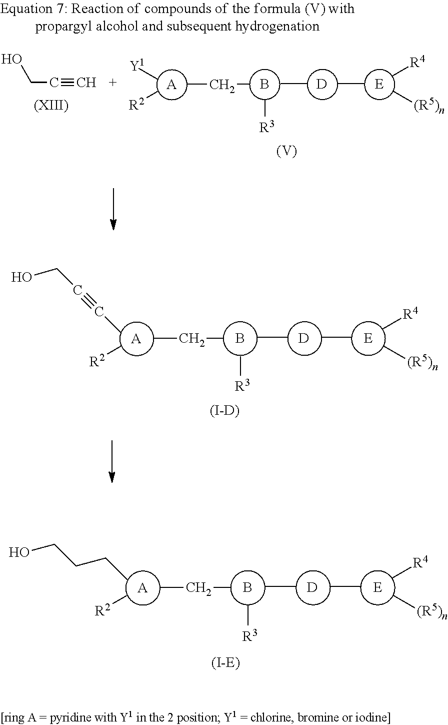

Processes with which the compounds of the formula (IV) shown in equation 1 and 2 can be prepared are described by way of example in the following:

Compounds of the formula (IV) in which the ring D has the meaning of a 1,2,4-oxadiazole and the ring B represents a 1H-pyrazole-1,3-diyl or 1H-imidazole-1,4-diyl group are built up by reacting compounds of the formula (VIII), in which E, R⁴, R⁵ and n have the meanings given above, and compounds of the formula (XIV), in which R³ has the meaning given above and ring B represents 1H-pyrazole-1,3-diyl or 1H-imidazole-1,4-diyl, with one another (see equation 8). This type of condensation reaction has already been described in process B.1 (equation 3) and is carried out here under completely analogous conditions.

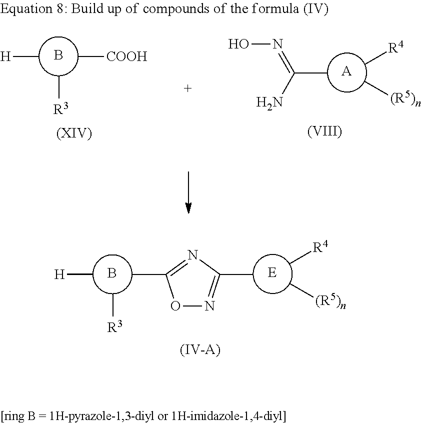

Processes with which the compounds of the formula (VI) shown in equation 3 and the compounds of the formula (VII) shown in equation 4 can be built up, depending on the nature of the ring B, are described in the following (see equations 9-11).

Equation 9 describes the preparation of pyrazole and imidazole derivatives of the formula (VII) starting from compounds of the formula (XV) in which the ring B represents 1H-pyrazole-1,3-diyl or 1H-imidazole-1,4-diyl, the hydrogen shown is bonded to the N¹ nitrogen atom of the ring B and R³ has the meaning given above. These compounds are reacted with the compounds of the formula (III) to give intermediates of the formula (XVI). The reactions conditions here are the same as those described in process A.2 (equation 2). The ester hydrolysis in the second reaction step is carried out under standard conditions, for example with sodium hydroxide solution in methanol or ethanol as the solvent at temperatures in a range of from room temperature to +60° C.

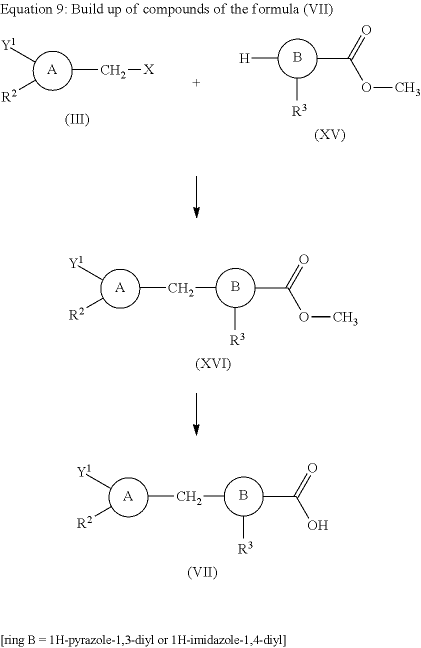

The preparation of corresponding pyrazole and imidazole derivatives of the formula (VI) is carried out analogously to the process described in equation 9, employing starting compounds of the formulae (II) and (XV).

For the preparation of compounds of the formula (VII) in which the ring B represents 1H-pyrrole-1,3-diyl, the procedure can be as shown in equation 10. γ-Keto esters of the formula (XVII), in which R³ has the meaning given above, are first converted with the aid of trimethyl orthoformate into acetals of the formula (XVIII), and these are then reacted with methyl formate in the presence of a base, such as, preferably, sodium hydride, in inert solvents, such as diethyl ether or pentane, optionally with the addition of protic solvents, such as methanol. Subsequent cleavage of the acetal under acid conditions, for example by means of an aqueous or methanolic hydrogen chloride solution, gives the intermediates of the formula (XIX). These are then subjected to a condensation reaction with amines of the formula (XX), in which A, R² and Y¹ have the meanings given above, to give compounds of the formula (XXI). This reaction is preferably carried out at room temperature or slightly elevated temperature in methanol as the solvent. The final ester hydrolysis is carried out under basic standard conditions.

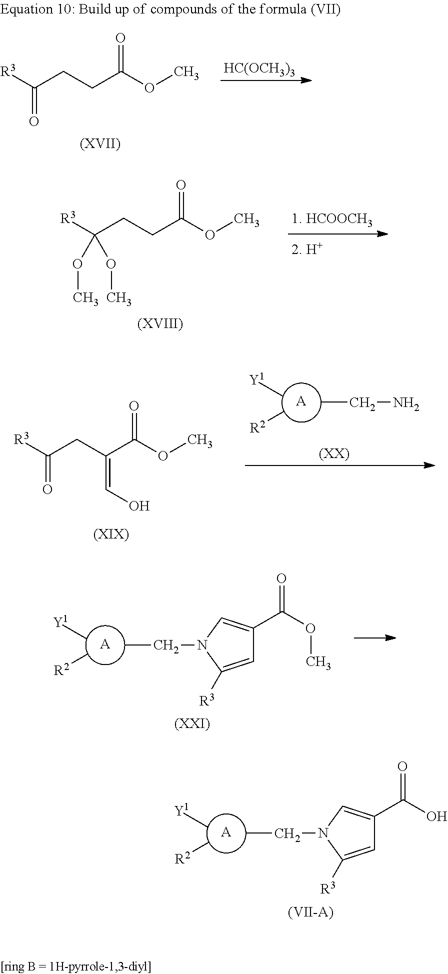

If instead of the amine of the formula (XX) the corresponding compound which already contains the substituent R¹ instead of the radical Y¹ is used, the pyrrole derivatives corresponding to the formula (VI) are obtained in an analogous manner by the process described above.

Compounds of the formula (VII) in which the ring B represents a pyrazole-3,5-diyl can be prepared by the process described in equation 11. In the first step, compounds of the formula (XXII), in which A, R² and Y¹ have the meanings given above, are reacted with oxalic acid diethyl ester using a base, such as, preferably, sodium hydride, in a protic solvent, such as ethanol, at temperatures between −10° C. and room temperature to give intermediates of the formula (XXIII). These are then subjected to a condensation reaction with hydrazine derivatives of the formula (XXIV), in which R³ has the meaning given above, in acetic acid at temperatures between room temperature and +100° C. to give compounds of the formula (XXV). The final ester hydrolysis is carried out by standard methods, for example by treatment with lithium hydroxide in ethanol at temperatures of from room temperature up to the boiling point of the solvent.

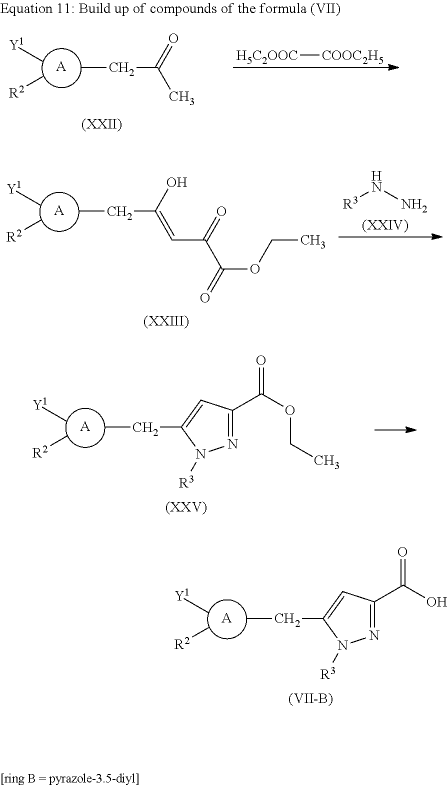

Pyrazole derivatives corresponding to the formula (VI) are obtained in an analogous manner starting from the corresponding compounds of the formula (XXII) in which the radical Y¹ is already exchanged for the substituent R¹.

For illustration by way of example of the process variants described above, the preparation of compounds of the formula (I-F) according to the invention

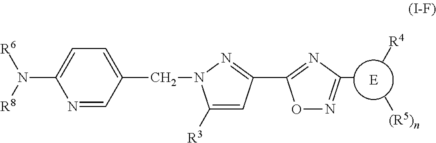

in which the ring E and R³, R⁴, R⁵, R⁶, R⁸ and n in each case have the meanings given above,
is explained in more detail in the following:

Such compounds of the formula (I-F) can be prepared by a procedure in which an N′-hydroxyamidine of the formula (VIII)

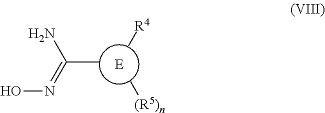

in which the ring E and R⁴, R⁵ and n have the meanings given above,
first can either be
[A] subjected to a condensation reaction with a pyrazolecarboxylic acid of the formula (XXVI)

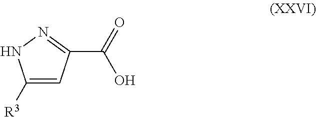

in which R³ has the meaning given above,
to give a 1,2,4-oxadiazole derivative of the formula (XXVII)

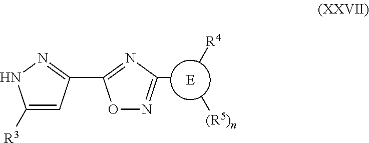

in which the ring E and R³, R⁴, R⁵ and n have the meanings given above,
and this is then alkylated in the presence of a base with a compound of the formula (XXVIII)

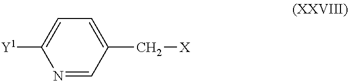

in which
Y¹ represents chlorine, bromine or iodine
and
X represents chlorine, bromine, iodine, mesylate, triflate or tosylate,
to give a compound of the formula (XXIX)

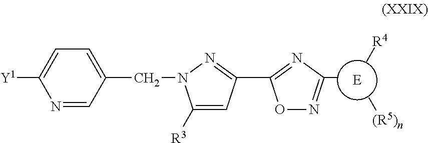

in which the ring E and R³, R⁴, R⁵, n and Y¹ have the meanings given above,
   or
   [B] subjected to a condensation reaction with a pyrazolecarboxylic acid of the formula (XXX)

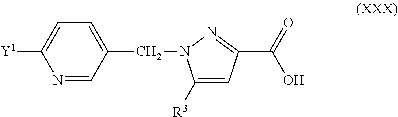

in which R³ has the meaning given above
and
Y¹ represents chlorine, bromine or iodine,
to give the compound of the formula (XXIX)

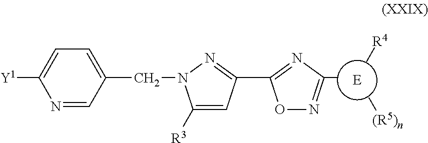

in which the ring E and R³, R⁴, R⁵, n and Y¹ have the meanings given above,
   and the compound of the formula (XXIX) obtained in this way in then reacted, optionally in the presence of an auxiliary base, with a compound of the formula (XII)

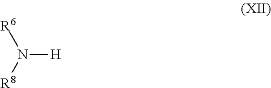

in which R⁶ and R⁸ have the meanings given above,
(in this context cf. processes A.2 and B.2 described above, in combination with the reactions shown in equation 6, 8 and 9 and the particular reaction parameters described there).

The starting compounds of the formulae (II), (III), (VIII), (IX), (XII), (XIII), (XIV), (XV), (XVII), (XX), (XXII) and (XXIV) are either commercially obtainable or described as such in the literature, or they can be prepared by routes evident to the person skilled in the art analogously to methods published in the literature. Numerous detailed instructions and literature information for the preparation of the starting materials are also to be found in the experimental part in the section for the preparation of the starting compounds and intermediates.

The compounds according to the invention are highly potent inhibitors of the HIF regulation pathway and have a good bioavailability following peroral administration.

On the basis of their action profile, the compounds according to the invention are suitable in particular for treatment of hyperproliferative diseases in humans and in mammals generally. The compounds can inhibit, block, reduce or lower cell proliferation and cell division and on the other hand increase apoptosis.

The hyperproliferative diseases for the treatment of which the compounds according to the invention can be employed include, inter alia, psoriasis, keloids, scar formation and other proliferative diseases of the skin, benign diseases, such as benign prostate hyperplasia (BPH), and in particular the group of tumour diseases. In the context of the present invention, these are understood as meaning, in particular, the following diseases, but without being limited to them: mammary carcinomas and mammary tumours (ductal and lobular forms, also in situ), tumours of the respiratory tract (parvicellular and non-parvicellular carcinoma, bronchial carcinoma), cerebral tumours (e.g. of the brain stem and of the hypothalamus, astrocytoma, medulloblastoma, ependymoma and neuro-ectodermal and pineal tumours), tumours of the digestive organs (oesophagus, stomach, gall bladder, small intestine, large intestine, rectum), liver tumours (inter alia hepatocellular carcinoma, cholangiocellular carcinoma and mixed hepatocellular and cholangiocellular carcinoma), tumours of the head and neck region (larynx, hypopharynx, nasopharynx, oropharynx, lips and oral cavity), skin tumours (squamous epithelial carcinoma, Kaposi sarcoma, malignant melanoma, Merkel cell skin cancer and nonmelanomatous skin cancer) tumours of soft tissue (inter alia soft tissue sarcomas, osteosarcomas, malignant fibrous histiocytomas, lymphosarcomas and rhabdomyosarcomas), tumours of the eyes (inter alia intraocular melanoma and retinoblastoma), tumours of the endocrine and exocrine glands (e.g. thyroid and parathyroid glands, pancreas and salivary gland), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testicles in men). These also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hair cell leukaemia, and AIDS-correlated lymphomas, Hodgkin's lymphomas, non-Hodgkin's lymphomas, cutaneous T cell lymphomas, Burkitt's lymphomas and lymphomas in the central nervous system.

These well-described diseases in humans can also occur with a comparable aetiology in other mammals and can be treated there with the compounds of the present invention.

In the context of this invention the term “treatment” or “treat” is used in the conventional sense and means attending to, caring for and nursing a patient with the aim of combating, reducing, attenuating or alleviating a disease or health abnormality and improving the living conditions impaired by this disease, such as, for example, with a cancer disease.

The compounds according to the invention act as modulators of the HIF regulation pathway and are therefore also suitable for treatment of diseases associated with a harmful expression of the HIF transcription factor. This applies in particular to the transcription factors HIF-1α and HIF-2α. The term “harmful expression of HIF” here means a non-normal physiological presence of HIF protein. This can be due to excessive synthesis of the protein (mRNA- or translation-related), reduced degradation or inadequate counter-regulation in the functioning of the transcription factor.

HIF-1α and HIF-2α regulate more than 100 genes. This applies to proteins which play a role in angiogenesis and are therefore directly relevant to tumours, and also those which influence glucose, amino acid and lipid metabolism as well as cell migration, metastasis and DNA repair, or improve the survival of tumour cells by suppressing apoptosis. Others act more indirectly via inhibition of the immune reaction and upwards regulation of angiogenic factors in inflammation cells. HIF also plays an important role in stem cells, and here in particular tumour stem cells, which are reported to have increased HIF levels. By the inhibition of the HIF regulation pathway by the compounds of the present invention, tumour stem cells, which do not have a high proliferation rate and therefore are affected only inadequately by cytotoxic substances, are therefore also influenced therapeutically (cf. Semenza, 2007; Weidemann and Johnson, 2008).

Changes in cell metabolism by HIF are not exclusive to tumours, but also occur with other hypoxic pathophysiological processes, whether chronic or transient. HIF inhibitors—such as the compounds of the present invention—are therapeutically helpful in those connections in which, for example, additional damage arises from adaptation of cells to hypoxic situations, since damaged cells can cause further damage if they do not function as intended. One example of this is the formation of epileptic foci in partly destroyed tissue following strokes. A similar situation is found with cardiovascular diseases if ischaemic processes occur in the heart or in the brain as a consequence of thromboembolic events, inflammations, wounds, intoxications or other causes. These can lead to damage such as a locally retarded action potential, which in turn can bring about arrhythmias or chronic heart failure. In a transient form, e.g. due to apnoea, under certain circumstances an essential hypertension may occur, which can lead to known secondary diseases, such as, for example, stroke and cardiac infarction.

Inhibition of the HIF regulation pathway such as is achieved by the compounds according to the invention can therefore also be helpful for diseases such as cardiac insufficiency, arrhythmia, cardiac infarction, apnoea-induced hypertension, pulmonary hypertension, transplant ischaemia, reperfusion damage, stroke and macular degeneration, as well as for recovery of nerve function after traumatic damage or severance.

Since HIF is one of the factors which control the transition from an epithelial to a mesenchymal cell type, which is of importance specifically for the lung and kidney, the compounds according to the invention can also be employed for preventing or controlling fibroses of the lung and kidney associated with HIF.

Further diseases for the treatment of which the compounds according to the invention can be used are inflammatory joint diseases, such as various forms of arthritis, and inflammatory intestinal diseases, such as, for example, Crohn's disease.

Chugwash polycythaemia is mediated by HIF-2α activity during erythropoiesis inter alia in the spleen. The compounds according to the invention, as inhibitors of the HIF regulation pathway, are therefore also suitable here for suppressing excessive erythrocyte formation and therefore for alleviating the effects of this disease.

The compounds of the present invention can furthermore be used for treatment of diseases associated with excessive or abnormal angiogenesis. These include, inter alia, diabetic retinopathy, ischaemic retinal vein occlusion and retinopathy in premature babies (cf. Aiello et al., 1994; Peer et al., 1995), age-related macular degeneration (AMD; cf. Lopex et al., 1996), neovascular glaucoma, psoriasis, retrolental fibroplasia, angiofibroma, inflammation, rheumatic arthritis (RA), restenosis, in-stent restenosis following vessel implantation.

An increased blood supply is furthermore associated with cancerous, neoplastic tissue and leads here to an accelerated tumour growth. The growth of new blood and lymph vessels moreover facilitates the formation of metastases and therefore the spread of the tumour. New lymph and blood vessels are also harmful for allografts in immunoprivileged tissues, such as the eye, which, for example, increases the susceptibility to rejection reactions. Compounds of the present invention can therefore also be employed for therapy of one of the abovementioned diseases, e.g. by an inhibition of the growth or a reduction in the number of blood vessels. This can be achieved via inhibition of endothelial cell proliferation or other mechanisms for preventing or lessening the formation of vessels and via a reduction of neoplastic cells by apoptosis.

The present invention furthermore provides the use of the compounds according to the invention for treatment and/or prevention of diseases, in particular the abovementioned diseases.

The present invention furthermore provides the use of the compounds according to the invention for the preparation of a medicament for treatment and/or prevention of diseases, in particular the abovementioned diseases.

The present invention furthermore provides the use of the compounds according to the invention in a method for treatment and/or prevention of diseases, in particular the abovementioned diseases.

The present invention furthermore provides a method for treatment and/or prevention of diseases, in particular the abovementioned diseases, using an active amount of at least one of the compounds according to the invention.

The compounds according to the invention can be employed by themselves or, if required, in combination with one or more other pharmacologically active substances, as long as this combination does not lead to undesirable and unacceptable side effects. The present invention furthermore therefore provides medicaments containing at least one of the compounds according to the invention and one or more further active compounds, in particular for treatment and/or prevention of the abovementioned diseases.

For example, the compounds of the present invention can be combined with known antihyperproliferative, cytostatic or cytotoxic substances for treatment of cancer diseases. The combination of the compounds according to the invention with other substances customary for cancer therapy or also with radiotherapy is therefore indicated in particular, since hypoxic regions of a tumour respond only weakly to the conventional therapies mentioned, whereas the compounds of the present invention display their activity there in particular.

Suitable active compounds in the combination which may be mentioned by way of example are:

aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulphan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluoruracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, streptozocin, strontium-89 chloride, synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulphan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, BAY 43-9006 (sorafenib), CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

aminoglutethimide, L-asparaginase, azathioprine, 5-azacytidine, bleomycin, busulphan, camptothecin, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenin, ethinylestradiol, etoposide, fludarabin phosphate, 5-fluorodeoxyuridine, 5-fluorodeoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphono-acetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds according to the invention can also be combined in a very promising manner with biological therapeutics, such as antibodies (e.g. avastin, rituxan, erbitux, herceptin) and recombinant proteins, which additively or synergistically intensify the effects of inhibition of the HIF signal pathway transmission.

Inhibitors of the HIF regulation pathway, such as the compounds according to the invention, can also achieve positive effects in combination with other therapies directed against angiogenesis, such as, for example, with avastin, axitinib, DAST, recentin, sorafenib or sunitinib. Combinations with inhibitors of the proteasome and of mTOR and antihormones and steroidal metabolic enzyme inhibitors are particularly suitable because of their favourable profile of side effects.

Generally, the following aims can be pursued with the combination of compounds of the present invention with other agents having a cytostatic or cytotoxic action:
an improved activity in slowing down the growth of a tumour, in reducing its size or even in its complete elimination compared with treatment with an individual active compound;
the possibility of employing the chemotherapeutics used in a lower dosage than in monotherapy;
the possibility of a more tolerable therapy with few side effects compared with individual administration;
the possibility of treatment of a broader spectrum of tumour diseases;
achievement of a higher rate of response to the therapy;
a longer survival time of the patient compared with present-day standard therapy.

The compounds according to the invention can moreover also be employed in combination with radiotherapy and/or surgical intervention.

The present invention furthermore provides medicaments which comprise at least one compound according to the invention, conventionally together with one or more inert, non-toxic, pharmaceutically suitable auxiliary substances, and the use thereof for the above-mentioned purposes.

The compounds according to the invention can act systemically and/or locally. They can be administered in a suitable manner for this purpose, such as e.g. orally, parenterally, pulmonally, nasally, sublingually, lingually, buccally, rectally, dermally, transdermally, conjunctivally, otically or as an implant or stent.

The compounds according to the invention can be administered in suitable administration forms for these administration routes.

Administration forms which function according to the prior art, release the compounds according to the invention rapidly and/or in a modified manner and contain the compounds according to the invention in crystalline and/or amorphized and/or dissolved form are suitable for oral administration, such as e.g. tablets (non-coated or coated tablets, for example with coatings which are resistant to gastric juice or dissolve in a delayed manner or are insoluble and control the release of the compound according to the invention), tablets or films/oblates, films/lyophilisates or capsules which disintegrate rapidly in the oral cavity (for example hard or soft gelatine capsules), sugar-coated tablets, granules, pellets, powders, emulsions, suspensions, aerosols or solutions.

Parenteral administration can be effected with bypassing of an absorption step (e.g. intravenously, intraarterially, intracardially, intraspinally or intralumbally) or with inclusion of an absorption (e.g. intramuscularly, subcutaneously, intracutaneously, percutaneously or intraperitoneally). Administration forms which are suitable for parenteral administration are, inter alia, injection and infusion formulations in the form of solutions, suspensions, emulsions, lyophilisates or sterile powders.

For the other administration routes e.g. inhalation medicament forms (inter alia powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents are suitable.

Oral and parenteral administration are preferred, in particular oral and intravenous administration.

The compounds according to the invention can be converted into the administration forms mentioned. This can be effected in a manner known per se by mixing with inert, non-toxic, pharmaceutically suitable auxiliary substances. These auxiliary substances include inter alia carrier substances (for example microcrystalline cellulose, lactose, mannitol), solvents (e.g. liquid polyethylene glycols), emulsifiers and dispersing or wetting agents (for example sodium dodecyl sulphate, polyoxysorbitan oleate), binders (for example polyvinylpyrrolidone), synthetic and natural polymers (for example albumin), stabilizers (e.g. antioxidants, such as, for example, ascorbic acid), dyestuffs (e.g. inorganic pigments, such as, for example, iron oxides) and flavour and/or smell correctants.

In general, it has proved advantageous in the case of parenteral administration to administer amounts of from about 0.001 to 1 mg/kg, preferably about 0.01 to 0.5 mg/kg of body weight to achieve effective results. In the case of oral administration the dosage is about 0.01 to 100 mg/kg, preferably about 0.01 to 20 mg/kg and very particularly preferably 0.1 to 10 mg/kg of body weight.

Nevertheless it may be necessary to deviate from the amounts mentioned, and in particular depending on the body weight, administration route, individual behaviour towards the active compound, nature of the formulation and point of time or interval at which administration takes place. Thus in some cases it may be sufficient to manage with less than the abovementioned minimum amount, while in other cases the upper limit mentioned must be exceeded. In the case where relatively large amounts are administered, it may be advisable to distribute these into several individual doses over the day.

The following embodiment examples illustrate the invention. The inventions is not limited to the examples.

The percentage data in the following tests and examples are percentages by weight, unless stated otherwise; parts are parts by weight. The solvent ratios, dilution ratios and concentration data of liquid/liquid solutions in each case relate to the volume.

### A. EXAMPLES

### Abbreviations and Acronyms:

abs. absolute
aq. aqueous
Boc tert-butoxycarbonyl
Ex. Example
Bu butyl
approx. circa, approximately
CI chemical ionization (in MS)
d doublet (in NMR)
d day(s)
TLC thin layer chromatography
DCI direct chemical ionization (in MS)
dd doublet of doublet (in NMR)
DMAP 4-N,N-dimethylaminopyridine
DME 1,2-dimethoxyethane
DMF dimethylformamide
DMSO dimethylsulphoxide
dt doublet of triplet (in NMR)
of th. of theory (chemical yield)
EDC N′-(3-dimethylaminopropyl)-N-ethylcarbodiimide hydrochloride
EI electron impact ionization (in MS)
eq. equivalent(s)
ESI electrospray ionization (in MS)
Et ethyl
GC gas chromatography
h hour(s)
HATU O-(7-azabenzotriazol-1-yl)-N,N,N′,N′-tetramethyluronium hexafluorophosphate
HOBt 1-hydroxy-1H-benzotriazole hydrate
HPLC high pressure, high performance liquid chromatography
ⁱPr isopropyl
LC-MS liquid chromatography-coupled mass spectrometry
m multiplet (in NMR)
min minute(s)
MPLC medium pressure liquid chromatography (over silica gel; also called “flash chromatography”)
MS mass spectrometry
NMP N-methyl-2-pyrrolidone
NMR nuclear magnetic resonance spectrometry
Pd/C palladium on active charcoal
PEG polyethylene glycol
Pr propyl
quart quartet (in NMR)
quint quintet (in NMR)
R_{f} retention index (in TLC)
RT room temperature
Rₜ retention time (in HPLC)
s singlet (in NMR)
sept septet (in NMR)
t triplet (in NMR)
^{t}Bu tert-butyl
TFA trifluoroacetic acid
THF tetrahydrofuran
UV ultraviolet spectrometry
v/v volume to volume ratio (of a solution)
tog. together

### HPLC Methods:

### Method A

Instrument: HP 1100 with DAD detection; column: Kromasil 100 RP-18, 60 mm×2.1 mm, 3.5 μm; eluent A: 5 ml of perchloric acid (70% strength)/1 of water, eluent B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### Method B

Instrument: HP 1100 with DAD detection; column: Kromasil 100 RP-18, 60 mm×2.1 mm, 3.5 μm; eluent A: 5 ml of perchloric acid (70% strength)/1 of water, eluent B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

### Method C (LC/MS):

Apparatus type MS: Micromass ZQ; apparatus type HPLC: HP 1100 Series; UV DAD; column: Phenomenex Gemini 3μ, 30 mm×3.00 mm; eluent A: 1 l of water+0.5 ml of 50% strength formic acid, eluent B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method D (LC/MS):

Apparatus type MS: Waters Micromass Quattro Micro; apparatus type HPLC: Agilent 1100 Series; column: Thermo Hypersil GOLD 3μ, 20 mm×4 mm; eluent A: 1 l of water+0.5 ml of 50% strength formic acid, eluent B: 1 l of acetonitrile+0.5 ml of 50% strength formic acid; gradient: 0.0 min 100% A→3.0 min 10% A→4.0 min 10% A→4.01 min 100% A→5.00 min 100% A; oven: 50° C.; flow rate: 2 ml/min; UV detection: 210 nm.

### Method E (LC/MS):

Apparatus type MS: Micromass ZQ; apparatus type HPLC: Waters Alliance 2795; column: Phenomenex Synergi 2.5μ MAX-RP 100A Mercury 20 mm×4 mm; eluent A: 1 l of water+0.5 ml of 50% strength formic acid, eluent B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

### Method F (LC/MS):

Instrument: Micromass Quattro Premier with Waters HPLC Acquity; column: Thermo Hypersil GOLD 1.9μ, 50 mm×1 mm; eluent A: 1 l of water+0.5 ml of 50% strength formic acid, eluent B: 1 l of acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm.

### Method G (LC/MS):

Instrument: Micromass Platform LCZ with HPLC Agilent Series 1100; column: Thermo Hypersil GOLD 3μ, 20 mm×4 mm; eluent A: 1 l of water+0.5 ml of 50% strength formic acid, eluent B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method H (LC/MS):

Instrument: Micromass Quattro LCZ with HPLC Agilent Series 1100; column: Phenomenex Synergi 2.5μ MAX-RP 100A Mercury 20 mm×4 mm; eluent A: 1 l of water+0.5 ml of 50% strength formic acid, eluent B: 1 l of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

### Method I (LC/MS):

Instrument: Waters Acquity SQD HPLC System; column: Waters Acquity HPLC HSS T3 1.8 μm, 50 mm×1 mm; Eluent A: 1 l of water+0.25 ml of 99% strength formic acid, eluent B: 1 l of acetonitrile+0.25 ml of 99% strength formic acid; gradient: 0.0 min 90% A→1.2 min 5% A→2.0 min 5% A; flow rate: 0.40 ml/min; oven: 50° C.; UV detection: 210-400 nm.

### Method J (LC/MS):

Instrument MS: Waters ZQ 2000; instrument HPLC: Agilent 1100, 2-column circuit; autosampler: HTC PAL; column: YMC-ODS-AQ, 50 mm×4.6 mm, 3.0 μm; eluent A: water+0.1% formic acid, eluent B: acetonitrile+0.1% formic acid; gradient: 0.0 min 100% A→0.2 min 95% A→1.8 min 25% A→1.9 min 10% A→2.0 min 5% A→3.2 min 5% A→3.21 min 100% A→3.35 min 100% A; oven: 40° C.; flow rate: 3.0 ml/min; UV detection: 210 nm.

### Method K (GC/MS):

Instrument: Micromass GCT, GC 6890; column: Restek RTX-35, 15 m×200 μm×0.33 μm; constant flow rate with helium: 0.88 ml/min; oven: 70° C.; inlet: 250° C.; gradient: 70° C., 30° C./min→310° C. (hold for 3 min)

### Method L (GC/MS):

Instrument: Micromass GCT, GC 6890; column: Restek RTX-35, 15 m×200 μm×0.33 μm; constant flow rate with helium: 0.88 ml/min; oven: 70° C.; inlet: 250° C.; gradient: 70° C., 30° C./min→310° C. (hold for 12 min)

### Method M (Preparative HPLC):

Column: GROM-SIL 1200DS-4 HE, 10 μm, 250 mm×30 mm; mobile phase and gradient programme: acetonitrile/0.1% aq. formic acid 10:90 (0-3 min), acetonitrile/0.1% aq. formic acid 10:90→95:5 (3-27 min), acetonitrile/0.1% aq. formic acid 95:5 (27-34 min), acetonitrile/0.1% aq. formic acid 10:90 (34-38 min); flow rate: 50 ml/min; temperature: 22° C.; UV detection: 254 nm.

### Method N (Preparative HPLC):

Column: Reprosil C18, 10 μm, 250 mm×30 mm; mobile phase and gradient programme: acetonitrile/0.1% aq. trifluoroacetic acid 10:90 (0-2 min), acetonitrile/0.1% aq. trifluoroacetic acid 10:90→90:10 (2-23 min), acetonitrile/0.1% aq. trifluoroacetic acid 90:10 (23-28 min), acetonitrile/0.1% aq. trifluoroacetic acid 10:90 (28-30 min); flow rate: 50 ml/min; temperature: 22° C.; UV detection: 210 nm.

### Method O (LC/MS):

Instrument MS: Waters SQD; Instrument HPLC: Waters HPLC; column: Zorbax SB-Aq (Agilent), 50 mm×2.1 mm, 1.8 μm; Eluent A: water+0.025% formic acid, eluent B: acetonitrile+0.025% formic acid; gradient: 0.0 min 98% A→0.9 min 25% A→1.0 min 5% A→1.4 min 5% A→1.41 min 98% A→1.5 min 98% A; oven: 40° C.; flow rate: 0.60 ml/min; UV detection: DAD, 210 nm.

### Method P (Preparative HPLC):

Column: Reprosil C18, 10 μm, 250 mm×30 mm; mobile phase and gradient programme: acetonitrile/0.1% aq. ammonia 20:80 (0-3 min), acetonitrile/0.1% aq. ammonia 20:80→98:2 (3-35 min), acetonitrile/0.1% aq. ammonia 98:2 (35-40 min); flow rate: 50 ml/min; temperature: 22° C.; UV detection: 210 nm.

### Method Q (LC/MS):

Apparatus type MS: Waters ZQ; apparatus type HPLC: Agilent 1100 Series; UV DAD; column: Thermo Hypersil GOLD 3μ, 20 mm×4 mm; eluent A: 1 l of water+0.5 ml of 50% strength formic acid, eluent B: 1 l of acetonitrile+0.5 ml of 50% strength formic acid; gradient 0.0 min 100% A→3.0 min 10% A→4.0 min 10% A→4.1 min 100% A (flow rate 2.5 ml/min); oven: 55° C.; flow rate: 2 ml/min; UV detection: 210 nm.

### Method R (Preparative HPLC):

Column: Sunfire C18 OBD, 5 μm, 19 mm×150 mm; mobile phase and gradient programme: water/methanol/1% aq. TFA 40:50:10 (0:00-1:15 min), water/methanol/1% aq. TFA 40:50:10→24:70:6 (1:15-1:30 min), water/methanol/1% aq. TFA 24:70:6→8:90:2 (1:30-8:30 min), water/methanol/1% aq. TFA 8:90:2→80:0:20 (8:30-9:00 min), water/methanol/1% aq. TFA 80:0:20 (9:00-11:30 min); flow rate: 25 ml/min; temperature: 40° C.; UV detection: 210 nm.

For all the reactants or reagents for which the preparation is not described explicitly in the following, they were obtained commercially from generally accessible sources. For all the other reactants or reagents for which the preparation likewise is not described in the following and which were not commercially obtainable or were obtained from sources which are not generally accessible, reference is made to the published literature in which their preparation is described.

### Starting Compounds and Intermediates:

### Example 1A

### N′-Hydroxy-4-(1,1,1-trifluoro-2-methylpropan-2-yl)benzenecarboximide amide

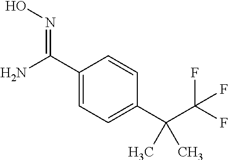

### Step 1: 2-(4-Bromophenyl)-1,1,1-trifluoropropan-2-ol

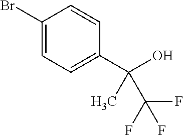

A suspension of dichloro(dimethyl)titanium in a heptane/methylene chloride mixture was first prepared as follows: 100 ml (100 mmol) of a 1 M solution of titanium tetrachloride in methylene chloride were cooled to −30° C., 100 ml (100 mmol) of a 1 M solution of dimethylzinc in heptane were added dropwise and the mixture was subsequently stirred at −30° C. for 30 min. This suspension was then cooled to −40° C. and a solution of 10 g (39.5 mmol) of 1-(4-bromophenyl)-2,2,2-trifluoroethanone in 50 ml of methylene chloride was added. The mixture was subsequently stirred at −40° C. for 5 min, the temperature was then allowed to come to RT and the mixture was stirred at RT for a further 2 h. 50 ml of water were slowly added dropwise, while cooling with ice, and the mixture was then diluted with a further 300 ml of water. It was extracted twice with methylene chloride, the combined methylene chloride phases were washed once with water, dried over anhydrous magnesium sulphate and filtered and the solvent was removed on a rotary evaporator. The residue was purified by column chromatography over silica gel (mobile phase: cyclohexane/ethyl acetate 85:15). 10.5 g (100% of th.) of the title compound were obtained which, according to ¹H-NMR, still contained residues of solvent.

¹H-NMR (400 MHz, CDCl₃, δ/ppm): 7.52 (d, 2H), 7.47 (d, 2H), 1.76 (s, 3H).

LC/MS (method C, ESIpos): Rₜ=2.27 min, m/z=268 [M+H]⁺.

### Step 2: 2-(4-Bromophenyl)-1,1,1-trifluoropropan-2-yl methanesulphonate

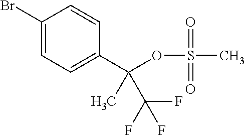

3.12 g (78.05 mmol, 60% strength in mineral oil) of sodium hydride were initially introduced into 45 ml of THF under argon and a solution of 10.5 g (39.03 mmol) of the compound obtained in Example 1A/step 1 in 20 ml of THF was added dropwise at RT. After the mixture had been stirred at RT for 1 h and at 40° C. for 30 min, a solution of 8.94 g (78.05 mmol) of methanesulphonyl chloride in 45 ml of THF was added dropwise and the reaction mixture was stirred at 40° C. for a further 60 min. 50 ml of water were then slowly added dropwise to the mixture and the mixture was diluted with saturated aqueous sodium bicarbonate solution and extracted twice with ethyl acetate. The combined ethyl acetate phases were dried over anhydrous magnesium sulphate and filtered and the solvent was removed on a rotary evaporator. The residue was stirred in hexane and the solid obtained was filtered off and dried in vacuo. 12.4 g (92% of th.) of the title compound were obtained.

¹H-NMR (400 MHz, CDCl₃, δ/ppm): 7.58 (d, 2H), 7.43 (d, 2H), 3.16 (s, 3H), 2.28 (s, 3H).

LC/MS (method D, ESIpos): Rₜ=2.32 min, m/z=364 [M+NH₄]⁺.

### Step 3: 1-Bromo-4-(1,1,1-trifluoro-2-methylpropan-2-yl)benzene

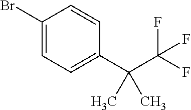

12.4 g (35.72 mmol) of the compound obtained in Example 1A/step 2 were initially introduced into 250 ml of methylene chloride and the mixture was cooled to 0° C. 35.7 ml (71.44 mmol) of a 2 M solution of trimethylaluminium were then slowly added dropwise at 0° C., while stirring, and the mixture was then allowed to come to RT and was subsequently stirred at RT for a further 1.5 h. 120 ml of a saturated aqueous sodium bicarbonate solution were slowly added dropwise to the mixture, followed by 40 ml of a saturated aqueous sodium chloride solution. The mixture was filtered over kieselguhr and the kieselguhr was rinsed twice with methylene chloride. The combined methylene chloride phases were washed once with saturated aqueous sodium chloride solution and dried over anhydrous magnesium sulphate and the solvent was removed on a rotary evaporator. 8.69 g (87% of th.) of the title compound were obtained in a purity of 95%.

¹H-NMR (400 MHz, CDCl₃, δ/ppm): 7.49 (d, 2H), 7.33 (d, 2H), 1.55 (s, 6H).

LC/MS (method E, ESIpos): Rₜ=2.54 min, no ionization.

GC/MS (method K, EI): Rₜ=3.48 min, m/z=266 [M]⁺.

### Step 4: 4-(1,1,1-Trifluoro-2-methylpropan-2-yl)benzenecarbonitrile

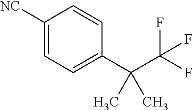

3.34 g (12.50 mmol) of the compound obtained in Example 1A/step 3 were initially introduced into 2.5 ml of degassed DMF under argon, 881 mg (7.50 mmol) of zinc cyanide and 867 mg (0.75 mmol) of tetrakis(triphenylphosphine)palladium(0) were added and the mixture was stirred at 80° C. overnight. After cooling to RT, the reaction mixture was diluted with ethyl acetate and solid constituents were filtered off. The filtrate was washed twice with 2 N aqueous ammonia solution and once with saturated aqueous sodium chloride solution, dried over anhydrous magnesium sulphate and freed from the solvent on a rotary evaporator. The residue was purified by column chromatography over silica gel (mobile phase: cyclohexane/ethyl acetate 85:15). 2.08 g (78% of th.) of the title compound were obtained.

¹H-NMR (400 MHz, CDCl₃, δ/ppm): 7.68 (d, 2H), 7.62 (d, 2H), 1.60 (s, 6H).

GC/MS (method K, EI): Rₜ=3.83 min, m/z=213 [M]⁺.

### Step 5: N′-Hydroxy-4-(1,1,1-trifluoro-2-methylpropan-2-yl)benzenecarboximide amide

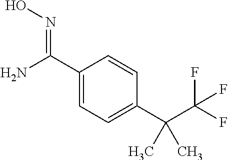

A mixture of 2.40 g (11.26 mmol) of the compound from Example 1A/step 4, 1.72 g (24.77 mmol) of hydroxylamine hydrochloride and 3.45 ml (24.77 mmol) of triethylamine in 60 ml of ethanol was stirred under reflux for 1 h. After cooling to RT, the solvent was removed on a rotary evaporator. Ethyl acetate was added to the residue and the solid present was filtered off. The ethyl acetate solution was washed successively with water and saturated aqueous sodium chloride solution, dried over anhydrous magnesium sulphate and filtered. After removal of the solvent, the oil obtained was triturated with petroleum ether. After the resulting solid had been filtered off with suction and dried under a high vacuum, 2.65 g (96% of th.) of the title compound were obtained.

¹H-NMR (400 MHz, CDCl₃, δ/ppm): 8.0 (s, broad, 1H), 7.62 (d, 2H), 7.52 (d, 2H), 4.88 (s, broad, 2H), 1.60 (s, 6H).

LC/MS (method D, ESIpos): Rₜ=1.34 min, m/z=247 [M+H]⁺.

### Example 2A

### 4-(2-Fluoropropan-2-yl)-N′-hydroxybenzenecarboximide amide

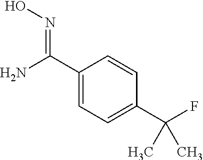

### Step 1: 4-(2-Fluoropropan-2-yl)benzenecarbonitrile

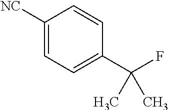

1.20 g (7.44 mmol) of diethylaminosulphur trifluoride (DAST) were added to a solution of 1.00 g (6.20 mmol) of 4-(2-hydroxypropan-2-yl)benzenecarbonitrile [obtained from 4-(propan-2-yl)benzenecarbonitrile in accordance with J. L. Tucker et al., Synth. Comm. 2006, 36 (15), 2145-2155] in 20 ml of methylene chloride at a temperature of 0° C. The reaction mixture was stirred at RT for 2 h and then diluted with water and extracted with methylene chloride. The organic phase was washed with water, dried over anhydrous magnesium sulphate and filtered. After removal of the solvent on a rotary evaporator, the residue was purified by means of MPLC (silica gel, mobile phase: cyclohexane/ethyl acetate 95:5). 675 mg (67% of th.) of the title compound were obtained.

¹H-NMR (400 MHz, CDCl₃, δ/ppm): 7.57 (d, 2H), 7.48 (d, 2H), 1.72 (s, 3H), 1.68 (s, 3H).

LC/MS (method D, ESIpos): Rₜ=2.12 min, m/z=163 [M+H]⁺.

### Step 2: 4-(2-Fluoropropan-2-yl)-N′-hydroxybenzenecarboximide amide

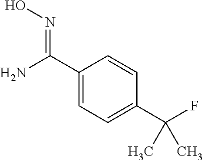

By the process described under Example 1A/step 5, 756 mg (93% of th.) of the title compound were obtained from 675 mg (4.14 mmol) of the compound from Example 2A/step 1.

¹H-NMR (400 MHz, CDCl₃, δ/ppm): 7.62 (d, 2H), 7.41 (d, 2H), 4.89 (s, broad, 2H), 1.72 (s, 3H), 1.68 (s, 3H).

LC/MS (method D, ESIpos): Rₜ=1.04 min, m/z=197 [M+H]⁺.

### Example 3A

### N′-Hydroxy-4-[(trifluoromethyl)sulphonyl]benzenecarboximide amide

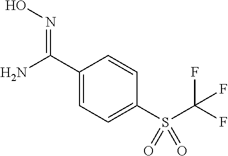

By the process described under Example 1A/step 5, 5.08 g (97% of th.) of the title compound were obtained from 4.60 g (19.56 mmol) of 4-[(trifluoromethyl)sulphonyl]benzenecarbonitrile [W. Su, Tetrahedron. Lett. 1994, 35 (28), 4955-4958].

¹H-NMR (400 MHz, DMSO-d₆, δ/ppm): 10.26 (s, 1H), 8.13 (dd, 4H), 6.12 (s, 2H).

LC/MS (method D, ESIpos): Rₜ=1.57 min, m/z=269 [M+H]⁺.

### Example 4A

### N′-Hydroxy-4-(3-methyloxetan-3-yl)benzenecarboximide amide

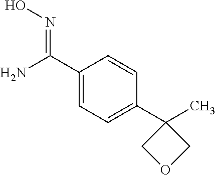

### Step 1: [4-(Dibenzylamino)phenyl]boronic acid

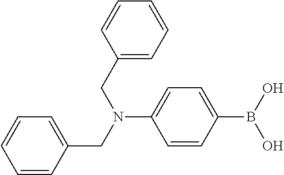

A solution of 6.0 g (17.03 mmol) of N,N-dibenzyl-4-bromoaniline [T. Saitoh et al., J. Am. Chem. Soc. 2005, 127 (27), 9696-9697] was initially introduced into a mixture of 75 ml of anhydrous diethyl ether and 75 ml of anhydrous THF under inert conditions. 13.9 ml (22.14 mmol) of a 1.6 M solution of n-butyllithium in hexane were added dropwise to this solution at −78° C. When the addition had ended, the mixture was stirred at −78° C. for 60 min, before 6.3 ml (27.25 mmol) of boric acid triisopropyl ester were added dropwise at the same temperature. After a further 15 min at −78° C., the reaction mixture was allowed to come to RT. After stirring at RT for 3 h, 18 ml of 2 M hydrochloric acid were added and the resulting mixture was stirred intensively at RT for 20 min. After dilution with approx. 200 ml of water, the mixture was extracted three times with approx. 200 ml of ethyl acetate each time. The combined organic extracts were washed successively with water and saturated sodium chloride solution. After drying over anhydrous magnesium sulphate, the mixture was filtered and the solvent was removed on a rotary evaporator. The oily residue obtained was triturated with a mixture of 50 ml of tert-butyl methyl ether and 50 ml of pentane. After the resulting solid had been filtered off with suction and dried under a high vacuum, 3.91 g (72% of th., purity of 90%) of the title compound were obtained, this being employed in the next stage without further purification.

¹H-NMR (400 MHz, DMSO-d₆, δ/ppm): 7.58 (d, 2H), 7.32-7.30 (m, 4H), 7.27-7.23 (m, 6H), 6.66 (d, 2H), 4.70 (s, 4H).

HPLC (method A): Rₜ=4.35 min.

MS (ESIpos): m/z=318 [M+H]⁺.

### Step 2: Ethyl {3-[4-(dibenzylamino)phenyl]oxetan-3-yl}acetate

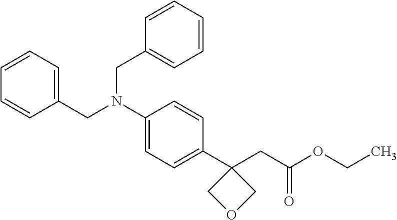

10.7 ml (16.0 mmol) of a 1.5 M potassium hydroxide solution were added to a solution of 304 mg (0.616 mmol) of (1,5-cyclooctadiene)rhodium(I) chloride dimer in 30 ml of 1,4-dioxane. Solutions of 1.75 g (12.31 mmol) of ethyl oxetan-3-ylideneacetate [G. Wuitschik et al., Angew. Chem. Int. Ed. Engl. 2006, 45 (46), 7736-7739] in 1 ml of 1,4-dioxane and 3.91 g (12.31 mmol) of the compound from Example 4A/step 1 in 60 ml of 1,4-dioxane were then added successively. The reaction mixture was stirred at RT for 6 h. It was then diluted with approx. 200 ml of water and extracted three times with approx. 200 ml of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by means of MPLC (silica gel, mobile phase: cyclohexane/ethyl acetate 20:1,→5:1). 3.51 g (67% of th.) of the title compound were obtained.

¹H-NMR (400 MHz, CDCl₃, δ/ppm): 7.33-7.30 (m, 4H), 7.27-7.23 (m, 6H), 6.97 (d, 2H), 6.69 (d, 2H), 4.94 (d, 2H), 4.81 (d, 2H), 4.62 (s, 4H), 4.00 (quart, 2H), 3.04 (s, 2H), 1.11 (t, 3H).

LC/MS (method E, ESIpos): Rₜ=2.57 min, m/z=416 [M+H]⁺.

### Step 3: 2-{3-[4-(Dibenzylamino)phenyl]oxetan-3-yl}ethanol

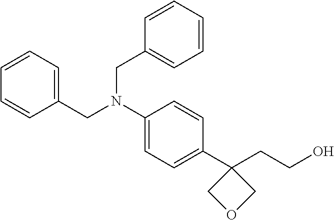

4.9 ml (4.88 mmol) of a 1 M solution of lithium aluminium hydride in THF were added dropwise to a solution of 2.90 g (6.98 mmol) of the compound from Example 4A/step 2 in 145 ml of anhydrous THF under inert conditions and at a temperature of 0° C. When the dropwise addition had ended, the reaction mixture was stirred at 0° C. for 1.5 h. 2 g of kieselguhr and 2 ml of water were then cautiously added. The heterogeneous mixture was filtered with suction over a paper filter. The filtrate was diluted with approx. 250 ml of water and extracted three times with approx. 250 ml of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by means of MPLC (silica gel, mobile phase: cyclohexane/ethyl acetate 4:1). 2.34 g (87% of th.) of the title compound were obtained.

¹H-NMR (400 MHz, CDCl₃, δ/ppm): 7.36-7.31 (m, 4H), 7.27-7.22 (m, 6H), 6.88 (d, 2H), 6.71 (d, 2H), 4.93 (d, 2H), 4.71 (d, 2H), 4.63 (s, 4H), 3.55 (quart, 2H), 2.29 (t, 2H), 1.12 (t, 1H).

HPLC (method B): Rₜ=3.98 min.

MS (DCI, NH₃): m/z=374 [M+H]⁺.

LC/MS (method E, ESIpos): Rₜ=2.15 min, m/z=374 [M+H]⁺.

### Step 4: {3-[4-(Dibenzylamino)phenyl]oxetan-3-yl}acetaldehyde

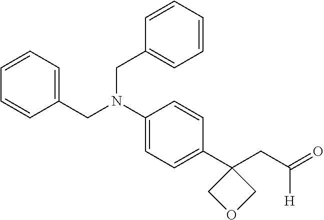

807 μl of anhydrous DMSO were added dropwise to a solution of 496 μl (5.68 mmol) of oxalyl chloride in 5 ml of anhydrous methylene chloride at −78° C. under inert conditions. After 20 min, a solution of 1.93 g (5.17 mmol) of the compound from Example 4A/step 3 in 5 ml of anhydrous methylene chloride was slowly added dropwise at the same temperature. After stirring at −78° C. for 60 min, 3.7 ml (26.87 mmol) of anhydrous triethylamine were added dropwise. After a further 10 min at this temperature, the reaction mixture was allowed to warm to RT. The mixture was then introduced into a suction filter filled with silica gel and elution was carried out first with cyclohexane and then with cyclohexane/ethyl acetate 7:1→1:1. The product fractions were combined and evaporated to dryness and the residue was taken up in ethyl acetate. Washing was carried out successively with saturated sodium bicarbonate solution, water and saturated sodium chloride solution. After drying over anhydrous magnesium sulphate, the mixture was filtered and the solvent was removed on a rotary evaporator. 1.81 g (92% of th.) of the title compound were obtained.

¹H-NMR (400 MHz, CDCl₃, δ/ppm): 9.69 (t, 1H), 7.34-7.31 (m, 4H), 7.28-7.23 (m, 6H), 6.97 (d, 2H), 6.70 (d, 2H), 5.00 (d, 2H), 4.72 (d, 2H), 4.63 (s, 4H), 3.18 (d, 2H).

HPLC (method B): Rₜ=4.61 min.

MS (DCI, NH₃): m/z=372 [M+H]⁺.

LC/MS (method F, ESIpos): Rₜ=1.43 min, m/z=372 [M+H]⁺.

### Step 5: N,N-Dibenzyl-4-(3-methyloxetan-3-yl)aniline

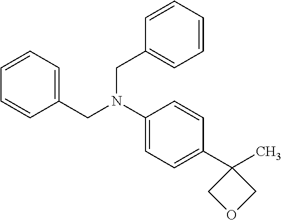

A solution of 1.81 g (4.87 mmol) of the compound from Example 4A/step 4 and 13.57 g (14.62 mmol) of tris(triphenylphosphine)rhodium(I) chloride in 240 ml of toluene was heated under reflux under inert conditions for one hour. After cooling to RT, insoluble constituents were filtered off. The solvent was removed on a rotary evaporator and the residue was purified by means of MPLC (silica gel, mobile phase: cyclohexane/ethyl acetate 20:1→5:1). 1.36 g (73% of th., purity of approx. 90%) of the title compound were obtained.

¹H-NMR (400 MHz, CDCl₃, δ/ppm): 7.35-7.31 (m, 4H), 7.27-7.24 (m, 6H), 7.07 (d, 2H), 6.72 (d, 2H), 4.90 (d, 2H), 4.64 (s, 4H), 4.55 (d, 2H), 1.96 (s, 3H).

LC/MS (method F, ESIpos): Rₜ=1.55 min, m/z=344 [M+H]⁺.

### Step 6: 4-(3-Methyloxetan-3-yl)aniline

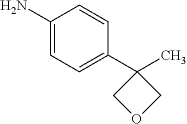

A solution of 1.35 g (3.93 mmol) of the compound from Example 4A/step 5 in 135 ml of ethanol was hydrogenated in a flow-through hydrogenation apparatus (“H-Cube” from ThalesNano, Budapest, Hungary) (conditions: 10% Pd/C catalyst, “full H₂” mode, 1 ml/min, 50° C.). After removal of the solvent on a rotary evaporator, the crude product was purified by means of MPLC (silica gel, mobile phase: cyclohexane/ethyl acetate 4:1→2:1). 386 mg (60% of th.) of the title compound were obtained.

¹H-NMR (400 MHz, CDCl₃, δ/ppm): 7.03 (d, 2H), 6.69 (d, 2H), 4.92 (d, 2H), 4.58 (d, 2H), 3.63 (s, broad, 2H), 1.69 (s, 3H).

LC/MS (method D, ESIpos): Rₜ=0.77 min, m/z=164 [M+H]⁺.

### Step 7: 4-(3-Methyloxetan-3-yl)benzenecarbonitrile

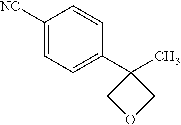

First 1.7 ml (20.7 mmol) of concentrated hydrochloric acid and then, dropwise, a solution of 159 mg (2.30 mmol) of sodium nitrite in 5 ml of water were added to a solution of 375 mg (2.30 mmol) of the compound from Example 4A/step 6 in 17 ml of water at 0° C. The mixture was stirred at 0° C. for 30 min, before 1.1 g (10.3 mmol) of solid sodium carbonate were added in portions. The solution obtained in this way was added dropwise to a solution of 257 mg (2.87 mmol) of copper(I) cyanide and 464 mg (7.12 mmol) of potassium cyanide in 16 ml of toluene/water (2:1) at 0° C. The reaction mixture was stirred at 0° C. for 1 h. The mixture was then allowed to warm to RT. The organic phase was then separated off and washed successively with water and saturated sodium chloride solution. After the solvent had been separated off on a rotary evaporator, the crude product was purified by means of MPLC (silica gel, mobile phase: cyclohexane/ethyl acetate 10:1→2:1). 390 mg (83% of th., purity of approx. 84%) of the title compound were obtained.

¹H-NMR (400 MHz, CDCl₃, δ/ppm): 7.66 (d, 2H), 7.31 (d, 2H), 4.92 (d, 2H), 4.68 (d, 2H), 1.73 (s, 3H).

GC/MS (method K, EIpos): Rₜ=5.45 min, m/z=173 (M)⁺.

### Step 8: N′-Hydroxy-4-(3-methyloxetan-3-yl)benzenecarboximide amide

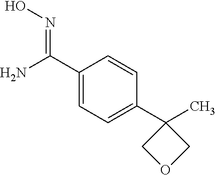

By the process described under Example 1A/step 5, 297 mg (74% of th.) of the title compound were obtained from 375 mg (1.83 mmol) of the compound from Example 4A/step 7.

¹H-NMR (400 MHz, DMSO-d₆, δ/ppm): 9.59 (s, 1H), 7.64 (d, 2H), 7.23 (d, 2H), 5.79 (s, broad, 2H), 4.80 (d, 2H), 4.53 (d, 2H), 1.62 (s, 3H).

HPLC (method A): Rₜ=2.74 min.

MS (DCI, NH₃): m/z=207 [M+H]⁺.

### Example 5A

### 4-(3-Fluoro-oxetan-3-yl)-N′-hydroxybenzenecarboximide amide

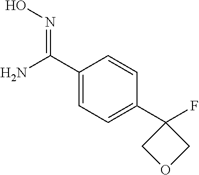

### Step 1: 4-(3-Hydroxyoxetan-3-yl)benzenecarbonitrile

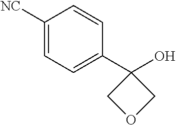

11 ml (21.8 mmol) of a 2 M solution of isopropylmagnesium chloride in diethyl ether were added dropwise to a solution of 5.0 g (21.8 mmol) of 4-iodobenzonitrile in 100 ml of anhydrous THF at −40° C. under inert conditions. After the mixture had been stirred at the same temperature for 1.5 h, it was cooled down to −78° C. and was slowly added to a solution, likewise cooled to −78° C., of 2.95 g (32.7 mmol, 80% in methylene chloride) of 3-oxooxetane [G. Wuitschik et al., Angew. Chem. Int. Ed. Engl. 2006, 45 (46), 7736-7739] in 100 ml of anhydrous THF with the aid of a cannula. When the addition had ended, the reaction mixture was stirred first at −78° C. for 10 min, then at 0° C. for 2 h and finally at RT for 30 min. A few ml of saturated aqueous ammonium chloride solution were then added. The solvent was then largely removed on a rotary evaporator. The residue obtained was diluted with 200 ml of water and extracted three times with approx. 200 ml of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by crystallization from cyclohexane/ethyl acetate 10:1. 2.42 g (63% of th.) of the title compound were obtained.

¹H-NMR (400 MHz, DMSO-d₆, δ/ppm): 7.88 (d, 2H), 7.80 (d, 2H), 6.63 (s, 1H), 4.79 (d, 2H), 4.65 (d, 2H).

HPLC (method A): Rₜ=3.09 min.

MS (DCI, NH₃): m/z=193 [M+NH₄]⁺.

### Step 2: 4-(3-Fluoro-oxetan-3-yl)benzenecarbonitrile

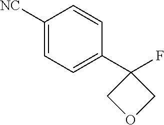

A solution of 662 mg (4.11 mmol) of diethylaminosulphur trifluoride (DAST) in 5 ml of methylene chloride was added dropwise to a suspension of 600 mg (3.43 mmol) of the compound from Example 5A/step 1 in 55 ml of methylene chloride at −78° C. under inert conditions. After 30 min at −78° C., the reaction mixture was warmed very rapidly to −20° C. with the aid of an ice/water bath. After approx. 30 seconds, 20 ml of 1 M sodium hydroxide solution were added and the mixture was allowed to warm to RT. After dilution with 150 ml of water, the mixture was extracted three times with approx. 50 ml of diethyl ether each time. The combined organic extracts were dried over anhydrous magnesium sulphate. After filtration, the solvent was removed on a rotary evaporator. The crude product was purified by means of MPLC (silica gel, mobile phase: cyclohexane/ethyl acetate 8:1). 495 mg (82% of th.) of the title compound were obtained.

¹H-NMR (400 MHz, CDCl₃, δ/ppm): 7.76 (d, 2H), 7.73 (d, 2H), 5.15 (dd, 2H), 4.81 (dd, 2H).

LC/MS (method D, ESIpos): Rₜ=1.59 min, m/z=178 [M+H]⁺.

### Step 3: 4-(3-Fluoro-oxetan-3-yl)-N′-hydroxybenzenecarboximide amide

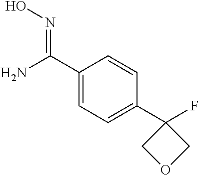

By the process described under Example 1A/step 5, 470 mg (86% of th.) of the title compound were obtained from 450 mg (2.54 mmol) of the compound from Example 5A/step 2.

¹H-NMR (400 MHz, DMSO-d₆, δ/ppm): 9.71 (s, 1H), 7.77 (d, 2H), 7.54 (d, 2H), 5.87 (broad s, 2H), 4.97 (dd, 2H), 4.91 (dd, 2H).

HPLC (method A): Rₜ=2.64 min.

MS (DCI, NH₃): m/z=211 [M+H]⁺.

LC/MS (method D, ESIpos): Rₜ=0.80 min, m/z=211 [M+H]⁺.

### Example 6A

### N′-Hydroxy-4-(3-methoxyoxetan-3-yl)benzenecarboximide amide

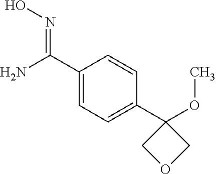

### Step 1: 4-(3-Methoxyoxetan-3-yl)benzenecarbonitrile

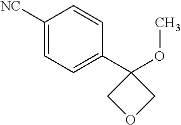

151 mg (3.77 mmol) of a 60% strength dispersion of sodium hydride in mineral oil were added to a solution of 600 mg (3.43 mmol) of the compound from Example 5A/step 1 in 12.5 ml of anhydrous DMF at 5° C. The mixture was stirred at 5° C. for 1 h, before 256 μl (4.11 mmol) of methyl iodide were added. The reaction mixture was then allowed to come to RT. After stirring for 15 h, 150 ml of water were added and the mixture was extracted twice with approx. 150 ml of diethyl ether each time. The combined organic extracts were dried over anhydrous magnesium sulphate. After filtration and removal of the solvent on a rotary evaporator, the residue obtained was purified by means of MPLC (silica gel, mobile phase: cyclohexane/ethyl acetate 20:1→4:1). 566 mg (87% of th.) of the title compound were obtained.

¹H-NMR (400 MHz, DMSO-d₆, δ/ppm): 7.92 (d, 2H), 7.68 (d, 2H), 4.81 (d, 2H), 4.74 (d, 2H), 3.07 (s, 3H).

HPLC (method A): Rₜ=3.63 min.

MS (DCI, NH₃): m/z=207 [M+NH₄]⁺.

LC/MS (method D, ESIpos): Rₜ=1.50 min, m/z=190 [M+H]⁺.

### Step 2: N′-Hydroxy-4-(3-methoxyoxetan-3-yl)benzenecarboximide amide

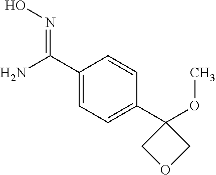

By the process described under Example 1A/step 5, 520 mg (89% of th.) of the title compound were obtained from 500 mg (2.64 mmol) of the compound from Example 6A/step 1.

¹H-NMR (400 MHz, DMSO-d₆, δ/ppm): 9.67 (s, 1H), 7.73 (d, 2H), 7.43 (d, 2H), 5.83 (broad s, 2H), 4.77 (m, 4H), 3.03 (s, 3H).

HPLC (method A): Rₜ=2.54 min.

MS (DCI, NH₃): m/z=223 [M+H]⁺.

### Example 7A

### 4-(4-Fluorotetrahydro-2H-pyran-4-yl)-N′-hydroxybenzenecarboximide amide

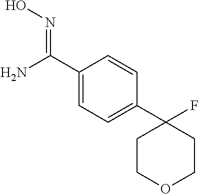

### Step 1: 4-(4-Hydroxytetrahydro-2H-pyran-4-yl)benzenecarbonitrile

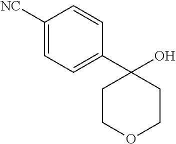

By the process described under Example 5A/step 1, 25.0 g (109 mmol) of 4-iodobenzonitrile were reacted with 16.4 g (164 mmol) of tetrahydro-4H-pyran-4-one to give 7.56 g (34% of th.) of the title compound.

¹H-NMR (400 MHz, DMSO-d₆, δ/ppm): 7.80 (d, 2H), 7.70 (d, 2H), 5.30 (s, 1H), 3.81-3.70 (m, 4H), 2.02-1.94 (m, 2H), 1.51-1.48 (m, 2H).

HPLC (method A): Rₜ=3.35 min.

MS (DCI, NH₃): m/z=204 [M+H]⁺, 221 [M+NH₄]⁺.

### Step 2: 4-(4-Fluorotetrahydro-2H-pyran-4-yl)benzenecarbonitrile

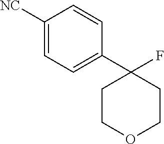

By the process described under Example 5A/step 2, 6.5 g (31.98 mmol) of the compound from Example 7A/step 1 were reacted to give 3.73 g (57% of th.) of the title compound.

¹H-NMR (400 MHz, CDCl₃, δ/ppm): 7.68 (d, 2H), 7.50 (d, 2H), 3.98-3.83 (m, 4H), 2.23-2.05 (m, 2H), 1.91-1.85 (m, 2H).

HPLC (method A): Rₜ=4.04 min.

MS (DCI, NH₃): m/z=223 [M+NH₄]⁺.

### Step 3: 4-(4-Fluorotetrahydro-2H-pyran-4-yl)-N′-hydroxybenzenecarboximide amide

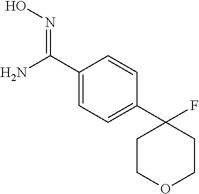

By the process described under Example 1A/step 5, 3.57 mg (88% of th.) of the title compound were obtained from 3.5 g (17.05 mmol) of the compound from Example 7A/step 2.

¹H-NMR (500 MHz, DMSO-d₆, δ/ppm): 9.64 (s, 1H), 7.70 (d, 2H), 7.44 (d, 2H), 5.81 (s, 2H), 3.88-3.83 (m, 2H), 3.73-3.67 (m, 2H), 2.23-2.06 (m, 2H), 1.87-1.81 (m, 2H).

HPLC (method A): Rₜ=3.06 min.

MS (DCI, NH₃): m/z=239 [M+H]⁺.

LC/MS (method F, ESIpos): Rₜ=0.40 min, m/z=239 [M+H]⁺.

### Example 8A

### N′-Hydroxy-4-(4-methoxytetrahydro-2H-pyran-4-yl)benzenecarboximide amide

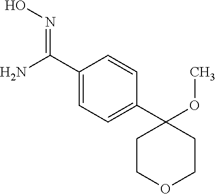

### Step 1: 4-(4-Methoxytetrahydro-2H-pyran-4-yl)benzenecarbonitrile

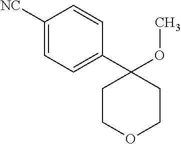

By the process described under Example 6A/step 1, 238 mg (74% of th.) of the title compound were obtained from 300 mg (1.48 mmol) of the compound from Example 7A/step 1 and 111 μl (1.77 mmol) of methyl iodide.

¹H-NMR (500 MHz, CDCl₃, δ/ppm): 7.68 (d, 2H), 7.51 (d, 2H), 3.89-3.82 (m, 4H), 2.99 (s, 3H), 2.03-1.98 (m, 2H), 1.94-1.91 (m, 2H).

HPLC (method A): Rₜ=3.99 min.

MS (DCI, NH₃): m/z=235 [M+NH₄]⁺.

GC/MS (method K, EIpos): Rₜ=6.57 min, m/z=217 (M)⁺.

### Step 2: N′-Hydroxy-4-(4-methoxytetrahydro-2H-pyran-4-yl)benzenecarboximide amide

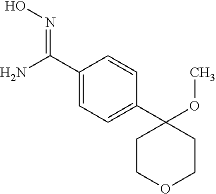

By the process described under Example 1A/step 5, 229 mg (99% of th.) of the title compound were obtained from 200 mg (0.921 mmol) of the compound from Example 8A/step 1.

¹H-NMR (400 MHz, DMSO-d₆, δ/ppm): 9.63 (s, 1H), 7.68 (d, 2H), 7.39 (d, 2H), 5.80 (s, 2H), 3.71-3.67 (m, 4H), 2.88 (m, 2H), 1.93-1.89 (m, 4H).

HPLC (method B): Rₜ=2.95 min.

MS (DCI, NH₃): m/z=251 [M+H]⁺.

LC/MS (method D, ESIpos): Rₜ=0.93 min, m/z=251 [M+H]⁺.

Analogously to the process described under Example 1A/step 5, the N′-hydroxybenzenecarboximide amides listed in the following table were prepared from the corresponding commercially obtainable benzonitriles. The benzonitriles which are not commercially obtainable were prepared in accordance with the following instructions in the literature: 4-cyclohexylbenzenecarbonitrile [E. Riguet et al., J. Organomet. Chem. 2001, 624 (1-2), 376-379], 4-(piperidin-1-yl)benzenecarbonitrile [A.-H. Kuthier et al., J. Org. Chem. 1987, 52 (9), 1710-1713], 4-(pentafluoro-λ⁶-sulphanyl)benzenecarbonitrile [P. J. Crowley et al., Chimia 2004, 58 (3), 138-142], 4-(trimethylsilyl)benzenecarbonitrile [P. di Raddo et al., J. Chen. Soc. Chem. Commun. 1984 (3), 159-160], 4-(2-hydroxypropan-2-yl)benzenecarbonitrile [J. L. Tucker et al., Synth. Comm. 2006, 36 (15), 2145-2155]:

| | | HPLC: | MS: m/z | LC/MS |
|---|---|---|---|---|
| Example | Structure | Rₜ [min] | [M + H]⁺ | method |
| 9A | 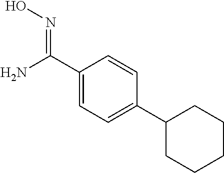 | 1.24 | 219 | H |
| | ¹H-NMR (400 MHz, DMSO-d₆, δ/ppm): 9.51 (s, 1H), 7.56 (d, 2H), 7.20 (d, | | | |
| | 2H), 5.72 (s, broad, 2H), 2.52-2.48 (m, 1H), 1.81-1.74 (m, 4H), 1.73-1.67 (m, | | | |
| | 1H), 1.45-1.31 (m, 4H), 1.28-1.19 (m, 1H). | | | |
| 10A | 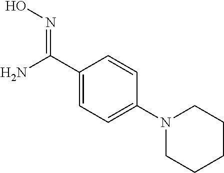 | 1.11 | 220 | D |
| | ¹H-NMR (400 MHz, CDCl₃, δ/ppm): 7.50 (d, 2H), 6.90 (d, 2H), 4.80 (s, broad, | | | |
| | 2H), 3.23-3.20 (m, 4H), 1.71-1.65 (m, 4H), 1.63-1.57 (m, 2H). | | | |
| 11A | 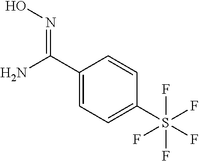 | 1.49 | 263 | D |
| | ¹H-NMR (400 MHz, DMSO-d₆, δ/ppm): 9.99 (s, 1H), 7.94-7.85 (m, 4H), 6.00 | | | |
| | (s, 2H). | | | |
| 12A | 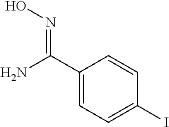 | 1.98 | 263 | G |
| | ¹H-NMR (400 MHz, DMSO-d₆, δ/ppm): 9.71 (s, 1H), 7.73 (d, 2H), 7.47 (d, | | | |
| | 1H), 5.84 (s, broad, 2H). | | | |
| 13A | 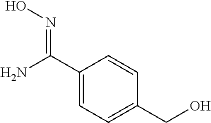 | 0.24 | 167 | D |
| | ¹H-NMR (400 MHz, DMSO-d₆, δ/ppm): 9.55 (s, 1H), 7.62 (d, 2H), 7.29 (d, | | | |
| | 2H), 5.78 (s, 2H), 5.20 (t, 1H), 4.50 (d, 2H). | | | |
| 14A | 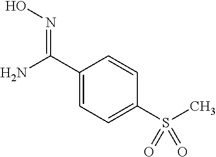 | 0.21 | 215 | F |
| | ¹H-NMR (400 MHz, DMSO-d₆, δ/ppm): 9.98 (s, 1H), 7.92 (s, 4H), 6.00 (s, | | | |
| | broad, 2H), 3.23 (s, 3H). | | | |
| 15A | 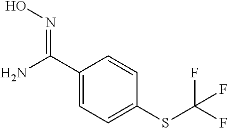 | 1.42 | 237 | D |
| | ¹H-NMR (400 MHz, DMSO-d₆, δ/ppm): 9.90 (s, 1H), 7.80 (d, 2H), 7.72 (d, | | | |
| | 2H), 5.94 (s, 2H). | | | |
| 16A | 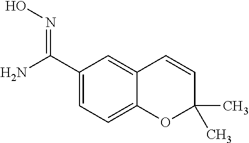 | 0.65 | 219 | F |
| | ¹H-NMR (400 MHz, CDCl₃, δ/ppm): 11.2 (very broad, 1H), 7.35 (dd, 1H), | | | |
| | 7.26 (d, 1H), 6.78 (d, 1H), 6.31 (d, 1H), 5.63 (d, 1H), 4.82 (broad, 2H), 1.43 (s, | | | |
| | 6H). | | | |
| 17A | 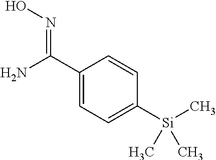 | 0.75 | 209 | I |
| | ¹H-NMR (400 MHz, CDCl3, δ/ppm): 7.95 (s, broad, 1H), 7.60 (d, 2H), 7.55 (d, | | | |
| | 2H), 4.86 (s, broad, 2H), 0.27 (s, 9H). | | | |
| 18A | 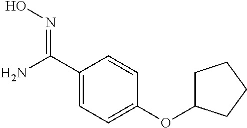 | 3.69 | 221 | A |
| | ¹H-NMR (400 MHz, DMSO-d₆, δ/ppm): 9.43 (s, 1H), 7.57 (d, 2H), 6.87 (d, | | | |
| | 2H), 5.70 (s, broad, 2H), 4.84-4.81 (m, 1H), 1.97-1.88 (m, 2H), 1.73-1.66 (m, | | | |
| | 4H), 1.62-1.53 (m, 2H). | | | |
| 19A | 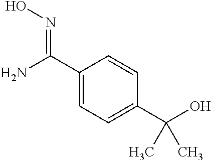 | 0.78 | 195 | D |
| | ¹H-NMR (400 MHz, DMSO-d₆, δ/ppm): 9.53 (s, 1H), 7.59 (d, 2H), 7.44 (d, | | | |
| | 2H), 5.74 (s, broad, 2H), 5.02 (s, 1H), 1.41 (s, 6H). | | | |
| 20A | 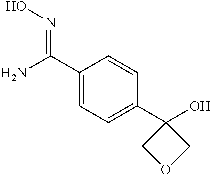 | 0.39 | 209 | D |
| | ¹H-NMR (400 MHz, DMSO-d₆, δ/ppm): 9.61 (s, 1H), 7.70 (d, 2H), 7.59 (d, | | | |
| | 2H), 6.37 (s, 1H), 5.79 (s, broad, 2H), 4.76 (d, 2H), 4.68 (d, 2H). | | | |
| 21A | 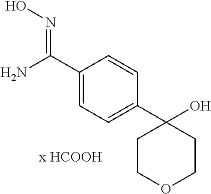 | 0.72 | 237 | D |

1H-NMR (400 MHz, DMSO-d₆, δ/ppm): 9.71 (broad, 1H), 9.59 (s, 1H), 7.62
(d, 2H), 7.48 (d, 2H), 7.17 (broad, 1H), 5.78 (s, broad, 2H), 5.06 (s, 1H), 3.78
(dd, 2H), 3.72-3.69 (m, 2H), 1.97 (dt, 2H), 1.52 (d, 2H).

### Example 22A

### 2-Amino-2-[4-(trifluoromethoxy)phenyl]ethanol

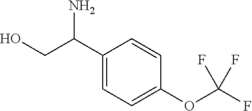

834 mg (38.3 mmol) of lithium borohydride and 1 ml (19.1 mmol) of concentrated sulphuric acid, dissolved in 1 ml of THF, were added successively to a solution of 3.0 g (12.8 mmol) of racemic 4-(trifluoromethoxy)phenylglycine in 20 ml of THF. The reaction mixture was stirred at RT for 24 h. 15 ml of methanol were then added and the mixture was stirred until a clear solution formed. 20 ml of 4 M sodium hydroxide solution were then added dropwise to this solution. A precipitate thereby precipitated out, and was filtered off with suction and discarded. The filtrate was freed from the organic solvents on a rotary evaporator. The residue was extracted three times with approx. 20 ml of toluene each time. The combined organic extracts were concentrated on a rotary evaporator. 2.25 g (80% of th.) of the title compound were obtained.

¹H-NMR (400 MHz, DMSO-d₆, δ/ppm): 7.48 (d, 2H), 7.31 (d, 2H), 5.63 and 5.51 (each broad, tog. 2H), 4.91 (broad, 1H), 3.71-3.67 (m, 1H), 3.66-3.59 (m, 2H).

MS (DCI, NH₃): m/z=222 [M+H]⁺.

### Example 23A

### 5-(5-Methyl-1H-pyrazol-3-yl)-3-[4-(trifluoromethoxy)phenyl]-1,2,4-oxadiazole

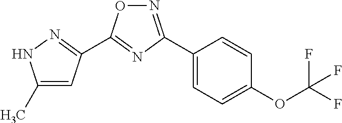

23.3 g (0.121 mol) of EDC, 16.4 g (0.121 mol) of HOBt and 26.7 g (0.121 mol) of N′-hydroxy-4-(trifluoromethoxy)benzenecarboximide amide were added successively to a solution of 15.3 g (0.121 mol) of 5-methyl-1H-pyrazole-3-carboxylic acid in 600 ml of anhydrous DMF at RT. The mixture was stirred first at RT for 2 h and then at 140° C. for 5 h. After cooling, the mixture was diluted with 2 litres of water and extracted three times with 1 litre of ethyl acetate each time. The combined organic extracts were washed successively with water and saturated sodium chloride solution. After drying over anhydrous magnesium sulphate, the mixture was filtered and the solvent was removed on a rotary evaporator. The crude product obtained was purified by means of filtration with suction over a suction filter filled with silica gel (eluent: cyclohexane/ethyl acetate 5:1→1:1). The product fractions were combined and the solvent was removed on a rotary evaporator to such an extent that the product just started to precipitate out. The precipitation was brought to completion at RT. By filtration and further concentration of the mother liquor, two fractions of solid were obtained, which were combined and dried under a high vacuum. 19.7 g (52% of th.) of the title compound were obtained in total in this way.

¹H-NMR (400 MHz, CDCl₃, δ/ppm): 10.75 (broad, 1H), 8.24 (d, 2H), 7.34 (d, 2H), 6.81 (s, 1H), 2.46 (s, 3H).

HPLC (method A): Rₜ=4.72 min.

MS (DCI, NH₃): m/z=311 [M+H]⁺.

LC/MS (method F, ESIpos): Rₜ=1.27 min, m/z=311 [M+H]⁺.

The compounds listed in the following table were prepared by the process described in Example 23A from 5-methyl-1H-pyrazole-3-carboxylic acid, 5-(trifluoromethyl)-1H-pyrazole-3-carboxylic acid, 5-nitro-1H-pyrazole-3-carboxylic acid or 2-methyl-1H-imidazole-4-carboxylic acid hydrate and the corresponding N′-hydroxybenzenecarboximide amides. The reaction time during which stirring was initially carried out at RT was 0.5 to 4 h, depending on the size of the batch. The mixture was subsequently heated at 140° C. for 1 to 15 h. Depending on the polarity of the product obtained, this already precipitated out on addition of water after the reaction had ended, and it was then washed and dried under a high vacuum. Alternatively, as described above, the mixture was worked up by extraction and the product was then purified by chromatography over silica gel; various mobile phases were used for the chromatography. In some cases it was possible to omit the chromatography and to purify the product directly by extraction by stirring in methylene chloride, ethyl acetate, acetonitrile or tert-butyl methyl ether. The compound in Example 36A was purified by means of preparative HPLC (method M).

| | | HPLC: | MS: m/z | LC/MS |
|---|---|---|---|---|
| Example | Structure | Rₜ [min] | [M + H]⁺ | method |
| 24A | 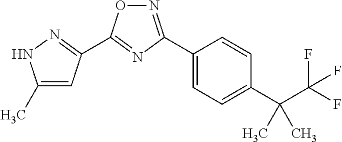 | 1.34 | 337 | F |
| | ¹H-NMR (400 MHz, DMSO-d₆, δ/ppm): 11.80 (s, broad, 1H), 8.17 (d, 2H), | | | |
| | 7.63 (d, 2H), 6.83 (s, 1H), 2.46 (s, 3H), 1.63 (s, 6H). | | | |
| 25A | 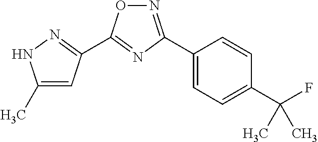 | 2.19 | 287 | D |
| | ¹H-NMR (400 MHz, DMSO-d₆, δ/ppm): 13.54 (s, broad, 1H), 8.08 (d, 2H), | | | |
| | 7.62 (d, 2H), 6.81 (s, 1H), 2.33 (s, 3H), 1.72 (s, 3H), 1.68 (s, 3H). | | | |
| 26A | 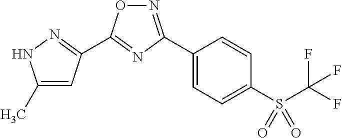 | 1.25 | 359 | F |
| | ¹H-NMR (400 MHz, DMSO-d₆, δ/ppm): 13.62 (s, broad, 1H), 8.49 (d, 2H), | | | |
| | 8.38 (d, 2H), 6.83 (s, 1H), 2.34 (s, 3H). | | | |
| 27A | 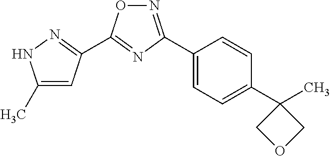 | 1.98 | 297 | C |
| | ¹H-NMR (400 MHz, CDCl₃, δ/ppm): 8.17 (d, 2H), 7.33 (d, 2H), 6.82 (s, 1H), | | | |
| | 5.00 (d, 2H), 4.68 (d, 2H), 2.45 (s, 3H), 1.77 (s, 3H). | | | |
| 28A | 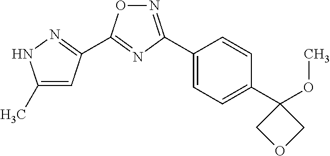 | 0.99 | 313 | F |
| | ¹H-NMR (400 MHz, DMSO-d₆, δ/ppm): 13.54 (s, broad, 1H), 8.14 (d, 2H), | | | |
| | 7.69 (d, 2H), 6.80 (s, 1H), 4.82 (d, 2H), 4.78 (d, 2H), 3.08 (s, 3H), 2.37 (s, | | | |
| | 3H). | | | |
| 29A | 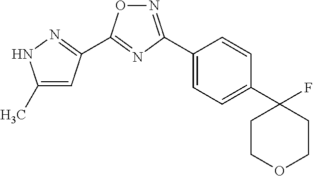 | 4.24 | 329 | C |
| | ¹H-NMR (400 MHz, CDCl₃, δ/ppm): 10.73 (broad, 1H), 8.20 (d, 2H), 7.52 (d, | | | |
| | 2H), 6.81 (s, 1H), 4.00-3.88 (m, 4H), 2.45 (s, 3H), 2.30-2.11 (m, 2H), 1.98- | | | |
| | 1.91 (m, 2H). | | | |
| 30A | 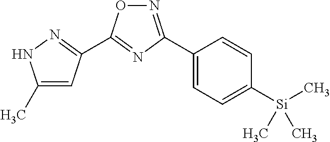 | 2.39 | 299 | E |
| | ¹H-NMR (400 MHz, CDCl₃, δ/ppm): 11.3 (s, broad, 1H), 8.12 (d, 2H), 7.63 (d, | | | |
| | 2H), 6.81 (s, 1H), 2.43 (s, 3H), 0.31 (s, 9H). | | | |
| 31A | 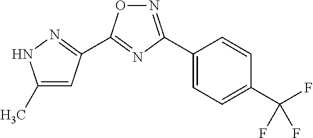 | 1.11 | 295 | I |
| | ¹H-NMR (400 MHz, CDCl₃, δ/ppm): 10.52 (broad, 1H), 8.32 (d, 2H), 7.77 (d, | | | |
| | 2H), 6.82 (s, 1H), 2.63 (s, 3H). | | | |
| 32A | 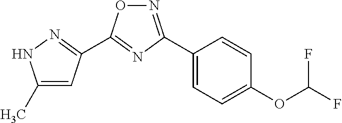 | 1.02 | 293 | I |
| | ¹H-NMR (400 MHz, CDCl₃, δ/ppm): 10.85 (broad, 1H), 8.20 (d, 2H), 7.23 (d, | | | |
| | 2H), 6.81 (s, 1H), 6.60 (t, 1H), 2.46 (s, 3H). | | | |
| 33A | 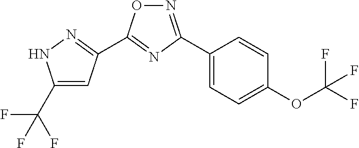 | 2.41 | 365 | E |
| | ¹H-NMR (400 MHz, CDCl₃, δ/ppm): 11.73 (broad, 1H), 8.19 (d, 2H), 7.38 (d, | | | |
| | 2H), 7.37 (s, 1H). | | | |
| 34A | 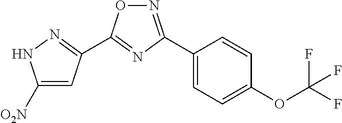 | 2.18 | 342 | E |
| | ¹H-NMR (500 MHz, DMSO-d₆, δ/ppm): 8.20 (d, 2H), 7.58 (d, 2H), 7.34 (s, | | | |
| | 1H). | | | |
| 35A | 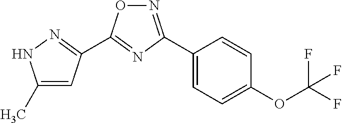 | 1.08 | 311 | F |
| | ¹H-NMR (400 MHz, CDCl₃, δ/ppm): 8.23 (d, 2H), 7.82 (d, 2H), 7.33 (s, 1H), | | | |
| | 2.56 (s, 3H). | | | |
| 36A | 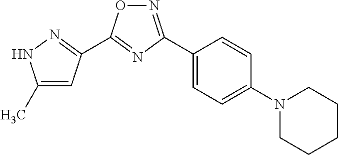 | 0.97 | 310 | F |

¹H-NMR (400 MHz, CDCl₃, δ/ppm): 9.61 (broad, 1H), 8.02 (d, 2H), 7.79 (s,
1H), 6.96 (d, 2H), 3.31-3.27 (m, 4H), 2.54 (s, 3H), 1.73-1.61 (m, 6H).

### Example 37A

### 3-{3-[4-(Trifluoromethoxy)phenyl]-1,2,4-oxadiazol-5-yl}-1H-pyrazol-5-amine

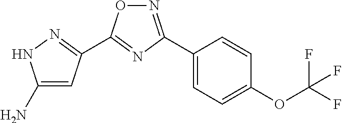

A solution of 342 mg (1.0 mmol) of the compound from Example 34A in 43 ml of ethyl acetate was hydrogenated in a flow-through hydrogenation apparatus (“H-Cube” from ThalesNano, Budapest, Hungary) (conditions: 10% Pd/C catalyst, 1 bar of H₂, 25° C., 1 ml/min). After removal of the solvent on a rotary evaporator, the crude product was purified by means of MPLC (silica gel, mobile phase: cyclohexane/ethyl acetate 1:1). 322 mg (93% of th.) of the title compound were obtained.

¹H-NMR (400 MHz, DMSO-d₆, δ/ppm): 12.49 (s, 1H), 8.19 (d, 2H), 7.49 (d, 2H), 5.93 (s, 1H), 5.44 (s, 2H).

MS (DCI, NH₃): m/z=312 [M+H]⁺.

LC/MS (method E, ESIpos): Rₜ=1.76 min, m/z=312 [M+H]⁺.

### Example 38A

### 2-Chloro-4-(chloromethyl)pyridine

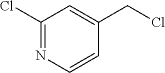

1.00 g (6.97 mmol) of (2-chloropyridin-4-yl)methanol was dissolved in 40 ml of methylene chloride, 10 ml of thionyl chloride were slowly added at RT and the mixture was stirred at RT overnight. The mixture was then concentrated on a rotary evaporator and the residue was stirred in a mixture of methylene chloride and aqueous sodium bicarbonate solution. The phases were separated and the methylene chloride phase was dried over anhydrous magnesium sulphate, filtered and concentrated on a rotary evaporator. 1.10 g (97% of th.) of the title compound were obtained.

¹H-NMR (400 MHz, CDCl₃, δ/ppm): 8.49 (d, 1H), 7.38 (s, 1H), 7.27-7.22 (m, 1H), 4.52 (s, 2H).

LC/MS (method E, ESIpos): Rₜ=1.43 min, m/z=162 [M+H]⁺.

### Example 39A

### 2-(Chloromethyl)-5-iodopyridine

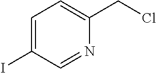

### Step 1: 2-(Hydroxymethyl)-5-iodopyridine

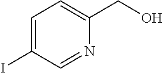

5.7 ml (9.07 mmol) of a 1.6 M solution of n-butyllithium in hexane were added dropwise to a solution of 2.50 g (7.56 mmol) of 2,5-diiodopyridine in 90 ml of toluene under inert conditions and at a temperature of −78° C. The mixture was stirred at −78° C. for 2.5 h and 756 μl of anhydrous DMF was then added at the same temperature. After a further 60 min at −78° C., the reaction mixture was allowed to warm to −10° C., 572 mg (15.11 mmol) of solid sodium borohydride were added and stirring was continued at 0° C. for 30 min. 25 ml of saturated aqueous ammonium chloride solution were then added and the mixture was warmed to RT. The organic phase was separated off and the solvent was removed on a rotary evaporator. The residue was purified by means of preparative HPLC. 890 mg (50% of th.) of the title compound (for the analytical data see below) and 243 mg (14% of th.) of the isomeric 5-(hydroxymethyl)-2-iodopyridine were obtained [preparative HPLC conditions: column: Sunfire C18 OBD 5 μm, 19 mm×150 mm; temperature: 40° C.; mobile phase: water/acetonitrile/1% strength aqueous TFA 76:5:19; flow rate: 25 ml/min; 1.3 g of crude product were dissolved in a mixture of 8 ml of 1% strength aqueous TFA and 4 ml of acetonitrile; injection volume: 1 ml].

¹H-NMR (400 MHz, CDCl₃, δ/ppm): 8.87 (d, 1H), 8.30 (dd, 1H), 7.38 (d, 1H), 5.43 (broad, 1H), 4.85 (s, 2H).

HPLC (method A): Rₜ=0.87 min.

MS (DCI, NH₃): m/z=236 [M+H]⁺.

LC/MS (method E, ESIpos): Rₜ=0.85 min, m/z=236 [M+H]⁺.

### Step 2: 2-(Chloromethyl)-5-iodopyridine

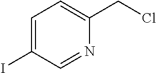

357 μl (4.88 mmol) of thionyl chloride were added dropwise to a solution of 765 mg (3.26 mmol) of the compound from Example 39A/step 1 in 12 ml of anhydrous methylene chloride at 0° C. The reaction mixture was then stirred at RT for 15 h. Approx. 50 ml of saturated aqueous sodium bicarbonate solution was then added and the mixture was extracted three times with approx. 50 ml of methylene chloride each time. The combined organic extracts were washed with saturated sodium chloride solution and dried over anhydrous magnesium sulphate. After filtration, the solvent was removed on a rotary evaporator. 541 mg (66% of th.) of the title compound were obtained.

¹H-NMR (400 MHz, CDCl₃, δ/ppm): 8.79 (d, 1H), 8.03 (dd, 1H), 7.29 (d, 1H), 4.61 (s, 2H).

MS (ESIpos): m/z=254/256 (³⁵Cl/³⁷Cl) [M+H]⁺.

LC/MS (method D, ESIpos): Rₜ=1.87 min, m/z=254/256 (³⁵Cl/³⁷Cl) [M+H]⁺.

### Example 40A

### 5-(Chloromethyl)pyridine-2-carbonitrile hydrochloride

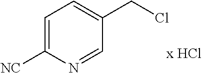

272 μl (3.73 mmol) of thionyl chloride were added to a solution of 250 mg (1.86 mmol) of 5-(hydroxymethyl)pyridine-2-carbonitrile [A. Ashimori et al., Chem. Pharm. Bull. 1990, 38 (9), 2446-2458] in 5 ml of anhydrous methylene chloride at 0° C. The reaction mixture was then stirred at RT for 6 h. All the volatile constituents were then removed on a rotary evaporator and the residue obtained was dried under a high vacuum. 263 mg (75% of th.) of the title compound were obtained.

¹H-NMR (400 MHz, CDCl₃, δ/ppm): 8.73 (d, 1H), 7.90 (dd, 1H), 7.72 (d, 1H), 4.63 (s, 2H).

MS (ESIpos): m/z=153/155 (³⁵Cl/³⁷Cl) [M+H]⁺.

LC/MS (method F, ESIpos): Rₜ=0.75 min, m/z=153/155 (³⁵Cl/³⁷Cl) [M+H]⁺.

### Example 41A

### (6-Cyanopyridin-3-yl)methyl methanesulphonate

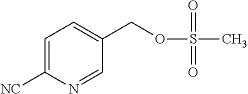

3.51 ml (27.14 mmol) of N,N-diisopropylethylamine and 2.87 ml (25.05 mmol) of methanesulphonic acid chloride were added successively to a solution of 2.8 g (20.87 mmol) of 5-(hydroxymethyl)pyridine-2-carbonitrile [A. Ashimori et al., Chem. Pharm. Bull. 1990, 38 (9), 2446-2458] in 50 ml of anhydrous methylene chloride at 0° C. The reaction mixture was then stirred at RT for 1 h. 10 ml of water were then added, the phases were separated and the aqueous phase was extracted twice with approx. 10 ml of methylene chloride each time. The combined organic extracts were washed with saturated sodium chloride solution, dried over anhydrous magnesium sulphate, filtered and freed from the solvent on a rotary evaporator. The residue obtained was separated into its components by means of MPLC (silica gel, mobile phase: cyclohexane/ethyl acetate 1:1). 2.12 g (48% of th.) of the title compound (for the analytical data see below) and 1.51 g (47% of th.) of the compound described in Example 40A were obtained.

¹H-NMR (400 MHz, CDCl₃, δ/ppm): 8.76 (d, 1H), 7.93 (dd, 1H), 7.78 (d, 1H), 5.32 (s, 2H), 3.10 (s, 3H).

MS (DCI, NH₃): m/z=213 [M+H]⁺, 230 [M+NH₄]⁺.

LC/MS (method F, ESIpos): Rₜ=0.57 min, m/z=213 [M+H]⁺.

### Example 42A

### [3-(Bromomethyl)phenoxy](tripropan-2-yl)silane

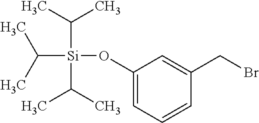

### Step 1: Ethyl 3-[(tripropan-2-ylsilyl)oxy]benzenecarboxylate

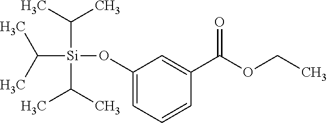

5.98 g (30.99 mmol) of triisopropylsilyl chloride were added dropwise to a solution of 5.0 g (30.09 mmol) of 3-hydroxybenzoic acid ethyl ester and 2.41 g (35.35 mmol) of imidazole in 20 ml of anhydrous DMF at 0° C. After the reaction mixture had been stirred at RT for 15 h, approx. 100 ml of water were added and the mixture was extracted three times with approx. 100 ml of diethyl ether each time. The combined organic extracts were washed successively with water and saturated sodium chloride solution. After drying over anhydrous magnesium sulphate and filtration, the solvent was removed on a rotary evaporator. The residue obtained was purified by filtration with suction over silica gel with cyclohexane/ethyl acetate 10:1→1:1 as the mobile phase. 9.70 g (100% of th.) of the title compound were obtained.

¹H-NMR (400 MHz, CDCl₃, δ/ppm): 7.62 (dd, 1H), 7.53 (m, 1H), 7.28 (dd, 1H), 7.06 (dd, 1H), 4.37 (quart, 2H), 1.39 (t, 3H), 1.28 (sept, 3H), 1.10 (d, 18H).

GC/MS (method K, EI): Rₜ=6.62 min, m/z=322 (M)⁺, 279 (M—C₃H₇)⁺.

### Step 2: {3-[(Tripropan-2-ylsilyl)oxy]phenyl}methanol

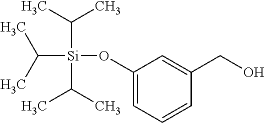

Under inert conditions, 50 ml (49.61 mmol) of a 1 M solution of lithium aluminium hydride in THF were diluted with 50 ml of anhydrous diethyl ether, and a solution of 8.0 g (24.80 mmol) of the compound from Example 42A/step 1 in 50 ml of anhydrous diethyl ether was then added dropwise at 0° C. The reaction mixture was stirred at RT for 1 h. A few ml of methanol were then first added in order to solvolyse excess hydride, and then approx. 150 ml of 0.1 M hydrochloric acid. The organic phase was separated off rapidly and the aqueous phase was extracted twice with approx. 50 ml of diethyl ether each time. The combined organic extracts were washed successively with water and saturated sodium chloride solution. After drying over anhydrous magnesium sulphate and subsequent filtration, the solvent was removed on a rotary evaporator. The residue obtained was purified by filtration with suction over silica gel with cyclohexane/ethyl acetate 5:1→1:1 as the mobile phase. 6.69 g (96% of th.) of the title compound were obtained.

¹H-NMR (400 MHz, CDCl₃, δ/ppm): 7.20 (dd, 1H), 6.93-6.90 (m, 2H), 6.80 (dd, 1H), 4.64 (d, 2H), 1.61 (t, 3H), 1.26 (sept, 3H), 1.09 (d, 18H).

GC/MS (method K, EI): Rₜ=6.38 min, m/z=280 (M)⁺, 237 (M—C₃H₇)⁺.

### Step 3: [3-(Bromomethyl)phenoxy](tripropan-2-yl)silane

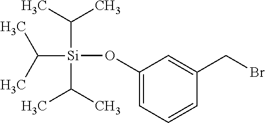

1.0 g (3.57 mmol) of the compound from Example 42A/step 2 was dissolved in 20 ml of anhydrous THF and 1.12 g (4.28 mmol) of triphenylphosphine were added. After this had dissolved, 1.42 g (4.28 mmol) of tetrabromomethane were added. The mixture was then stirred at RT for 20 h. The precipitate which had precipitated out was then filtered off and the filtrate was freed from the solvent on a rotary evaporator. The crude product was purified by means of MPLC (silica gel, mobile phase: cyclohexane/ethyl acetate 50:1). 1.10 g (90% of th., purity of approx. 90%) of the title compound were obtained, this being used without further purification.

¹H-NMR (400 MHz, CDCl₃, δ/ppm): 7.18 (dd, 1H), 6.95 (dd, 1H), 6.91 (m, 1H), 6.80 (dd, 1H), 4.43 (s, 2H), 1.25 (sept, 3H), 1.10 (d, 18H).

HPLC (method B): Rₜ=6.17 min.

GC/MS (method K, EI): Rₜ=6.56 min, m/z=342/344 (⁷⁹Br/⁸¹Br) (M)⁺.

### Example 43A

### Ethyl (4-{[(methylsulphonyl)oxy]methyl}phenyl)acetate

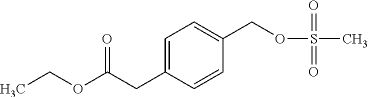

A solution of 1.1 g (5.66 mmol) of [4-(hydroxymethyl)phenyl]acetic acid ethyl ester [G. Biagi et al., Farmaco Ed. Sci. 1988, 43 (7/8), 597-612] and 1.03 ml (7.36 mmol) of triethylamine in 10 ml of anhydrous THF was cooled to 0° C. A solution of 526 μl (6.80 mmol) of methanesulphonic acid chloride in 5 ml of anhydrous THF was then added dropwise. After 15 min at 0° C., the mixture was warmed to RT. After a further hour, approx. 60 ml of water were added and the mixture was extracted twice with approx. 50 ml of ethyl acetate each time. The combined organic extracts were washed with saturated sodium chloride solution. After drying over anhydrous magnesium sulphate and filtration, the solvent was removed on a rotary evaporator. The crude product was purified by means of MPLC (silica gel, mobile phase: cyclohexane/ethyl acetate 7:3). 1.19 g (56% of th., purity of approx. 73%) of the title compound were obtained, this being used without further purification.

MS (DCI, NH₃): m/z=290 [M+NH₄]⁺.

LC/MS (method C, ESIpos): Rₜ=1.96 min, m/z=177 (M—CH₃SO₂O)⁺.

### Example 44A

### 3-[4-(Chloromethyl)phenyl]propan-1-ol

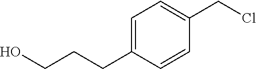

483 μl (6.62 mmol) of thionyl chloride and 717 mg (6.02 mmol) of HOBt were added to a solution of 1.0 g (6.02 mmol) of 3-[4-(hydroxymethyl)phenyl]propan-1-ol [K. Tanaka et al., Org. Lett. 2007, 9 (7), 1215-1218] in 12 ml of anhydrous methylene chloride at RT. After 5 min, a solution of 999 mg (6.02 mmol) of potassium iodide in 12 ml of DMF was added. After the reaction mixture had been stirred at RT for 16 h, it was diluted with 36 ml of water and extracted three times with approx. 25 ml of diethyl ether each time. The combined organic extracts were washed successively with 5% strength aqueous sodium thiosulphate solution, water and saturated sodium chloride solution. After drying over anhydrous magnesium sulphate, filtration and subsequent evaporation of the solvent on a rotary evaporator, the crude product was purified by means of MPLC (silica gel, mobile phase: cyclohexane/ethyl acetate 2:1). 236 mg (21% of th.) of the title compound were obtained.

¹H-NMR (400 MHz, CDCl₃, δ/ppm): 7.31 (d, 2H), 7.20 (d, 2H), 4.57 (s, 2H), 3.68 (t, 2H), 2.71 (t, 2H), 1.89 (quint, 2H), 1.31 (s, broad, 1H).

MS (DCI, NH₃): m/z=202 [M+NH₄]⁺.

GC/MS (method K, Rₜ=5.51 min, m/z=184 [M]⁺.

### Example 45A

### 1-[(6-Chloropyridin-3-yl)methyl]-5-methyl-1H-pyrazole-3-carboxylic acid

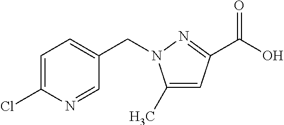

### Step 1: Ethyl 1-[(6-chloropyridin-3-yl)methyl]-5-methyl-1H-pyrazole-3-carboxylate

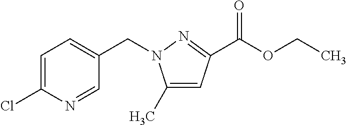

9.46 g (84.3 mmol) of potassium tert-butylate were added to a solution of 10.0 g (64.9 mmol) of ethyl 3-methyl-1H-pyrazole-5-carboxylate and 13.66 g (84.3 mmol) of 2-chloro-5-(chloromethyl)pyridine in 162 ml of anhydrous THF at 0° C. The mixture was allowed to come to RT and was stirred at RT for a further 18 h. It was then diluted with 200 ml of ethyl acetate and 350 ml of water, the phases were mixed thoroughly and the aqueous phase, which was separated off, was extracted twice more with 200 ml of ethyl acetate each time. The combined organic phases were dried over anhydrous sodium sulphate, filtered and concentrated on a rotary evaporator. The residue was purified by column chromatography over silica gel (mobile phase: cyclohexane/ethyl acetate 4:1→2:1). After drying in vacuo, 12.4 g (65% of th.) of the title compound were obtained in a purity of 95%.

¹H-NMR (400 MHz, DMSO-d₆, δ/ppm): 8.30 (d, 1H), 7.58 (dd, 1H), 7.52 (d, 1H), 6.60 (s, 1H), 5.45 (s, 2H), 4.24 (quart, 2H), 2.28 (s, 3H), 1.27 (t, 3H).

LC/MS (method C, ESIpos): Rₜ=1.88 min, m/z=280 [M+H]⁺.

### Step 2: 1-[(6-Chloropyridin-3-yl)methyl]-5-methyl-1H-pyrazole-3-carboxylic acid

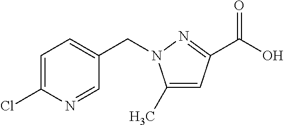

3.39 g (84.7 mmol) of sodium hydroxide, dissolved in 100 ml of water, were added to a solution of 11.85 g (42.36 mmol) of the compound from Example 45A/step 1 in 100 ml of THF and the mixture was stirred at RT for 5 h. The mixture was then diluted with 150 ml of water and washed once with 100 ml of ethyl acetate. The aqueous phase was adjusted to a pH of approx. 3 with 1 N hydrochloric acid and extracted three times with 150 ml of ethyl acetate each time. The latter ethyl acetate phases were combined, dried over anhydrous sodium sulphate, filtered and concentrated on a rotary evaporator. After the residue had been dried in vacuo, 9.72 g (91% of th.) of the title compound were obtained.

¹H-NMR (400 MHz, DMSO-d₆, δ/ppm): 12.60 (s, broad, 1H), 8.31 (d, 1H), 7.60 (dd, 1H), 7.52 (d, 1H), 6.53 (s, 1H), 5.42 (s, 2H), 2.28 (s, 3H).

LC/MS (method F, ESIpos): Rₜ=0.75 min, m/z=252 [M+H]⁺.

### Example 46A

### 1-[(6-Chloropyridin-3-yl)methyl]-5-methyl-1H-pyrrole-3-carboxylic acid

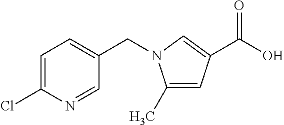

### Step 1: Methyl 2-(hydroxymethylidene)-4-oxopentanoate

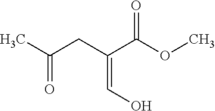

7.63 g (190.7 mmol) of a 60% strength suspension of sodium hydride in mineral oil were deoiled with pentane under inert conditions. 150 ml of anhydrous diethyl ether and, at 0° C., 138 μl (3.4 mmol) of methanol were then added. After stirring at RT for 10 min, the mixture was cooled to 0° C. again and a mixture of 12.6 ml (204.3 mmol) of formic acid methyl ester and 30.0 g (170.2 mmol) of methyl 4,4-dimethoxypentanoate [C. Meister et al., Liebigs Ann. Chem. 1983 (6), 913-921] was slowly added. The reaction mixture was stirred at RT for 16 h. Approx. 60 ml of ice-water were then added and the mixture was extracted with 100 ml of diethyl ether. The organic extract was discarded and the aqueous phase was brought to a pH of 2-3 with 3 M hydrochloric acid. It was extracted four times with approx. 50 ml of tert-butyl methyl ether each time. The combined organic extracts were dried over anhydrous magnesium sulphate, filtered and freed from the solvent on a rotary evaporator. 4.2 g (13% of th., purity of 85%) of the title compound were obtained, this being employed without further purification.

GC/MS (method K, EI): Rₜ=3.33 min, m/z=158 [M]⁺, 140 [M—H₂O]⁺.

### Step 2: Methyl 1-[(6-chloropyridin-3-yl)methyl]-5-methyl-1H-pyrrole-3-carboxylate

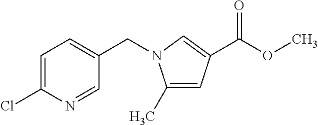

A mixture of 4.20 g (22.73 mmol, purity of 85%) of the compound from Example 46A/step 1 and 3.24 g (22.73 mmol) of 5-(aminomethyl)-2-chloropyridine in 42 ml of methanol was stirred at RT for three days. The solvent was then removed on a rotary evaporator and the crude product was purified by means of MPLC (silica gel, mobile phase: cyclohexane/ethyl acetate 2:1). 3.37 g (56% of th.) of the title compound were obtained.

¹H-NMR (400 MHz, CDCl₃, δ/ppm): 8.19 (d, 1H), 7.30-7.20 (m, 3H), 6.38 (d, 1H), 5.03 (s, 2H), 3.79 (s, 3H), 2.12 (s, 3H).

HPLC (method A): Rₜ=4.10 min.

MS (DCI, NH₃): m/z=265 [M+H]⁺.

### Step 3: 1-[(6-Chloropyridin-3-yl)methyl]-5-methyl-1H-pyrrole-3-carboxylic acid

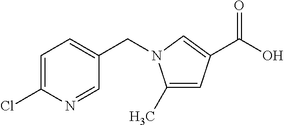

14.5 ml (14.5 mmol) of 1 M sodium hydroxide solution were added dropwise to a solution of 1.93 g (7.29 mmol) of the compound from Example 46A/step 2 in 38 ml of methanol. The reaction mixture was heated under reflux for 15 h. After cooling to RT, the methanol was mostly removed on a rotary evaporator. The residue was first diluted with 100 ml of water and then acidified with 2 M hydrochloric acid. The precipitate which had precipitated out was filtered off, rinsed with water and dried under a high vacuum. 1.41 g (76% of th.) of the title compound were obtained.

¹H-NMR (400 MHz, DMSO-d₆, δ/ppm): 11.67 (s, 1H), 8.23 (s, 1H), 7.51 (d, 2H), 7.45 (d, 2H), 6.18 (d, 1H), 5.19 (s, 2H), 2.07 (s, 3H).

### Example 47A

### 5-Methyl-1-(4-methylbenzyl)-1H-pyrrole-3-carboxylic acid

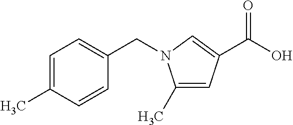

### Step 1: Methyl 5-methyl-1-(4-methylbenzyl)-1H-pyrrole-3-carboxylate

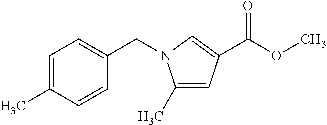

13.25 g (36.03 mmol) of the compound from Example 46A/step 1 and 4.6 ml (36.03 mmol) of 4-methylbenzylamine were dissolved in 100 ml of methanol. This solution was divided into seven portions and heated at 100° C. in a microwave oven (CEM Discover, initial irradiation power 100 W) for 10 min. The reaction mixtures were then combined again and freed from the solvent on a rotary evaporator. The title compound was isolated by means of filtration with suction over silica gel (cyclohexane/ethyl acetate gradient 7:1→6:1→5:1). 7.25 g (83% of th.) were obtained.

¹H-NMR (400 MHz, CDCl₃, δ/ppm): 7.26 (d, 1H), 7.13 (d, 2H), 6.92 (d, 2H), 6.34 (d, 1H), 4.97 (s, 2H), 3.77 (s, 3H), 2.33 (s, 3H), 2.11 (s, 3H).

LC/MS (method D, ESIpos): Rₜ=2.35 min, m/z=244 [M+H]⁺.

### Step 2: 5-Methyl-1-(4-methylbenzyl)-1H-pyrrole-3-carboxylic acid

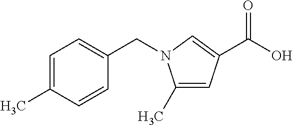

By the process described under Example 46A/step 3, 1.78 g (94% of th.) of the title compound were obtained from 2.0 g (8.22 mmol) of the compound from Example 47A/step 1.

¹H-NMR (400 MHz, DMSO-d₆, δ/ppm): 11.58 (s, broad, 1H), 7.36 (d, 1H), 7.15 (d, 2H), 6.99 (d, 2H), 6.14 (d, 1H), 5.05 (s, 2H), 2.28 (s, 3H), 2.05 (s, 3H).

HPLC (method A): Rₜ=4.22 min.

MS (DCI, NH₃): m/z=230 [M+H]⁺, 247 [M+NH₄]⁺.

LC/MS (method C, ESIpos): Rₜ=2.12 min, m/z=230 [M+H]⁺.

### Example 48A

### 1-Methyl-5-(4-methylbenzyl)-1H-pyrrole-3-carboxylic acid

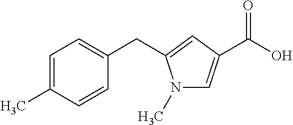

### Step 1: Methyl 5-bromo-1-methyl-1H-pyrrole-3-carboxylate

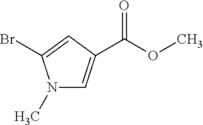

3.40 g (30.26 mmol) of potassium tert-butylate were added to a solution of 4.75 g (23.28 mmol) of methyl 5-bromo-1H-pyrrole-3-carboxylate [H. J. Anderson et al., Can. J. Chem. 1967 (45), 897-902] in 45 ml of anhydrous DMF and the mixture was stirred at RT for 15 min. 1.9 ml (30.26 mmol) of methyl iodide were then added dropwise. The reaction mixture was stirred at RT for 90 min. It was then poured onto 150 ml of ice/water. The precipitate which had precipitated out was filtered off with suction, washed with water and dried under a high vacuum. 3.76 g (74% of th.) of the title compound were obtained.

¹H-NMR (400 MHz, CDCl₃, δ/ppm): 7.31 (d, 1H), 6.60 (d, 1H), 3.79 (s, 3H), 3.62 (s, 3H).

HPLC (method A): Rₜ=3.85 min.

MS (ESIpos): m/z=218/220 (⁷⁹Br/⁸¹Br) [M+H]⁺.

LC/MS (method F, ESIpos): Rₜ=1.02 min, m/z=218/220 (⁷⁹Br/⁸¹Br) [M+H]⁺.

### Step 2: Methyl 5-[hydroxy(4-methylphenyl)methyl]-1-methyl-1H-pyrrole-3-carboxylate

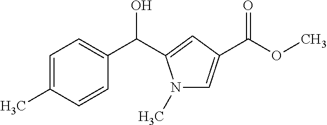

1.3 ml (2.52 mmol) of a 2 M solution of isopropylmagnesium chloride in THF were added dropwise to a solution of 500 mg (2.29 mmol) of the compound from Example 48A/step 1 in 10 ml of anhydrous THF under inert conditions and at −30° C. When the addition had ended, the reaction mixture was stirred at 0° C. for approx. 45 min 307 μl (2.6 mmol) of 4-methylbenzaldehyde were then added at this temperature. After the reaction mixture had been stirred at RT for 15 h, 40 ml of water were added and the mixture was extracted three times with approx. 20 ml of ethyl acetate each time. The combined organic extracts were washed successively with water and saturated sodium chloride solution. After drying over anhydrous magnesium sulphate, the mixture was filtered and the filtrate was freed from the solvent on a rotary evaporator. The residue obtained was purified by means of MPLC (silica gel, mobile phase: methylene chloride→methylene chloride/methanol 20:1). 328 mg (55% of th.) of the title compound were obtained.

¹H-NMR (400 MHz, CDCl₃, δ/ppm): 7.27 (d, 2H), 7.23 (d, 1H), 7.18 (d, 2H), 6.26 (d, 1H), 5.80 (d, 1H), 3.75 (s, 3H), 3.60 (s, 3H), 2.37 (s, 3H), 2.19 (d, 1H).

HPLC (method A): Rₜ=3.95 min.

MS (DCI, NH₃): m/z=260 [M+H]⁺.

LC/MS (method F, ESIpos): Rₜ=1.06 min, m/z=260 [M+H]⁺.

### Step 3: Methyl 1-methyl-5-(4-methylbenzyl)-1H-pyrrole-3-carboxylate

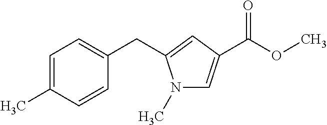

217 μl (1.36 mmol) of triethylsilane and 2.5 ml (13.6 mmol) of trifluoromethanesulphonic acid trimethylsilyl ester were added successively to a solution of 321 mg (1.23 mmol) of the compound from Example 48A/step 2 in 20 ml of anhydrous methylene chloride at 0° C. After the reaction mixture had been stirred at RT for 2 h, it was diluted with methylene chloride and washed with saturated aqueous sodium bicarbonate solution. The solvent was removed on a rotary evaporator and the residue obtained was purified by means of MPLC (silica gel, mobile phase: methylene chloride). 159 mg (52% of th.) of the title compound were obtained.

¹H-NMR (400 MHz, CDCl₃, δ/ppm): 7.21 (d, 1H), 7.10 (d, 2H), 7.13 (d, 2H), 6.33 (d, 1H), 3.87 (s, 2H), 3.77 (s, 3H), 3.42 (s, 3H), 2.32 (s, 3H).

HPLC (method A): Rₜ=4.44 min.

MS (DCI, NH₃): m/z=244 [M+H]⁺.

LC/MS (method F, ESIpos): Rₜ=1.28 min, m/z=244 [M+H]⁺.

### Step 4: 1-Methyl-5-(4-methylbenzyl)-1H-pyrrole-3-carboxylic acid

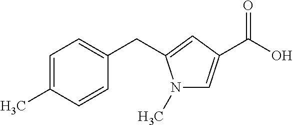

By the process described under Example 46A/step 3, 139 mg (98% of th.) of the title compound were obtained from 155 mg (0.637 mmol) of the compound from Example 48A/step 3.

¹H-NMR (400 MHz, DMSO-d₆, δ/ppm): 11.53 (s, 1H), 7.28 (d, 1H), 7.11 (d, 2H), 7.05 (d, 2H), 6.02 (d, 1H), 3.84 (s, 2H), 3.44 (s, 3H), 2.28 (s, 3H).

HPLC (method A): Rₜ=4.05 min.

MS (DCI, NH₃): m/z=230 [M+H]⁺.

LC/MS (method F, ESIpos): Rₜ=1.08 min, m/z=230 [M+H]⁺.

### Example 49A

### 1-Methyl-5-(4-methylbenzyl)-1H-pyrazole-3-carboxylic acid

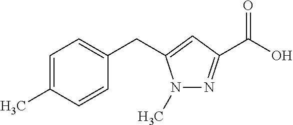

### Step 1: Ethyl 4-hydroxy-5-(4-methylphenyl)-2-oxopent-3-enoate

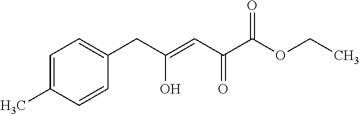

A sodium ethanolate solution was prepared from 935 mg (23.4 mmol) of a 60% strength suspension of sodium hydride in mineral oil and 30 ml of anhydrous ethanol. First 2.76 ml (20.3 mmol) of oxalic acid diethyl ester and then a solution of 3.01 g (20.3 mmol) of 1-(4-methylphenyl)propan-2-one [S. Sugai et al., Chem. Lett. 1982, 597-600] in a further 10 ml of ethanol were added dropwise to this solution at 0° C. After 1 h at 0° C., the reaction mixture was allowed to warm to RT and stirring was continued for a further 5 h. The ethanol was then removed on a rotary evaporator and the residue was taken up in approx. 50 ml of water. The mixture was acidified with 1 M hydrochloric acid, while cooling with ice, and then extracted with methylene chloride. After drying of the organic phase over anhydrous magnesium sulphate, the solvent was removed on a rotary evaporator. 4.48 g (89% of theory) of a product mixture were obtained which, in addition to the E/Z mixture of the title compound, also contained the isomeric ethyl 4-hydroxy-3-(4-methylphenyl)-2-oxo-pent-3-enoate. This mixture was used for the following reaction without further purification.

¹H-NMR (400 MHz, CDCl₃, δ/ppm): 14.30 (broad, 1H), 7.17-7.11 (m, 4H), 6.36 (s, 1H), 4.31 (quart, 2H), 3.73 (s, 2H), 2.33 (s, 3H), 1.34 (t, 3H).

MS (DCI, NH₃): m/z=249 [M+H]⁺, 266 [M+NH₄]⁺.

### Step 2: Ethyl 1-methyl-5-(4-methylbenzyl)-1H-pyrazole-3-carboxylate

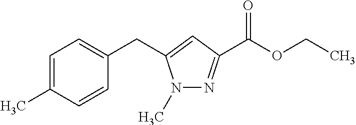

A mixture of 330 mg (1.33 mmol) of the compound from Example 49A/step 1 and 78 μl (1.46 mmol) of methylhydrazine in 3 ml of glacial acetic acid was stirred at 90° C. for 4 h. The acetic acid was then removed on a rotary evaporator and the residue obtained was purified by means of MPLC (silica gel, mobile phase: cyclohexane/ethyl acetate 1:1). 270 mg (79% of th.) of the title compound were obtained.

¹H-NMR (400 MHz, CDCl₃, δ/ppm): 7.12 (d, 2H), 7.02 (d, 2H), 6.58 (s, 1H), 4.38 (quart, 2H), 3.95 (s, 2H), 3.78 (s, 3H), 2.33 (s, 3H), 1.39 (t, 3H).

LC/MS (method C, ESIpos): Rₜ=2.26 min, m/z=259 [M+H]⁺.

### Step 3: 1-Methyl-5-(4-methylbenzyl)-1H-pyrazole-3-carboxylic acid

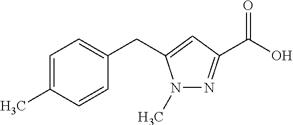

9.6 ml (4.84 mmol) of 0.5 M lithium hydroxide solution in water were added to a solution of 250 mg (0.968 mmol) of the compound from Example 49A/step 2 in 5 ml of ethanol. After the reaction mixture had been stirred at 40° C. for 1 h, it was allowed to cool to RT and 2.9 ml (5.81 mmol) of 2 M hydrochloric acid were added. The precipitate which thereby precipitated out was filtered off with suction, washed with water and dried under a high vacuum. 203 mg (91% of th.) of the title compound were obtained.

¹H-NMR (400 MHz, DMSO-d₆, δ/ppm): 12.51 (broad, 1H), 7.13 (d, 2H), 7.10 (d, 2H), 6.37 (s, 1H), 3.99 (s, 2H), 3.73 (s, 3H), 2.28 (s, 3H).

LC/MS (method C, ESIpos): Rₜ=1.92 min, m/z=231 [M+H]⁺.

### Example 50A

### 2-Methyl-1-(4-methylbenzyl)-1H-imidazole-4-carboxylic acid

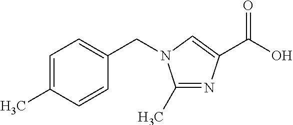

### Step 1: 2-Methyl-1-(4-methylbenzyl)-1H-imidazole-4-carbaldehyde

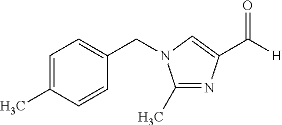

2.52 g (13.6 mmol) of 4-methylbenzyl bromide and 1.12 g (9.99 mmol) of solid potassium tert-butylate were added successively to a solution of 1.0 g (9.08 mmol) of 2-methyl-1H-imidazole-4-carbaldehyde in 20 ml of anhydrous THF. The reaction mixture was stirred at RT for 15 h. Approx. 100 ml of water were then added and the mixture was extracted three times with approx. 100 ml of ethyl acetate each time. The combined organic extracts were washed successively with water and saturated sodium chloride solution. After drying over anhydrous magnesium sulphate and filtering, the solvent was removed on a rotary evaporator. The crude product which remained was purified by means of MPLC (silica gel, mobile phase: cyclohexane/ethyl acetate 10:1→1:2). 1.24 g (64% of th.) of the title compound were obtained, containing approx. 9% of the isomeric 2-methyl-1-(4-methylbenzyl)-1H-imidazole-5-carbaldehyde.

¹H-NMR (400 MHz, CDCl₃, δ/ppm): 9.79 (s, 1H), 7.53 (s, 1H), 7.19 (d, 2H), 7.01 (d, 2H), 5.04 (s, 2H), 2.40 (s, 3H), 2.36 (s, 3H).

HPLC (method A): Rₜ=3.39 min.

MS (DCI, NH₃): m/z=215 [M+H]⁺.

LC/MS (method C, ESIpos): Rₜ=1.43 min, m/z=215 [M+H]⁺.

### Step 2: 2-Methyl-1-(4-methylbenzyl)-1H-imidazole-4-carboxylic acid

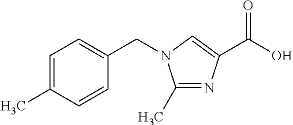

25 ml (50.8 mmol) of a 2 M solution of 2-methyl-2-butene in THF and a solution of 5.48 g (48.5 mmol, 80% strength) of sodium chlorite and 4.93 g (35.7 mmol) of sodium dihydrogen phosphate in 45 ml of water were added to a solution of 980 mg (4.57 mmol) of the compound from Example 50A/step 1 in 90 ml of isobutanol at RT. The reaction mixture was stirred at RT for 2 h. The mixture was then extracted three times with approx. 100 ml of ethyl acetate each time. The combined organic extracts were dried over anhydrous magnesium sulphate, filtered and freed from the solvent on a rotary evaporator. The crude product which remained was purified by means of MPLC (silica gel, mobile phase: methylene chloride/methanol 5:1). 1.23 g (99% of th. at a purity of 85%) of the title compound were obtained, containing approx. 8% of the isomeric 2-methyl-1-(4-methylbenzyl)-1H-imidazole-5-carboxylic acid as the main impurity.

¹H-NMR (400 MHz, DMSO-d₆, δ/ppm): 7.80 (s, 1H), 7.18 (d, 2H), 7.10 (d, 2H), 5.13 (s, 2H), 3.46 (broad, 1H), 2.29 (s, 3H), 2.23 (s, 3H).

HPLC (method A): Rₜ=3.43 min.

MS (ESIpos): m/z=231 [M+H]⁺.

LC/MS (method F, ESIpos): Rₜ=0.60 min, m/z=231 [M+H]⁺.

### Example 51A

### N′-Hydroxy-4-(1-hydroxycyclobutyl)benzenecarboximide amide

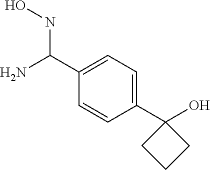

### Step 1: 4-(1-Hydroxycyclobutyl)benzenecarbonitrile

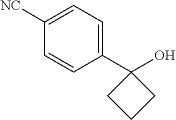

Analogously to the process described under Example 5A/step 1, 9.47 g (83% of th.) of the title compound were obtained from 15.0 g (65.5 mmol) of 4-iodobenzonitrile, 34.4 ml (68.8 mmol) of isopropylmagnesium chloride solution (2 M in diethyl ether) and 7.4 ml (98.2 mmol) of cyclobutanone. The purification of the product was carried out by means of MPLC (silica gel, mobile phase: cyclohexane/ethyl acetate 10:1→4:1).

¹H-NMR (400 MHz, CDCl₃, δ/ppm): 7.67 (d, 2H), 7.62 (d, 2H), 2.58-2.51 (m, 2H), 2.44-2.37 (m, 2H), 2.23-2.04 (m, 2H), 1.83-1.72 (m, 1H).

HPLC (method A): Rₜ=3.47 min.

MS (DCI, NH₃): m/z=191 [M+NH₄]⁺.

### Step 2: N′-Hydroxy-4-(1-hydroxycyclobutyl)benzenecarboximide amide

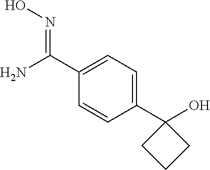

Analogously to the process described under Example 1A/step 5, 1.1 g of the title compound (92% of th.) were obtained starting from 1.0 g (5.77 mmol) of the compound from Example 51A/step 1. In contrast to that described under Example 1A/step 5, however, after removal of the solvent approx. 50 ml of water were added to the residue and the mixture was extracted three times with approx. 50 ml of ethyl acetate each time. The combined organic extracts were washed with saturated sodium chloride solution and dried over anhydrous magnesium sulphate. After filtration, the solvent was removed on a rotary evaporator and the residue obtained was purified by means of MPLC (silica gel; mobile phase: methylene chloride/methanol 50:1→10:1).

¹H-NMR (400 MHz, DMSO-d₆, δ/ppm): 9.57 (s, 1H), 7.63 (d, 2H), 7.47 (d, 2H), 5.79 (s, broad, 2H), 5.50 (s, 1H), 2.42-2.33 (m, 2H), 2.30-2.22 (m, 2H), 1.97-1.60 (m, 1H), 1.70-1.59 (m, 1H).

HPLC (method A): Rₜ=2.26 min.

MS (EIpos): m/z=207 [M+H]⁺.

LC/MS (method I, ESIpos): Rₜ=0.25 min, m/z=207 [M+H]⁺.

### Example 52A

### N′-Hydroxy-4-(1-methoxycyclobutyl)benzenecarboximide amide

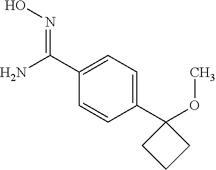

### Step 1: 4-(1-Methoxycyclobutyl)benzenecarbonitrile

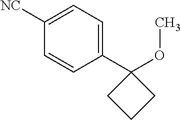

Analogously to the process described under Example 6A/step 1, 1.27 g (59% of th.) of the title compound were obtained from 2.0 g (11.5 mmol) of the compound from Example 51A/step 1, 508 mg (12.7 mmol) of a 60% strength dispersion of sodium hydride in mineral oil and 863 μl (13.9 mmol) of methyl iodide. The purification of the product was carried out by means of MPLC (silica gel; mobile phase: cyclohexane/ethyl acetate 20:1→4:1).

¹H-NMR (400 MHz, CDCl₃, δ/ppm): 7.68 (d, 2H), 7.54 (d, 2H), 2.95 (s, 3H), 2.46-2.32 (m, 4H), 2.03-1.93 (m, 1H), 1.76-1.63 (m, 1H).

MS (DCI, NH₃): m/z=205 [M+NH₄]⁺.

### Step 2: N′-Hydroxy-4-(1-methoxycyclobutyl)benzenecarboximide amide

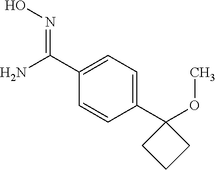

Analogously to the process described under Example 1A/step 5, 1.28 g of the title compound (98% of th.) were obtained starting from 1.1 g (5.87 mmol) of the compound from Example 52A/step 1.

¹H-NMR (400 MHz, DMSO-d₆, δ/ppm): 9.62 (s, 1H), 7.68 (d, 2H), 7.40 (d, 2H), 5.80 (s, broad, 2H), 2.83 (s, 3H), 2.37-2.24 (m, 4H), 1.91-1.81 (m, 1H), 1.65-1.53 (m, 1H).

HPLC (method A): Rₜ=3.02 min.

MS (DCI, NH₃): m/z=221 [M+H]⁺.

### Example 53A

### 4-(1-Fluorocyclobutyl)-N′-hydroxybenzenecarboximide amide

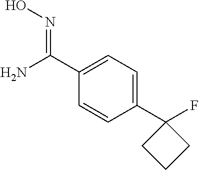

### Step 1: 4-(1-Fluorocyclobutyl)benzenecarbonitrile

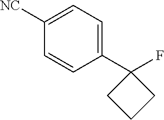

Analogously to the process described under Example 5A/step 2, 1.39 g (69% of th.) of the title compound were obtained from 2.0 g (11.5 mmol) of the compound from Example 51A/step 1 and 1.8 ml (13.9 mmol) of diethylaminosulphur trifluoride (DAST). The purification of the product was carried out by means of MPLC (silica gel; mobile phase: cyclohexane/ethyl acetate 10:1→5:1).

¹H-NMR (400 MHz, CDCl₃, δ/ppm): 7.69 (d, 2H), 7.57 (d, 2H), 2.78-2.62 (m, 2H), 2.58-2.48 (m, 2H), 2.20-2.09 (m, 1H), 1.87-1.75 (m, 1H).

GC/MS (method K, EIpos): Rₜ=4.71 min, m/z=155 [M—HF]⁺.

### Step 2: 4-(1-Fluorocyclobutyl)-N′-hydroxybenzenecarboximide amide

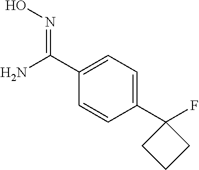

Analogously to the process described under Example 1A/step 5, 1.16 g of the title compound (78% of th.) were obtained starting from 1.25 g (7.13 mmol) of the compound from Example 53A/step 1.

¹H-NMR (400 MHz, CDCl₃, δ/ppm): 7.67 (d, 2H), 7.50 (d, 2H), 4.87 (s, broad, 2H), 2.72-2.52 (m, 5H), 2.16-2.05 (m, 1H), 1.82-1.71 (m, 1H).

HPLC (method A): Rₜ=3.17 min.

MS (DCI, NH₃): m/z=209 [M+H]⁺.

### Example 54A

### N′-Hydroxy-4-(2,2,2-trifluoroethoxy)benzenecarboximide amide

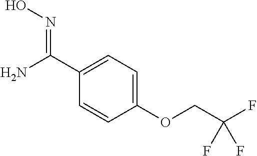

Analogously to the process described under Example 1A/step 5, starting from 7.0 g (34.8 mmol) of 4-(2,2,2-trifluoroethoxy)benzenecarbonitrile [J. T. Gupton et al., Synth. Commun. 1982, 12 (9), 695-700], 6.61 g of the title compound (81% of th.) were obtained.

¹H-NMR (400 MHz, DMSO-d₆, δ/ppm): 9.51 (s, 1H), 7.64 (d, 2H), 7.06 (d, 2H), 5.77 (s, broad, 2H), 4.79 (quart, 2H).

HPLC (method A): Rₜ=3.08 min.

MS (DCI, NH₃): m/z=235 [M+H]⁺.

LC/MS (method I, ESIpos): Rₜ=0.51 min, m/z=235 [M+H]⁺.

### Example 55A

### N′-Hydroxy-4-(1H-pyrrol-1-ylmethyl)benzenecarboximide amide

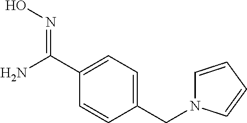

Analogously to the process described under Example 1A/step 5, 702 mg of the title compound (86% of th.) were obtained from 670 mg (3.68 mmol) of 4-(1H-pyrrol-1-ylmethyl)benzenecarbonitrile [M. Artico et al., Eur. J. Med. Chem. 1992, 27 (3), 219-228].

¹H-NMR (400 MHz, CDCl₃, δ/ppm): 7.76 (broad, 1H), 7.58 (d, 2H), 7.13 (d, 2H), 6.68 (dd, 2H), 6.20 (dd, 2H), 5.09 (s, 2H), 4.84 (s, broad, 2H).

LC/MS (method I, ESIpos): Rₜ=0.54 min, m/z=216 [M+H]⁺.

### Example 56A

### (2-Carbamoylpyridin-4-yl)methyl methanesulphonate

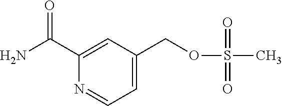

Analogously to the process described under Example 43A, 1.45 g of the title compound (90% of th.) were obtained from 1.07 g (7.00 mmol) of 4-(hydroxymethyl)pyridine-2-carboxamide [I. Martin et al., Acta Chem. Scand. 1995, 49 (3), 230-232].

¹H-NMR (400 MHz, CDCl₃, δ/ppm): 8.62 (d, 1H), 8.21 (s, 1H), 7.83 (s, broad, 1H), 7.51 (d, 1H), 5.70 (s, broad, 1H), 5.31 (s, 2H), 3.10 (s, 3H).

LC/MS (method I, ESIpos): Rₜ=0.43 min, m/z=231 [M+H]⁺.

### Example 57A

### tert-Butyl ({1-[4-(N′-hydroxycarbamimidoyl)phenyl]cyclobutyl}oxy)acetate

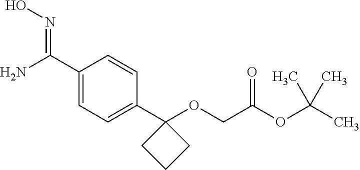

### Step 1: tert-Butyl {[1-(4-cyanophenyl)cyclobutyl]oxy}acetate

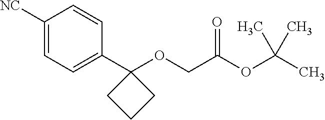

508 mg (12.7 mmol) of sodium hydride (60% strength suspension in mineral oil) were added to a solution of 2.0 g (11.5 mmol) of the compound from Example 51A/step 1 in 40 ml of anhydrous DMF at a temperature of approx. 5° C. After stirring at this temperature for 1 h, 2.0 ml (13.9 mmol) of bromoacetic acid tert-butyl ester were added dropwise. The reaction mixture was allowed to warm to RT and was stirred overnight. Thereafter, a further 1.5 ml (10.2 mmol) of bromoacetic acid tert-butyl ester were added and stirring was continued for a further 4 h. The reaction mixture was then poured on to approx. 150 ml of water and it was extracted with approx. 300 ml of diethyl ether in total. The combined organic extracts were washed successively with water and saturated sodium chloride solution. After drying over anhydrous magnesium sulphate, the mixture was filtered and the solvent was removed on a rotary evaporator. The crude product obtained was purified by means of filtration with suction over silica gel with cyclohexane/ethyl acetate 100:0→80:20 as the mobile phase. 767 mg of the title compound were obtained (33% of th., based on the conversion) and 581 mg of the starting material (compound from Example 51A/step 1) were recovered.

¹H-NMR (400 MHz, CDCl₃, δ/ppm): 7.68 (d, 2H), 7.59 (d, 2H), 3.58 (s, 2H), 2.56-2.49 (m, 2H), 2.40-2.33 (m, 2H), 2.08-1.98 (m, 1H), 1.75-1.63 (m, 1H), 1.43 (s, 9H).

LC/MS (method F, ESIpos): Rₜ=1.35 min, m/z=288 [M+H]⁺.

### Step 2: tert-Butyl ({1-[4-(N′-hydroxycarbamimidoyl)phenyl]cyclobutyl}oxy)acetate

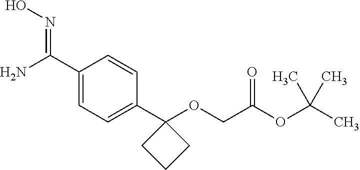

Analogously to the process described under Example 1A/step 5, 720 mg (2.51 mmol) of the compound from Example 57A/step 1 were reacted to give 584 mg (73% of th.) of the title compound.

¹H-NMR (400 MHz, CDCl₃, δ/ppm): 9.63 (s, 1H), 7.69 (d, 2H), 7.43 (d, 2H), 5.81 (s, broad, 2H), 3.57 (s, 2H), 2.39-2.32 (m, 4H), 1.94-1.85 (m, 1H), 1.63-1.52 (m, 1H), 1.34 (s, 9H).

LC/MS (method I, ESIpos): Rₜ=0.78 min, m/z=321 [M+H]⁺.

### Example 58A

### N′-Hydroxy-4-(tetrahydro-2H-pyran-4-yl)benzenecarboximide amide

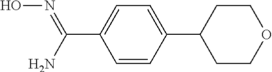

### Step 1: 4-(Tetrahydro-2H-pyran-4-yl)benzonitrile

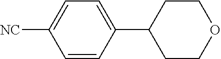

186 mg (0.594 mmol) of nickel(II) iodide, 90 mg (0.594 mmol) of trans-2-aminocyclohexanol hydrochloride and 3.63 g (19.8 mmol) of sodium hexamethyldisilazide were added to a solution of 2.91 g (19.8 mmol) of 4-cyanophenylboronic acid [M. Nishimura et al., Tetrahedron 2002, 58 (29), 5779-5788] in 20 ml of isopropanol. The suspension obtained in this way was stirred at RT under an argon atmosphere for 5 min. 2.1 g (9.90 mmol) of 4-iodotetrahydropyran [Heuberger et al., J. Chem. Soc. 1952, 910] were then added. After the reaction mixture had been stirred at a temperature of 75° C. for 15 h, it was cooled to RT and largely freed from inorganic salts with methylene chloride by filtration over approx. 50 g of silica gel. The crude product was purified by

MPLC (silica gel, mobile phase: methylene chloride). 986 mg (53% of th.) of the title compound were obtained in this way.

¹H-NMR (400 MHz, CDCl₃, δ/ppm): 7.60 (d, 2H), 7.32 (d, 2H), 4.12-4.07 (m, 2H), 3.56-3.50 (m, 2H), 2.87-2.79 (m, 1H), 1.86-1.73 (m, 4H).

GC/MS (method K, EIpos): Rₜ=5.97 min, m/z=187 [M]⁺.

### Step 2: N′-Hydroxy-4-(tetrahydro-2H-pyran-4-yl)benzenecarboximide amide

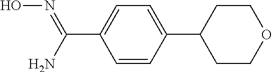

Analogously to the process described under Example 1A/step 5, 480 mg (2.56 mmol) of the compound from Example 58A/step 1 were reacted to give 525 mg (93% of th.) of the title compound.

¹H-NMR (400 MHz, CDCl₃, δ/ppm): 7.58 (d, 2H), 7.26 (d, 2H), 6.79 (broad, 1H), 4.82 (s, broad, 2H), 4.11-4.05 (m, 2H), 3.57-3.50 (m, 2H), 2.83-2.74 (m, 1H), 1.87-1.73 (m, 4H).

LC/MS (method D, ESIpos): Rₜ=0.92 min, m/z=221 [M+H]⁺.

### Example 59A

### N′-Hydroxy-4-isobutylbenzenecarboximide amide

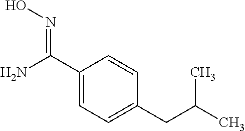

### Step 1: 4-Isobutylbenzonitrile

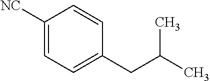

A mixture of 5.0 g (23.5 mmol) of 1-bromo-4-isobutylbenzene, 3.14 g (26.7 mmol) of zinc cyanide, 963 mg (2.35 mmol) of dicyclohexyl-(2′,6′-dimethoxybiphenyl-2-yl)phosphane and 1.08 g (1.17 mmol) of tris(dibenzylidene-acetone)dipalladium in 230 ml of DMF/water (99:1) was heated at 120° C. under inert, oxygen-free conditions for 1 h. After cooling to RT, the mixture was diluted with approx. 1,000 ml of water and extracted three times with approx. 150 ml of ethyl acetate each time. The combined organic extracts were washed successively with water and saturated sodium chloride solution. After drying over anhydrous magnesium sulphate, the mixture was filtered and the filtrate was freed from the solvent on a rotary evaporator. The residue obtained was purified by means of filtration with suction over silica gel with cyclohexane/ethyl acetate 10:1 as the mobile phase. 3.04 g (81% of th.) of the title compound were obtained.

¹H-NMR (400 MHz, CDCl₃, δ/ppm): 7.56 (d, 2H), 7.23 (d, 2H), 2.53 (d, 2H), 1.94-1.83 (m, 1H), 0.90 (d, 6H).

GC/MS (method K, EIpos): Rₜ=4.05 min, m/z=159 [M]⁺.

### Step 2: N′-Hydroxy-4-isobutylbenzenecarboximide amide

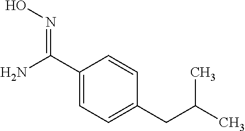

Analogously to the process described under Example 1A/step 5, 3.03 g (19.0 mmol) of the compound from Example 59A/step 1 were reacted to give 3.39 g (93% of th.) of the title compound.

¹H-NMR (400 MHz, DMSO-d₆, δ/ppm): 9.53 (s, 1H), 7.57 (d, 2H), 7.14 (d, 2H), 5.74 (broad, 2H), 2.46 (d, 2H), 1.89-1.79 (m, 1H), 0.87 (d, 6H).

LC/MS (method I, ESIpos): Rₜ=0.68 min, m/z=193 [M+H]⁺.

### Example 60A

### N′-Hydroxy-4-isopropylbenzenecarboximide amide

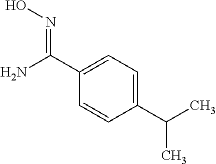

Analogously to the process described under Example 1A/step 5, 4.65 g (71% of th., purity of 94%) of the title compound were obtained from 5.0 g (34.4 mmol) of 4-isopropylbenzonitrile.

¹H-NMR (400 MHz, DMSO-d₆, δ/ppm): 9.53 (s, 1H), 7.58 (d, 2H), 7.23 (d, 2H), 5.74 (s, broad, 2H), 2.89 (sept, 1H), 1.20 (d, 6H).

LC/MS (method F, ESIpos): Rₜ=0.64 min, m/z=179 [M+H]⁺.

### Example 61A

### N′-Hydroxy-4-[1-(methoxymethyl)cyclobutyl]benzenecarboximide amide

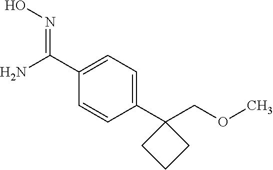

### Step 1: Ethyl 1-(4-bromophenyl)cyclobutanecarboxylate

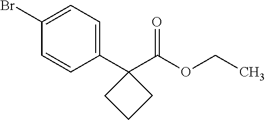

45 ml (45.2 mmol) of a 1 M solution of lithium hexamethyldisilazide in THF were added to a solution of 10.0 g (41.1 mmol) of 4-bromophenylacetic acid ethyl ester in 250 ml of anhydrous THF at 0° C. After 15 min, 5.4 ml (53.5 mmol) of 1,3-dibromopropane were added. The reaction mixture was allowed to warm to RT and was subsequently stirred at this temperature for 1 h. It was then cooled again to 0° C. and a further 45 ml (45.2 mmol) of lithium hexamethyldisilazide solution (1 M in THF) were added. Thereafter, the mixture was warmed again to RT. After 1 h, the reaction was ended by addition of approx. 10 ml of saturated aqueous ammonium chloride solution. The THF was largely removed on a rotary evaporator. The residue was diluted with water and extracted with ethyl acetate. The organic extract was washed successively with water and saturated sodium chloride solution. After drying over anhydrous magnesium sulphate, the mixture was filtered and the filtrate was freed from the solvent on a rotary evaporator. The crude product obtained in this way was coarsely purified by means of filtration with suction over approx. 300 g of silica gel with cyclohexane/ethyl acetate 3:1 as the mobile phase. 7.1 g (44% of th., purity of 73%) of the title compound were obtained, this being reacted further in this form.

¹H-NMR (400 MHz, CDCl₃, δ/ppm): 7.44 (d, 2H), 7.17 (d, 2H), 4.10 (quart, 2H), 2.85-2.79 (m, 2H), 2.49-2.41 (m, 2H), 2.10-1.98 (m, 1H), 1.91-1.81 (m, 1H), 1.18 (t, 3H).

MS (DCI, NH₃): m/z=300/302 [M+NH₄]⁺.

LC/MS (method D, ESIpos): Rₜ=2.70 min, m/z=283/285 [M+H]⁺.

### Step 2: [1-(4-Bromophenyl)cyclobutyl]methanol

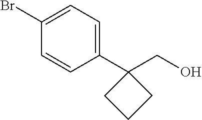

7.20 g (25.4 mmol) of the compound from Example 61A/step 1 were dissolved in 150 ml of anhydrous THF, and 25 ml (25 mmol) of a 1 M solution of lithium aluminium hydride in THF were added dropwise at 0° C. When the addition had ended, the ice/water bath was removed and stirring was continued at RT. After 1 h, the reaction was ended by—initially cautious—addition of approx. 450 ml of saturated aqueous ammonium chloride solution. The mixture was then extracted with ethyl acetate. After drying of the organic extract over anhydrous magnesium sulphate and subsequent filtration, the solvent was removed on a rotary evaporator. 6.04 g (88% of th., purity of 90%) of the title compound were obtained.

¹H-NMR (400 MHz, CDCl₃, δ/ppm): 7.43 (d, 2H), 7.02 (d, 2H), 3.72 (d, 2H), 2.33-2.20 (m, 4H), 2.13-2.01 (m, 1H), 1.93-1.83 (m, 1H).

MS (DCI, NH₃): m/z=258/260 [M+NH₄]⁺.

GC/MS (method K, ESIpos): Rₜ=5.77 min, m/z=240/242 [M]⁺.

### Step 3: 1-Bromo-4-[1-(methoxymethyl)cyclobutyl]benzene

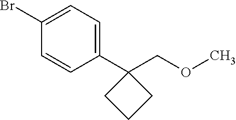

1.28 g (31.9 mmol) of a 60% strength suspension of sodium hydride in mineral oil were added to a solution of 7.0 g (29.0 mmol) of the compound from Example 61A/step 2 in 120 ml of anhydrous DMF at approx. 5° C. After the mixture had been stirred at this temperature for 1 h, 2.2 ml (34.8 mmol) of methyl iodide were added. The reaction mixture was allowed to warm to RT and stirring was continued for 15 h. The reaction mixture was then concentrated to a volume of approx. 20 ml on a rotary evaporator. Approx. 500 ml of water were added and the mixture was extracted three times with approx. 200 ml of diethyl ether each time. The combined organic extracts were washed with saturated sodium chloride solution and dried over anhydrous magnesium sulphate. After filtration and removal of the solvent on a rotary evaporator, the crude product obtained was purified by means of filtration with suction over approx. 200 g of silica gel with cyclohexane/ethyl acetate 50:1 as the mobile phase. 4.92 g (66% of th.) of the title compound were obtained.

¹H-NMR (400 MHz, CDCl₃, δ/ppm): 7.41 (d, 2H), 7.04 (d, 2H), 3.48 (s, 2H), 3.27 (s, 3H), 2.32-2.22 (m, 4H), 2.12-2.00 (m, 1H), 1.90-1.80 (m, 1H).

MS (DCI, NH₃): m/z=272/274 [M+NH₄]⁺.

GC/MS (method K, ESIpos): Rₜ=5.25 min, m/z=254/256 [M]⁺.

### Step 4: 4-[1-(Methoxymethyl)cyclobutyl]benzonitrile

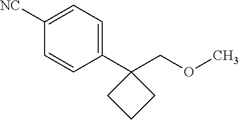

Analogously to the process described under Example 59A/step 1, 1.82 g (48% of th.) of the title compound were obtained from 4.80 g (18.8 mmol) of the compound from Example 61A/step 3.

¹H-NMR (400 MHz, CDCl₃, δ/ppm): 7.58 (d, 2H), 7.24 (d, 2H), 3.52 (s, 2H), 3.26 (s, 3H), 2.34-2.24 (m, 4H), 2.16-2.03 (m, 1H), 1.92-1.83 (m, 1H).

LC/MS (method F, ESIpos): Rₜ=1.22 min, m/z=202 [M+H]⁺.

### Step 5: N′-Hydroxy-4-[1-(methoxymethyl)cyclobutyl]benzenecarboximide amide

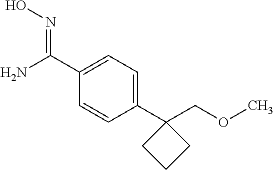

Analogously to the process described under Example 1A/step 5, 2.04 g (96% of th.) of the title compound were obtained from 1.82 g (9.04 mmol) of the compound from Example 61A/step 4.

¹H-NMR (400 MHz, CDCl₃, δ/ppm): 7.55 (d, 2H), 7.20 (d, 2H), 7.10 (broad, 1H), 4.83 (broad, 2H), 3.51 (s, 2H), 3.27 (s, 3H), 2.36-2.25 (m, 4H), 2.12-2.01 (m, 1H), 1.90-1.81 (m, 1H).

LC/MS (method I, ESIpos): Rₜ=0.61 min, m/z=235 [M+H]⁺.

### Example 62A

### N′-Hydroxy-4-(methoxymethyl)benzenecarboximide amide

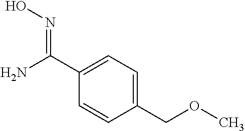

Analogously to the process described under Example 1A/step 5, 3.11 g (91% of th.) of the title compound were obtained from 2.80 g (19.0 mmol) of 4-(methoxymethyl)benzonitrile [H. Nakata et al., Org. Mass Spec. 1990, 25 (12), 649-654].

LC/MS (method D, ESIpos): Rₜ=0.77 min, m/z=181 [M+H]⁺.

### Example 63A

### 3-Fluoro-N′-hydroxy-4-methoxybenzenecarboximide amide

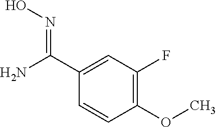

Analogously to the process described under Example 1A/step 5, 3.8 g (78% of th.) of the title compound were obtained from 4.0 g (26.5 mmol) of 3-fluoro-4-methoxybenzonitrile.

¹H-NMR (400 MHz, DMSO-d₆, δ/ppm): 9.60 (s, broad, 1H), 7.48 (d, 2H), 7.17 (t, 1H), 5.84 (s, broad, 2H), 3.86 (s, 3H).

LC/MS (method D, ESIpos): Rₜ=0.69 min, m/z=185 [M+H]⁺.

### Example 64A

### N′-Hydroxy-3-methyl-4-(tetrahydro-2H-pyran-4-yl)benzenecarboximide amide

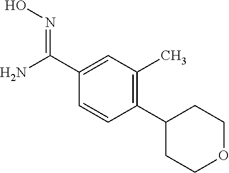

### Step 1: 3-Methyl-4-(tetrahydro-2H-pyran-4-yl)benzonitrile

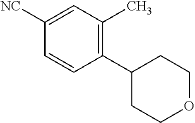

Analogously to the process described under Example 58A, step 1, 481 mg (18% of th.) of the title compound were obtained from 4.17 g (25.9 mmol) of 4-cyano-2-methylphenylboronic acid [D. Stones et al., Chem. Eur. J. 2004, 10 (1), 92-100] and 2.75 g (13.0 mmol) of 4-iodotetrahydropyran [Heuberger et al., J. Chem. Soc. 1952, 910].

¹H-NMR (400 MHz, CDCl₃, δ/ppm): 7.49 (dd, 1H), 7.43 (d, 1H), 7.31 (d, 1H), 4.12-4.09 (m, 2H), 3.59-3.52 (m, 2H), 3.05-2.97 (m, 1H), 2.39 (s, 3H), 1.86-1.75 (m, 2H), 1.69-1.64 (m, 2H).

GC/MS (method K, EIpos): Rₜ=6.31 min, m/z=201 [M]⁺.

### Step 2: N′-Hydroxy-3-methyl-4-(tetrahydro-2H-pyran-4-yl)benzenecarboximide amide

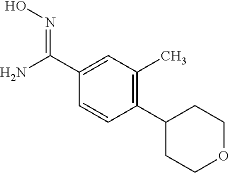

Analogously to the process described under Example 1A/step 5, 492 mg (84% of th.) of the title compound were obtained from 500 mg (2.48 mmol) of the compound from Example 64A/step 1.

¹H-NMR (400 MHz, DMSO-d₆, δ/ppm): 9.49 (s, 1H), 7.45 (d, 1H), 7.44 (s, 1H), 7.21 (d, 1H), 5.69 (s, broad, 2H), 3.97-3.93 (m, 2H), 3.50-3.43 (m, 2H), 3.00-2.92 (m, 1H), 2.33 (s, 3H), 1.72-1.57 (m, 4H).

LC/MS (method I, ESIpos): Rₜ=0.49 min, m/z=235 [M+H]⁺.

### Example 65A

### 4-[(Diisopropylamino)methyl]-N′-hydroxybenzenecarboximide amide

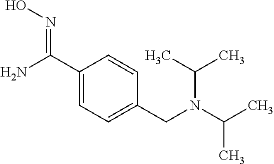

Step 1: 4-[(Diisopropylamino)methyl]benzonitrile

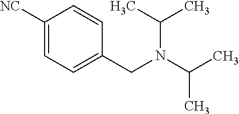

A mixture of 4.00 g (20.4 mmol) of 4-(bromomethyl)benzonitrile and 6.19 g (61.2 mmol) of diisopropylamine in 40 ml of toluene was heated in two portions at 150° C. in a microwave apparatus (CEM Discover, initial irradiation power 250 W) for in each case 3 h. After cooling to RT, the solid formed was filtered off and the filtrate was concentrated to obtain 4.52 g (92% of th., purity of 90%) of the title compound in this way.

LC/MS (method F, ESIpos): Rₜ=0.30 min, m/z=217 [M+H]⁺.

### Step 2: 4-[(Diisopropylamino)methyl]-N′-hydroxybenzenecarboximide amide

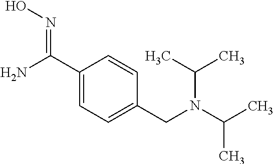

Analogously to the process described under Example 1A/step 5, 4.93 g (70% of th.) of the title compound were obtained from 6.80 g (28.3 mmol, purity of 90%) of the compound from Example 65A/step 1.

¹H-NMR (400 MHz, CDCl₃, δ/ppm): 7.52 (d, 2H), 7.41 (d, 2H), 4.84 (s, broad, 2H), 3.64 (s, 2H), 3.05-2.95 (m, 2H), 1.01 (d, 12H).

LC/MS (method I, ESIpos): Rₜ=0.18 min, m/z=250 [M+H]⁺.

### Example 66A

### 3-Chloro-N′-hydroxy-4-(trifluoromethoxy)benzenecarboximide amide

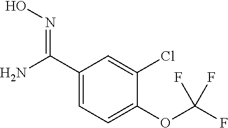

Analogously to the process described under Example 1A/step 5, 842 mg (73% of th.) of the title compound were obtained from 1.00 g (4.51 mmol) of 3-chloro-4-(trifluoromethoxy)benzonitrile.

¹H-NMR (400 MHz, CDCl₃, δ/ppm): 7.77 (d, 1H), 7.58-7.55 (dd, 1H), 7.37-7.33 (m, 1H), 4.82 (s, broad, 1H).

LC/MS (method D, ESIpos): Rₜ=1.64 min, m/z=255/257 [M+H]⁺.

### Example 67A

### N′-Hydroxy-4-[1-(trifluoromethyl)cyclopropyl]benzenecarboximide amide

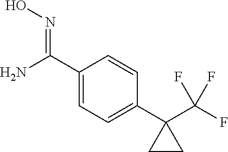

### Step 1: 1-Bromo-4-[1-(trifluoromethyl)cyclopropyl]benzene

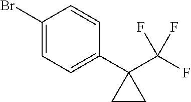

Activated zinc bromide on montmorillonite was first prepared as follows: 1.40 g (6.22 mmol) of zinc bromide were initially introduced into 56 ml of methanol, 5.64 g of montmorillonite K10 were added and the mixture was stirred at RT for 1 h. After removal of the methanol, the powder which remained was heated in a sand bath at a bath temperature of 200° C. for 1 h and then allowed to cool under argon.

The title compound was then prepared as follows: 10.0 g (53.7 mmol) of 1-phenyl-1-(trifluoromethyl)cyclopropane were initially introduced into 50 ml of pentane. 6.1 g (5.37 mmol) of the activated zinc bromide on montmorillonite obtained above were added and 27.7 ml (537 mmol) of bromine were then slowly added dropwise in the dark, while stirring. The mixture was then stirred further at RT in the dark overnight. 150 ml of a saturated aqueous sodium sulphite solution were subsequently slowly added dropwise, while cooling with ice, and the mixture was stirred at RT for a further approx. 30 min until it was decolorized. The solid was filtered off and rinsed twice with pentane. After separation of the filtrate phases, the aqueous phase was extracted twice with 200 ml of pentane each time. The combined organic phases were dried over sodium sulphate, filtered and concentrated under gentle conditions (significant volatility of the target compound). 17.1 g (>100% of th.) of the title compound were obtained in this manner, and according to ¹H-NMR still contained pentane.

¹H-NMR (400 MHz, CDCl₃, δ/ppm): 7.47 (d, 2H), 7.32 (s, 2H), 1.39-1.30 (m, 2H), 1.04-0.95 (m, 2H).

GC/MS (method K, ESIpos): Rₜ=3.45 min, m/z=264/266 [M+H]⁺.

### Step 2: 4-[1-(Trifluoromethyl)cyclopropyl]benzonitrile

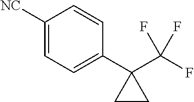

6.00 g (22.6 mmol) of the compound from Example 67A/step 1 were initially introduced into 30 ml of DMF under argon, 1.86 g (15.8 mmol) of zinc cyanide and 1.57 g (1.36 mmol) of tetrakis(triphenylphosphine)palladium(0) were added and the mixture was stirred at 80° C. overnight. After cooling to RT, a further 4.0 g (34.1 mmol) of zinc cyanide and 3.0 g (2.56 mmol) of tetrakis(triphenylphosphine)palladium(0) were added and the mixture was heated again at 120° C. for 5 h, while stirring. After cooling to RT, the solid present was filtered off and washed once with DMF. The filtrate, combined with the wash solution, was concentrated. The residue was taken up in 200 ml of ethyl acetate and the solution obtained was washed twice with 2 M aqueous ammonia solution and once with saturated aqueous sodium chloride solution. After drying over sodium sulphate, filtration and concentration, the residue obtained was purified by flash chromatography (silica gel, mobile phase: cyclohexane/ethyl acetate 40:1). After brief drying in vacuo, 3.46 g (72% of th.) of the title compound were obtained.

¹H-NMR (400 MHz, CDCl₃, δ/ppm): 7.66 (d, 2H), 7.58 (d, 2H), 1.47-1.41 (m, 2H), 1.09-1.03 (m, 2H).

GC/MS (method K, ESIpos): Rₜ=3.81 min, m/z=212 [M+H]⁺.

### Step 3: N′-Hydroxy-4-[1-(trifluoromethyl)cyclopropyl]benzenecarboximide amide

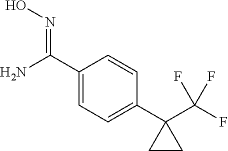

Analogously to the process described under Example 1A/step 5, 3.82 g (98% of th.) of the title compound were obtained from 3.40 g (16.1 mmol) of the compound from Example 67A/step 2.

¹H-NMR (400 MHz, CDCl₃, δ/ppm): 7.62 (d, 2H), 7.50 (d, 2H), 4.88 (s, broad, 2H), 1.42-1.36 (m, 2H), 1.06-1.00 (m, 2H).

LC/MS (method F, ESIpos): Rₜ=0.81 min, m/z=245 [M+H]⁺.

### Example 68A

### N′-Hydroxy-4-[N-methyl-S-(trifluoromethyl)sulphonimidoyl]benzenecarboximide amide (racemate)

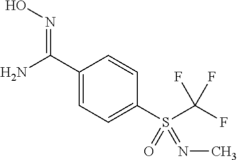

### Step 1: 4-[S-(Trifluoromethyl)sulphonimidoyl]benzonitrile (racemate)

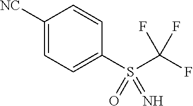

150 mg (0.66 mmol) of 1-fluoro-4-[S-(trifluoromethyl)sulphonimidoyl]benzene [N. V. Kondratenko, Zhurnal Organicheskoi Khimii 1986, 22 (8), 1716-1721; ibid. 1984, 20 (10), 2250-2252] were dissolved in 20 ml of DMSO, and 115 mg (0.83 mmol) of potassium carbonate, 140 mg (0.84 mmol) of potassium iodide and 130 mg (2.0 mmol) of potassium cyanide were added. The mixture was heated at 110° C. overnight, while stirring. After cooling to RT, approx. 10 ml of water were added to the mixture and the mixture was extracted with ethyl acetate. After concentration of the organic phase, the residue was purified by means of flash chromatography over silica gel. 50 mg (33% of th.) of the title compound were obtained.

### Step 2: 4-[N-Methyl-S-(trifluoromethyl)sulphonimidoyl]benzonitrile (racemate)

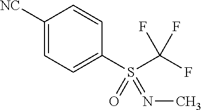

400 mg (1.60 mmol) of the compound from Example 68A/step 1 were dissolved in 8 ml of THF under argon and 224 mg (2.0 mmol) of potassium tert-butylate were added. The mixture was first stirred at RT for 1 h, 283 mg (2.0 mmol) of iodomethane were then added and the mixture was stirred further at RT overnight. Water was then added to the batch and the mixture was extracted with ethyl acetate. The organic extract was washed with saturated aqueous sodium chloride solution, dried over magnesium sulphate and concentrated. The residue was purified by means of flash chromatography over silica gel. 298 mg (70% of th.) of the title compound were obtained.

¹H-NMR (400 MHz, CDCl₃, δ/ppm): 8.22 (d, 2H), 7.90 (d, 2H), 3.10 (s, 3H).

LC/MS (method D, ESIpos): Rₜ=2.17 min, m/z=249 [M+H]⁺.

### Step 3: N′-Hydroxy-4-[N-methyl-S-(trifluoromethyl)sulphonimidoyl]benzenecarboximide amide (racemate)

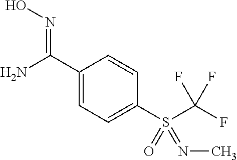

1.00 g (4.03 mmol) of the compound from Example 68A/step 2 were initially introduced into 20 ml of ethanol. 616 mg (8.86 mmol) of hydroxylamine hydrochloride and 1.2 ml (8.86 mmol) of triethylamine were added and the mixture was heated under reflux for 1 h. It was then concentrated and the residue was taken up in a mixture of ethyl acetate and water. The phases were separated and the aqueous phase was extracted once with ethyl acetate. The combined ethyl acetate phases were washed once with saturated aqueous sodium chloride solution, dried over magnesium sulphate, filtered and concentrated. The residue was purified by means of column chromatography (silica gel, mobile phase: cyclohexane/ethyl acetate 7:3). The combined product fractions were concentrated and the residue was stirred with pentane. The resulting solid was filtered off and dried in vacuo. 775 mg (66% of th.) of the title compound were obtained.

¹H-NMR (400 MHz, CDCl₃, δ/ppm): 8.12 (d, 2H), 8.04 (s, broad, 1H), 7.87 (d, 2H), 4.93 (s, 2H), 3.10 (s, 3H).

LC/MS (method I, ESIpos): Rₜ=0.76 min, m/z=282 [M+H]⁺.

### Example 69A

### N′-Hydroxy-4-[S-(trifluoromethyl)sulphonimidoyl]benzenecarboximide amide (racemate)

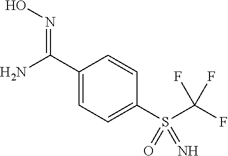

Analogously to the process described under Example 68A/step 3, 2.21 g (67% of th., purity of 90%) of the title compound were obtained from 2.60 g (11.1 mmol) of the compound from Example 68A/step 1.

¹H-NMR (400 MHz, CDCl₃, δ/ppm): 8.19 (d, 2H), 7.90 (d, 2H), 4.92 (s, broad, 2H), 3.69 (s, broad, 1H).

LC/MS (method I, ESIpos): Rₜ=0.51 min, m/z=268 [M+H]⁺.

### Example 70A

### 3-Fluoro-N′-hydroxy-4-(trifluoromethoxy)benzenecarboximide amide

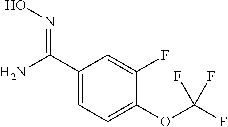

Analogously to the process described under Example 1A/step 5, 5.7 g (99% of th.) of the title compound were obtained from 5.0 g (23.9 mmol) of 3-fluoro-4-(trifluoromethoxy)benzonitrile.

¹H-NMR (400 MHz, CDCl₃, δ/ppm): 7.53-7.49 (dd, 1H), 7.45-7.41 (m, 1H), 7.37-7.31 (t, 1H), 4.87 (s, broad, 2H).

LC/MS (method I, ESIpos): Rₜ=0.74 min, m/z=239 [M+H]⁺.

### Example 71A

### Ethyl 4-[4-(N′-hydroxycarbamimidoyl)phenyl]tetrahydro-2H-pyran-4-carboxylate

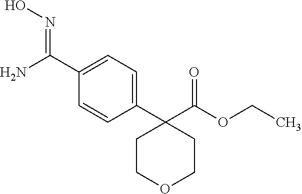

### Step 1: Ethyl 4-(4-bromophenyl)tetrahydro-2H-pyran-4-carboxylate

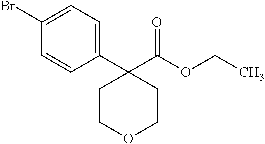

6.0 g (24.7 mmol) of ethyl 4-bromophenylacetate were dissolved in 120 ml of abs. DMF under argon, 1.48 g (37.0 mmol, 60% strength) of sodium hydride were added, while cooling in an ice bath, and the mixture was stirred for 30 min. 5.72 g (24.7 mmol) of bis(2-bromoethyl)ether were then added, while constantly cooling in an ice bath, and the mixture was stirred at approx. 0° C. for 1 h. After renewed addition of 1.48 g of 60% strength sodium hydride, the mixture was stirred again for 1 h, while cooling in an ice bath. Saturated aqueous ammonium chloride was then added and the mixture was extracted with ethyl acetate. The organic phase was washed with water and with saturated sodium chloride solution, dried over magnesium sulphate, filtered and concentrated on a rotary evaporator. The residue was purified by column chromatography over silica gel (mobile phase: cyclohexane/ethyl acetate 10:1). 2.62 g (33% of th.) of the title compound were obtained.

¹H-NMR (400 MHz, CDCl₃, δ/ppm): 7.47 (d, 2H), 7.25 (d, 2H), 4.14 (q, 2H), 3.93 (dt, 2H), 3.56 (td, 2H), 2.59 (dd, 2H), 1.93 (m, 2H), 1.19 (t, 3H).

MS (DCI, NH₃): m/z=329/331 [M+NH₄]⁺.

LC/MS (method F, ESIpos): Rₜ=1.33 min, no ionization.

### Step 2: Ethyl 4-(4-cyanophenyl)tetrahydro-2H-pyran-4-carboxylate

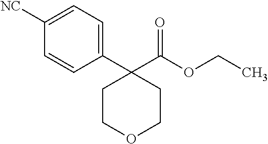

0.50 g (1.60 mmol) of the compound from Example 71A/step 1 were initially introduced into 2.5 ml of degassed DMF under argon, 112 mg (0.96 mmol) of zinc cyanide and 110 mg (0.09 mmol) of tetrakis(triphenylphosphine)palladium(0) were added and the mixture was stirred at 100° C. in a microwave oven for 1 h. After cooling to RT, the solid was filtered off and the filtrate was purified directly by means of preparative HPLC (method P). 250 mg (60% of th.) of the title compound were obtained.

¹H-NMR (400 MHz, DMSO-d₆, δ/ppm): 7.84 (d, 2H), 7.58 (d, 2H), 4.10 (q, 2H), 3.81 (m, 2H), 3.44 (t, 2H), 2.39 (d, 2H), 1.90 (m, 2H), 1.11 (t, 3H).

GC/MS (method K, EIpos): Rₜ=7.00 min, m/z=215 [M—OCH₂CH₃]⁺.

### Step 3: Ethyl 4-[4-(N′-hydroxycarbamimidoyl)phenyl]tetrahydro-2H-pyran-4-carboxylate

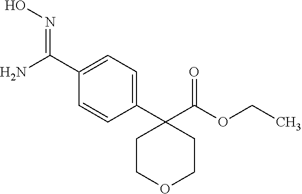

A mixture of 240 mg (0.93 mmol) of the compound from Example 71A/step 2, 141 mg (2.04 mmol) of hydroxylamine hydrochloride and 0.28 ml (2.04 mmol) of triethylamine in 4.5 ml of ethanol was stirred at 60° C. for 2 h. After cooling to RT, the solvent was removed virtually completely on a rotary evaporator. The residue was then suspended in 20 ml of water under ultrasound irradiation. The white solid was filtered off, washed with a little water and dried under a high vacuum. 245 mg (91% of th.) of the title compound were obtained in this way.

LC/MS (method I, ESIpos): Rₜ=0.56 min, m/z=293 [M+H]⁺.

### Example 72A

### 4-[4-(N′-Hydroxycarbamimidoyl)phenyl]-N,N-dimethyl-tetrahydro-2H-pyran-4-carboxamide

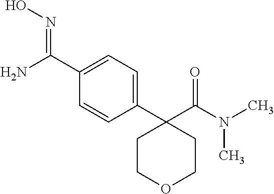

### Step 1: 4-(4-Bromophenyl)tetrahydro-2H-pyran-4-carboxylic acid

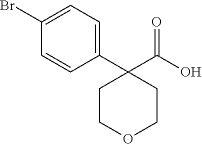

1.3 g (4.15 mmol) of the compound from Example 71A/step 1 were dissolved in 45 ml of dioxane, 9.1 ml of 1 N sodium hydroxide solution were added and the mixture was stirred under reflux. After 18 h, a further 8.3 ml of 1 N sodium hydroxide solution were added and the mixture was stirred under reflux for a further 24 h. After cooling, approx. 19 ml of 1 N hydrochloric acid were added and the mixture was stirred at RT for 15 min. The precipitate formed was filtered off, washed with water and dried in vacuo. 1.22 g (99% of th.) of the title compound were obtained.

¹H-NMR (400 MHz, DMSO-d₆, δ/ppm): 7.55 (d, 2H), 7.34 (d, 2H), 3.79 (m, 2H), 3.45 (t, 2H), 2.34 (d, 2H), 1.79 (m, 2H).

### Step 2: 4-(4-Bromophenyl)tetrahydro-2H-pyran-4-carboxylic acid chloride

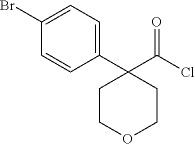

1.34 g (4.70 mmol) of the compound from Example 72A/step 1 were stirred in 6.5 ml of thionyl chloride under reflux for 2 h. The batch was then concentrated on a rotary evaporator, the residue was taken up in toluene and the mixture was concentrated again. The resulting residue was then stirred in a mixture of methylene chloride and pentane (1:2), the solid which remained was filtered off and the filtrate was freed from the solvent. The filtrate residue obtained was dried in vacuo. 1.49 g (>100% of th.) of the target compound were isolated, this being employed in subsequent stages without further purification.

### Step 3: 4-(4-Bromophenyl)-N,N-dimethyl-tetrahydro-2H-pyran-4-carboxamide

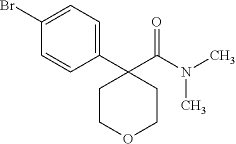

3.29 ml (6.59 mmol) of dimethylamine were added dropwise to a solution of 1.0 g (3.29 mmol) of the compound from Example 72A/step 2 in 33 ml of methylene chloride, while cooling in an ice bath, and the mixture was subsequently stirred at RT for 1 h. The mixture was then freed from the solvent on a rotary evaporator. The residue was suspended in 50 ml of 1 N sodium hydroxide solution under ultrasound treatment and the suspension was then filtered. The filter cake was washed with water and dried in vacuo. 820 mg (90% of th.) of the title compound were obtained.

¹H-NMR (400 MHz, DMSO-d₆, δ/ppm): 7.56 (d, 2H), 7.19 (d, 2H), 3.74 (dt, 2H), 3.58 (t, 2H), 2.52 (s, 6H, hidden under DMSO signal), 2.16 (d, 2H), 1.87 (m, 2H).

LC/MS (method F, ESIpos): Rₜ=1.07 min, m/z=312/314 [M+H]⁺.

### Step 4: 4-(4-Cyanophenyl)-N,N-dimethyl-tetrahydro-2H-pyran-4-carboxamide

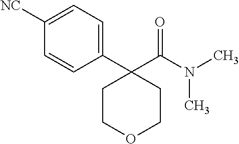

0.40 g (1.28 mmol) of the compound obtained in Example 72A/step 3 were initially introduced into 2.0 ml of degassed DMF under argon, 90 mg (0.77 mmol) of zinc cyanide and 89 mg (0.08 mmol) of tetrakis(triphenylphosphine)palladium(0) were added and the mixture was stirred at 110° C. in a microwave oven for 1 h. After cooling to RT, the solid was filtered off and the filtrate was purified directly by means of preparative HPLC (method P). 230 mg (68% of th.) of the title compound were obtained.

¹H-NMR (400 MHz, DMSO-d₆, δ/ppm): 7.85 (d, 2H), 7.44 (d, 2H), 3.76 (dt, 2H), 3.59 (t, 2H), 2.52

(s, 6H, hidden under DMSO signal), 2.17 (d, 2H), 1.91 (m, 2H).

LC/MS (method I, ESIpos): Rₜ=0.75 min, m/z=259 [M+H]⁺.

### Step 5: 4-[4-(N′-Hydroxycarbamimidoyl)phenyl]-N,N-dimethyl-tetrahydro-2H-pyran-4-carboxamide

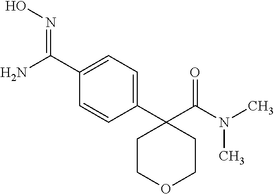

A mixture of 333 mg (1.28 mmol) of the compound from Example 72A/step 4, 186 mg (2.68 mmol) of hydroxylamine hydrochloride and 0.37 ml (2.68 mmol) of triethylamine in 6.2 ml of ethanol was stirred at 80° C. for 2 h. After cooling, the precipitate formed was filtered off, washed with a little ethanol and dried under a high vacuum. 180 mg (47% of th.) of the title compound were obtained.

LC/MS (method F, ESIpos): Rₜ=0.27 min, m/z=291 [M+H]⁺.

### Example 73A

### 4-[4-(N′-Hydroxycarbamimidoyl)phenyl]-N-methyl-tetrahydro-2H-pyran-4-carboxamide

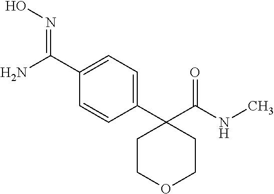

### Step 1: 4-(4-Bromophenyl)-N-methyl-tetrahydro-2H-pyran-4-carboxamide

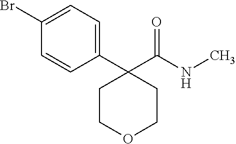

Analogously to the process described under Example 72A/step 3, 1.0 g (3.29 mmol) of the compound from Example 72A/step 2 and 3.29 ml (6.58 mmol) of a 2 M solution of methylamine in THF were reacted to give 680 mg (69% of th.) of the title compound.

¹H-NMR (400 MHz, DMSO-d₆, δ/ppm): 7.62 (q, 1H), 7.52 (d, 1H), 7.28 (d, 2H), 3.71 (m, 2H), 3.43 (t, 2H), 2.54 (d, 3H), 2.39 (d, 2H), 1.81 (m, 2H).

LC/MS (method I, ESIpos): Rₜ=0.82 min, m/z=297/299 [M+H]⁺.

### Step 2: 4-(4-Cyanophenyl)-N-methyl-tetrahydro-2H-pyran-4-carboxamide

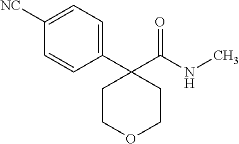

Analogously to the process described under Example 72A/step 4, 660 mg (2.21 mmol) of the compound from Example 73A/step 1 were reacted to give 390 mg (72% of th.) of the title compound.

¹H-NMR (400 MHz, DMSO-d₆, δ/ppm): 7.80 (d, 2H), 7.66 (d, 1H), 7.53 (d, 2H), 3.73 (d, 2H), 3.45 (t, 2H), 2.55 (d, 3H), 2.41 (d, 2H), 1.85 (m, 2H).

LC/MS (method I, ESIpos): Rₜ=0.63 min, m/z=245 [M+H]⁺.

### Step 3: 4-[4-(N′-Hydroxycarbamimidoyl)phenyl]-N-methyl-tetrahydro-2H-pyran-4-carboxamide

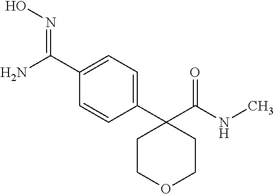

Analogously to the process described under Example 72A/step 5, 380 mg (0.16 mmol) of the compound from Example 73A/step 2 were reacted to give 360 mg (83% of th.) of the title compound.

¹H-NMR (400 MHz, DMSO-d₆, δ/ppm): 9.57 (s, 1H), 7.62 (d, 2H), 7.59 (m, 1H), 7.33 (d, 2H), 5.76 (s, 2H), 3.71 (m, 2H), 3.45 (t, 2H), 2.54 (d, 3H), 2.41 (d, 2H), 1.84 (m, 2H).

LC/MS (method I, ESIpos): Rₜ=0.53 min, m/z=253 [M+H]⁺.

### Example 74A

### 5-(5-Methyl-1H-pyrazol-3-yl)-3-[4-(tetrahydro-2H-pyran-4-yl)phenyl]-1,2,4-oxadiazole

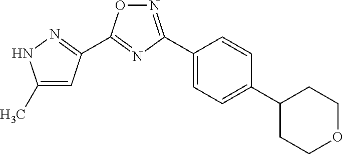

Analogously to the process described under Example 23A, from 469 mg (3.72 mmol) of 5-methyl-1H-pyrazole-3-carboxylic acid and 820 mg (3.72 mmol) of the compound from Example 58A, 450 mg of the title compound were obtained after extraction of the crude product by stirring in acetonitrile, and a further 97 mg of the title compound were obtained after purification of the mother liquor by preparative HPLC (method M) (yield 47% of th. in total).

¹H-NMR (400 MHz, DMSO-d₆, δ/ppm): 13.52 (s, 1H), 8.01 (d, 2H), 7.49 (d, 2H), 6.79 (s, 1H), 3.99-3.95 (m, 2H), 3.49-3.42 (m, 2H), 2.92-2.84 (m, 1H), 2.34 (s, 3H), 1.77-1.65 (m, 4H).

LC/MS (method I, ESIpos): Rₜ=0.98 min, m/z=311 [M+H]⁺.

### Example 75A

### 5-(5-Methyl-1H-pyrazol-3-yl)-3-[3-methyl-4-(tetrahydro-2H-pyran-4-yl)phenyl]-1,2,4-oxadiazole

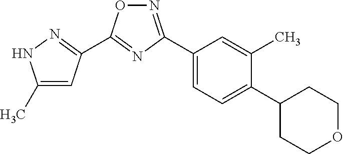

Analogously to the process described under Example 23A, 180 mg (1.43 mmol) of 5-methyl-1H-pyrazole-3-carboxylic acid and 335 mg (1.43 mmol) of the compound from Example 64A were reacted to give 189 mg (39% of th.) of the title compound. The reaction mixture was stirred here first at RT for 16 h and then at 140° C. for 30 min. The purification of the product was carried out by means of preparative HPLC (method M).

¹H-NMR (400 MHz, CDCl₃, δ/ppm): 10.63 (broad, 1H), 8.00 (d, 1H), 7.99 (s, 1H), 7.36 (d, 1H), 6.80 (s, 1H), 4.13-4.10 (m, 2H), 3.61-3.54 (m, 2H), 3.07-3.00 (m, 1H), 2.45 (s, 3H), 2.43 (s, 3H), 1.92-1.80 (m, 2H), 1.73-1.68 (m, 2H).

LC/MS (method I, ESIpos): Rₜ=1.02 min, m/z=325 [M+H]⁺.

### Example 76A

### 3-(4-Isobutylphenyl)-5-(5-methyl-1H-pyrazol-3-yl)-1,2,4-oxadiazole

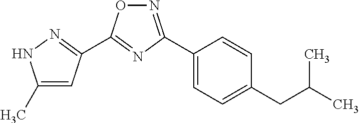

3.19 g (16.7 mmol) of EDC, 2.55 g (16.7 mmol) of HOBt and 3.35 g (17.4 mmol) of the compound from Example 59A were added successively to a solution of 2.0 g (15.9 mmol) of 5-methyl-1H-pyrazole-3-carboxylic acid in 80 ml of anhydrous DMF. The mixture was stirred at RT for 1 h, before it was heated at 140° C. for 30 min. After cooling to RT, the solvent was mostly stripped off on a rotary evaporator. Approx. 500 ml of water were added to the residue and the mixture was extracted three times with approx. 200 ml of diethyl ether each time. The combined organic extracts were washed successively with water and saturated sodium chloride solution. After drying over anhydrous magnesium sulphate, the mixture was filtered and the filtrate was concentrated on a rotary evaporator. The residue obtained was purified by means of MPLC (silica gel, mobile phase: cyclohexane/ethyl acetate 2:1). After final stirring with approx. 50 ml of pentane, 1.7 g (38% of th.) of the title compound were obtained.

¹H-NMR (400 MHz, CDCl₃, δ/ppm): 10.84 (broad, 1H), 8.08 (d, 2H), 7.27 (d, 2H), 6.81 (s, 1H), 2.54 (d, 2H), 2.44 (s, 3H), 1.97-1.87 (m, 1H), 0.93 (d, 6H).

LC/MS (method Q, ESIpos): Rₜ=2.59 min, m/z=283 [M+H]⁺.

### Example 77A

### 3-{4-[1-(Methoxymethyl)cyclobutyl]phenyl}-5-(5-methyl-1H-pyrazol-3-yl)-1,2,4-oxadiazole

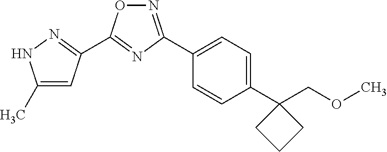

Analogously to the process described under Example 76A, 1.08 g (8.52 mmol) of 5-methyl-1H-pyrazole-3-carboxylic acid and 2.0 g (8.52 mmol) of the compound from Example 61A were reacted to give 1.87 g (46% of th.) of the title compound. For the purification of the crude product by MPLC, a mobile phase gradient of cyclohexane/ethyl acetate (5:1→1:1) was used.

¹H-NMR (400 MHz, CDCl₃, δ/ppm): 11.57 (broad, 1H), 8.10 (d, 2H), 7.30 (d, 2H), 6.81 (s, 1H), 3.57 (s, 2H), 3.29 (s, 3H), 2.45 (s, 3H), 2.41-2.28 (m, 4H), 2.15-2.03 (m, 1H), 1.93-1.84 (m, 1H).

LC/MS (method F, ESIpos): Rₜ=1.28 min, m/z=325 [M+H]⁺.

### Example 78A

### 3-[4-(Methoxymethyl)phenyl]-5-(5-methyl-1H-pyrazol-3-yl)-1,2,4-oxadiazole

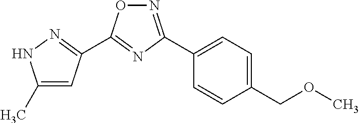

Analogously to the process described under Example 76A, 1.50 g (11.9 mmol) of 5-methyl-1H-pyrazole-3-carboxylic acid and 2.36 g (13.1 mmol) of the compound from Example 62A were reacted to give 346 mg (11% of th.) of the title compound. For the purification of the crude product by MPLC, cyclohexane/ethyl acetate 2:1 was used as the mobile phase.

LC/MS (method I, ESIpos): Rₜ=0.95 min, m/z=271 [M+H]⁺.

### Example 79A

### 3-(3-Fluoro-4-methoxyphenyl)-5-(5-methyl-1H-pyrazol-3-yl)-1,2,4-oxadiazole

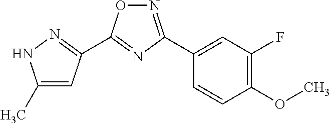

Analogously to the process described under Example 76A, 1.0 g (7.93 mmol) of 5-methyl-1H-pyrazole-3-carboxylic acid and 1.61 g (8.72 mmol) of the compound from Example 63A were reacted to give 559 mg (26% of th.) of the title compound.

¹H-NMR (400 MHz, CDCl₃, δ/ppm): 10.81 (broad, 1H), 7.96-7.89 (m, 2H), 7.06 (t, 1H), 6.80 (s, 1H), 3.97 (s, 3H), 2.47 (s, 3H).

LC/MS (method I, ESIpos): Rₜ=0.96 min, m/z=275 [M+H]⁺.

### Example 80A

### 3-(4-Methoxyphenyl)-5-(5-methyl-1H-pyrazol-3-yl)-1,2,4-oxadiazole

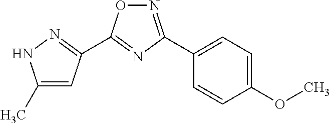

Analogously to the process described under Example 76A, 1.50 g (11.9 mmol) of 5-methyl-1H-pyrazole-3-carboxylic acid and 2.17 g (13.1 mmol) of N′-hydroxy-4-methoxybenzenecarboximide amide [A. Renodon-Corniere et al., J. Med. Chem. 2002, 45 (4), 944-954] were reacted to give 1.71 g (56% of th.) of the title compound.

¹H-NMR (400 MHz, CDCl₃, δ/ppm): 8.11 (d, 2H), 7.00 (d, 2H), 6.80 (s, 1H), 3.88 (s, 3H), 2.47 (s, 3H).

LC/MS (method I, ESIpos): Rₜ=0.94 min, m/z=257 [M+H]⁺.

### Example 81A

### 3-(4-Isopropylphenyl)-5-(5-methyl-1H-pyrazol-3-yl)-1,2,4-oxadiazole

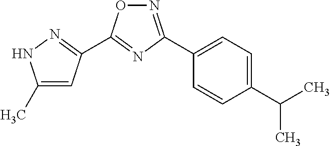

Analogously to the process described under Example 76A, 2.0 g (15.9 mmol) of 5-methyl-1H-pyrazole-3-carboxylic acid and 3.11 g (17.4 mmol) of the compound from Example 60A were reacted to give 2.20 g (52% of th.) of the title compound.

¹H-NMR (400 MHz, CDCl₃, δ/ppm): 8.10 (d, 2H), 7.36 (d, 2H), 6.81 (s, 1H), 2.97 (sept, 1H), 2.43 (s, 3H), 1.29 (d, 6H).

LC/MS (method Q, ESIpos): Rₜ=2.42 min, m/z=269 [M+H]⁺.

### Example 82A

### 3-(4-tert-Butylphenyl)-5-(5-methyl-1H-pyrazol-3-yl)-1,2,4-oxadiazole

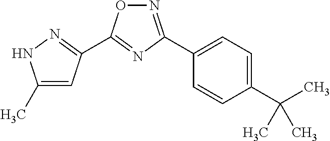

Analogously to the process described under Example 76A, 2.50 g (19.8 mmol) of 5-methyl-1H-pyrazole-3-carboxylic acid and 4.19 g (21.8 mmol) of 4-tert-butyl-N′-hydroxy-benzenecarboximide amide were reacted to give 2.60 g (46% of th.) of the title compound.

¹H-NMR (400 MHz, CDCl₃, δ/ppm): 11.08 (s, broad, 1H), 8.10 (d, 2H), 7.51 (d, 2H), 6.81 (s, 1H), 2.46 (s, 3H), 1.37 (s, 9H).

LC/MS (method I, ESIpos): Rₜ=1.21 min, m/z=283 [M+H]⁺.

### Example 83A

### N-Isopropyl-N-{4-[5-(5-methyl-1H-pyrazol-3-yl)-1,2,4-oxadiazol-3-yl]benzyl}propan-2-amine

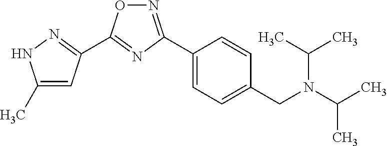

Analogously to the process described under Example 23A, 2.00 g (15.9 mmol) of 5-methyl-1H-pyrazole-3-carboxylic acid and 3.95 g (15.9 mmol) of the compound from Example 65A were reacted to give 1.49 g (26% of th., purity of 93%) of the title compound.

¹H-NMR (400 MHz, CDCl₃, δ/ppm): 11.50 (s, broad, 1H), 8.08 (d, 2H), 7.51 (d, 2H), 6.81 (s, 1H), 3.70 (s, 2H), 3.10-2.98 (m, 2H), 2.42 (s, 3H), 1.02 (d, 12H).

LC/MS (method F, ESIpos): Rₜ=0.73 min, m/z=340 [M+H]⁺.

### Example 84A

### 3-[3-Chloro-4-(trifluoromethoxy)phenyl]-5-(5-methyl-1H-pyrazol-3-yl)-1,2,4-oxadiazole

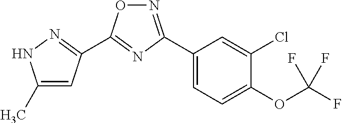

Analogously to the process described under Example 23A, 631 mg (5.00 mmol) of 5-methyl-1H-pyrazole-3-carboxylic acid and 1.27 g (5.00 mmol) of the compound from Example 66A were reacted to give 1.08 g (60% of th., purity of 95%) of the title compound. The reaction times here were approx. 30 min at RT and approx. 1 h at 150° C. The product was obtained by a procedure in which after the reaction had ended the solid which had precipitated out after addition of water was filtered off, washed with water and dried in vacuo.

LC/MS (method I, ESIpos): Rₜ=1.20 min, m/z=345/347 [M+H]⁺.

### Example 85A

### 3-[3-Fluoro-4-(trifluoromethoxy)phenyl]-5-(5-methyl-1H-pyrazol-3-yl)-1,2,4-oxadiazole

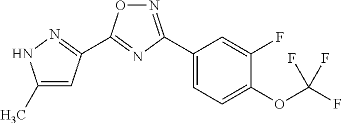

Analogously to the process described under Example 23A, 2.0 g (15.9 mmol) of 5-methyl-1H-pyrazole-3-carboxylic acid and 3.78 g (15.9 mmol) of the compound from Example 70A were reacted to give 3.15 g (56% of th., purity of 92%) of the title compound. In this case the product was obtained not via purification by chromatography but by washing the crude product with water and pentane and subsequent drying in vacuo.

¹H-NMR (400 MHz, CDCl₃, δ/ppm): 12.0-9.5 (s, broad, 1H), 8.10-7.97 (m, 2H), 7.46-7.41 (t, 1H), 6.81 (s, 1H), 2.47 (s, 3H).

LC/MS (method I, ESIpos): Rₜ=1.16 min, m/z=329 [M+H]⁺.

### Example 86A

### 5-(5-Methyl-1H-pyrazol-3-yl)-3-{4-[1-(trifluoromethyl)cyclopropyl]phenyl}-1,2,4-oxadiazole

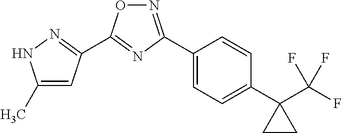

Analogously to the process described under Example 23A, 1.19 g (9.42 mmol) of 5-methyl-1H-pyrazole-3-carboxylic acid and 2.30 g (9.42 mmol) of the compound from Example 67A were reacted to give 1.05 g (62% of th.) of the title compound. The purification of the crude product was carried out via preparative HPLC (method N).

¹H-NMR (400 MHz, CDCl₃, δ/ppm): 11.0-10.5 (s, broad, 1H), 8.16 (d, 2H), 7.60 (d, 2H), 6.82 (s, 1H), 1.43-1.39 (m, 2H), 1.12-1.08 (m, 2H).

LC/MS (method I, ESIpos): Rₜ=1.17 min, m/z=335 [M+H]⁺.

### Example 87A

### 2-Bromo-4-(bromomethyl)pyridine

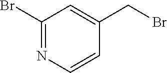

Analogously to the process described under Example 42A/step 3, 1.83 g (95% of th.) of the title compound were prepared from 1.50 g (7.66 mmol) of 2-bromo-4-(hydroxymethyl)pyridine.

¹H-NMR (400 MHz, CDCl₃, δ/ppm): 8.36 (d, 1H), 7.52 (s, 1H), 7.27 (d, 1H), 4.32 (s, 2H).

HPLC (method A): Rₜ=3.47 min.

MS (DCI, NH₃): m/z=250/252/254 [M+H]⁺.

### Example 88A

### [2-(4-Hydroxytetrahydro-2H-pyran-4-yl)pyridin-4-yl]methyl methanesulphonate

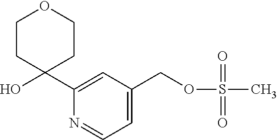

### Step 1: 2-Bromo-4-({[tert-butyl(dimethyl)silyl]oxy}methyl)pyridine

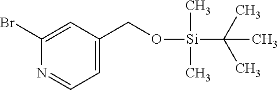

4.65 g (24.7 mmol) of 2-bromo-4-(hydroxymethyl)pyridine and 3.91 g (260 mmol) of tert-butyldimethylsilyl chloride were initially introduced into 46 ml of methylene chloride, 2.02 g (29.7 mmol) of imidazole were added, while cooling in an ice bath, and the mixture was stirred at RT for 2 h. The precipitate formed was subsequently filtered off and the filtrate was washed successively with water, 1 N sodium hydroxide solution, water again and saturated sodium chloride solution. The organic phase was dried over magnesium sulphate and filtered and the solvent was removed on a rotary evaporator. After the residue had been dried in vacuo, 6.92 g (93% of th.) of the title compound were obtained.

¹H-NMR (400 MHz, DMSO-d₆, δ/ppm): 8.34 (d, 1H), 7.53 (s, 1H), 7.36 (d, 1H), 4.77 (s, 2H), 0.92 (s, 9H), 0.10 (s, 6H).

LC/MS (method I, ESIpos): Rₜ=1.40 min, m/z=302/304 [M+H]⁺.

### Step 2: 4-[4-({[tert-Butyl(dimethyl)silyl]oxy}methyl)pyridin-2-yl]tetrahydro-2H-pyran-4-ol

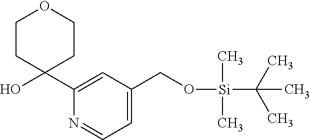

500 mg (1.65 mmol) of the compound from Example 88A/step 1 were dissolved in 16 ml of absolute THF under argon, and 1.14 ml (1.82 mmol) of a 1.6 M solution of n-butyllithium in THF were added dropwise at −78° C. The mixture was stirred for 20 min while cooling with dry ice. A solution of 182 mg (1.82 mmol) of tetrahydro-4H-pyran-4-one in 2.0 ml of THF was then added at −78° C. and the mixture was subsequently stirred at this temperature for 30 min. Saturated aqueous ammonium chloride was subsequently added and the mixture was extracted with ethyl acetate. The organic phase was washed with water and saturated sodium chloride solution, dried over magnesium sulphate and filtered. After removal of the solvent on a rotary evaporator, the residue was purified by means of preparative HPLC (method P). 110 mg (17% of th.) of the title compound were obtained.

LC/MS (method I, ESIpos): Rₜ=1.00 min, m/z=324 [M+H]⁺.

### Step 3: 4-[4-(Hydroxymethyl)pyridin-2-yl]tetrahydro-2H-pyran-4-ol

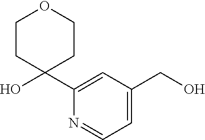

0.65 ml (0.65 mmol) of a 1 M solution of tetra-n-butylammonium fluoride in THF were added to a solution of 105 mg (0.33 mmol) of the compound from Example 88A/step 2 in 6.5 ml of THF and the mixture was stirred at RT for 30 min. The batch was subsequently concentrated on a rotary evaporator, the residue was taken up in ethyl acetate and the mixture was washed successively with 1 N sodium hydroxide solution, water and saturated sodium chloride solution. The organic phase was dried over sodium sulphate and filtered and the solvent was removed on a rotary evaporator.

After drying of the residue in vacuo, 45 mg (33% of th., purity of approx. 50%) of the title compound were obtained, this being employed in the subsequent stage in this form.

LC/MS (method F, ESIpos): Rₜ=0.21 min, m/z=210 [M+H]⁺.

### Step 4: [2-(4-Hydroxytetrahydro-2H-pyran-4-yl)pyridin-4-yl]methyl methanesulphonate

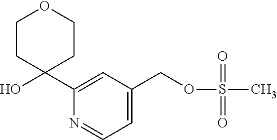

40 mg (approx. 0.1 mmol) of the compound from Example 88A/step 3 were dissolved in 1.9 ml of methylene chloride, 16 μl (0.21 mmol) of methanesulphonic acid chloride and 29 μl (0.21 mmol) of triethylamine were added and the mixture was stirred at RT for 1 h. Thereafter, it was diluted with ethyl acetate and the solution was washed successively with water and saturated sodium chloride solution. The organic phase was dried over magnesium sulphate and filtered and the solvent was removed on a rotary evaporator. After drying of the residue in vacuo, 44 mg of the title compound in the still contaminated form was obtained; this product was used further without further purification.

MS (DCI, NH₃): m/z=288 [M+H]⁺.

### Example 89A

### [2-(2-Hydroxypropan-2-yl)pyridin-4-yl]methyl methanesulphonate

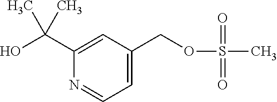

### Step 1: 2-[4-({[tert-Butyl(dimethyl)silyl]oxy}methyl)pyridin-2-yl]propan-2-ol

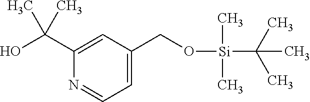

Analogously to the process described under Example 88A/step 2, 1.0 g (3.3 mmol) of the compound from Example 88A/step 1 and 0.29 ml (3.97 mmol) of acetone were reacted to give 0.95 g (88% of th., purity of 87%) of the title compound. The acetone was employed here in the pure form, without the addition of THF as a solvent. The product obtained was further processed in the subsequent stage as such, without further purification by HPLC.

LC/MS (method D, ESIpos): Rₜ=1.82 min, m/z=281 [M+H]⁺.

### Step 2: 2-[4-(Hydroxymethyl)pyridin-2-yl]propan-2-ol

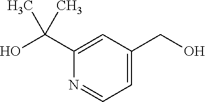

6.75 ml (6.75 mmol) of a 1 M solution of tetra-n-butylammonium fluoride in THF were added to a solution of 0.95 g (3.37 mmol) of the compound from Example 89A/step 1 in 68 ml of THF and the mixture was stirred at RT for 1 h. 4.2 g of the ion exchanger Dowex 50WX8-400 and 1.4 g (14.0 mmol) of calcium carbonate were then added to the batch and the mixture was stirred at RT for 1 h. The solid was filtered off and the filtrate was concentrated. A two-phase residue was obtained, the upper phase of which was separated off and discarded. The lower phase was diluted with ethyl acetate and extracted with water. The aqueous phase was concentrated and the residue was purified by means of preparative HPLC (method P). 166 mg (29% of th.) of the title compound were obtained.

¹H-NMR (400 MHz, DMSO-d₆, δ/ppm): 8.39 (d, 1H), 7.62 (s, 1H), 7.14 (d, 1H), 5.38 (t, 1H), 5.17 (s, 1H), 4.53 (d, 2H), 1.42 (s, 6H).

MS (DCI, NH₃): m/z=168 [M+H]⁺.

### Step 3: [2-(2-Hydroxypropan-2-yl)pyridin-4-yl]methyl methanesulphonate

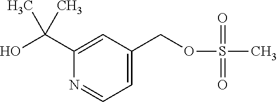

Analogously to the process described under Example 88A/step 4, 160 mg (0.96 mmol) of the compound from Example 89A/step 2 were reacted to give 177 mg (75% of th.) of the title compound.

MS (DCI, NH₃): m/z=246 [M+H]⁺.

### Example 90A

### 5-(Chloromethyl)-N-(3,4-dimethoxybenzyl)-N-methylpyridin-2-amine dihydrochloride

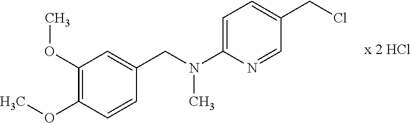

### Step 1: 6-[(3,4-Dimethoxybenzyl)(methyl)amino]nicotinic acid

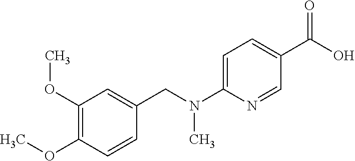

A mixture of 5.0 g (31.7 mmol) of 6-chloronicotinic acid and 15.1 ml (79.4 mmol) of 3,4-dimethoxy-N-methylbenzylamine was heated at 150° C. overnight, while stirring. After cooling to RT, 300 ml of ethyl acetate and 600 ml of water were added. The solid formed was filtered off in the course of the phase separation and dried in vacuo. 7.38 g (77% of th.) of the title compound were obtained.

¹H-NMR (400 MHz, CDCl₃, δ/ppm): 8.91 (d, 1H), 8.07-8.02 (dd, 1H), 6.81 (d, 1H), 6.78-6.73 (m, 2H), 6.52 (d, 1H), 4.82 (d, 2H), 3.86 (s, 3H), 3.82 (s, 3H), 3.12 (s, 3H).

LC/MS (method I, ESIpos): Rₜ=0.74 min, m/z=303 [M+H]⁺.

### Step 2: {6-[(3,4-Dimethoxybenzyl)(methyl)amino]pyridin-3-yl}methanol

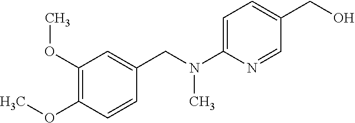

7.38 g (24.4 mmol) of the compound from Example 90A/step 1 were initially introduced into 225 ml of THF at 0° C. under argon, 20.3 ml (48.8 mmol) of a 2,4 M solution of lithium aluminium hydride in THF were slowly added dropwise and the mixture was subsequently stirred at RT for 2 h. Thereafter, 2 ml of water and 2 ml of 15% strength sodium hydroxide solution were slowly added, while cooling with ice. The mixture was diluted with 200 ml of tert-butyl methyl ether and the solid present was filtered off and washed three times with 100 ml of tert-butyl methyl ether each time. The filtrate and wash solutions were combined and concentrated and the resulting residue was dried in vacuo. 6.20 g (87% of th.) of the title compound were obtained.

¹H-NMR (400 MHz, CDCl₃, δ/ppm): 8.15 (d, 1H), 7.51-7.48 (dd, 1H), 6.81-6.72 (m, 3H), 6.52 (d, 1H), 4.72 (s, 2H), 4.54 (d, 2H), 3.85 (s, 3H), 3.82 (s, 3H), 3.05 (s, 3H), 1.65-1.60 (m, 1H).

LC/MS (method I, ESIpos): Rₜ=0.48 min, m/z=289 [M+H]⁺.

### Step 3: 5-(Chloromethyl)-N-(3,4-dimethoxybenzyl)-N-methylpyridin-2-amine dihydrochloride

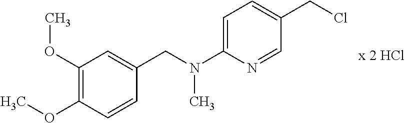

1.8 ml (24.5 mmol) of thionyl chloride were added to a solution of 3.54 g (12.3 mmol) of the compound from Example 90A/step 2 in 22 ml of methylene chloride at RT and the mixture was stirred at this temperature for 2 h. The batch was subsequently concentrated and the residue was dried in vacuo. 4.64 g (99% of th.) of the title compound were obtained.

¹H-NMR (400 MHz, CDCl₃, δ/ppm): 15.7 (s, broad, 1H), 8.31 (s, 1H), 7.85 (d, 1H), 6.90 (d, 1H), 6.84 (d, 1H), 6.80-6.72 (m, 2H), 4.84 (s, 2H), 4.49 (s, 2H), 3.88 (s, 6H), 3.55 (s, 3H).

LC/MS (method D, ESIpos): Rₜ=1.05 min, m/z=289/291 [M+H]⁺.

### Example 91A

### 1-[(6-Chloropyridin-3-yl)methyl]-N′-hydroxy-5-methyl-1H-pyrazole-3-carboximide amide

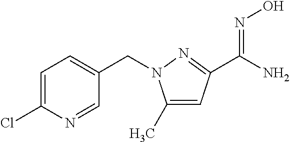

### Step 1: 1-[(6-Chloropyridin-3-yl)methyl]-5-methyl-1H-pyrazole-3-carboxamide

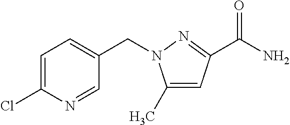

500 mg (1.99 mmol) of the compound from Example 45A were initially introduced into 15 ml of methylene chloride under argon and 867 μl (9.93 mmol) of oxalyl chloride were slowly added, followed by one drop of DMF. The mixture was stirred at RT for 1 h and subsequently concentrated. The residue was taken up in 4 ml of dioxane and the solution obtained was slowly added dropwise to 5.8 ml (99.3 mmol) of 33% strength aqueous ammonia solution at 0° C. The mixture was stirred at RT for 30 min and the solid formed was then filtered off and washed twice with 3 ml of water each time. After drying in vacuo, 423 mg (85% of th.) of the title compound were obtained.

¹H-NMR (400 MHz, CDCl₃, δ/ppm): 8.28 (d, 1H), 7.40-7.36 (dd, 1H), 7.31 (d, 1H), 6.68 (s, broad, 1H), 6.63 (s, 1H), 5.38 (s, broad, 1H), 5.26 (s, 2H), 2.26 (s, 3H).

LC/MS (method I, ESIpos): Rₜ=0.64 min, m/z=251/253 [M+H]⁺.

### Step 2: 1-[(6-Chloropyridin-3-yl)methyl]-5-methyl-1H-pyrazole-3-carbonitrile

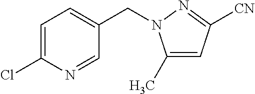

486 μl (2.87 mmol) of trifluoromethanesulphonic acid anhydride were slowly added dropwise to a solution of 400 mg (1.60 mmol) of the compound from Example 91A/step 1 and 1.4 ml (7.98 mmol) of N,N-diisopropylethylamine in 15 ml of methylene chloride under argon, while cooling with ice. The mixture was first stirred at 0° C. for 16 h, a further 486 μl (2.87 mmol) of trifluoromethanesulphonic acid anhydride and 1.4 ml (7.98 mmol) of N,N-diisopropylethylamine were then added and the mixture was stirred again at RT for 72 h. The mixture was then concentrated and the residue was pre-purified by column chromatography (silica gel, mobile phase: methylene chloride→methylene chloride/methanol 95:5). The product obtained was taken up in 50 ml of methylene chloride and the solution was washed once with 50 ml of water, dried over magnesium sulphate, filtered and concentrated again. The residue was purified again by column chromatography (silica gel, mobile phase: cyclohexane/ethyl acetate 1:1). 302 mg (81% of th.) of the title compound were obtained in this way.

¹H-NMR (400 MHz, CDCl₃, δ/ppm): 8.28 (d, 1H), 7.49-7.46 (dd, 1H), 7.33 (d, 1H), 6.47 (s, 1H), 5.30 (s, 2H), 2.28 (s, 3H).

LC/MS (method I, ESIpos): Rₜ=0.87 min, m/z=233/235 [M+H]⁺.

### Step 3: 1-[(6-Chloropyridin-3-yl)methyl]-N′-hydroxy-5-methyl-1H-pyrazole-3-carboximide amide

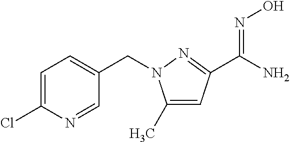

Analogously to the process described under Example 1A/step 5, 222 mg (63% of th., purity of 97%) of the title compound were obtained from 300 mg (1.29 mmol) of the compound from Example 91A/step 2.

¹H-NMR (400 MHz, CDCl₃, δ/ppm): 8.26 (d, 1H), 7.38-7.36 (dd, 1H), 7.27 (d, 1H), 6.36 (s, 1H), 5.25 (s, 2H), 5.12 (s, 2H), 2.25 (s, 3H).

LC/MS (method C, ESIpos): Rₜ=0.52 min, m/z=266/288 [M+H]⁺.

### Example 92A

### 1-({6-[(3,4-Dimethoxybenzyl)(methyl)amino]pyridin-3-yl}methyl)-5-methyl-1H-pyrazole-3-carboxylic acid

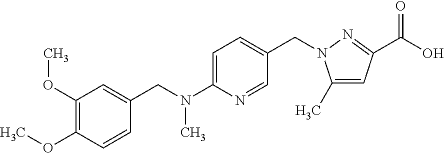

A mixture of 1.0 g (3.97 mmol) of the compound from Example 45A and 3.8 ml (19.9 mmol) of 3,4-dimethoxy-N-methylbenzylamine was heated at 150° C. under argon overnight, while stirring. After cooling to RT, ethyl acetate and water were added, the phases were separated and the aqueous phase was extracted three times with ethyl acetate. These ethyl acetate extracts were discarded. The aqueous phase was then first adjusted to a pH of 3 with 1 M hydrochloric acid, extracted four times with ethyl acetate, then adjusted to a pH of 7 with sodium bicarbonate solution and extracted again three times with ethyl acetate. The combined ethyl acetate phases were dried over magnesium sulphate, filtered and concentrated. After the residue had been dried in vacuo, 839 mg (49% of th., purity of 92%) of the title compound were obtained.

¹H-NMR (400 MHz, CDCl₃, δ/ppm): 8.10 (s, 1H), 7.32 (d, 1H), 6.81-6.70 (m, 3H), 6.61 (s, 1H), 6.46 (d, 1H), 5.21 (s, 2H), 4.70 (s, 2H), 3.84 (s, 3H), 3.81 (s, 3H), 3.04 (s, 3H), 2.25 (s, 3H).

LC/MS (method I, ESIpos): Rₜ=0.66 min, m/z=397 [M+H]⁺.

### EMBODIMENT EXAMPLES

### Example 1

### 5-[(5-Methyl-3-{3-[4-(trifluoromethoxy)phenyl]-1,2,4-oxadiazol-5-yl}-1H-pyrazol-1-yl)methyl]-pyridine-2-carbonitrile

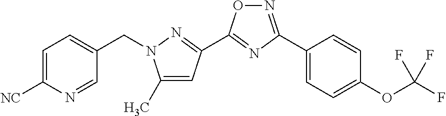

1.05 g (9.73 mmol) of solid potassium tert-butylate were added to a solution of 2.42 g (7.81 mmol) of the compound from Example 23A and 2.16 g (10.2 mmol) of the compound from Example 41A in 80 ml of anhydrous THF at 0° C. under inert conditions. The reaction mixture was then stirred at RT for 3 h. Approx. 350 ml of water were then added and the mixture was extracted three times with approx. 250 ml of ethyl acetate each time. The combined organic extracts were washed successively with water and saturated sodium chloride solution. After drying over anhydrous magnesium sulphate and filtering, the solvent was removed on a rotary evaporator. The crude product was purified by means of MPLC (silica gel, mobile phase: cyclohexane/ethyl acetate 5:1→1:1). 2.1 g (63% of th.) of the title compound were obtained.

¹H-NMR (400 MHz, CDCl₃, δ/ppm): 8.62 (d, 1H), 8.24 (d, 2H), 7.70 (d, 1H), 7.63 (dd, 1H), 7.34 (d, 2H), 6.87 (s, 1H), 5.53 (s, 2H), 2.34 (s, 3H).

HPLC (method A): Rₜ=4.96 min.

MS (DCI, NH₃): m/z=427 [M+H]⁺.

LC/MS (method C, ESIpos): Rₜ=2.70 min, m/z=427 [M+H]⁺.

### Example 2

### 2-Chloro-5-[(5-methyl-3-{3-[4-(trifluoromethoxy)phenyl]-1,2,4-oxadiazol-5-yl}-1H-pyrazol-1-yl)-methyl]pyridine

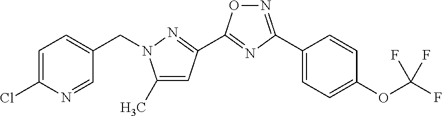

1.99 g (17.7 mmol) of potassium tert-butylate were added to a solution, cooled to 0° C., of 5.0 g (16.1 mmol) of the compound from Example 23A and 3.54 g (20.9 mmol) of 2-chloro-5-(chloromethyl)pyridine in 150 ml of THF and the mixture was then allowed to come to RT. It was stirred at RT overnight and thereafter at 45° C. for a further 4.5 h. The mixture was then diluted with water and extracted twice with ethyl acetate. The combined ethyl acetate phases were washed once with saturated aqueous sodium chloride solution, dried over magnesium sulphate, filtered and concentrated. The residue was purified by column chromatography over silica gel (mobile phase: cyclohexane/ethyl acetate 3:2). After drying in vacuo, 4.65 g (66% of th.) of the title compound were obtained.

¹H-NMR (400 MHz, CDCl₃, δ/ppm): 8.31 (d, 1H), 8.25 (d, 2H), 7.51 (dd, 1H), 7.36-7.30 (m, 3H), 6.82 (s, 1H), 5.43 (s, 2H), 2.32 (s, 3H).

HPLC (method A): Rₜ=4.91 min.

MS (DCI, NH₃): m/z=436 [M+H]⁺.

LC/MS (method C, ESIpos): Rₜ=2.83 min, m/z=436 [M+H]⁺.

### Example 3

### 2-Chloro-4-[(5-methyl-3-{3-[4-(trifluoromethoxy)phenyl]-1,2,4-oxadiazol-5-yl}-1H-pyrazol-1-yl)-methyl]pyridine

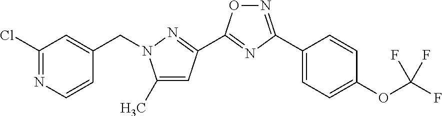

4.73 g (42.1 mmol) of potassium tert-butylate were added to a solution, cooled to 0° C., of 11.88 g (38.3 mmol) of the compound from Example 23A and 8.4 g (49.8 mmol) of the compound from Example 38A in 350 ml of THF and the mixture was then allowed to come to RT. It was stirred at RT overnight and then under reflux for a further 4 h. The mixture was then diluted with water and extracted twice with ethyl acetate. The combined ethyl acetate phases were washed once with saturated aqueous sodium chloride solution, dried over magnesium sulphate, filtered and concentrated. The residue was purified by column chromatography over silica gel (mobile phase: cyclohexane/ethyl acetate 3:2). The combined product fractions were concentrated and the residue was stirred in hexane, filtered off and dried in vacuo. 8.2 g (49% of th.) of the title compound were obtained.

¹H-NMR (400 MHz, CDCl₃, δ/ppm): 8.37 (d, 1H), 8.28-8.22 (m, 2H), 7.34 (d, 2H), 7.05 (s, 1H), 6.97 (d, 1H), 6.88 (s, 1H), 5.43 (s, 2H), 2.32 (s, 3H).

LC/MS (method F, ESIpos): Rₜ=1.47 min, m/z=436 [M+H]⁺.

The compounds in the following table were prepared from the corresponding educts analogously to the processes described in Examples 1 to 3. Depending on the polarity of the compounds, these were either isolated by extraction by stirring the crude product in methylene chloride, ethyl acetate, acetonitrile or diethyl ether, or obtained by means of preparative HPLC or by means of MPLC over silica gel with cyclohexane/ethyl acetate mixtures as the mobile phase. The arylmethyl chlorides, bromides or methanesulphonates used as educts were either commercially obtainable, or their preparation is described in the literature, or they were prepared as described above.

| | | HPLC: | MS: m/z | LC/MS |
|---|---|---|---|---|
| Example | Structure | Rₜ [min] | [M + H]⁺ | method |
| 4 | 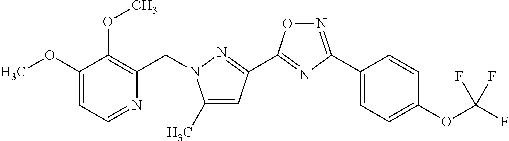 | 2.27 | 462 | J |
| 5 | 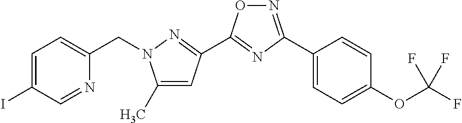 | 5.25 | 528 | B |
| | ¹H-NMR (400 MHz, DMSO-d₆, δ/ppm): 8.79 (d, 1H), 8.24 (d, 2H), 7.97 (dd, 1H), | | | |
| | 7.33 (d, 2H), 6.86 (d, 1H), 6.83 (s, 1H), 5.50 (s, 2H), 2.36 (s, 3H). | | | |
| 6 | 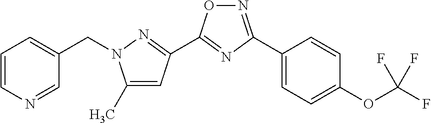 | 2.43 | 402 | C |
| | ¹H-NMR (400 MHz, DMSO-d₆, δ/ppm): 8.57 (d, 1H), 8.52 (s, 1H), 8.24 (d, 2H), 7.52 | | | |
| | (d, 1H), 7.32 (d, 2H), 7.30-7.25 (m, 1H), 6.82 (s, 1H), 5.46 (s, 2H), 2.32 (s, 3H). | | | |
| 7 | 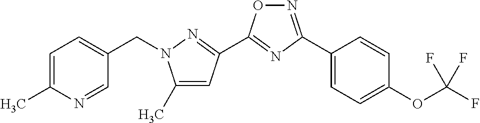 | 2.21 | 416 | C |
| | ¹H-NMR (400 MHz, DMSO-d₆, δ/ppm): 8.81 (s, 1H), 8.23 (d, 2H), 7.92 (d, 1H), 7.48 | | | |
| | (d, 1H), 7.32 (d, 2H), 6.82 (s, 1H), 5.51 (s, 2H), 2.77 (s, 3H), 2.37 (s, 3H). | | | |
| 8 | 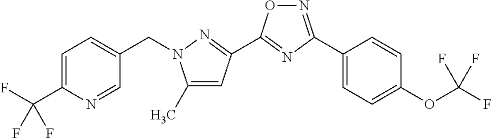 | 2.43 | 470 | J |
| 9 | 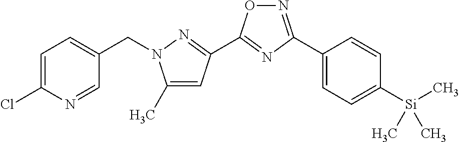 | 1.45 | 424 | I |
| | ¹H-NMR (400 MHz, DMSO-d₆, δ/ppm): 8.32 (dd, 1H), 8.16 (d, 2H), 7.63 (d, 2H), | | | |
| | 7.52-7.49 (dd, 1H), 7.31 (d, 1H), 6.82 (s, 1H), 5.42 (s, 2H), 2.32 (s, 3H), 0.31 (s, 9H). | | | |
| 10 | 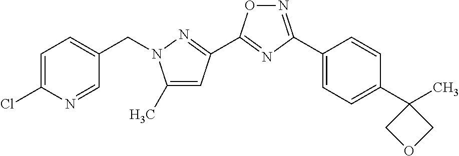 | 1.26 | 422 | F |
| | ¹H-NMR (400 MHz, DMSO-d₆, δ/ppm): 8.32 (d, 1H), 8.20 (d, 2H), 7.51 (dd, 1H), | | | |
| | 7.33 (d, 2H), 7.31 (d, 1H), 6.83 (s, 1H), 5.44 (s, 2H), 5.00 (d, 2H), 4.58 (d, 2H), 2.33 | | | |
| | (s, 3H), 1.77 (s, 3H). | | | |
| 11 | 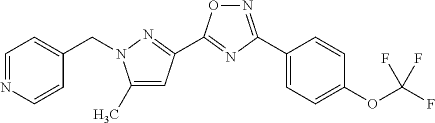 | 2.33 | 402 | C |
| | ¹H-NMR (400 MHz, CDCl₃, δ/ppm): 8.59 (d, 2H), 8.25 (d, 2H), 7.32 (d, 2H), 7.02 (d, | | | |
| | 2H), 6.86 (s, 1H), 5.47 (s, 2H), 2.29 (s, 3H). | | | |
| 12 | 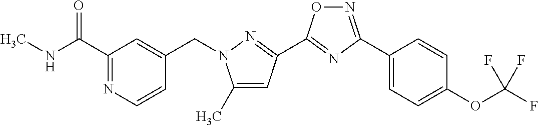 | 2.54 | 459 | C |
| | ¹H-NMR (400 MHz, CDCl₃, δ/ppm): 8.50 (d, 1H), 8.27-8.22 (m, 2H), 8.02 (s, 1H), | | | |
| | 8.02-7.96 (m, 1H), 7.33 (d, 2H), 7.09-7.06 (dd, 1H), 6.86 (s, 1H), 5.52 (s, 2H), 3.02 (d, | | | |
| | 3H), 2.30 (s, 3H). | | | |
| 13 | 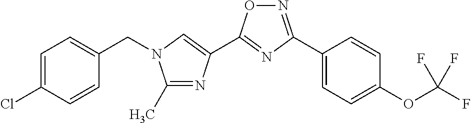 | 1.47 | 435 | F |
| | ¹H-NMR (400 MHz, CDCl₃, δ/ppm): 8.23 (d, 2H), 7.70 (s, 1H), 7.38 (d, 2H), 7.32 (d, | | | |
| | 2H), 7.09 (d, 2H), 5.12 (s, 2H), 2.47 (s, 3H). | | | |
| 14 | 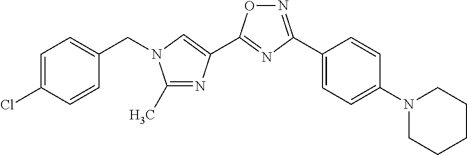 | 2.30 | 434 | E |
| | ¹H-NMR (400 MHz, CDCₗ3, δ/ppm): 8.03 (d, 2H), 7.68 (s, 1H), 7.37 (d, 2H), 7.07 (d, | | | |
| | 2H), 6.95 (d, 2H), 5.10 (s, 2H), 3.30 (m, 4H), 2.44 (s, 3H), 1.73-1.61 (m, 6H). | | | |
| 15 | 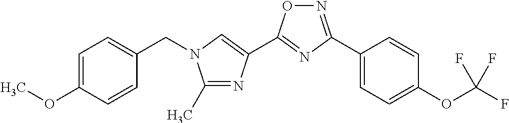 | 4.60 | 431 | A |
| | ¹H-NMR (400 MHz, CDCl₃, δ/ppm): 8.23 (d, 2H), 7.67 (s, 1H), 7.31 (d, 2H) 7.10 (d, | | | |
| | 2H), 6.92 (d, 2H), 5.06 (s, 2H), 3.82 (s, 3H), 2.49 (s, 3H). | | | |
| 16 | 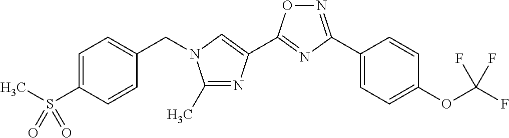 | 1.22 | 479 | F |

¹H-NMR (400 MHz, DMSO-d₆, δ/ppm): 8.39 (s, 1H), 8.18 (d, 2H), 7.96 (d, 2H), 7.59
(d, 2H), 7.50 (d, 2H), 5.47 (s, 2H), 3.22 (s, 3H), 2.37 (s, 3H).

### Example 17

### 2-Bromo-5-[(5-methyl-3-{3-[4-(trifluoromethoxy)phenyl]-1,2,4-oxadiazol-5-yl}-1H-pyrazol-1-yl)-methyl]pyridine

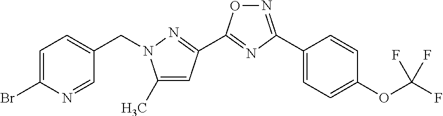

A mixture of 1.95 g (4.47 mmol) of the compound from Example 2 and 1.37 g (8.95 mmol) of bromo(trimethyl)silane in 0.5 ml of propionitrile was heated at 120° C. in a microwave apparatus (CEM Discover, initial irradiation power 250 W) for 70 min, while stirring. During this operation a relatively marked increase in pressure and temperature was to be observed in the first 10 min. After cooling to RT, a further 350 mg (2.29 mmol) of bromo(trimethyl)silane were added and the mixture was heated again at 120° C. in the microwave oven for 60 min. During this operation a relatively marked increase in pressure and temperature was again to be observed in the first 10 min. After cooling to RT, the mixture was diluted with 100 ml of water and 100 ml of ethyl acetate and the phases were separated. The combined organic phase was washed once with 100 ml of water, dried over sodium sulphate, filtered and concentrated on a rotary evaporator. The residue was purified by column chromatography over silica gel (mobile phase: cyclohexane/ethyl acetate 3:2). 1.45 g (65% of th., purity of 86% according to LC-MS) of the title compound were obtained.

¹H-NMR (400 MHz, CDCl₃, δ/ppm): 8.31 (d, 1H), 8.23 (d, 2H), 7.47 (d, 1H), 7.40 (dd, 1H), 7.33 (d, 2H), 6.82 (s, 1H), 5.41 (s, 2H), 2.32 (s, 3H).

LC/MS (method E, ESIpos): Rₜ=2.54 min, m/z=480 [M+H]⁺.

### Example 18

### 3-{5-[(5-Methyl-3-{3-[4-(trifluoromethoxy)phenyl]-1,2,4-oxadiazol-5-yl}-1H-pyrazol-1-yl)-methyl]pyridin-2-yl}prop-2-yn-1-ol

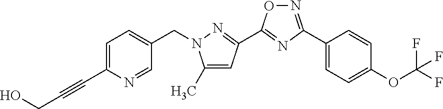

11.4 ml of triethylamine, 385 mg (6.87 mmol) of prop-2-yn-1-ol, 132 mg (0.115 mmol) of tetrakis(triphenylphosphine)palladium(0) and 44 mg (0.229 mmol) of copper(I) iodide were added to a solution of 1.10 g (2.29 mmol) of the compound from Example 17 in 23 ml of degassed THF under argon at RT and the mixture was stirred at RT for 16 h. The mixture was then concentrated on a rotary evaporator, the residue was dissolved in 8 ml of acetonitrile and 20 ml of water were added. The solid formed was filtered off after stirring at RT for 30 min and was washed in each case twice with water and with ethyl acetate. The wash phases were combined and concentrated and the residue and the solid obtained previously were each purified via preparative HPLC (method N). The product-containing fractions were in each case combined, saturated aqueous sodium bicarbonate solution was added and the mixture was concentrated to a small residual volume of solvent and extracted three times with ethyl acetate. The combined organic phases were in each case dried over sodium sulphate and concentrated. 634 mg (59% of th.) of the title compound were obtained from the two purification operations together.

¹H-NMR (400 MHz, CDCl₃, δ/ppm): 8.25 (d, 2H), 7.48-7.45 (dd, 1H), 7.40 (d, 1H), 7.33 (d, 2H), 6.82 (s, 1H), 5.46 (s, 2H), 4.51 (d, 2H), 2.31 (s, 3H), 1.96-1.91 (t, 1H).

LC/MS (method E, ESIpos): Rₜ=2.08 min, m/z=456 [M+H]⁺.

### Example 19

### 3-{5-[(5-Methyl-3-{3-[4-(trifluoromethoxy)phenyl]-1,2,4-oxadiazol-5-yl}-1H-pyrazol-1-yl)-methyl]pyridin-2-yl}propan-1-ol

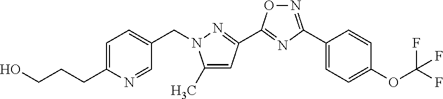

633 mg (1.39 mmol) of the compound from Example 18 were dissolved in a mixture of 7.5 ml of ethanol and 7.5 ml of THF, 358 μl (2.57 mmol) of triethylamine and 32 mg (0.139 mmol) of platinum(IV) oxide were added and hydrogenation was carried out at RT under normal pressure for 4 h. The reaction mixture was then filtered and the filtrate was concentrated on a rotary evaporator. The residue was purified by means of preparative HPLC (method N). The product-containing fractions were combined and saturated aqueous sodium bicarbonate solution was added. After removal of some of the solvent on a rotary evaporator, the remaining portion was extracted three times with 40 ml of ethyl acetate each time. The combined organic phases were dried over sodium sulphate and filtered and the solvent was removed. 390 mg (61% of th.) of the title compound were obtained.

¹H-NMR (400 MHz, CDCl₃, δ/ppm): 8.41 (s, 1H), 8.24 (d, 2H), 7.48 (dd, 1H), 7.32 (d, 2H), 7.18 (d, 1H), 6.82 (s, 1H), 5.42 (s, 2H), 3.70 (t, 2H), 2.96 (t, 2H), 2.31 (s, 3H), 2.01-1.93 (m, 2H).

LC/MS (method D, ESIpos): Rₜ=2.04 min, m/z=460 [M+H]⁺.

### Example 20

### 2-Ethynyl-5-[(5-methyl-3-{3-[4-(trifluoromethoxy)phenyl]-1,2,4-oxadiazol-5-yl}-1H-pyrazol-1-yl)methyl]pyridine

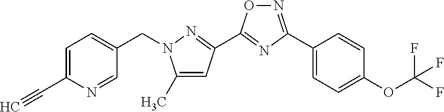

22 mg (0.117 mmol) of copper(I) iodide were dissolved in 1.6 ml of triethylamine at 40° C. under argon, the solution was cooled to RT, 26 μl (0.185 mmol) of ethynyl(trimethyl)silane were then added and the mixture was stirred at RT for 10 min. 45 mg (0.065 mmol) of bis(triphenylphosphine)palladium(II) chloride were then added and the mixture was stirred at RT for a further 10 min. Finally, 622 mg (1.30 mmol) of the compound from Example 17, followed by 14.4 ml of triethylamine and 235 μl (1.67 mmol) of ethynyl(trimethyl)silane were added. The mixture was subsequently heated at 100° C. for 16 h. After cooling to RT, the mixture was taken up in 50 ml of methylene chloride and 70 ml of water, the phases were separated and the aqueous phase was extracted twice more with 30 ml of ethyl acetate each time. The combined organic phases were dried over sodium sulphate, filtered and concentrated on a rotary evaporator. The residue was dissolved in 16 ml of THF, 16 ml of a 0.1 M sodium hydroxide solution were added at RT and the mixture was stirred at RT for 4 h. 100 ml of ethyl acetate and 150 ml of water were then added to the reaction mixture, the phases were separated and the aqueous phase was extracted twice more with 100 ml of ethyl acetate each time. The organic phases were combined, dried over sodium sulphate and filtered and the solvent was removed. The residue was purified by means of preparative HPLC (method N). The product-containing fractions were combined, 20 ml of a saturated aqueous sodium bicarbonate solution were added, some of the solvent was removed, 30 ml of water were added and the mixture was extracted three times with 50 ml of ethyl acetate each time. The combined organic phases were dried over sodium sulphate and filtered and the solvent was removed. 119 mg (22% of th.) of the title compound were obtained in this manner.

¹H-NMR (400 MHz, CDCl₃, δ/ppm): 8.51 (s, 1H), 8.25 (d, 2H), 7.50-7.44 (m, 2H), 7.34 (d, 2H), 6.83 (s, 1H), 5.48 (s, 2H), 3.19 (s, 1H), 2.32 (s, 3H).

LC/MS (method C, ESIpos): Rₜ=2.71 min, m/z=426 [M+H]⁺.

### Example 21

### 5-[(5-Methyl-3-{3-[4-(trifluoromethoxy)phenyl]-1,2,4-oxadiazol-5-yl}-1H-pyrazol-1-yl)methyl]-2-[(trimethylsilyl)ethynyl]pyridine

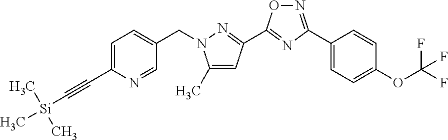

3.4 mg (0.018 mmol) of copper(I) iodide were dissolved in 0.3 ml of triethylamine at 40° C. under argon, the solution was cooled to RT, 4 μl (0.029 mmol) of ethynyl(trimethyl)silane were then added and the mixture was stirred at RT for 10 min. 7.0 mg (0.010 mmol) of bis(triphenylphosphine)palladium(II) chloride were then added and the mixture was stirred at RT for a further 10 min. Finally, 96 mg (0.20 mmol) of the compound from Example 17, followed by 2.7 ml of triethylamine and 36 μl (0.257 mmol) of ethynyl(trimethyl)silane were added. The mixture was subsequently heated at 100° C. for 16 h. After cooling to RT, the mixture was taken up in 300 ml of ethyl acetate and 50 ml of water, the phases were separated and the aqueous phase was extracted three times more with 30 ml of ethyl acetate each time. The combined organic phases were dried over sodium sulphate, filtered and concentrated on a rotary evaporator. The residue was purified by means of preparative HPLC (method N). 53 mg (51% of th.) of the title compound were obtained.

¹H-NMR (400 MHz, CDCl₃, δ/ppm): 8.51 (s, 1H), 8.49 (s, 2H), 8.25 (d, 2H), 7.48-7.40 (m, 2H), 7.34 (d, 2H), 6.83 (s, 1H), 5.46 (s, 2H), 2.30 (s, 3H), 0.26 (s, 9H).

LC/MS (method F, ESIpos): Rₜ=1.68 min, m/z=498 [M+H]⁺.

### Example 22

### 2-Iodo-5-[(5-methyl-3-{3-[4-(trifluoromethoxy)phenyl]-1,2,4-oxadiazol-5-yl}-1H-pyrazol-1-yl)-methyl]pyridine

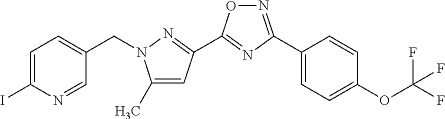

103 mg (0.688 mmol) of sodium iodide and 27 mg (0.252 mmol) of chloro(trimethyl)silane were added to a solution of 100 mg (0.229 mmol) of the compound from Example 2 in 0.5 ml of propionitrile in a microwave reaction vessel at RT, after which the reaction mixture rapidly assumed a solid consistency. The mixture was then heated at 120° C. in a microwave apparatus for 1 h (CEM Discover, initial irradiation power 250 W). After cooling to RT, the reaction mixture was diluted with 2 ml of acetonitrile and 1 ml of water. Two phases formed, which were separated from one another. The organic phase was purified by means of preparative HPLC (method N) without further treatment. 61 mg (50% of th.) of the title compound were obtained.

¹H-NMR (400 MHz, CDCl₃, δ/ppm): 8.29 (d, 1H), 8.24 (d, 2H), 7.71 (d, 1H), 7.32 (d, 2H), 7.18 (dd, 1H), 6.82 (s, 1H), 5.39 (s, 2H), 2.31 (s, 3H).

LC/MS (method F, ESIpos): Rₜ=1.52 min, m/z=528 [M+H]⁺.

### Example 23

### 4-[(5-Methyl-3-{3-[4-(trifluoromethoxy)phenyl]-1,2,4-oxadiazol-5-yl}-1H-pyrazol-1-yl)methyl]-pyridine-2-carbonitrile

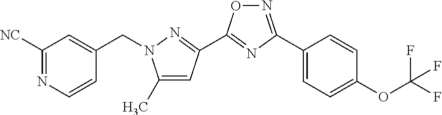

200 mg (0.459 mmol) of the compound from Example 3 were initially introduced into 3.4 ml of dimethylacetamide, 31 mg (0.266 mmol) of zinc cyanide, 6.7 mg (0.020 mmol) of palladium(II) trifluoroacetate, 16 mg (0.040 mmol) of racemic 2-(di-tert-butylphosphino)-1,1′-binaphthyl and 6 mg (0.092 mmol) of zinc powder (97.5%, 325 mesh) were added successively and the mixture was stirred at 90° C. overnight. After cooling to RT, a further 6.7 g (0.020 mmol) of palladium(II) trifluoroacetate were added and the mixture was stirred again at 90° C. for 24 h. After cooling to RT, 6.7 mg (0.020 mmol) of palladium(II) trifluoroacetate, 16 mg (0.040 mmol) of racemic 2-(di-tert-butylphosphino)-1,1′-binaphthyl and 6 mg (0.092 mmol) of zinc powder (97.5%, 325 mesh) were again added and the mixture was stirred again at 90° C. overnight. After cooling to RT, the solid constituents were then filtered off and the mixture which remained was purified by means of preparative HPLC (method N). The combined product-containing fractions were concentrated on a rotary evaporator to a small residual volume and sodium bicarbonate was then added, after which a solid precipitated out. The solid was filtered off, dried in vacuo and 21 mg (11% of th.) of the title compound were obtained in this way.

¹H-NMR (400 MHz, CDCl₃, δ/ppm): 8.71 (d, 1H), 8.24 (d, 2H), 7.41 (s, 1H), 7.34 (d, 2H), 7.24 (s, 1H), 6.90 (s, 1H), 5.51 (s, 2H), 2.32 (s, 3H).

LC/MS (method D, ESIpos): Rₜ=2.52 min, m/z=427 [M+H]⁺.

### Example 24

### N-Methyl-5-[(5-methyl-3-{3-[4-(trifluoromethoxy)phenyl]-1,2,4-oxadiazol-5-yl}-1H-pyrazol-1-yl)methyl]pyridin-2-amine

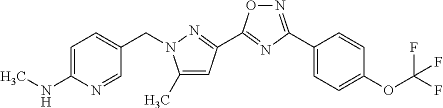

A mixture of 200 mg (0.459 mmol) of the compound from Example 2 and 285 mg (9.179 mmol) of methylamine was heated in a microwave apparatus (CEM Discover, initial irradiation power 250 W) first at 160° C. for 3 h and then at 170° C. for 6 h, while stirring. After cooling to RT, the mixture was purified directly by means of preparative HPLC (method N). The combined product-containing fractions were concentrated on a rotary evaporator to a residual volume, saturated aqueous sodium bicarbonate solution was added and the mixture was extracted twice with ethyl acetate. The combined organic phases were dried over magnesium sulphate, filtered and concentrated. After drying in vacuo, 99 mg (50% of th.) of the title compound were obtained.

¹H-NMR (400 MHz, CDCl₃, δ/ppm): 8.25 (d, 2H), 8.02 (d, 1H), 7.37 (dd, 1H), 7.32 (d, 2H), 6.77 (s, 1H), 6.35 (d, 1H), 5.29 (s, 2H), 4.65-4.58 (m, broad, 1H), 2.90 (d, 3H), 2.31 (s, 3H).

LC/MS (method F, ESIpos): Rₜ=1.08 min, m/z=431 [M+H]⁺.

The examples in the following table were prepared analogously to the process described under Example 24 using the particular corresponding amine and the corresponding 2-chloropyridine compound from Example 2 or 3. In contrast to that described in Example 24, these reactions were usually carried out in DMSO as the solvent (approx. 0.5 ml of DMSO per 0.10 mmol of the 2-chloropyridine educt). For the preparation of some examples it was necessary to prolong the duration of the reaction by up to a further 10 hours and/or to increase the amount of educt amine employed by up to a further 10 equivalents, based on the 2-chloropyridine derivative employed. In some examples it was furthermore necessary to carry out two purifications by means of preparative HPLC. Most of the amine components employed were commercially obtainable; some were prepared by processes described in the literature.

| Ex- | | HPLC: | MS: m/z | LC/MS |
|---|---|---|---|---|
| ample | Structure | Rₜ [min] | [M + H]⁺ | method |
| 25 | 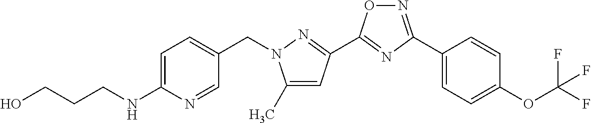 | 1.67 | 475 | C |
| | ¹H-NMR (400 MHz, DMSO-d₆, δ/ppm): 8.20 (d, 2H), 7.98 (s, 1H), 7.59 (d, 2H), 7.29 | | | |
| | (d, 1H), 6.88 (s, 1H), 6.59 (t, 1H), 6.42 (d, 1H), 5.26 (s, 2H), 4.45 (s, broad, 1H), 3.44 | | | |
| | (t, 2H), 3.26-3.22 (m, 2H), 2.39 (s, 3H), 1.68-1.60 (m, 2H). | | | |
| 26 | 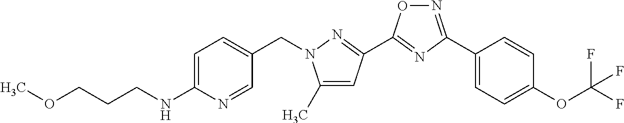 | 1.80 | 489 | C |
| | ¹H-NMR (400 MHz, DMSO-d₆, δ/ppm): 8.20 (d, 2H), 7.98 (s, 1H), 7.59 (d, 2H), 7.29 | | | |
| | (d, 1H), 6.88 (s, 1H), 6.61 (t, 1H), 6.42 (d, 1H), 5.28 (s, 2H), 3.40-3.20 (m, 7H), 2.39 | | | |
| | (s, 3H), 1.76-1.68 (m, 2H). | | | |
| 27 | 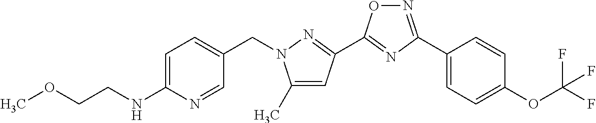 | 1.14 | 475 | F |
| | ¹H-NMR (400 MHz, DMSO-d₆, δ/ppm): 8.20 (d, 2H), 7.98 (s, 1H), 7.59 (d, 2H), 7.29 | | | |
| | (d, 1H), 6.88 (s, 1H), 6.67 (t, 1H), 6.48 (d, 1H), 5.28 (s, 2H), 3.45-3.36 (m, 4H), 3.24 | | | |
| | (s, 3H), 2.39 (s, 3H). | | | |
| 28 | 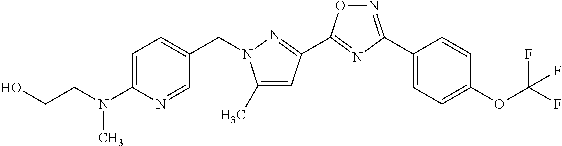 | 1.49 | 475 | E |
| | ¹H-NMR (400 MHz, DMSO-d₆, δ/ppm): 8.20 (d, 2H), 8.07 (d, 1H), 7.59 (d, 2H), 7.40 | | | |
| | (dd, 1H), 6.88 (s, 1H), 6.60 (d, 1H), 5.30 (s, 2H), 4.65 (t, 1H), 3.58-3.49 (m, 4H), 3.00 | | | |
| | (s, 3H), 2.39 (s, 3H). | | | |
| 29 | 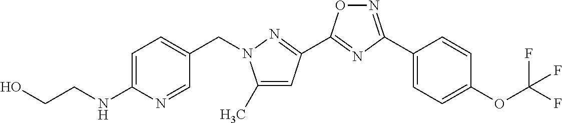 | 1.06 | 461 | F |
| | ¹H-NMR (400 MHz, DMSO-d₆, δ/ppm): 8.20 (d, 2H), 7.96 (s, 1H), 7.59 (d, 2H), 7.30 | | | |
| | (d, 1H), 6.88 (s, 1H), 6.60 (t, 1H), 6.48 (d, 1H), 5.27 (s, 2H), 4.69 (t, 1H), 3.57-3.47 | | | |
| | (m, 2H), 3.33-3.26 (m, 2H), 2.39 (s, 3H). | | | |
| 30 | 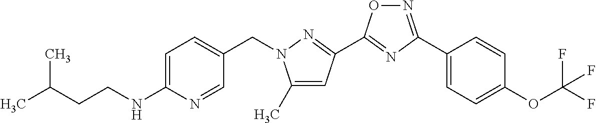 | 1.80 | 487 | E |
| | ¹H-NMR (400 MHz, CDCl₃, δ/ppm): 8.25 (d, 2H), 8.01 (s, 1H), 7.37-7.31 (m, 3H), | | | |
| | 6.76 (s, 1H), 6.32 (d, 1H), 5.29 (s, 2H), 4.50 (t, 1H), 3.28-3.22 (m, 2H), 2.31 (s, 3H), | | | |
| | 1.75-1.65 (m, 1H), 1.50 (m, 2H), 0.92 (d, 6H). | | | |
| 31 | 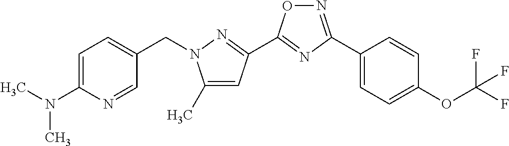 | 1.88 | 445 | C |
| | ¹H-NMR (400 MHz, CDCl₃, δ/ppm): 8.25 (d, 2H), 8.10 (s, 1H), 7.39 (d, 1H), 7.32 (d, | | | |
| | 2H), 6.76 (s, 1H), 6.46 (d, 1H), 5.30 (s, 2H), 3.08 (s, 6H), 2.31 (s, 3H). | | | |
| 32 | 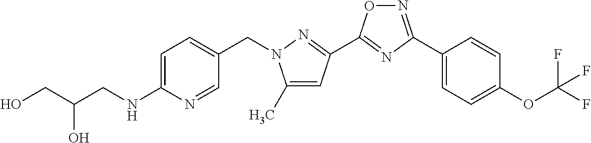 | 1.38 | 491 | E |
| | ¹H-NMR (400 MHz, CDCl₃, δ/ppm): 8.20 (d, 2H), 7.95 (s, 1H), 7.60 (d, 2H), 7.30 (d, | | | |
| | 1H), 6.88 (s, 1H), 6.58 (t, 1H), 6.50 (d, 1H), 5.28 (s, 2H), 4.88 (s, broad, 1H), 4.62 (s, | | | |
| | broad, 1H), 3.62-3.54 (m, 1H), 3.40-3.20 (m, 3H), 3.18-3.10 (m, 1H), 2.38 (s, 3H). | | | |
| 33 | 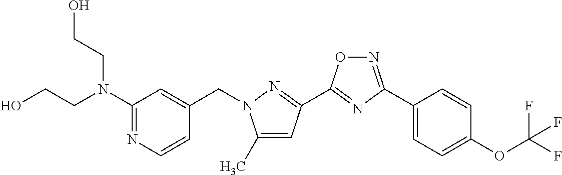 | 1.42 | 505 | E |
| | ¹H-NMR (400 MHz, DMSO-d₆, δ/ppm): 8.20 (d, 2H), 7.92 (d, 1H), 7.61 (d, 2H), 6.98 | | | |
| | (s, 1H), 6.90 (m, 1H), 6.39 (d, 1H), 5.52 (s, 2H), 3.85-3.40 (m, 8H), 2.36 (s, 3H). | | | |
| 34 | 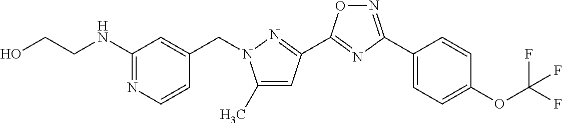 | 1.03 | 461 | F |
| | ¹H-NMR (400 MHz, CDCl₃, δ/ppm): 8.23 (d, 2H), 7.92 (d, 1H), 7.32 (d, 2H), 6.82 (s, | | | |
| | 1H), 6.39 (d, 1H), 6.12 (s, 1H), 5.88 (s, broad, 1H), 5.32 (s, 2H), 3.82-3.78 (m, 2H), | | | |
| | 3.48-3.42 (m, 2H), 2.30 (s, 3H). | | | |
| 35 | 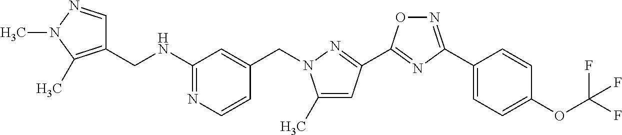 | 1.68 | 525 | J |
| 36 | 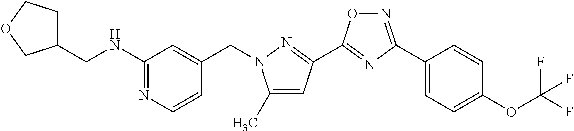 | 1.68 | 501 | J |
| 37 | 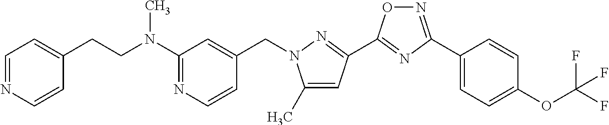 | 1.54 | 536 | J |
| 38 | 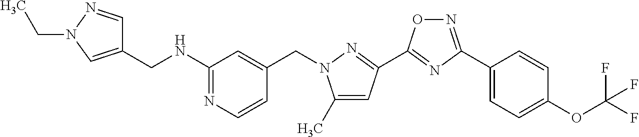 | 1.69 | 525 | J |
| 39 | 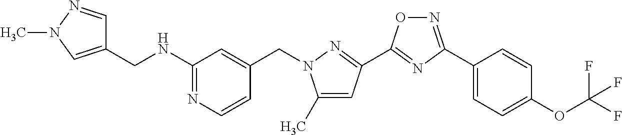 | 1.66 | 511 | J |
| 40 | 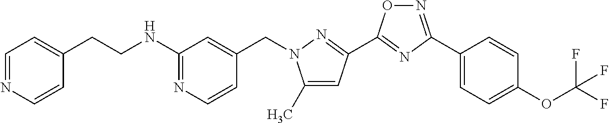 | 1.46 | 522 | J |
| 41 | 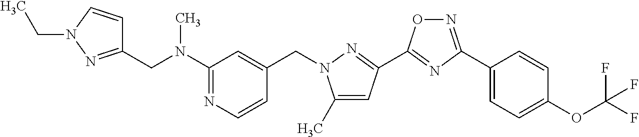 | 1.79 | 539 | J |
| 42 | 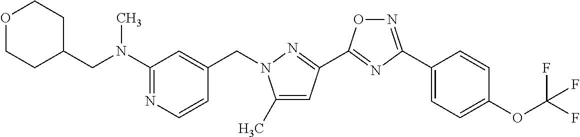 | 1.68 | 515 | J |
| 43 | 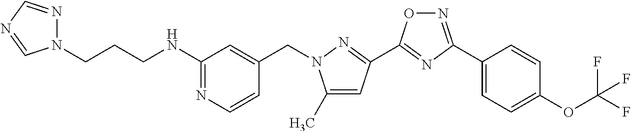 | 1.63 | 526 | J |
| 44 | 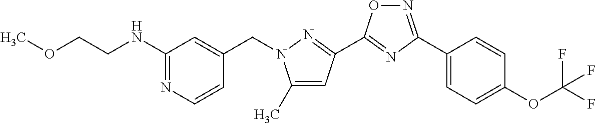 | 1.68 | 475 | J |
| 45 | 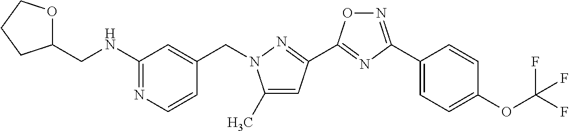 | 1.71 | 501 | J |
| 46 | 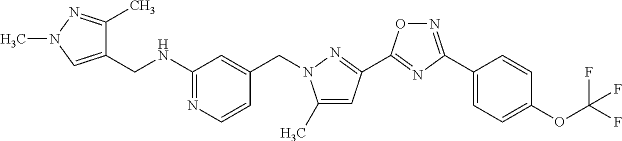 | 1.68 | 525 | J |
| 47 | 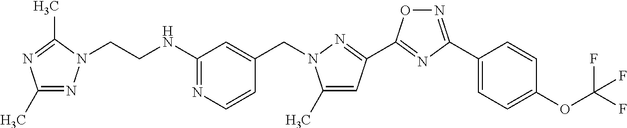 | 1.66 | 540 | J |
| 48 | 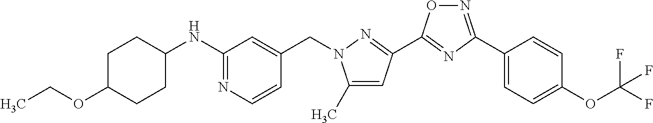 | 1.76 | 543 | J |
| 49 | 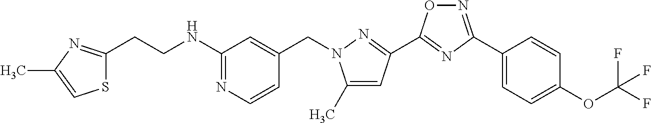 | 1.76 | 542 | J |
| 50 | 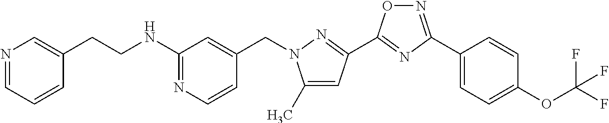 | 1.50 | 522 | J |
| 51 | 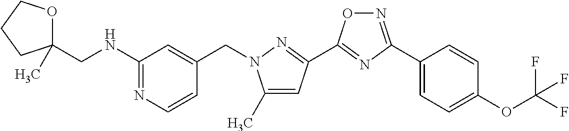 | 1.73 | 515 | J |
| 52 | 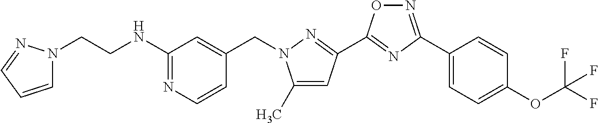 | 1.71 | 511 | J |
| 53 | 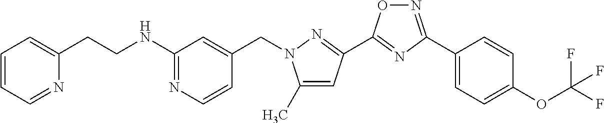 | 1.60 | 522 | J |
| 54 | 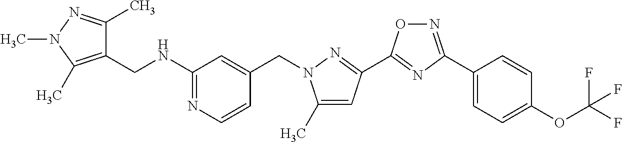 | 1.69 | 539 | J |
| 55 | 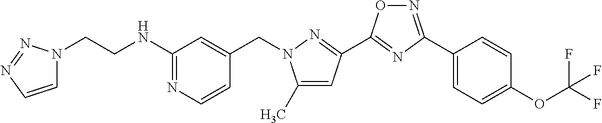 | 1.68 | 512 | J |
| 56 | 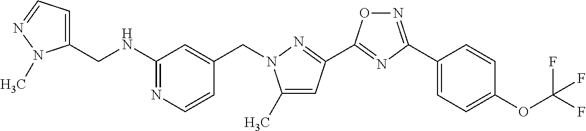 | 1.73 | 511 | J |
| 57 | 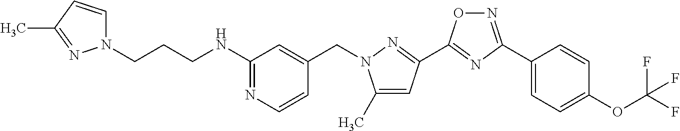 | 1.72 | 539 | J |
| 58 | 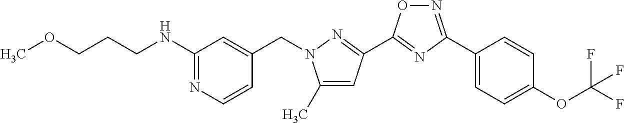 | 1.69 | 489 | J |
| 59 | 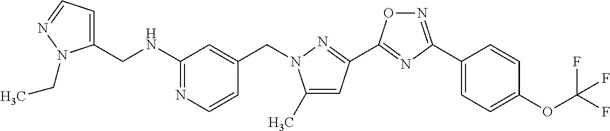 | 1.77 | 525 | J |
| 60 | 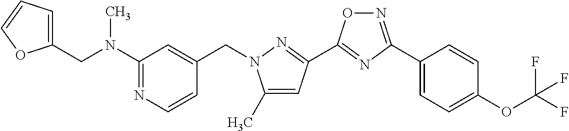 | 1.98 | 511 | J |
| 61 | 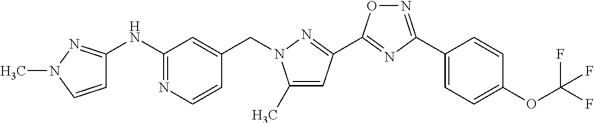 | 1.73 | 497 | J |
| 62 | 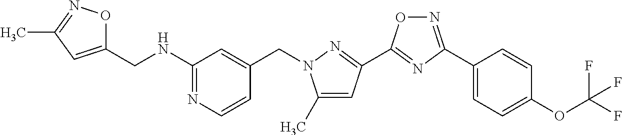 | 1.87 | 512 | J |
| 63 | 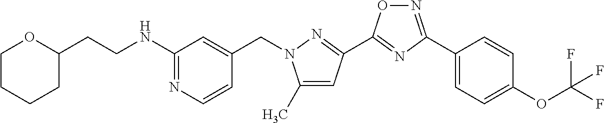 | 1.77 | 529 | J |
| 64 | 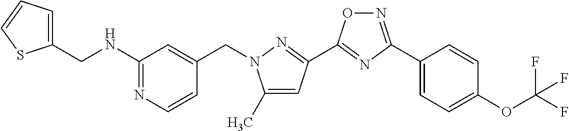 | 1.86 | 513 | J |

### Example 65

### 2-Chloro-5-[(3-{3-[4-(2-fluoropropan-2-yl)phenyl]-1,2,4-oxadiazol-5-yl}-5-methyl-1H-pyrazol-1-yl)methyl]pyridine

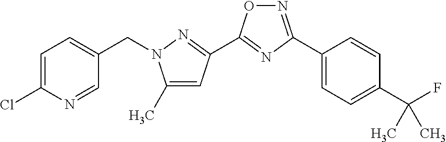

508 mg (2.65 mmol) of EDC and 358 mg (2.65 mmol) of HOBt were added to a solution of 667 mg (2.65 mmol) of the compound from Example 45A in 10 ml of anhydrous DMF at RT. After 30 min, 520 mg (2.65 mmol) of the compound from Example 2A, dissolved in 5 ml of DMF, were added. The mixture was stirred first at RT for 1 h and then at 140° C. for 1 h. After cooling, the majority of the solvent was removed on a rotary evaporator. 50 ml each of water and ethyl acetate were added. After separation of the phases, the organic phase was washed successively with 50 ml each of 10% strength aqueous citric acid, saturated sodium bicarbonate solution and saturated sodium chloride solution. After drying over anhydrous sodium sulphate, the mixture was filtered and the solvent was removed on a rotary evaporator. The crude product obtained was purified by means of MPLC (silica gel, mobile phase: cyclohexane/ethyl acetate 2:1). 418 mg (36% of th., purity of 93%) of the title compound were obtained, this being employed without further purification.

¹H-NMR (400 MHz, DMSO-d₆, δ/ppm): 8.39 (d, 1H), 8.08 (d, 2H), 7.68 (dd, 1H), 7.62 (d, 2H), 7.52 (d, 1H), 6.93 (s, 1H), 5.56 (s, 2H), 2.39 (s, 3H), 1.72 (s, 3H), 1.86 (s, 3H).

LC/MS (method F, ESIpos): Rₜ=1.43 min, m/z=412 [M+H]⁺.

### Example 66

### 2-Chloro-5-[(3-{3-[4-(3-fluoro-oxetan-3-yl)phenyl]-1,2,4-oxadiazol-5-yl}-5-methyl-1H-pyrazol-1-yl)methyl]pyridine

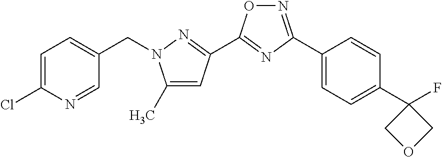

83 μl (0.954 mmol) of oxalyl chloride were added to a solution of 80 mg (0.318 mmol) of the compound from Example 45A in 3 ml of anhydrous methylene chloride at 0° C. under inert conditions. The reaction mixture was stirred at RT for 2 h. All the volatile constituents were then removed on a rotary evaporator and the residue obtained in this way was dried under a high vacuum for 20 min, before being dissolved again in 2 ml of methylene chloride. This solution was added dropwise to a solution of 80 mg (0.381 mmol) of the compound from Example 5A and 89 μl (0.636 mmol) of triethylamine in 1 ml of methylene chloride at 0° C. After the reaction mixture had been stirred at RT for 1 h, all the volatile constituents were again removed on a rotary evaporator and the residue was dissolved in 4 ml of DMSO. This solution was heated in a microwave oven at 80° C. for 30 min and then at 100° C. for a further 30 min (CEM Discover, initial irradiation power 250 W). After cooling to RT, the reaction mixture was purified directly by means of preparative HPLC (method M). 78 mg (58% of th.) of the title compound were obtained.

¹H-NMR (400 MHz, CDCl₃, δ/ppm): 8.32 (d, 1H), 8.28 (d, 2H), 7.72 (d, 2H), 7.52 (dd, 1H), 7.33 (d, 1H), 6.84 (s, 1H), 5.45 (s, 2H), 5.05 (dd, 2H), 5.00 (dd, 2H), 2.33 (s, 3H).

HPLC (method A): Rₜ=4.45 min.

MS (DCI, NH₃): m/z=426 [M+H]⁺.

LC/MS (method I, ESIpos): Rₜ=1.14 min, m/z=426 [M+H]⁺.

The compounds in the following table were prepared from the corresponding precursors analogously to one of the processes described under Example 65 and Example 66. The preparation of most of the N′-hydroxycarboximide amides (hydroxyamidines) employed has been described above; a few were commercially obtainable or are described in the literature.

| | | HPLC: | MS: m/z | LC/MS |
|---|---|---|---|---|
| Example | Structure | Rₜ [min] | [M + H]⁺ | method |
| 67 | 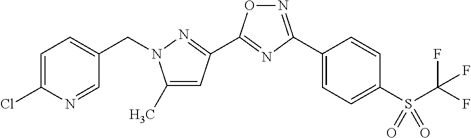 | 2.39 | 484 | E |
| | ¹H-NMR (400 MHz, DMSO-d₆ , δ/ppm): 8.50 (d, 2H), 8.42-8.33 (m, 3H), 7.70 (dd, | | | |
| | 1H), 7.53 (d, 2H), 6.98 (s, 1H), 5.56 (s, 2H), 2.39 (s, 3H). | | | |
| 68 | 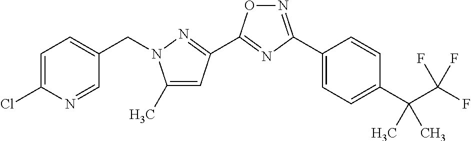 | 1.43 | 412 | F |
| | ¹H-NMR (400 MHz, DMSO-d₆ , δ/ppm): 8.39 (d, 1H), 8.08 (d, 2H), 7.68 (dd 1H), | | | |
| | 7.62 (d, 2H), 7.52 (d, 1H), 6.93 (s, 1H), 5.56 (s, 2H), 2.39 (s, 3H), 1.72 (s, 3H), 1.86 | | | |
| | (s, 3H). | | | |
| 69 | 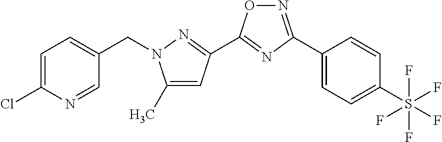 | 1.50 | 478 | F |
| | ¹H-NMR (400 MHz, CDCl₃, δ/ppm): 8.31 (m, 3H), 7.89 (d, 2H), 7.52 (dd, 1H), 7.32 | | | |
| | (d, 2H), 6.84 (s, 1H), 5.44 (s, 2H), 2.32 (s, 3H). | | | |
| 70 | 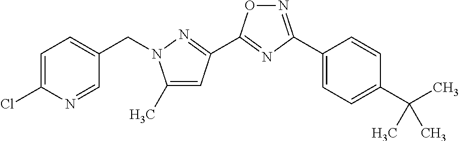 | 5.10 | 408 | A |
| | ¹H-NMR (400 MHz, CDCl₃, δ/ppm): 8.31 (d, 1H), 8.13 (d, 2H), 7.51 (d, 2H), 7.51 (dd, | | | |
| | 1H), 7.32 (d, 1H), 6.83 (s, 1H), 5.44 (s, 2H), 2.32 (s, 3H), 1.36 (s, 9H). | | | |
| 71 | 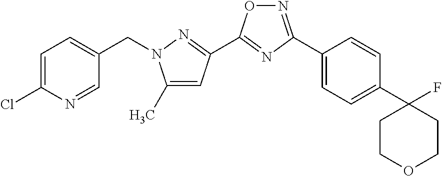 | 4.60 | 454 | A |

¹H-NMR (400 MHz, CDCl₃, δ/ppm): 8.32 (d, 1H), 8.23 (d, 2H), 7.53 (d, 2H), 7.51 (dd,
1H), 7.32 (d, 1H), 6.83 (s, 1H), 5.44 (s, 2H), 4.00-3.86 (m, 4H), 2.33 (s, 3H), 2.29-
2.12 (m, 2H), 1.98-1.92 (m, 2H).

### Example 72

### 5-({4-[3-(4-tert-Butylphenyl)-1,2,4-oxadiazol-5-yl]-2-methyl-1H-pyrrol-1-yl}methyl)-2-chloro-pyridine

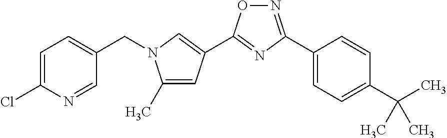

104 μl (1.20 mmol) of oxalyl chloride were added to a solution of 100 mg (0.399 mmol) of the compound from Example 46A in 3 ml of anhydrous methylene chloride at 0° C. under inert conditions. The reaction mixture was stirred at RT for 2 h. All the volatile constituents were then removed on a rotary evaporator and the residue obtained in this way was dried under a high vacuum for 20 min, before being dissolved again in 2 ml of methylene chloride. This solution was added dropwise to a solution of 92 mg (0.479 mmol) of 4-tert-butyl-N′-hydroxybenzenecarboximide amide and 111 μl (0.798 mmol) of triethylamine in 1 ml of methylene chloride at 0° C. After the reaction mixture had been stirred at RT for 1 h, all the volatile constituents were again removed on a rotary evaporator and the residue was dissolved in 4 ml of DMSO. This solution was heated at 120° C. in a microwave oven for 30 min (CEM Discover, initial irradiation power 250 W). After cooling to RT, the reaction mixture was purified directly by means of preparative HPLC (method M). 71 mg (44% of th.) of the title compound were obtained.

¹H-NMR (400 MHz, CDCl₃, δ/ppm): 8.25 (d, 1H), 8.03 (d, 2H), 7.50 (d, 2H), 7.47 (d, 1H), 7.33 (d, 1H), 7.30 (dd, 1H), 6.60 (d, 1H), 5.10 (s, 2H), 2.20 (s, 3H), 1.37 (s, 9H).

HPLC (method A): Rₜ=5.20 min.

MS (DCI, NH₃): m/z=407 [M+H]⁺.

### Example 73

### 3-(4-tert-Butylphenyl)-5-[5-methyl-1-(4-methylbenzyl)-1H-pyrrol-3-yl]-1,2,4-oxadiazole

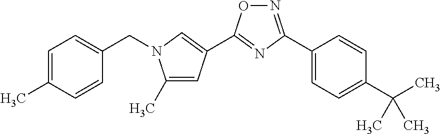

A mixture of 300 mg (1.31 mmol) of the compound from Example 47A, 177 mg (1.31 mmol) of HOBt and 251 mg (1.31 mmol) of EDC in 12 ml of anhydrous DMF was first stirred at RT for 30 min, and 252 mg (1.31 mmol) of 4-tert-butyl-N′-hydroxybenzenecarboximide amide were then added. After the reaction mixture had been stirred at RT for 2 h, it was heated at 170° C. in a microwave oven for 2 min (CEM Discover, initial irradiation power 250 W). After cooling to RT, the reaction mixture was purified directly by means of preparative HPLC (method M). 34 mg (7% of th.) of the title compound were obtained.

¹H-NMR (500 MHz, CDCl₃, δ/ppm): 8.04 (d, 2H), 7.49 (d, 2H), 7.47 (d, 1H), 7.15 (d, 2H), 6.98 (d, 2H), 6.55 (d, 1H), 5.03 (s, 2H), 2.34 (s, 3H), 2.20 (s, 3H), 1.36 (s, 9H).

LC/MS (method F, ESIpos): Rₜ=1.78 min, m/z=386 [M+H]⁺.

The compounds in the following table were prepared from the corresponding precursors analogously to one of the processes described under Example 72 and Example 73. The preparation of most of the N′-hydroxycarboximide amides (hydroxyamidines) employed has been described above; a few were commercially obtainable or are described in the literature.

| | | HPLC: | MS: m/z | LC/MS |
|---|---|---|---|---|
| Example | Structure | Rₜ [min] | [M + H]⁺ | method |
| 74 | 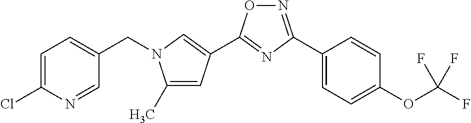 | 3.01 | 435 | C |
| | ¹H-NMR (400 MHz, CDCl₃, δ/ppm): 8.25 (d, 1H), 8.17 (d, 2H), 7.48 (d, 1H), 7.33- | | | |
| | 7.28 (m, 4H), 6.60 (d, 1H), 5.11 (s, 2H), 2.20 (s, 3H). | | | |
| 75 | 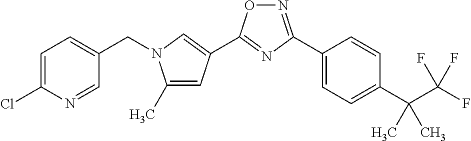 | 1.40 | 461 | I |
| | ¹H-NMR (400 MHz, CDCl₃, δ/ppm): 8.24 (d, 1H), 8.10 (d, 2H), 7.61 (d, 2H), 7.50 (d, | | | |
| | 1H), 7.34-7.30 (m, 2H), 6.60 (d, 1H), 5.13 (s, 2H), 2.31 (s, 6H), 2.21 (s, 3H). | | | |
| 76 | 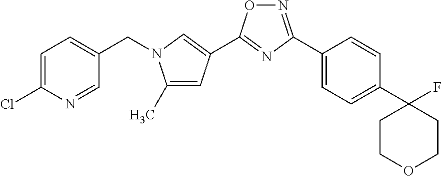 | 4.74 | 453 | A |
| | ¹H-NMR (400 MHz, CDCl₃, δ/ppm): 8.25 (d, 1H), 8.14 (d, 2H), 7.51 (d, 2H), 7.48 (d, | | | |
| | 1H), 7.33 (d, 1H), 7.28 (dd, 1H), 6.60 (d, 1H), 5.11 (s, 2H), 4.00-3.87 (m, 4H), 2.29- | | | |
| | 2.11 (m, 2H), 2.21 (s, 3H), 1.98-1.91 (m, 2H). | | | |
| 77 | 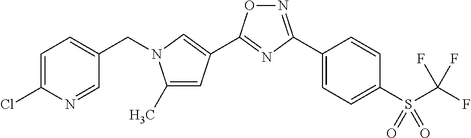 | 4.95 | 483 | A |
| | ¹H-NMR (400 MHz, CDCl₃, δ/ppm): 8.43 (d, 2H), 8.24 (d, 1H), 7.16 (d, 2H), 7.50 (d, | | | |
| | 1H), 7.33 (d, 1H), 7.30 (dd, 1H), 6.61 (d, 1H), 5.12 (s, 2H), 2.22 (s, 3H). | | | |
| 78 | 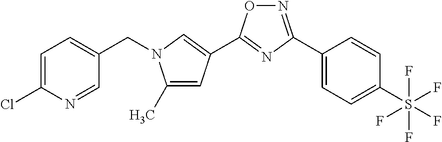 | 1.40 | 477 | I |
| | ¹H-NMR (400 MHz, CDCl₃, δ/ppm): 8.25-8.20 (m, 3H), 7.87 (d, 2H), 7.49 (d, 1H), | | | |
| | 7.33 (d, 1H), 7.29 (dd, 1H), 6.60 (d, 1H), 5.12 (s, 2H), 2.21 (s, 3H). | | | |
| 79 | 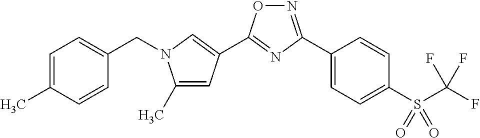 | 1.68 | 462 | F |
| | ¹H-NMR (400 MHz, CDCl₃, δ/ppm): 8.43 (d, 2H), 8.15 (d, 2H), 7.48 (d, 1H), 7.16 (d, | | | |
| | 2H), 6.99 (d, 2H), 6.56 (d, 1H), 5.06 (s, 2H), 2.34 (s, 3H), 2.21 (s, 3H). | | | |
| 80 | 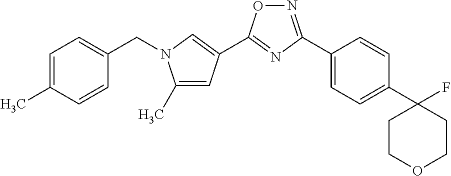 | 2.64 | 432 | E |
| | ¹H-NMR (400 MHz, CDCl₃, δ/ppm): 8.13 (d, 2H), 7.50 (d, 2H), 7.47 (s, 1H), 7.15 (d, | | | |
| | 2H), 6.98 (d, 2H), 6.57 (s, 1H), 5.04 (s, 2H), 4.00-3.87 (m, 4H), 2.33 (s, 3H), 2.29- | | | |
| | 2.11 (m, 2H), 2.20 (s, 3H), 1.97-1.90 (m, 2H). | | | |
| 81 | 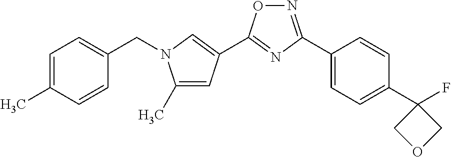 | 2.95 | 404 | C |
| | ¹H-NMR (400 MHz, CDCl₃, δ/ppm): 8.20 (d, 2H), 7.69 (d, 2H), 7 48 (s, 1H) 7.15 (d, | | | |
| | 2H), 6.99 (d, 2H), 6.57 (s, 1H), 5.05 (dd, 2H), 5.03 (s, 2H), 5.00 (dd, 2H), 2.34 (s, 3H), | | | |
| | 2.20 (s, 3H). | | | |
| 82 | 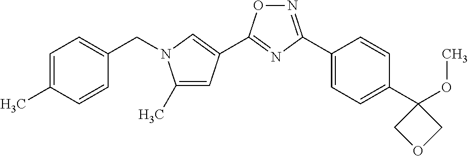 | 2.85 | 416 | C |
| | ¹H-NMR (400 MHz, CDCl₃, δ/ppm): 8.18 (d, 2H), 7.58 (d, 2H), 7.47 (s, 1H), 7.16 (d, | | | |
| | 2H), 6.99 (d, 2H), 6.57 (s, 1H), 5.04 (s, 2H), 4.96 (d, 2H), 4.85 (d, 2H), 3.17 (s, 3H), | | | |
| | 2.34 (s, 3H), 2.20 (s, 3H). | | | |
| 83 | 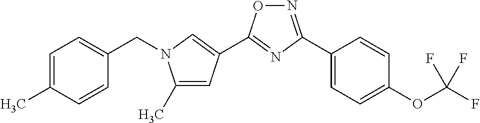 | 5.87 | 414 | B |
| | ¹H-NMR (400 MHz, CDCl₃, δ/ppm): 8.17 (d, 2H), 7.47 (d, 1H), 7.32 (d, 2H), 7.16 (d, | | | |
| | 2H), 6.98 (d, 2H), 6.54 (d, 1H), 5.04 (s, 2H), 2.36 (s, 3H), 2.20 (s, 3H). | | | |
| 84 | 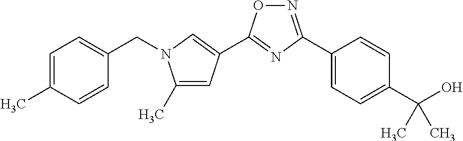 | 2.37 | 388 | E |
| | ¹H-NMR (400 MHz, CDCl₃, δ/ppm): 8.09 (d, 2H), 7.60 (d, 2H), 7.47 (d, 1H), 7.15 (d, | | | |
| | 2H), 6.98 (d, 2H), 6.55 (d, 1H), 5.03 (s, 2H), 2.34 (s, 3H), 2.20 (s, 3H), 1.81 (broad, | | | |
| | 1H), 1.62 (s, 6H). | | | |
| 85 | 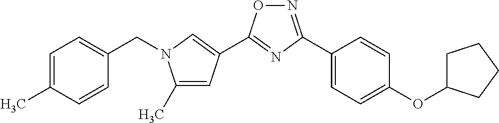 | 3.41 | 414 | C |
| | ¹H-NMR (400 MHz, CDCl₃, δ/ppm): 8.02 (d, 2H), 7.45 (d, 1H), 7.15 (d, 2H), 6.98 (d, | | | |
| | 2H), 6.94 (d, 2H), 6.54 (d, 1H), 5.03 (s, 2H), 4.84-4.80 (m, 1H), 2.33 (s, 3H), 2.19 (s, | | | |
| | 3H), 1.99-1.77 (m, 6H), 1.68-1.61 (m, 2H). | | | |
| 86 | 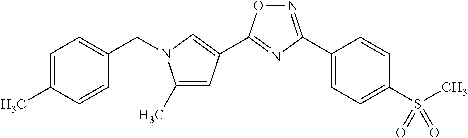 | 2.79 | 408 | C |
| | ¹H-NMR (400 MHz, CDCl₃, δ/ppm): 8.33 (d, 2H), 8.05 (d, 2H), 7.48 (d, 1H), 7.17 (d, | | | |
| | 2H), 6.99 (d, 2H), 6.56 (d, 1H), 5.05 (s, 2H), 3.10 (s, 3H), 2.35 (s, 3H), 2.21 (s, 3H). | | | |
| 87 | 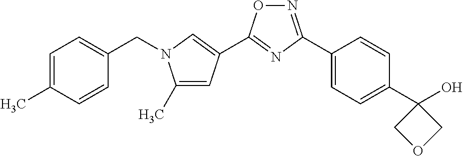 | 2.54 | 402 | C |
| | ¹H-NMR (400 MHz, CDCl₃, δ/ppm): 8.17 (d, 2H), 7.72 (d, 2H), 7.48 (s, 1H), 7.16 (d, | | | |
| | 2H), 6.99 (d, 2H), 6.56 (s, 1H), 5.03 (s, 2H), 4.97-4.91 (m, 4H), 2.35 (s, 3H), 2.20 (s, | | | |
| | 3H). | | | |
| 88 | 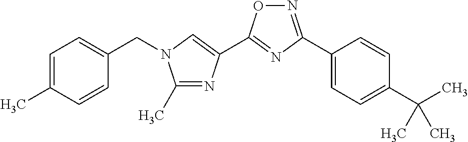 | 2.99 | 387 | C |
| | ¹H-NMR (400 MHz, CDCl₃, δ/ppm): 8.11 (d, 2H), 7.69 (s, 1H), 7.49 (d, 2H), 7.19 (d, | | | |
| | 2H), 7.03 (d, 2H), 5.09 (s, 2H), 2.48 (s, 3H), 2.37 (s, 3H), 1.36 (s, 9H). | | | |
| 89 | 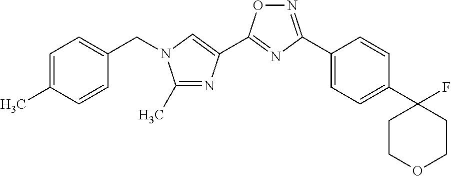 | 2.58 | 433 | C |
| | ¹H-NMR (500 MHz, CDCl₃, δ/ppm): 8.20 (d, 2H), 7.69 (s, 1H), 7.50 (d, 2H) 7.20 (d, | | | |
| | 2H), 7.04 (d, 2H), 5.10 (s, 2H), 3.99-3.95 (m, 2H), 3.90 (dd, 2H), 2.48 (s, 3H), 2.37 (s, | | | |
| | 3H), 2.27-2.12 (m, 2H), 1.94 (dd, 2H). | | | |
| 90 | 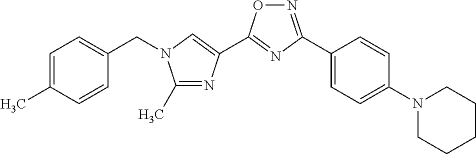 | 1.42 | 414 | F |
| | ¹H-NMR (400 MHz, CDCl₃, δ/ppm): 8.03 (d, 2H), 7.66 (s, 1H), 7.19 (d, 2H), 7.03 (d, | | | |
| | 2H), 6.96 (d, 2H), 5.08 (s, 2H), 3.31-3.27 (m, 4H), 2.47 (s, 3H), 2.36 (s, 3H), 1.73- | | | |
| | 1.58 (m, 6H). | | | |
| 91 | 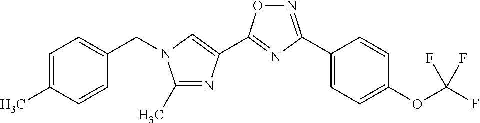 | 2.90 | 415 | C |
| | ¹H-NMR (400 MHz, DMSO-d₆ , δ/ppm): 8.30 (s, 1H), 8.18 (d, 2H), 7.58 (d, 2H), 7.21 | | | |
| | (d, 2H), 7.17 (d, 2H), 5.25 (s, 2H), 2.34 (s, 3H), 2.29 (s, 3H). | | | |
| 92 | 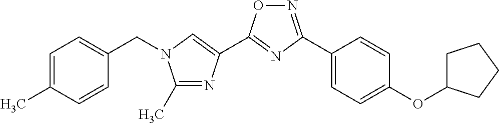 | 2.53 | 415 | E |
| | ¹H-NMR (400 MHz, CDCl₃, δ/ppm): 8.08 (d, 2H), 7.67 (s, 1H), 7.19 (d, 2H) 7.03 (d, | | | |
| | 2H), 6.94 (d, 2H), 5.09 (s, 2H), 4.84-4.80 (m, 1H), 2.47 (s, 3H), 2.37 (s, 3H), 1.98- | | | |
| | 1.77 (m, 6H), 1.68-1.59 (m, 2H). | | | |
| 93 | 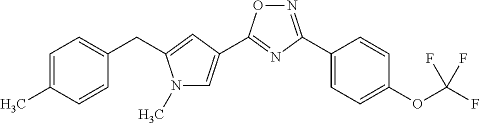 | 1.68 | 414 | F |
| | ¹H-NMR (400 MHz, CDCl₃, δ/ppm): 8.17 (d, 2H), 7.42 (d, 1H), 7.31 (d, 2H), 7.13 (d, | | | |
| | 2H), 7.06 (d, 2H), 6.52 (d, 1H), 3.93 (s, 2H), 3.52 (s, 3H), 2.33 (s, 3H). | | | |
| 94 | 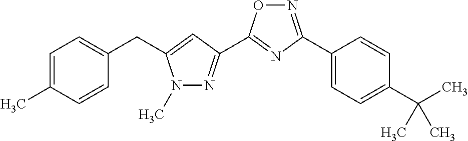 | 1.72 | 387 | F |
| | ¹H-NMR (400 MHz, CDCl₃, δ/ppm): 8.11 (d, 2H), 7.50 (d, 2H), 7.16 (d, 2H), 7.07 (d, | | | |
| | 2H), 6.73 (s, 1H), 4.02 (s, 2H), 3.87 (s, 3H), 2.35 (s, 3H), 1.37 (s, 9H). | | | |
| 95 | 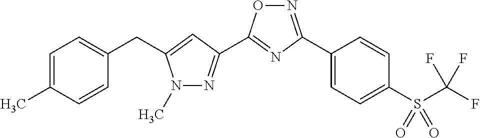 | 2.84 | 463 | D |
| | ¹H-NMR (400 MHz, DMSO-d₆ , δ/ppm): 8.49 (d, 2H), 8.35 (d, 2H), 7.18 (s, 4H), 6.78 | | | |
| | (s, 1H), 4.11 (s, 2H), 3.88 (s, 3H), 2.30 (s, 3H). | | | |
| 96 | 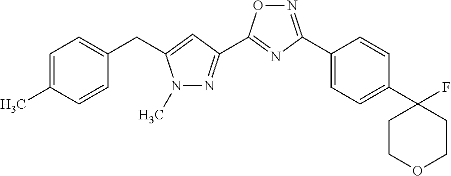 | 2.90 | 433 | C |
| | ¹H-NMR (400 MHz, CDCl₃, δ/ppm): 8.21 (d, 2H), 7.51 (d, 2H), 7.15 (d, 2H), 7.07 (d, | | | |
| | 2H), 6.74 (s, 1H), 4.03 (s, 2H), 3.99-3.87 (m, 4H), 3.87 (s, 3H), 2.36 (s, 3H), 2.29-2.10 | | | |
| | (m, 2H), 1.97-1.91 (m, 2H). | | | |
| 97 | 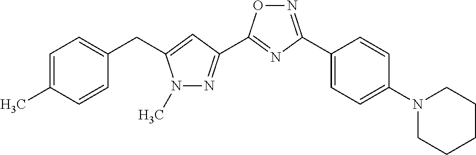 | 1.63 | 414 | F |
| | ¹H-NMR (400 MHz, DMSO-d₆ , δ/ppm): 7.85 (d, 2H), 7.17 (s, 4H), 7.03 (d, 2H), 6.70 | | | |
| | (s, 1H), 4.09 (s, 2H), 3.86 (s, 3H), 3.33-3.30 (m, 4H), 2.29 (s, 3H) 1.62-1.57 (m, 6H). | | | |
| 98 | 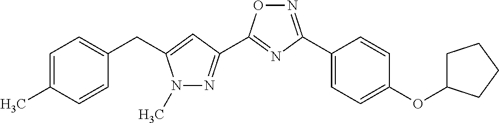 | 3.25 | 415 | C |
| | ¹H-NMR (400 MHz, CDCl₃, δ/ppm): 8.10 (d, 2H), 7.15 (d, 2H), 7.07 (d, 2H), 6.94 (d, | | | |
| | 2H), 6.72 (s, 1H), 4.85-4.80 (m, 1H), 4.02 (s, 2H), 3.85 (s, 3H), 2.34 (s, 3H), 1.99-1.77 | | | |
| | (m, 6H), 1.69-1.60 (m, 2H). | | | |
| 99 | 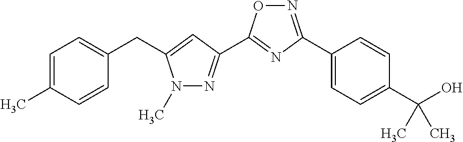 | 2.41 | 389 | D |

¹H-NMR (400 MHz, CDCl₃, δ/ppm): 8.17 (d, 2H), 7.60 (d, 2H), 7.15 (d, 2H), 7.07 (d,
2H), 6.74 (s, 1H), 4.02 (s, 2H), 3.88 (s, 3H), 2.34 (s, 3H), 1.78 (s, 1H), 1.62 (s, 6H).

### Example 100

### 2-({5-[(2-Methyl-4-{3-[4-(trifluoromethoxy)phenyl]-1,2,4-oxadiazol-5-yl}-1H-pyrrol-1-yl)-methyl]pyridin-2-yl}amino)ethanol

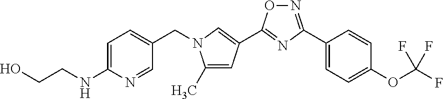

A solution of 120 mg (0.276 mmol) of the compound from Example 74 and 968 mg (5.52 mmol) of 2-{[tert-butyl(dimethyl)silyl]oxy}ethanamine in 1 ml of diethylene glycol dimethyl ether was heated at 180° C. in a microwave oven for 8 h (CEM Discover, initial irradiation power 250 W). After cooling to RT, 6.1 ml (6.07 mmol) of a 1 M solution of tetra-n-butylammonium fluoride in THF were added to the reaction mixture at 0° C. and the mixture was stirred at this temperature for 30 min. The complete reaction mixture was then purified directly by means of preparative HPLC (method M). The product fractions were combined and concentrated, the residue was taken up again in methanol and the mixture was freed from the formic acid from the HPLC chromatography over a bicarbonate cartridge (Polymerlabs, Stratospheres SPE, PL-HCO₃ MP SPE, capacity 0.9 mmol). After removal of the solvent on a rotary evaporator, 45 mg (36% of th.) of the title compound were obtained.

¹H-NMR (400 MHz, CDCl₃, δ/ppm): 8.17 (d, 2H), 7.92 (d, 1H), 7.42 (d, 1H), 7.31 (d, 2H), 7.17 (dd, 1H), 6.53 (d, 1H), 6.46 (d, 1H), 4.91 (s, 2H), 4.90 (broad, 1H), 4.13 (broad, 1H), 3.81 (dd, 2H), 3.55-3.51 (m, 2H), 2.24 (s, 3H).

LC/MS (method D, ESIpos): Rₜ=1.96 min, m/z=460 [M+H]⁺.

Analogously to the process described under Example 100 (but without the use of tetra-n-butylammonium fluoride), the compounds in the following table were obtained from the corresponding educts. The amine components employed were commercially obtainable.

| | | HPLC: | MS: m/z | LC/MS |
|---|---|---|---|---|
| Example | Structure | Rₜ [min] | [M + H]⁺ | method |
| 101 | 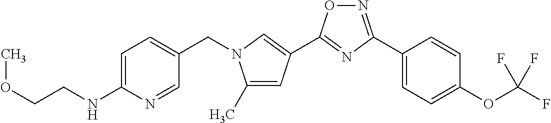 | 1.64 | 474 | E |
| | ¹H-NMR (400 MHz, CDCl₃, δ/ppm): 8.17 (d, 2H), 7.97 (d, 1H), 7.41 (d, 1H), 7.31 (d, | | | |
| | 2H), 7.17 (dd, 1H), 6.52 (d, 1H), 6.40 (d, 1H), 4.92 (s, 2H), 4.87 (broad, 1H), 3.60- | | | |
| | 3.57 (m, 2H), 3.53-3.50 (m, 2H), 3.40 (s, 3H), 2.25 (s, 3H). | | | |
| 102 | 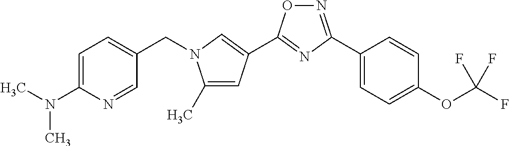 | 1.99 | 444 | C |

¹H-NMR (400 MHz, CDCl₃, δ/ppm): 8.16 (d, 2H), 8.04 (d, 1H), 7.41 (d, 1H), 7.31 (d,
2H), 7.21 (dd, 1H), 6.52 (d, 1H), 6.50 (d, 1H), 4.92 (s, 2H), 3.09 (s, 6H), 2.25 (s, 3H).

### Example 103

### 2-[1-Methyl-5-(4-methylbenzyl)-1H-pyrazol-3-yl]-4-[4-(trifluoromethoxy)phenyl]-1,3-oxazole

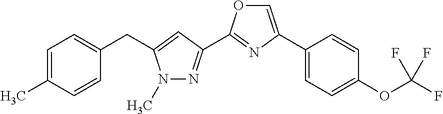

### Step 1: N-{2-Hydroxy-1-[4-(trifluoromethoxy)phenyl]ethyl}-1-methyl-5-(4-methylbenzyl)-1H-pyrazole-3-carboxamide

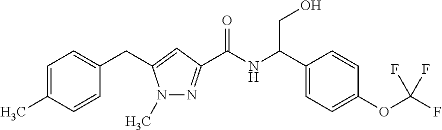

867 mg (2.28 mmol) of HATU were added to a solution of 350 mg (1.52 mmol) of the compound from Example 49A in 7 ml of anhydrous DMF and the mixture was stirred at RT for 30 min. A solution of 504 mg (2.28 mmol) of the compound from Example 22A and 1.7 ml (12.2 mmol) of triethylamine in a further 7 ml of anhydrous DMF was then added and the mixture was stirred at RT for 16 h. Approx. 300 ml of water were then added and the mixture was extracted three times with approx. 200 ml of ethyl acetate each time. The organic extract was washed successively with water and saturated sodium chloride solution. After drying over anhydrous magnesium sulphate, the mixture was filtered and the solvent was removed on a rotary evaporator. The residue obtained was first prepurified by means of MPLC (silica gel, mobile phase: cyclohexane/ethyl acetate 1:1). The pure title compound was then obtained by means of preparative HPLC (method M) (516 mg, 78% of th.).

¹H-NMR (400 MHz, CDCl₃, δ/ppm): 7.45 (d, 1H), 7.42 (d, 2H), 7.20 (d, 2H), 7.10 (d, 2H), 7.01 (d, 2H), 6.57 (s, 1H), 5.24-5.20 (m, 1H), 3.98 (dd, 2H), 3.94 (s, 2H), 3.70 (s, 3H), 2.32 (s, 3H).

HPLC (method B): Rₜ=4.58 min.

MS (DCI, NH₃): m/z=434 [M+H]⁺.

LC/MS (method E, ESIpos): Rₜ=2.12 min, m/z=434 [M+H]⁺.

### Step 2: 2-[1-Methyl-5-(4-methylbenzyl)-1H-pyrazol-3-yl]-4-[4-(trifluoromethoxy)phenyl]-4,5-dihydro-1,3-oxazole

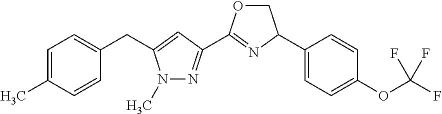

A solution of 485 mg (1.12 mmol) of the compound from Example 103/step 1 and 320 mg (1.34 mmol) of Burgess reagent [methoxycarbonylsulphamoyl-triethylammonium-N-betaine] in 10 ml of anhydrous THF was heated at 70° C. for 1 h. After cooling to RT, approx. 40 ml of water were added and the mixture was extracted three times with approx. 20 ml of ethyl acetate each time. The combined organic extract was washed successively with water and saturated sodium chloride solution. After drying over anhydrous magnesium sulphate and filtration, the solvent was removed on a rotary evaporator. The residue obtained was purified by means of MPLC (silica gel, mobile phase: cyclohexane/ethyl acetate 2:1). 398 mg (86% of th.) of the title compound were obtained.

¹H-NMR (400 MHz, CDCl₃, δ/ppm): 7.33 (d, 2H), 7.19 (d, 2H), 7.11 (d, 2H), 7.03 (d, 2H), 6.57 (s, 1H), 5.35 (dd, 1H), 4.79 (dd, 1H), 4.24 (dd, 1H), 3.96 (s, 2H), 3.79 (s, 3H), 2.33 (s, 3H).

MS (DCI, NH₃): m/z=416 [M+H]⁺.

LC/MS (method F, ESIpos): Rₜ=2.12 min, m/z=416 [M+H]⁺.

### Step 3: 2-[1-Methyl-5-(4-methylbenzyl)-1H-pyrazol-3-yl]-4-[4-(trifluoromethoxy)phenyl]-1,3-oxazole

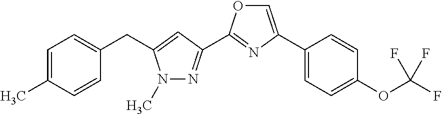

250 mg (0.602 mmol) of the compound from Example 103/step 2 were dissolved in 6 ml of THF and 209 mg (2.41 mmol) of manganese dioxide (“precipitated, active” quality) were added. After the reaction mixture had been heated under reflux for 2.5 h, the same amount of manganese dioxide was again added and the mixture was heated under reflux for a further 2.5 h. After cooling to RT, the mixture was diluted with THF and filtered over kieselguhr. The filtrate was freed from the solvent on a rotary evaporator. The residue was dissolved in methylene chloride and the solution was washed in each case once with 1 M hydrochloric acid and water. After drying over anhydrous magnesium sulphate and filtration, the solvent was removed on a rotary evaporator. The crude product was purified by means of preparative HPLC (method M). 107 mg (43% of th.) of the title compound were obtained.

¹H-NMR (400 MHz, CDCl₃, δ/ppm): 7.92 (s, 1H), 7.84 (d, 2H), 7.25 (d, 2H), 7.13 (d, 2H), 7.07 (d, 2H), 6.62 (s, 1H), 4.00 (s, 2H), 3.82 (s, 3H), 2.34 (s, 3H).

HPLC (method B): Rₜ=5.32 min.

MS (ESIpos): m/z=414 [M+H]⁺.

LC/MS (method D, ESIpos): Rₜ=2.81 min, m/z=414 [M+H]⁺.

The compounds in the following table were prepared from the corresponding educts analogously to the processes described in Examples 1 to 3. Depending on the polarity of the compounds, these were either isolated by extraction by stirring in methylene chloride, ethyl acetate, acetonitrile or diethyl ether, or they were purified by means of preparative HPLC or by means of MPLC over silica gel with cyclohexane/ethyl acetate mixtures as the mobile phase. The arylmethyl chlorides, bromides or methanesulphonates used as educts were either commercially obtainable or were prepared as described above, or their preparation is described in the literature:

| | | HPLC: | MS: m/z | LC/MS |
|---|---|---|---|---|
| Example | Structure | Rₜ [min] | [M + H]⁺ | method |
| 104 | 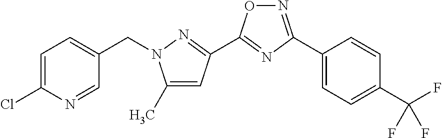 | 1.30 | 420 | I |
| | ¹H-NMR (400 MHz, CDCl₃, δ/ppm): 8.34-8.30 (m, 3H), 7.77 (d, 2H), 7.52 (dd, 1H), | | | |
| | 7.31 (d, 1H), 6.84 (s, 1H), 5.44 (s, 2H), 2.32 (s, 3H). | | | |
| 105 | 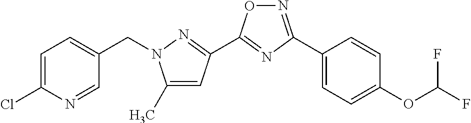 | 1.21 | 418 | I |
| | ¹H-NMR (400 MHz, CDCl₃, δ/ppm): 8.32 (d, 1H), 8.21 (d, 2H), 7.51 (dd, 1H), 7.31 (d, | | | |
| | 1H), 7.22 (d, 2H), 6.82 (s, 1H), 6.60 (t, 1H), 5.43 (s, 2H), 2.32 (s, 3H). | | | |
| 106 | 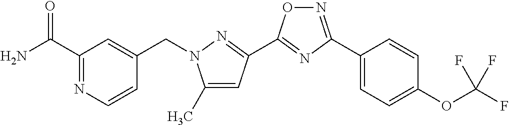 | 1.17 | 445 | I |
| | ¹H-NMR (400 MHz, CDCl₃, δ/ppm): 8.53 (d, 1H), 8.25 (d, 2H), 8.03 (s, 1H), 7.82 (s, | | | |
| | broad, 1H), 7.32 (d, 2H), 7.12 (d, 1H), 6.86 (s, 1H), 5.64 (s, broad, 1H), 5.52 (s, 2H), | | | |
| | 2.30 (s, 3H). | | | |
| 107 | 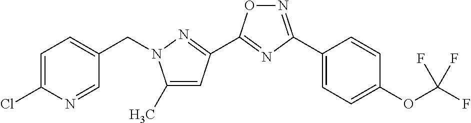 | 1.29 | 436 | F |

¹H-NMR (400 MHz, CDCl₃, δ/ppm): 8.33 (s, 1H), 8.22 (d, 2H), 7.71 (s, 1H) 7.39 (s,
2H), 7.32 (d, 2H), 5.18 (s, 2H), 2.50 (s, 3H).

The compounds in the following table were prepared from the corresponding precursors analogously to one of the processes described under Example 65, 66, 72 and 73. The preparation of most of the N′-hydroxycarboximide amides (hydroxyamidines) used has been described above; a few were commercially obtainable or are described in the literature.

| | | HPLC: | MS: m/z | LC/MS |
|---|---|---|---|---|
| Example | Structure | Rₜ [min] | [M + H]⁺ | method |
| 108 | 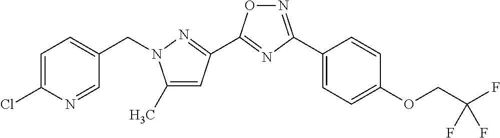 | 1.23 | 450 | I |
| | ¹H-NMR (400 MHz, CDCl₃, δ/ppm): 8.32 (d, 1H), 8.18 (d, 2H), 7.51 (dd, 1H), 7.32 (d, | | | |
| | 1H), 7.05 (d, 2H), 6.82 (s, 1H), 5.44 (s, 2H), 4.43 (quart, 2H), 2.33 (s, 3H). | | | |
| 109 | 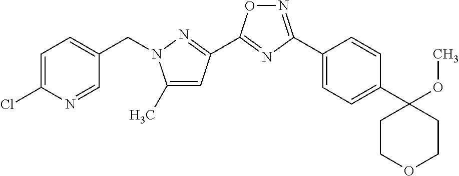 | 1.18 | 466 | I |
| | ¹H-NMR (400 MHz, CDCl₃, δ/ppm): 8.32 (d, 1H), 8.21 (d, 2H), 7.53 (d, 2H), 7.52 (dd, | | | |
| | 1H), 7.32 (d, 1H), 6.84 (s, 1H), 5.44 (s, 2H), 3.93-3.83 (m, 4H), 3.01 (s, 3H), 2.33 (s, | | | |
| | 3H), 2.11-1.98 (m, 4H). | | | |
| 110 | 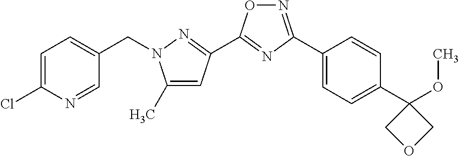 | 1.11 | 438 | I |
| | ¹H-NMR (400 MHz, CDCl₃, δ/ppm): 8.32 (d, 1H), 8.26 (d, 2H), 7.61 (d, 2H), 7.52 (dd, | | | |
| | 1H), 7.32 (d, 1H), 6.84 (s, 1H), 5.45 (s, 2H), 4.97 (d, 2H), 4.85 (d, 2H), 3.19 (s, 3H), | | | |
| | 2.33 (s, 3H). | | | |
| 111 | 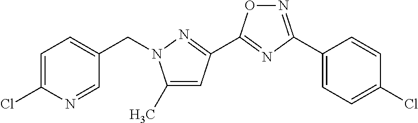 | 1.42 | 386 | F |
| | ¹H-NMR (400 MHz, CDCl₃, δ/ppm): 8.31 (d, 1H), 8.13 (d, 2H), 7.52-7.46 (m, 3H), | | | |
| | 7.31 (d, 1H), 6.82 (s, 1H), 5.43 (s, 2H), 2.32 (s, 3H). | | | |
| 112 | 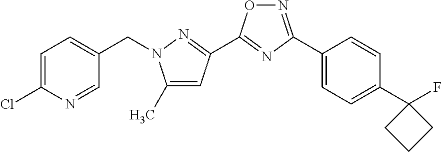 | 1.32 | 424 | I |
| | ¹H-NMR (400 MHz, CDCl₃, δ/ppm): 8.32 (d, 1H), 8.22 (d, 2H), 7.60 (d, 2H), 7.51 (dd, | | | |
| | 1H), 7.32 (d, 1H), 6.84 (s, 1H), 5.44 (s, 2H), 2.77-2.55 (m, 4H), 2.33 (s, 3H), 2.20- | | | |
| | 2.08 (m, 1H), 1.87-1.75 (m, 1H). | | | |
| 113 | 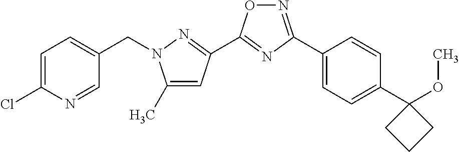 | 1.30 | 436 | I |
| | ¹H-NMR (400 MHz, CDCl₃, δ/ppm): 8.32 (d, 1H), 8.20 (d, 2H), 7.56 (d, 2H), 7.52 (dd, | | | |
| | 1H), 7.32 (d, 1H), 6.84 (s, 1H), 5.44 (s, 2H), 2.97 (s, 3H), 2.44-2.41 (m, 4H), 2.33 (s, | | | |
| | 3H), 2.03-1.93 (m, 1H), 1.78-1.67 (m, 1H). | | | |
| 114 | 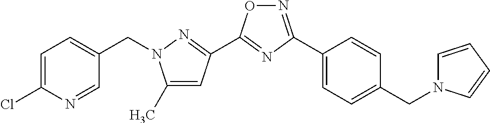 | 1.24 | 431 | I |
| | ¹H-NMR (400 MHz, CDCl₃, δ/ppm): 8.31 (d, 1H), 8.13 (d, 2H), 7.50 (dd, 1H), 7.31 (d, | | | |
| | 1H), 7.22 (d, 2H), 6.82 (s, 1H), 6.72 (s, 2H), 6.22 (s, 2H), 5.42 (s, 2H), 5.13 (s, 2H), | | | |
| | 2.31 (s, 3H). | | | |
| 115 | 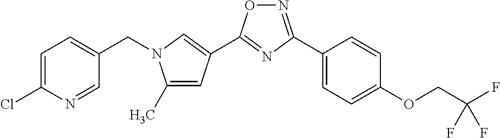 | 1.30 | 449 | I |
| | ¹H-NMR (400 MHz, CDCl₃, δ/ppm): 8.25 (d, 1H), 8.10 (d, 2H), 7.46 (d, 1H), 7.33 (d, | | | |
| | 1H), 7.27 (dd, 1H), 7.04 (d, 2H), 6.59 (d, 1H), 5.10 (s, 2H), 5.03 (s, 2H), 4.42 (quart, | | | |
| | 2H), 2.20 (s, 3H). | | | |
| 116 | 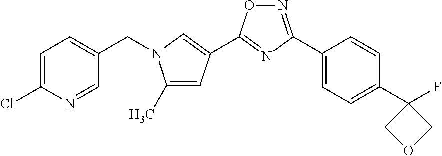 | 1.21 | 425 | I |
| | ¹H-NMR (400 MHz, CDCl₃, δ/ppm): 8.25 (d, 1H), 8.20 (d, 2H), 7.70 (d, 2H), 7.49 (d, | | | |
| | 1H), 7.33 (d, 1H), 7.28 (dd, 1H), 6.61 (d, 1H), 5.11 (s, 2H), 5.05 (dd, 2H), 5.00 (dd, | | | |
| | 2H), 2.21 (s, 3H). | | | |
| 117 | 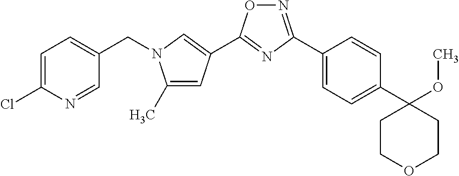 | 1.38 | 465 | F |
| | ¹H-NMR (400 MHz, CDCl₃, δ/ppm): 8.25 (d, 1H), 8.12 (d, 2H), 7.51 (d, 2H), 7.48 (d, | | | |
| | 1H), 7.33 (d, 1H), 7.28 (dd, 1H), 6.60 (d, 1H), 5.11 (s, 2H), 3.94-3.81 (m, 4H), 3.01 (s, | | | |
| | 3H), 2.20 (s, 3H), 2.11-1.97 (m, 4H). | | | |
| 118 | 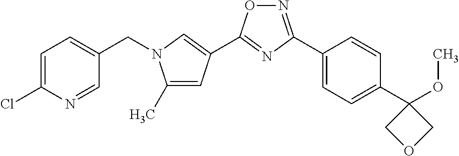 | 1.17 | 437 | I |
| | ¹H-NMR (400 MHz, CDCl₃, δ/ppm): 8.25 (d, 1H), 8.18 (d, 2H), 7.59 (d, 2H), 7.48 (d, | | | |
| | 1H), 7.33 (d, 1H), 7.29 (dd, 1H), 6.61 (d, 1H), 5.11 (s, 2H), 4.96 (d, 2H), 4.85 (d, 2H), | | | |
| | 3.17 (s, 3H), 2.21 (s, 3H). | | | |
| 119 | 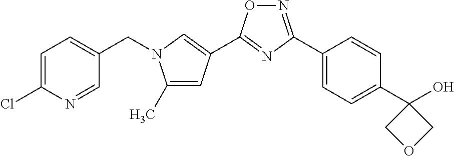 | 4.17 | 423 | A |
| | ¹H-NMR (400 MHz, CDCl₃, δ/ppm): 8.25 (d, 1H), 8.17 (d, 2H), 7.75 (d, 2H), 7.48 (d, | | | |
| | 1H), 7.33 (d, 1H), 7.28 (dd, 1H), 6.61 (d, 1H), 5.11 (s, 2H), 4.97-4.94 (m, 4H), 2.78 | | | |
| | (broad, 1H), 2.20 (s, 3H). | | | |
| 120 | 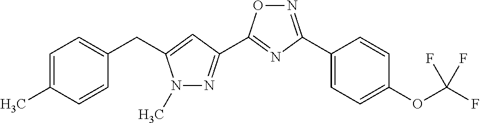 | 2.73 | 415 | E |

¹H-NMR (400 MHz, CDCl₃, δ/ppm): 8.24 (d, 2H), 7.33 (d, 2H), 7.16 (d, 2H), 7.07 (d,
2H), 6.75 (s, 1H), 4.03 (s, 2H), 3.87 (s, 3H), 2.35 (s, 3H).

The examples in the following table were prepared analogously to the processes described under Example 24 or Example 100 using the particular corresponding amine and the corresponding 2-chloropyridine compound from Examples 2, 3, 112 or 113. In contrast to that described in Example 24, these reactions were usually carried out in DMSO as the solvent (approx. 0.5 ml of DMSO per 0.10 mmol of the 2-chloropyridine educt). For the preparation of some examples it was necessary to prolong the duration of the reaction by up to a further 10 hours and/or to increase the amount of educt amine employed by up to a further 10 equivalents, based on the 2-chloropyridine derivative employed. In some examples it was furthermore necessary to carry out two purifications by means of preparative HPLC. Most of the amine components employed were commercially obtainable; some were prepared by processes described in the literature.

| | | HPLC: | MS: m/z | LC/MS |
|---|---|---|---|---|
| Example | Structure | Rₜ [min] | [M + H]⁺ | method |
| 121 | 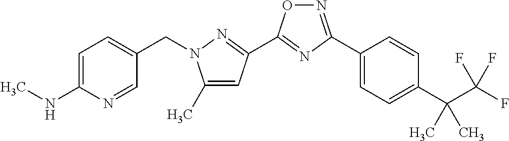 | 0.97 | 457 | I |
| | ¹H-NMR (400 MHz, CDCl₃, δ/ppm): 8.19 (d, 2H), 8.02 (d, 1H), 7.61 (d, 2H), 7.38 (dd, | | | |
| | 1H), 6.77 (s, 1H), 6.35 (d, 1H), 5.29 (s, 2H), 4.55-4.48 (m, 1H), 2.90 (d, 3H), 2.31 (s, | | | |
| | 3H), 1.61 (s, 6H). | | | |
| 122 | 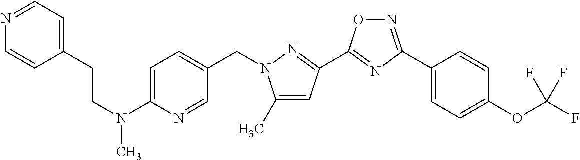 | 0.99 | 536 | O |
| 123 | 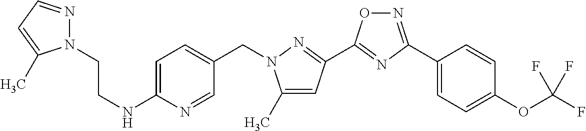 | 1.05 | 525 | O |
| 124 | 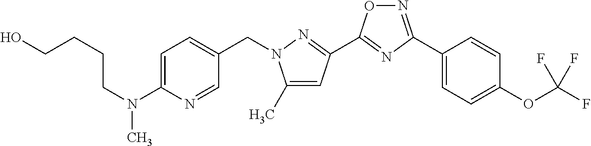 | 0.96 | 503 | O |
| 125 | 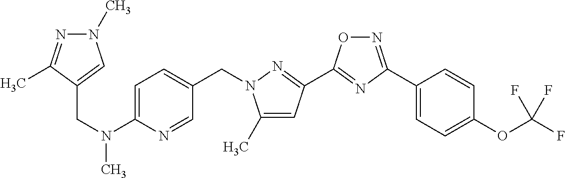 | 1.11 | 539 | O |
| 126 | 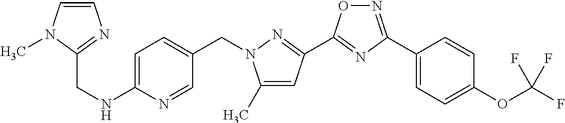 | 0.95 | 511 | O |
| 127 | 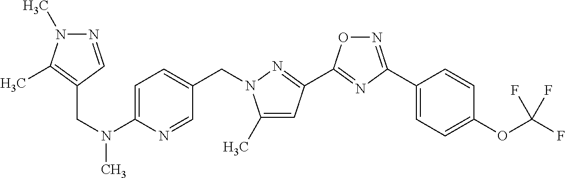 | 1.12 | 539 | O |
| 128 | 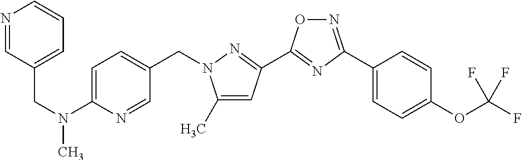 | 1.15 | 523 | O |
| 129 | 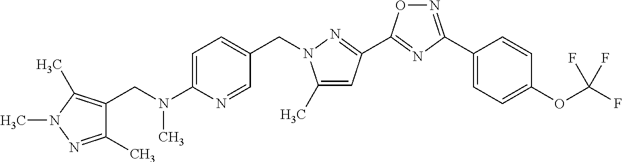 | 1.13 | 553 | O |
| 130 | 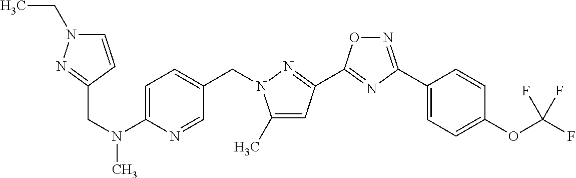 | 1.11 | 539 | O |
| 131 | 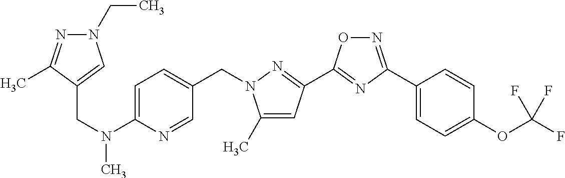 | 1.12 | 553 | O |
| 132 | 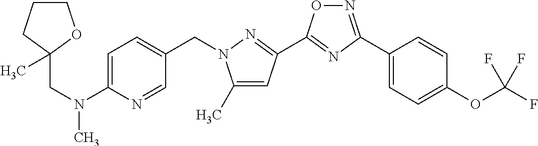 | 1.07 | 529 | O |
| 133 | 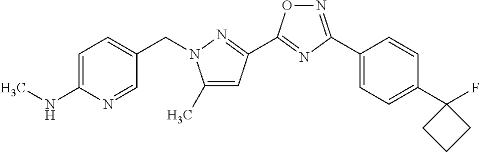 | 1.07 | 419 | F |
| | ¹H-NMR (400 MHz, CDCl₃, δ/ppm): 8.23 (d, 2H), 8.01 (d, 1H), 7.59 (d, 2H), 7.40 (dd, | | | |
| | 1H), 6.78 (s, 1H), 6.37 (d, 1H), 5.30 (s, 2H), 4.96 (broad, 1H), 2.91 (s, 3H), 2.79-2.55 | | | |
| | (m, 4H), 2.32 (s, 3H), 2.17-2.05 (m, 1H), 1.87-1.75 (m, 1H). | | | |
| 134 | 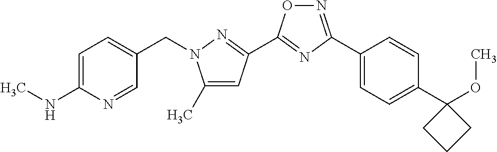 | 1.02 | 431 | F |

¹H-NMR (400 MHz, CDCl₃, δ/ppm): 8.21 (d, 2H), 8.04 (d, 1H), 7.56 (d, 2H), 7.39 (dd,
1H), 6.78 (s, 1H), 6.36 (d, 1H), 5.30 (s, 2H), 4.63 (broad, 1H), 2.97 (s, 3H), 2.91 (d,
3H), 2.45-2.39 (m, 4H), 2.32 (s, 3H), 2.03-1.93 (m, 1H), 1.78-1.67 (m, 1H).

### Example 135

### 5-[(5-Methyl-3-{3-[4-(trifluoromethoxy)phenyl]-1,2,4-oxadiazol-5-yl}-1H-pyrazol-1-yl)methyl]-pyridin-2-amine

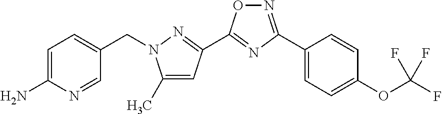

### Step 1: 2-Hydrazinyl-5-[(5-methyl-3-{3-[4-(trifluoromethoxy)phenyl]-1,2,4-oxadiazol-5-yl}-1H-pyrazol-1-yl)methyl]pyridine

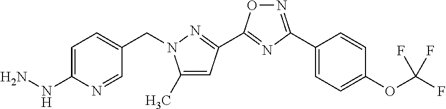

5.0 ml (103 mmol) of hydrazine hydrate were added to 1.0 g (2.29 mmol) of the compound from Example 2 under argon at RT. The mixture was heated under reflux for 16 h, while stirring, a further 5.0 ml of hydrazine hydrate were then added and the mixture was stirred under reflux again for 16 h. The solid formed was dissolved by addition of 10 ml of ethanol and the mixture was then heated under reflux for a further 24 h, while stirring. After cooling to RT, the solid which had precipitated out on cooling was filtered off with suction, washed once with a 1:1 mixture of water and ethanol and dried in vacuo. 788 mg (80% of th.) of the title compound were obtained.

¹H-NMR (400 MHz, CDCl₃, δ/ppm): 8.25 (d, 2H), 8.06 (d, 1H), 7.41 (dd, 1H), 7.32 (d, 2H), 6.78 (s, 1H), 6.69 (d, 1H), 5.88 (s, 1H), 5.31 (s, 2H), 3.80 (s, broad, 2H), 2.31 (s, 3H).

LC/MS (method I, ESIpos): Rₜ=0.90 min, m/z=432 [M+H]⁺.

### Step 2: 2-Azido-5-[(5-methyl-3-{3-[4-(trifluoromethoxy)phenyl]-1,2,4-oxadiazol-5-yl}-1H-pyrazol-1-yl)methyl]pyridine

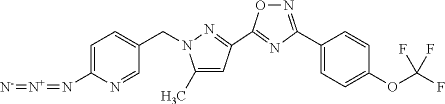

450 mg (1.04 mmol) of the compound from Example 135/step 1 were initially introduced into 10 ml of concentrated hydrochloric acid and the mixture was cooled with an ice bath. A solution of 576 mg (8.35 mmol) of sodium nitrite in 20 ml of water was slowly added dropwise, while stirring, and the mixture was then allowed to come to RT and was stirred at RT for a further 5 h. The mixture was then rendered alkaline by addition of 10% strength sodium hydroxide solution. The solid formed was filtered off, washed twice with water and dried in vacuo. 459 mg (99% of th.) of the title compound were obtained.

¹H-NMR (400 MHz, CD₃OD, δ/ppm): 9.10 (s, 1H), 8.22 (d, 2H), 8.09 (d, 1H), 7.79 (d, 1H), 7.46 (d, 2H), 6.92 (s, 1H), 5.65 (s, 2H), 2.49 (s, 3H).

LC/MS (method I, ESIpos): Rₜ=1.17 min, m/z=443 [M+H]⁺.

### Step 3: 5-[(5-Methyl-3-{3-[4-(trifluoromethoxy)phenyl]-1,2,4-oxadiazol-5-yl}-1H-pyrazol-1-yl)methyl]pyridin-2-amine

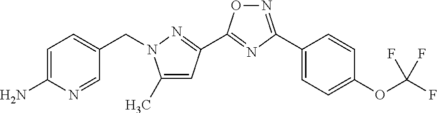

450 mg (1.02 mmol) of the compound from Example 135/step 2 were initially introduced into 20 ml of a 7:3 mixture of methanol and water under argon. 1.03 g (5.99 mmol) of tributylphosphane were added and the mixture was heated under reflux for 2 h, while stirring. After cooling to RT, the mixture was filtered and the filtrate was concentrated. The residue obtained was dissolved in acetonitrile and purified by means of preparative HPLC (method N). The combined product-containing fractions were concentrated to a low residual volume of solvent. A little sodium bicarbonate was added, after which a solid precipitated out. This was filtered off, washed twice with water and dried in vacuo. 339 mg (80% of th.) of the title compound were obtained.

¹H-NMR (400 MHz, CDCl₃, δ/ppm): 8.24 (d, 2H), 8.01 (d, 1H), 7.38-7.31 (m, 3H), 6.79 (s, 1H), 6.48 (d, 1H), 5.30 (s, 2H), 4.50 (s, 2H), 2.32 (s, 3H).

LC/MS (method F, ESIpos): Rₜ=1.05 min, m/z=417 [M+H]⁺.

### Example 136

### 5-[(5-Methyl-3-{3-[4-(trifluoromethoxy)phenyl]-1,2,4-oxadiazol-5-yl}-1H-pyrazol-1-yl)methyl]-pyridine 1-oxide

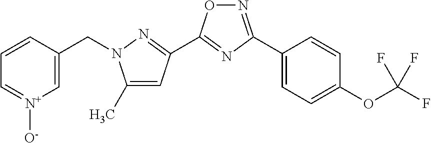

70 mg (0.262 mmol) of hydrogen peroxide-urea complex were added to a solution of 50 mg (0.125 mmol) of the compound from Example 6 in 2 ml of THF and the mixture was cooled to 0° C. 39 μl (0.274 mmol) of trifluoroacetic anhydride were slowly added to the mixture, while stirring, and the mixture was stirred at 0° C. for 30 min. It was then warmed to RT and approx. 2 ml of saturated aqueous sodium thiosulphate solution and approx. 1 ml of 0.5 M hydrochloric acid were added. The mixture was then extracted with methylene chloride. After drying of the organic phase over anhydrous magnesium sulphate and filtration, the solvent was removed on a rotary evaporator. The crude product obtained in this way was purified by stirring with diethyl ether. 30 mg (59% of th.) of the title compound were obtained.

¹H-NMR (400 MHz, CDCl₃, δ/ppm): 8.24 (d, 2H), 8.15 (d, 1H), 8.04 (s, 1H), 7.33 (d, 2H), 7.28 (t, 1H), 7.10 (d, 1H), 6.87 (s, 1H), 5.41 (s, 2H), 2.37 (s, 3H).

HPLC (method A): Rₜ=4.39 min.

MS (DCI, NH₃): m/z=418 [M+H]⁺, 435 [M+NH₄]⁺.

LC/MS (method F, ESIpos): Rₜ=1.18 min, m/z=418 [M+H]⁺, 835 [2M+H]⁺.

### Example 137

### 2-Methyl-5-[(5-methyl-3-{3-[4-(trifluoromethoxy)phenyl]-1,2,4-oxadiazol-5-yl}-1H-pyrazol-1-yl)-methyl]pyridine 1-oxide

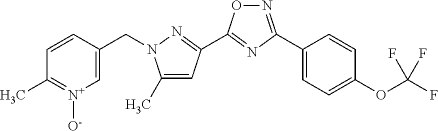

Analogously to the process described under Example 136, 24 mg (39% of th.) of the title compound were obtained from 60 mg (0.144 mmol) of the compound from Example 7.

¹H-NMR (400 MHz, CDCl₃, δ/ppm): 8.25 (d, 2H), 8.12 (s, 1H), 7.34 (d, 2H), 7.23 (d, 1H), 7.02 (d, 1H), 6.83 (s, 1H), 5.38 (s, 2H), 2.50 (s, 3H), 2.33 (s, 3H).

MS (DCI, NH₃): m/z=432 [M+H]⁺.

### Example 138

### 2-Chloro-5-[(5-methyl-3-{3-[4-(trifluoromethoxy)phenyl]-1,2,4-oxadiazol-5-yl}-1H-pyrazol-1-yl)-methyl]pyridine 1-oxide

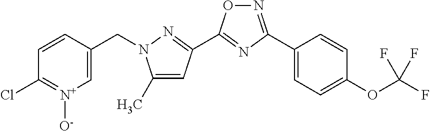

A solution of 500 mg (1.15 mmol) of the compound from Example 2 and 1.06 g (4.59 mmol, content approx. 75%) of meta-chloroperbenzoic acid (MCPBA) in 10 ml of methylene chloride was stirred at RT for 3 days. It was then diluted with 50 ml of methylene chloride and washed successively in each case once with 50 ml of 1 N sodium hydroxide solution, water and saturated sodium chloride solution. The organic phase was dried over magnesium sulphate and filtered and the solvent was removed. After the residue had been dried in vacuo, 482 mg (93% of th.) of the title compound were obtained.

¹H-NMR (400 MHz, CDCl₃, δ/ppm): 8.24 (d, 2H), 8.19 (d, 1H), 7.48 (d, 1H), 7.33 (d, 2H), 7.09-7.05 (dd, 1H), 6.85 (s, 1H), 5.39 (s, 2H), 2.35 (s, 3H).

LC/MS (method F, ESIpos): Rₜ=1.26 min, m/z=452/454 [M+H]⁺.

### Example 139

### N-Methyl-5-[(5-methyl-3-{3-[4-(1,1,1-trifluoro-2-methylpropan-2-yl)phenyl]-1,2,4-oxadiazol-5-yl}-1H-pyrazol-1-yl)methyl]pyridin-2-amine 1-oxide

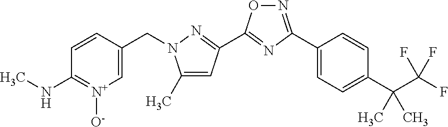

A mixture of 100 mg (0.219 mmol) of the compound from Example 121 and 151 mg (0.676 mmol, content approx. 75%) of meta-chloroperbenzoic acid (MCPBA) in 4 ml of methylene chloride was stirred at RT for 30 min. The mixture was then diluted with 20 ml of methylene chloride and washed twice with 20 ml of saturate aqueous sodium bicarbonate solution, and the organic phase was dried over magnesium sulphate, filtered and concentrated. The residue was purified by means of preparative HPLC (method N). The combined product fractions were concentrated to a residual volume of aqueous phase. Saturated sodium bicarbonate solution was added and the mixture was extracted twice with methylene chloride. The combined methylene chloride phases were dried over magnesium sulphate, filtered and concentrated. The residue was triturated with 0.5 ml of diethyl ether and the solvent was removed again on a rotary evaporator. After drying in vacuo, 50 mg (47% of th., purity of 98%) of the title compound were obtained.

¹H-NMR (400 MHz, CDCl₃, δ/ppm): 8.19 (d, 2H), 8.05 (s, broad, 1H), 7.63 (d, 2H), 7.15 (d, 1H), 6.80 (s, 1H), 6.80-6.70 (m, 1H), 6.56 (d, 1H), 5.29 (s, 2H), 3.08-2.95 (d, 3H), 2.33 (s, 3H), 1.63 (s, 6H).

LC/MS (method I, ESIpos): Rₜ=1.12 min, m/z=473 [M+H]⁺.

### Example 140

### tert-Butyl ({1-[4-(5-{1-[(6-chloropyridin-3-yl)methyl]-5-methyl-1H-pyrazol-3-yl}-1,2,4-oxa-diazol-3-yl)phenyl]cyclobutyl}oxy)acetate

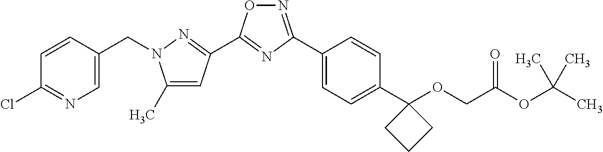

Analogously to the process described under Example 66, 192 mg (60% of th.) of the title compound were prepared from 150 mg (0.596 mmol) of the compound from Example 45A and 210 mg (0.656 mmol) of the compound from Example 57A.

¹H-NMR (400 MHz, CDCl₃, δ/ppm): 8.32 (d, 1H), 8.20 (d, 2H), 7.60 (d, 2H), 7.52 (dd, 1H), 7.32 (d, 1H), 6.83 (s, 1H), 5.43 (s, 2H), 3.60 (s, 2H), 2.56-2.41 (m, 4H), 2.33 (s, 3H), 2.08-1.99 (m, 1H), 1.78-1.66 (m, 1H), 1.43 (s, 9H).

HPLC (method A): Rₜ=4.95 min.

MS (DCI, NH₃): m/z=536 [M+H]⁺, 553 [M+NH₄]⁺.

LC/MS (method F, ESIpos): Rₜ=1.56 min, m/z=536/538 [M+H]⁺.

### Example 141

### 2-Chloro-5-[(5-methyl-3-{3-[4-(piperidin-1-yl)phenyl]-1,2,4-oxadiazol-5-yl}-1H-pyrazol-1-yl)-methyl]pyridine

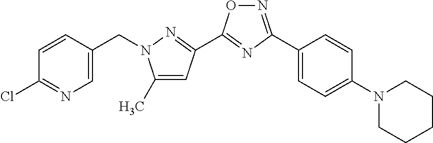

Analogously to the process described under Example 66, 33 mg (14% of th., purity of 94%) of the title compound were prepared from 125 mg (0.497 mmol) of the compound from Example 45A and 184 mg (0.546 mmol) of the compound from Example 10A.

¹H-NMR (400 MHz, CDCl₃, δ/ppm): 8.31 (d, 1H), 8.04 (d, 2H), 7.50 (dd, 1H), 7.31 (d, 1H), 6.96 (d, 2H), 6.80 (s, 1H), 5.42 (s, 2H), 3.32-3.28 (m, 4H), 2.30 (s, 3H), 1.73-1.67 (m, 4H), 1.65-1.61 (m, 2H).

LC/MS (method I, ESIpos): Rₜ=1.33 min, m/z=435/437 [M+H]⁺.

### Example 142

### 2-Chloro-5-[(5-methyl-3-{3-[4-(tetrahydro-2H-pyran-4-yl)phenyl]-1,2,4-oxadiazol-5-yl}-1H-pyrazol-1-yl)methyl]pyridine

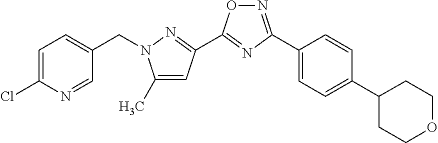

Analogously to the process described under Example 2, 106 mg (0.628 mmol) of 2-chloro-5-(chloromethyl)pyridine and 150 mg (0.483 mmol) of the compound from Example 74A were reacted to give 74 mg (34% of th., purity of 95%) of the title compound. The product was isolated by means of preparative HPLC (method M).

¹H-NMR (400 MHz, CDCl₃, δ/ppm): 8.32 (d, 1H), 8.14 (d, 2H), 7.50 (dd, 1H), 7.35 (d, 2H), 7.31 (d, 1H), 6.82 (s, 1H), 5.42 (s, 2H), 4.12-4.07 (m, 2H), 3.58-3.51 (m, 2H), 2.87-2.80 (m, 1H), 2.32 (s, 3H), 1.91-1.79 (m, 4H).

LC/MS (method I, ESIpos): Rₜ=1.18 min, m/z=436/438 [M+H]⁺.

### Example 143

### 1-[4-(5-{1-[(6-Chloropyridin-3-yl)methyl]-5-methyl-1H-pyrazol-3-yl}-1,2,4-oxadiazol-3-yl)-phenyl]cyclobutanol

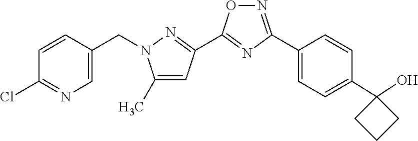

Analogously to the process described under Example 65, 135 mg (32% of th.) of the title compound were prepared from 250 mg (0.993 mmol) of the compound from Example 45A and 225 mg (1.09 mmol) of the compound from Example 51A. The product was isolated by means of preparative HPLC (method M).

¹H-NMR (400 MHz, CDCl₃, δ/ppm): 8.32 (d, 1H), 8.20 (d, 2H), 7.63 (d, 2H), 7.51 (dd, 1H), 7.32 (d, 1H), 6.83 (s, 1H), 5.43 (s, 2H), 2.64-2.58 (m, 2H), 2.45-2.38 (m, 2H), 2.33 (s, 3H), 2.14-2.03 (m, 2H), 1.82-1.71 (m, 1H).

LC/MS (method I, ESIpos): Rₜ=1.11 min, m/z=422/424 [M+H]⁺.

### Example 144

### 2-Chloro-5-[(5-methyl-3-{3-[4-(methylsulphonyl)phenyl]-1,2,4-oxadiazol-5-yl}-1H-pyrazol-1-yl)-methyl]pyridine

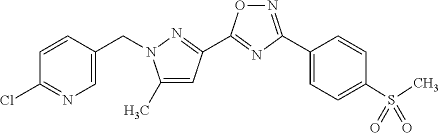

Analogously to the process described under Example 66, 140 mg (66% of th.) of the title compound were prepared from 125 mg (0.497 mmol) of the compound from Example 45A and 117 mg (0.546 mmol) of the compound from Example 14A.

¹H-NMR (400 MHz, CDCl₃, δ/ppm): 8.42 (d, 2H), 8.32 (d, 1H), 8.09 (d, 2H), 7.52 (dd, 1H), 7.33 (d, 1H), 7.31 (d, 1H), 6.85 (s, 1H), 5.45 (s, 2H), 3.11 (s, 3H), 2.34 (s, 3H).

LC/MS (method F, ESIpos): Rₜ=1.13 min, m/z=430/432 [M+H]⁺.

### Example 145

### 2-Chloro-5-({3-[3-(4-isobutylphenyl)-1,2,4-oxadiazol-5-yl]-5-methyl-1H-pyrazol-1-yl}methyl)-pyridine

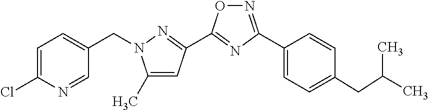

Analogously to the process described under Example 3, 689 mg (4.25 mmol) of 2-chloro-5-(chloromethyl)pyridine and 600 mg (2.13 mmol) of the compound from Example 76A were reacted to give 585 mg (67% of th.) of the title compound.

¹H-NMR (400 MHz, CDCl₃, δ/ppm): 8.33 (d, 1H), 8.10 (d, 2H), 7.51 (dd, 1H), 7.31 (d, 1H), 7.27 (d, 2H), 6.82 (s, 1H), 5.43 (s, 2H), 2.54 (d, 2H), 2.32 (s, 3H), 1.97-1.87 (m, 1H), 0.93 (d, 6H).

LC/MS (method I, ESIpos): Rₜ=1.41 min, m/z=408/410 [M+H]⁺.

### Example 146

### 2-Chloro-5-{[3-(3-{4-[1-(methoxymethyl)cyclobutyl]phenyl}-1,2,4-oxadiazol-5-yl)-5-methyl-1H-pyrazol-1-yl]methyl}pyridine

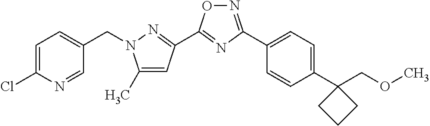

Analogously to the process described under Example 3, 500 mg (3.08 mmol) of 2-chloro-5-(chloromethyl)pyridine and 500 mg (1.54 mmol) of the compound from Example 77A were reacted to give 341 mg (49% of th.) of the title compound.

¹H-NMR (400 MHz, CDCl₃, δ/ppm): 8.32 (d, 1H), 8.13 (d, 2H), 7.51 (dd, 1H), 7.32 (d, 1H), 7.30 (d, 2H), 6.82 (s, 1H), 5.43 (s, 2H), 3.55 (s, 2H), 3.28 (s, 3H), 2.43-2.28 (m, 4H), 2.31 (s, 3H), 2.15-2.03 (m, 1H), 1.93-1.83 (m, 1H).

LC/MS (method I, ESIpos): Rₜ=1.33 min, m/z=450/452 [M+H]⁺.

### Example 147

### 2-Chloro-5-[(3-{3-[4-(methoxymethyl)phenyl]-1,2,4-oxadiazol-5-yl}-5-methyl-1H-pyrazol-1-yl)-methyl]pyridine

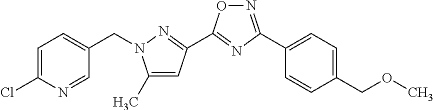

Analogously to the process described under Example 3, 300 mg (1.85 mmol) of 2-chloro-5-(chloromethyl)pyridine and 250 mg (0.925 mmol) of the compound from Example 78A were reacted to give 121 mg (33% of th.) of the title compound.

¹H-NMR (400 MHz, CDCl₃, δ/ppm): 8.32 (d, 1H), 8.19 (d, 2H), 7.51 (dd, 1H), 7.47 (d, 2H), 7.31 (d, 1H), 6.83 (s, 1H), 5.43 (s, 2H), 3.53 (s, 2H), 3.43 (s, 3H), 2.33 (s, 3H).

LC/MS (method I, ESIpos): Rₜ=1.14 min, m/z=396/398 [M+H]⁺.

### Example 148

### 2-Chloro-5-({3-[3-(3-fluoro-4-methoxyphenyl)-1,2,4-oxadiazol-5-yl]-5-methyl-1H-pyrazol-1-yl}-methyl)pyridine

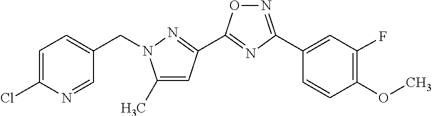

Analogously to the process described under Example 3, 354 mg (2.19 mmol) of 2-chloro-5-(chloromethyl)pyridine and 300 mg (1.09 mmol) of the compound from Example 79A were reacted to give 150 mg (34% of th.) of the title compound.

¹H-NMR (400 MHz, CDCl₃, δ/ppm): 8.31 (d, 1H), 7.97-7.91 (m, 2H), 7.51 (dd, 1H), 7.32 (d, 1H), 7.06 (t, 1H), 6.82 (s, 1H), 5.43 (s, 2H), 3.97 (s, 3H), 2.33 (s, 3H).

LC/MS (method I, ESIpos): Rₜ=1.19 min, m/z=400/402 [M+H]⁺.

### Example 149

### 2-Chloro-5-({3-[3-(4-methoxyphenyl)-1,2,4-oxadiazol-5-yl]-5-methyl-1H-pyrazol-1-yl}-methyl)pyridine

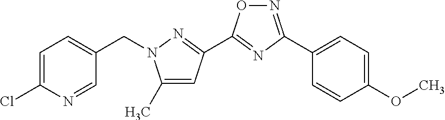

Analogously to the process described under Example 3, 474 mg (2.93 mmol) of 2-chloro-5-(chloromethyl)pyridine and 500 mg (1.95 mmol) of the compound from Example 80A were reacted to give 203 mg (27% of th.) of the title compound.

¹H-NMR (400 MHz, CDCl₃, δ/ppm): 8.31 (d, 1H), 8.13 (d, 2H), 7.51 (dd, 1H), 7.31 (d, 1H), 7.00 (d, 2H), 6.81 (s, 1H), 5.43 (s, 2H), 3.88 (s, 3H), 2.32 (s, 3H).

LC/MS (method F, ESIpos): Rₜ=1.27 min, m/z=382/384 [M+H]⁺.

### Example 150

### 2-Chloro-5-({3-[3-(4-isopropylphenyl)-1,2,4-oxadiazol-5-yl]-5-methyl-1H-pyrazol-1-yl}methyl)-pyridine

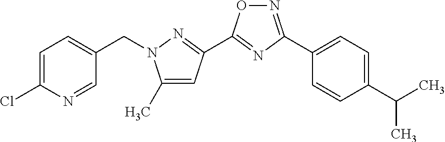

Analogously to the process described under Example 3, 1.21 g (7.45 mmol) of 2-chloro-5-(chloromethyl)pyridine and 1.0 g (3.73 mmol) of the compound from Example 81A were reacted to give 300 mg (20% of th., purity of 96%) of the title compound.

¹H-NMR (400 MHz, CDCl₃, δ/ppm): 8.32 (d, 1H), 8.11 (d, 2H), 7.51 (dd, 1H), 7.34 (d, 2H), 7.32 (d, 1H), 6.82 (s, 1H), 5.44 (s, 2H), 2.98 (sept, 1H), 2.31 (s, 3H), 1.30 (d, 6H).

LC/MS (method I, ESIpos): Rₜ=1.34 min, m/z=394/396 [M+H]⁺.

### Example 151

### 2-Chloro-5-{[5-methyl-3-(3-{4-[1-(trifluoromethyl)cyclopropyl]phenyl}-1,2,4-oxadiazol-5-yl)-1H-pyrazol-1-yl]methyl}pyridine

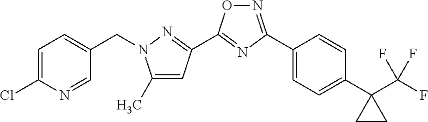

166 mg (1.48 mmol) of potassium tert-butylate were added to a mixture of 450 mg (1.35 mmol) of the compound from Example 86A and 328 mg (1.48 mmol) of (6-chloropyridin-3-yl)methyl methanesulphonate [K. C. Lee et al., J. Org. Chem. 1999, 64 (23), 8576-8581] in 10 ml of THF at 0° C. and the mixture was then allowed to come to RT, while stirring. After stirring at RT for 1 h, a further 100 mg (0.299 mmol) of (6-chloropyridin-3-yl)methyl methanesulphonate and 60 mg (0.535 mmol) of potassium tert-butylate were added and the mixture was stirred again at RT for 2 h. Water and ethyl acetate were subsequently added, the phases were separated and the aqueous phase was extracted once with ethyl acetate. The combined organic phases were washed once with saturated sodium chloride solution, dried over magnesium sulphate, filtered and concentrated. The residue was purified by means of column chromatography (silica gel, mobile phase: cyclohexane/ethyl acetate 7:3). 275 mg (39% of th., purity of 88%) of the title compound were obtained in this way.

¹H-NMR (400 MHz, CDCl₃, δ/ppm): 8.31 (s, 1H), 8.18 (d, 2H), 7.59 (d, 2H), 7.51 (d, 1H), 7.31 (d, 1H), 6.82 (s, 1H), 5.42 (s, 2H), 2.31 (s, 3H), 1.42-1.38 (m, 2H), 1.10 (s, broad, 2H).

LC/MS (method F, ESIpos): Rₜ=1.50 min, m/z=460/462 [M+H]⁺.

### Example 152

### N-[4-(5-{1-[(6-Chloropyridin-3-yl)methyl]-5-methyl-1H-pyrazol-3-yl}-1,2,4-oxadiazol-3-yl)-benzyl]-N-isopropylpropan-2-amine

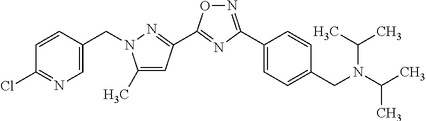

A mixture of 679 mg (2.0 mmol) of the compound from Example 83A, 421 mg (2.60 mmol) of 2-chloro-5-(chloromethyl)pyridine and 292 mg (2.60 mmol) of potassium tert-butylate in 20 ml of THF was heated under reflux overnight, while stirring. After cooling to RT, 100 mg (0.891 mmol) of potassium tert-butylate were again added and the mixture was then heated under reflux for a further 5 h, while stirring. After cooling to RT, ethyl acetate and water were added to the mixture. The phases were separated and the aqueous phase was extracted once with ethyl acetate. The combined organic phases were washed once with saturated sodium chloride solution, dried over magnesium sulphate, filtered and concentrated. The residue was purified by means of column chromatography (silica gel, mobile phase: cyclohexane/ethyl acetate 6:4). After drying in vacuo, 387 mg (40% of th.) of the title compound were obtained.

¹H-NMR (400 MHz, CDCl₃, δ/ppm): 8.31 (d, 1H), 8.23 (d, 2H), 7.76 (d, 2H), 7.53-7.49 (dd, 1H), 7.31 (d, 1H), 6.82 (s, 1H), 5.42 (s, 2H), 4.30 (s, 2H), 3.85-3.76 (m, 2H), 2.32 (s, 3H), 1.44 (d, 12H).

LC/MS (method F, ESIpos): Rₜ=0.93 min, m/z=465/467 [M+H]⁺.

### Example 153

### Ethyl 4-[4-(5-{1-[(6-chloropyridin-3-yl)methyl]-5-methyl-1H-pyrazol-3-yl}-1,2,4-oxadiazol-3-yl)-phenyl]tetrahydro-2H-pyran-4-carboxylate

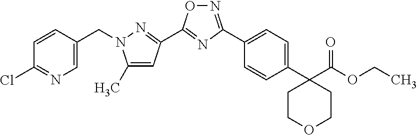

Analogously to the process described under Example 65, 344 mg (1.37 mmol) of the compound from Example 45A and 400 mg (1.37 mmol) of the compound from Example 71A were reacted to give 190 mg (26% of th.) of the title compound. The product was isolated directly from the reaction mixture by means of preparative HPLC (method P).

¹H-NMR (400 MHz, DMSO-d₆, δ/ppm): 8.38 (d, 1H), 8.06 (d, 2H), 7.69 (dd, 1H), 7.60 (d, 2H), 7.54 (d, 1H), 6.94 (s, 1H), 5.56 (s, 2H), 4.12 (q, 2H), 3.84 (m, 2H), 3.46 (t, 2H), 2.42 (m, 2H), 2.39 (s, 3H), 1.94 (m, 2H), 1.12 (t, 3H).

LC/MS (method I, ESIpos): Rₜ=1.21 min, m/z=508/510 [M+H]⁺.

### Example 154

### 2-Chloro-5-[(3-{3-[3-chloro-4-(trifluoromethoxy)phenyl]-1,2,4-oxadiazol-5-yl}-5-methyl-1H-pyrazol-1-yl)methyl]pyridine

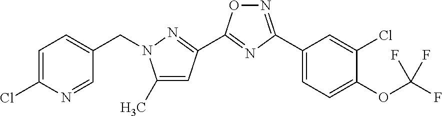

170 mg (1.52 mmol) of potassium tert-butylate were added to a mixture of 500 mg (1.38 mmol, purity of 95%) of the compound from Example 84A and 336 mg (1.52 mmol) of (6-chloropyridin-3-yl)methyl methanesulphonate [K. C. Lee et al., J. Org. Chem. 1999, 64 (23), 8576-8581] in 10 ml of THF at 0° C. and the mixture was then allowed to come to RT, while stirring. After 1 h, a further 336 mg (1.52 mmol) of (6-chloropyridin-3-yl)methyl methanesulphonate and 170 mg (1.52 mmol) of potassium tert-butylate were added and the mixture was stirred at RT for a further 2 h. Water and ethyl acetate were subsequently added, the phases were separated and the aqueous phase was extracted once with ethyl acetate. The combined organic phases were washed once with saturated sodium chloride solution, dried over magnesium sulphate, filtered and concentrated. The residue was purified by means of column chromatography (silica gel, mobile phase: cyclohexane/ethyl acetate 7:3). 248 mg (33% of th., purity of 86%) of the title compound were obtained.

LC/MS (method I, ESIpos): Rₜ=1.40 min, m/z=470/472 [M+H]⁺.

### Example 155

### {4-[4-(5-{1-[(6-Chloropyridin-3-yl)methyl]-5-methyl-1H-pyrazol-3-yl}-1,2,4-oxadiazol-3-yl)-phenyl]tetrahydro-2H-pyran-4-yl}methanol

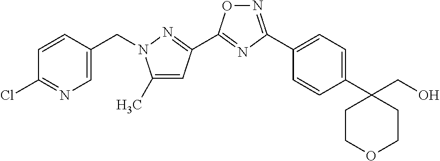

90 mg (0.18 mmol) of the compound from Example 153 were dissolved in 1.8 ml of THF, 0.18 ml (0.18 mmol) of a 1 M solution of lithium aluminium hydride in THF was added at 0° C. and the mixture was stirred for 1 h, while cooling in an ice bath. Saturated aqueous ammonium chloride solution was then added dropwise and the mixture was diluted with ethyl acetate. The organic phase was washed successively with 1 N sodium hydroxide solution, water and saturated sodium chloride solution, dried over magnesium sulphate, filtered and concentrated on a rotary evaporator. After the residue had been dried in vacuo, 59 mg (68% of th.) of the title compound were obtained.

¹H-NMR (400 MHz, DMSO-d₆, δ/ppm): 8.38 (d, 1H), 8.02 (d, 2H), 7.69 (dd, 1H), 7.57 (d, 2H), 7.54 (d, 1H), 6.94 (s, 1H), 5.58 (s, 2H), 4.70 (t, 1H), 3.71 (m, 2H), 3.44 (d, 2H), 3.38 (t, 2H), 2.39 (s, 3H), 2.02 (m, 2H), 1.94 (m, 2H).

LC/MS (method I, ESIpos): Rₜ=0.98 min, m/z=466/468 [M+H]⁺.

### Example 156

### 4-[4-(5-{1-[(6-Chloropyridin-3-yl)methyl]-5-methyl-1H-pyrazol-3-yl}-1,2,4-oxadiazol-3-yl)-phenyl]-N,N-dimethyl-tetrahydro-2H-pyran-4-carboxamide

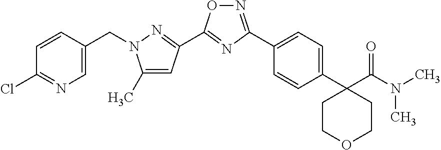

Analogously to the process described under Example 65, 155 mg (0.62 mmol) of the compound from Example 45A and 180 mg (0.62 mmol) of the compound from Example 72A were reacted to give 126 mg (40% of th.) of the title compound. The product was isolated directly from the reaction mixture by means of preparative HPLC (method P).

¹H-NMR (400 MHz, DMSO-d₆, δ/ppm): 8.38 (d, 1H), 8.07 (d, 2H), 7.69 (dd, 1H), 7.60 (d, 2H), 7.53 (d, 1H), 7.46 (d, 2H), 6.93 (s, 1H), 5.56 (s, 2H), 3.78 (d, 2H), 3.61 (t, 2H), 2.38 (s, 3H), 2.21 (d, 2H), 1.95 (m, 2H).

LC/MS (method I, ESIpos): Rₜ=1.06 min, m/z=507/509 [M+H]⁺.

### Example 157

### 4-[4-(5-{1-[(6-Chloropyridin-3-yl)methyl]-5-methyl-1H-pyrazol-3-yl}-1,2,4-oxadiazol-3-yl)-phenyl]-N-methyl-tetrahydro-2H-pyran-4-carboxamide

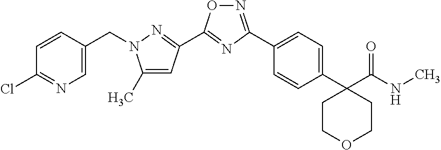

Analogously to the process described under Example 65, 163 mg (0.65 mmol) of the compound from Example 45A and 180 mg (0.65 mmol) of the compound from Example 73A were reacted to give 120 mg (35% of th.) of the title compound. The product was isolated directly from the reaction mixture by means of preparative HPLC (method P).

¹H-NMR (400 MHz, DMSO-d₆, δ/ppm): 8.38 (d, 1H), 8.02 (d, 2H), 7.68 (m, 2H), 7.54 (m, 3H), 6.93 (s, 1H), 5.56 (s, 2H), 3.74 (m, 2H), 3.48 (t, 2H), 2.56 (d, 3H), 2.46 (d, 2H), 2.38 (s, 3H), 1.89 (m, 2H).

LC/MS (method I, ESIpos): Rₜ=0.96 min, m/z=493/495 [M+H]⁺.

### Example 158

### 2-Iodo-5-[(5-methyl-3-{3-[4-(trifluoromethoxy)phenyl]-1,2,4-oxadiazol-5-yl}-1H-pyrazol-1-yl)-methyl]pyridine

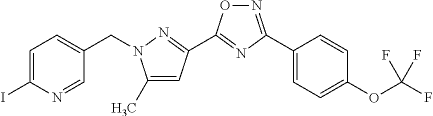

103 mg (0.688 mmol) of sodium iodide and 32 μl (0.252 mmol) of chloro(trimethyl)silane were added to a solution of 100 mg (0.229 mmol) of the compound from Example 2 in 0.5 ml of propionitrile at RT and the mixture was then heated at 120° C. in a microwave apparatus (CEM Discover, initial irradiation power 250 W) for 1 h. After cooling to RT, the mixture was diluted with 2 ml of acetonitrile and 1 ml of water. Two liquid phases formed, which were separated from one another. The organic phase was purified directly by means of preparative HPLC (method N). 61 mg (50% of th.) of the title compound were obtained.

¹H-NMR (400 MHz, CDCl₃, δ/ppm): 8.29 (d, 1H), 8.24 (d, 2H), 7.71 (d, 1H), 7.32 (d, 2H), 7.20-7.16 (dd, 1H), 6.82 (s, 1H), 5.39 (s, 2H), 2.31 (s, 3H).

LC/MS (method F, ESIpos): Rₜ=1.52 min, m/z=528 [M+H]⁺.

### Example 159

### 2-Chloro-5-[(5-methyl-3-{5-[4-(trifluoromethoxy)phenyl]-1,2,4-oxadiazol-3-yl}-1H-pyrazol-1-yl)-methyl]pyridine

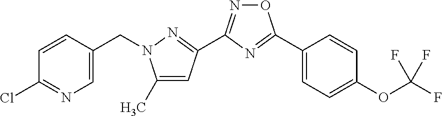

215 mg (0.809 mmol) of the compound from Example 91A and 169 μl (1.21 mmol) of triethylamine were initially introduced into 8 ml of methylene chloride, 182 mg (0.809 mmol) of 4-(trifluoromethoxy)benzoyl chloride then were added at 0° C. and the mixture was stirred at RT for 1 h. It was then concentrated, the residue was taken up in 5 ml of DMSO and the mixture was heated at 120° C. in a microwave apparatus (CEM Discover, initial irradiation power 250 W) for 30 min. After cooling to RT, 5 ml of water were added and the solid formed was filtered off and this was washed twice with 2 ml of water and dried in vacuo. 220 mg (62% of th.) of the title compound were obtained.

¹H-NMR (400 MHz, CDCl₃, δ/ppm): 8.33-8.28 (m, 3H), 7.50-7.46 (dd, 1H), 7.38 (d, 2H), 7.29 (d, 1H), 6.74 (s, 1H), 5.42 (s, 2H), 2.30 (s, 3H).

LC/MS (method I, ESIpos): Rₜ=1.27 min, m/z=436/438 [M+H]⁺.

### Example 160

### 2-Chloro-5-[(4-{3-[4-(2-fluoropropan-2-yl)phenyl]-1,2,4-oxadiazol-5-yl}-2-methyl-1H-pyrrol-1-yl)methyl]pyridine

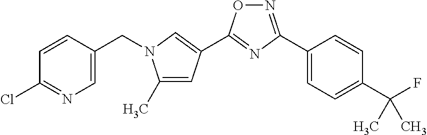

Analogously to the process described under Example 72, 200 mg (0.798 mmol) of the compound from Example 46A and 157 mg (0.798 mmol) of the compound from Example 2A were reacted to give 78 mg (24% of th.) of the title compound. After purification of the crude product by preparative HPLC (method N), the combined product fractions were concentrated to a residual volume of aqueous phase, saturated aqueous sodium bicarbonate solution was added to the residue and the mixture was extracted twice with ethyl acetate. The combined ethyl acetate phases were then dried over magnesium sulphate, filtered and concentrated and the resulting residue was dried in vacuo.

¹H-NMR (400 MHz, CDCl₃, δ/ppm): 8.25 (d, 1H), 8.11 (d, 2H), 7.52-7.47 (m, 3H), 7.33-7.28 (m, 2H), 6.60 (s, 1H), 5.11 (s, 2H), 2.20 (s, 3H), 1.72 (s, 3H), 1.70 (s, 3H).

LC/MS (method F, ESIpos): Rₜ=1.50 min, m/z=411/413 [M+H]⁺.

### Example 161

### 2-Chloro-5-[(2-methyl-4-{3-[4-(trifluoromethyl)phenyl]-1,2,4-oxadiazol-5-yl}-1H-pyrrol-1-yl)-methyl]pyridine

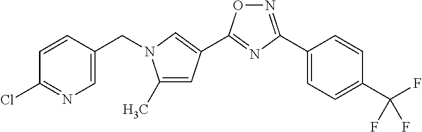

Analogously to the process described under Example 72, 200 mg (0.798 mmol) of the compound from Example 46A and 162 mg (0.798 mmol) of N′-hydroxy-4-(trifluoromethyl)-benzenecarboximide amide were reacted to give 102 mg (30% of th.) of the title compound. After purification of the crude product by preparative HPLC (method N), the combined product fractions were concentrated to a residual volume of aqueous phase, saturated aqueous sodium bicarbonate solution was added to the residue and the mixture was extracted twice with ethyl acetate. The combined ethyl acetate phases were then dried over magnesium sulphate, filtered and concentrated and the resulting residue was dried in vacuo.

¹H-NMR (400 MHz, CDCl₃, δ/ppm): 8.28-8.23 (m, 3H), 7.75 (d, 2H), 7.49 (s, 1H), 7.33-7.28 (m, 2H), 6.61 (s, 1H), 5.11 (s, 2H), 2.21 (s, 3H).

LC/MS (method F, ESIpos): Rₜ=1.55 min, m/z=419/421 [M+H]⁺.

### Example 162

### 2-Chloro-5-[(2-methyl-4-{3-[4-(trimethylsilyl)phenyl]-1,2,4-oxadiazol-5-yl}-1H-pyrrol-1-yl)-methyl]pyridine

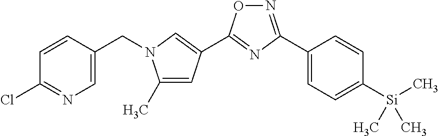

Analogously to the process described under Example 72, 200 mg (0.798 mmol) of the compound from Example 46A and 166 mg (0.798 mmol) of the compound from Example 17A were reacted to give 83 mg (25% of th.) of the title compound. After purification of the crude product by preparative HPLC (method N), the combined product fractions were concentrated to a residual volume of aqueous phase, saturated aqueous sodium bicarbonate solution was added to the residue and the mixture was extracted twice with ethyl acetate. The combined ethyl acetate phases were then dried over magnesium sulphate, filtered and concentrated and the resulting residue was dried in vacuo.

¹H-NMR (400 MHz, CDCl₃, δ/ppm): 8.25 (s, 1H), 8.08 (d, 2H), 7.63 (d, 2H), 7.48 (d, 1H), 7.35-7.27 (m, 2H), 6.60 (s, 1H), 5.11 (s, 2H), 2.20 (s, 3H), 0.30 (s, 9H).

LC/MS (method F, ESIpos): Rₜ=1.71 min, m/z=423/425 [M+H]⁺.

### Example 163

### N-[4-(5-{1-[(6-Chloropyridin-3-yl)methyl]-5-methyl-1H-pyrrol-3-yl}-1,2,4-oxadiazol-3-yl)-benzyl]-N-isopropylpropan-2-amine

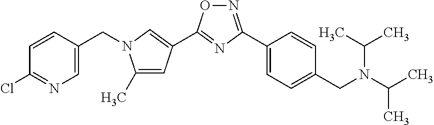

Analogously to the process described under Example 72, 200 mg (0.798 mmol) of the compound from Example 46A and 172 mg (0.798 mmol) of the compound from Example 65A were reacted to give 80 mg (22% of th.) of the title compound. After purification of the crude product by preparative HPLC (method N), the combined product fractions were concentrated to a residual volume of aqueous phase, saturated aqueous sodium bicarbonate solution was added to the residue and the mixture was extracted twice with ethyl acetate. The combined ethyl acetate phases were then dried over magnesium sulphate, filtered and concentrated and the resulting residue was dried in vacuo.

¹H-NMR (400 MHz, CDCl₃, δ/ppm): 8.25 (d, 1H), 8.02 (d, 2H), 7.52-7.47 (m, 3H), 7.33-7.27 (m, 2H), 6.60 (s, 1H), 5.10 (s, 2H), 3.70 (s, 2H), 3.08-2.98 (m, 2H), 2.20 (s, 3H), 1.02 (d, 12H).

LC/MS (method F, ESIpos): Rₜ=1.03 min, m/z=464/466 [M+H]⁺.

### Example 164

### 2-Chloro-5-[(2-methyl-4-{3-[4-(1H-pyrrol-1-ylmethyl)phenyl]-1,2,4-oxadiazol-5-yl}-1H-pyrrol-1-yl)methyl]pyridine

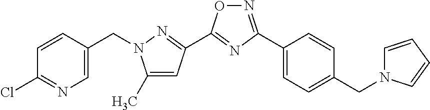

Analogously to the process described under Example 72, 200 mg (0.798 mmol) of the compound from Example 46A and 199 mg (0.798 mmol) of the compound from Example 55A were reacted to give 57 mg (16% of th.) of the title compound. After purification of the crude product by preparative HPLC (method N), the combined product fractions were concentrated to a residual volume of aqueous phase, saturated aqueous sodium bicarbonate solution was added to the residue and the mixture was extracted twice with ethyl acetate. The combined ethyl acetate phases were then dried over magnesium sulphate, filtered and concentrated and the resulting residue was dried in vacuo.

¹H-NMR (400 MHz, CDCl₃, δ/ppm): 8.25 (d, 1H), 8.08 (d, 2H), 7.46 (s, 1H), 7.32-7.24 (m, 2H), 7.21 (d, 2H), 6.72-6.70 (t, 2H), 6.59 (s, 1H), 6.22-6.20 (t, 2H), 5.13 (s, 2H), 5.11 (s, 2H), 2.20 (s, 3H).

LC/MS (method D, ESIpos): Rₜ=2.70 min, m/z=430/432 [M+H]⁺.

### Example 165

### tert-Butyl [(1-{4-[5-(5-methyl-1-{[6-(methylamino)pyridin-3-yl]methyl}-1H-pyrazol-3-yl)-1,2,4-oxadiazol-3-yl]phenyl}cyclobutyl)oxy]acetate

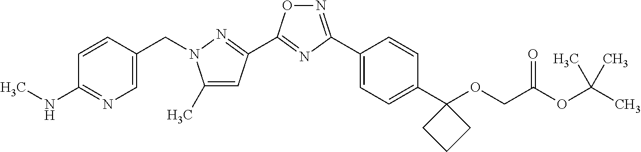

110 mg (0.205 mmol) of the compound from Example 140 were dissolved in 3 ml of a 33% strength solution of methylamine in ethanol and the solution was stirred at 150° C. in a microwave oven (CEM Discover, initial irradiation power 250 W) for 5 h. After cooling to RT, the reaction mixture was freed from all the volatile components on a rotary evaporator. The residue obtained was separated into its components by means of preparative HPLC (method M). 11 mg (10% of th.) of the title compound were obtained as a by-product of the reaction (cf. Example 169).

¹H-NMR (400 MHz, CDCl₃, δ/ppm): 8.21 (d, 2H), 8.01 (d, 1H), 7.59 (d, 2H), 7.41 (dd, 1H), 6.78 (s, 1H), 6.37 (d, 1H), 5.30 (s, 2H), 4.91 (broad, 1H), 3.60 (s, 1H), 2.91 (s, 3H), 2.55-2.41 (m, 4H), 2.31 (s, 3H), 2.07-1.98 (m, 1H), 1.77-1.67 (m, 1H), 1.41 (s, 9H).

LC/MS (method I, ESIpos): Rₜ=1.04 min, m/z=531 [M+H]⁺.

### Example 166

### N-Methyl-5-[(5-methyl-3-{3-[4-(tetrahydro-2H-pyran-4-yl)phenyl]-1,2,4-oxadiazol-5-yl}-1H-pyrazol-1-yl)methyl]pyridin-2-amine

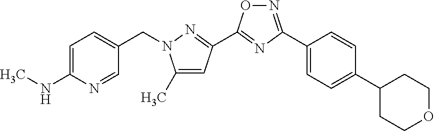

66 mg (0.151 mmol) of the compound from Example 142 were dissolved in 4 ml of a 33% strength solution of methylamine in ethanol and the solution was stirred at 150° C. in a microwave oven (CEM Discover, initial irradiation power 250 W) for 5 h. After cooling to RT, the reaction mixture was freed from all the volatile components on a rotary evaporator. The residue obtained was stirred with acetonitrile. 34 mg (53% of th.) of the title compound were obtained.

¹H-NMR (400 MHz, CDCl₃, δ/ppm): 8.14 (d, 2H), 8.03 (d, 1H), 7.38 (dd, 1H), 7.34 (d, 2H), 6.77 (s, 1H), 6.35 (d, 1H), 5.29 (s, 2H), 4.59-4.53 (m, broad, 1H), 4.12-4.08 (m, 2H), 3.59-3.52 (m, 2H), 2.91 (d, 3H), 2.87-2.79 (m, 1H), 2.31 (s, 3H), 1.92-1.79 (m, 4H).

LC/MS (method F, ESIpos): Rₜ=0.93 min, m/z=431 [M+H]⁺.

### Example 167

### 1-{4-[5-(5-Methyl-1-{[6-(methylamino)pyridin-3-yl]methyl}-1H-pyrazol-3-yl)-1,2,4-oxadiazol-3-yl]phenyl}cyclobutanol

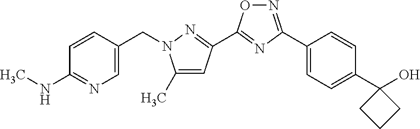

Analogously to the process described under Example 165, 50 mg (84% of th.) of the title compound were obtained from 60 mg (0.142 mmol) of the compound from Example 143.

¹H-NMR (400 MHz, CDCl₃, δ/ppm): 8.20 (d, 2H), 7.91 (d, 1H), 7.63 (d, 2H), 7.49 (dd, 1H), 6.79 (s, 1H), 6.43 (d, 1H), 5.29 (s, 2H), 2.90 (s, 3H), 2.63-2.57 (m, 2H), 2.44-2.38 (m, 2H), 2.32 (s, 3H), 2.13-2.03 (m, 1H), 1.82-1.71 (m, 1H).

LC/MS (method D, ESIpos): Rₜ=1.56 min, m/z=417 [M+H]⁺.

### Example 168

### N-Methyl-5-[(5-methyl-3-{3-[4-(methylsulphonyl)phenyl]-1,2,4-oxadiazol-5-yl}-1H-pyrazol-1-yl)-methyl]pyridin-2-amine

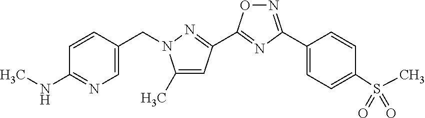

Analogously to the process described under Example 165, 15 mg (28% of th.) of the title compound were obtained from 54 mg (0.126 mmol) of the compound from Example 144.

¹H-NMR (400 MHz, CDCl₃, δ/ppm): 8.42 (d, 2H), 8.08 (d, 2H), 7.98 (d, 1H), 7.43 (dd, 1H), 6.80 (s, 1H), 6.40 (d, 1H), 5.31 (broad, 1H), 5.30 (s, 2H), 3.10 (s, 3H), 2.90 (s, 3H), 2.33 (s, 3H).

LC/MS (method I, ESIpos): Rₜ=0.70 min, m/z=425 [M+H]⁺.

### Example 169

### N-Methyl-2-[(1-{4-[5-(5-methyl-1-{[6-(methylamino)pyridin-3-yl]methyl}-1H-pyrazol-3-yl)-1,2,4-oxadiazol-3-yl]phenyl}cyclobutyl)oxy]acetamide

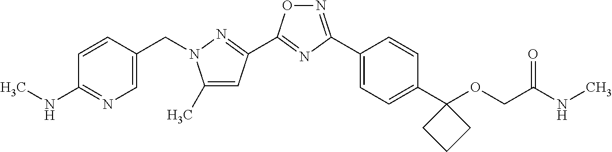

110 mg (0.205 mmol) of the compound from Example 140 were dissolved in 3 ml of a 33% strength solution of methylamine in ethanol and the solution was stirred at 150° C. in a microwave oven (CEM Discover, initial irradiation power 250 W) for 5 h. After cooling to RT, the reaction mixture was freed from all the volatile components on a rotary evaporator. The residue obtained was separated into its components by means of preparative HPLC (method M). 87 mg (87% of th.) of the title compound were obtained.

¹H-NMR (400 MHz, CDCl₃, δ/ppm): 8.21 (d, 2H), 8.00 (d, 1H), 7.51 (d, 2H), 7.42 (dd, 1H), 6.79 (s, 1H), 6.67-6.62 (m, 1H), 6.39 (d, 1H), 5.30 (s, 2H), 5.20 (broad, 1H), 3.61 (s, 2H), 2.91 (s, 3H), 2.85 (d, 3H), 2.53-2.40 (m, 4H), 2.32 (s, 3H), 2.08-1.97 (m, 1H), 1.80-1.70 (m, 1H).

LC/MS (method I, ESIpos): Rₜ=0.80 min, m/z=488 [M+H]⁺.

### Example 170

### 5-({3-[3-(4-tert-Butylphenyl)-1,2,4-oxadiazol-5-yl]-5-methyl-1H-pyrazol-1-yl}methyl)-N-methyl-pyridin-2-amine

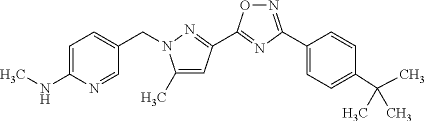

125 mg (0.306 mmol) of the compound from Example 70 were dissolved in 2.3 ml (18.4 mmol) of an 8 M solution of methylamine in ethanol. The reaction mixture was automatically controlled at 140° C. in a microwave oven (Biotage Initiator 2.5, automatic control of the irradiation power). After 140° C. was reached, the temperature was increased to 160° C. under manual control over a period of 3 min. After the reaction mixture had been kept at 160° C. for 4 h, it was allowed to cool to RT. All the volatile constituents were removed on a rotary evaporator. The residue obtained was purified by means of MPLC (15 g of silica gel, mobile phase: cyclohexane/ethyl acetate 1:1). 120 mg (97% of th.) of the title compound were obtained.

¹H-NMR (400 MHz, CDCl₃, δ/ppm): 8.13 (d, 2H), 8.03 (d, 1H), 7.51 (d, 2H), 7.38 (dd, 1H), 6.77 (s, 1H), 6.36 (d, 1H), 5.29 (s, 2H), 4.58 (broad, 1H), 2.91 (d, 3H), 2.31 (s, 3H), 1.37 (s, 9H).

LC/MS (method I, ESIpos): Rₜ=0.99 min, m/z=403 [M+H]⁺.

### Example 171

### 5-{[3-(3-{4-[1-(Methoxymethyl)cyclobutyl]phenyl}-1,2,4-oxadiazol-5-yl)-5-methyl-1H-pyrazol-1-yl]methyl}-N-methylpyridin-2-amine

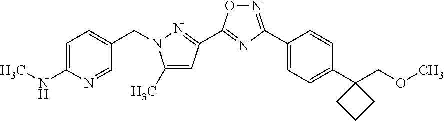

Analogously to the process described under Example 170, 125 mg (0.278 mmol) of the compound from Example 146 were reacted to give 100 mg (81% of th.) of the title compound.

¹H-NMR (400 MHz, CDCl₃, δ/ppm): 8.13 (d, 2H), 8.03 (d, 1H), 7.38 (dd, 1H), 7.29 (d, 2H), 6.77 (s, 1H), 6.35 (d, 1H), 5.29 (s, 2H), 4.57 (broad, 1H), 3.55 (s, 2H), 3.28 (s, 3H), 2.91 (d, 3H), 2.41-2.29 (m, 4H), 2.31 (s, 3H), 2.15-2.03 (m, 1H), 1.93-1.83 (m, 1H).

LC/MS (method I, ESIpos): Rₜ=0.94 min, m/z=445 [M+H]⁺.

### Example 172

### 5-[(3-{3-[4-(Methoxymethyl)phenyl]-1,2,4-oxadiazol-5-yl}-5-methyl-1H-pyrazol-1-yl)methyl]-N-methylpyridin-2-amine

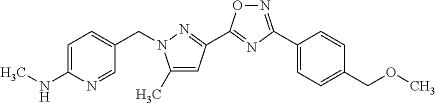

Analogously to the process described under Example 170, 100 mg (0.253 mmol) of the compound from Example 147 were reacted to give 71 mg (72% of th.) of the title compound.

¹H-NMR (400 MHz, CDCl₃, δ/ppm): 8.20 (d, 2H), 8.04 (d, 1H), 7.47 (d, 2H), 7.38 (dd, 1H), 6.78 (s, 1H), 6.36 (d, 1H), 5.30 (s, 2H), 4.59 (broad, 1H), 3.52 (s, 2H), 3.43 (s, 3H), 2.91 (d, 3H), 2.31 (s, 3H).

LC/MS (method I, ESIpos): Rₜ=0.79 min, m/z=391 [M+H]⁺.

### Example 173

### 5-({3-[3-(4-Methoxyphenyl)-1,2,4-oxadiazol-5-yl]-5-methyl-1H-pyrazol-1-yl}methyl)-N-methyl-pyridin-2-amine

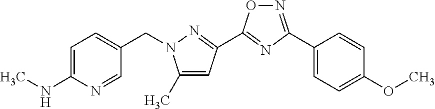

Analogously to the process described under Example 170, 100 mg (0.262 mmol) of the compound from Example 149 were reacted to give 80 mg (81% of th.) of the title compound.

¹H-NMR (400 MHz, CDCl₃, δ/ppm): 8.14 (d, 2H), 8.03 (d, 1H), 7.38 (dd, 1H), 7.00 (d, 2H), 6.76 (s, 1H), 6.35 (d, 1H), 5.29 (s, 2H), 4.60 (broad, 1H), 3.88 (s, 3H), 2.90 (d, 3H), 2.31 (s, 3H).

LC/MS (method I, ESIpos): Rₜ=0.81 min, m/z=377 [M+H]⁺.

### Example 174

### 5-({3-[3-(3-Fluoro-4-methoxyphenyl)-1,2,4-oxadiazol-5-yl]-5-methyl-1H-pyrazol-1-yl}methyl)-N-methylpyridin-2-amine

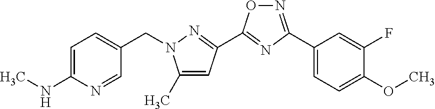

Analogously to the process described under Example 170, 100 mg (0.250 mmol) of the compound from Example 148 were reacted to give 42 mg (41% of th.) of the title compound.

¹H-NMR (400 MHz, CDCl₃, δ/ppm): 8.03 (d, 1H), 7.99-7.91 (m, 2H), 7.38 (dd, 1H), 7.05 (t, 1H), 6.77 (s, 1H), 6.37 (d, 1H), 5.29 (s, 2H), 4.61 (broad, 1H), 3.97 (s, 3H), 2.91 (d, 3H), 2.32 (s, 3H).

LC/MS (method I, ESIpos): Rₜ=0.83 min, m/z=395 [M+H]⁺.

### Example 175

### 5-({3-[3-(4-Isobutylphenyl)-1,2,4-oxadiazol-5-yl]-5-methyl-1H-pyrazol-1-yl}methyl)-N-methyl-pyridin-2-amine

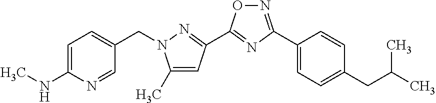

Analogously to the process described under Example 170, 125 mg (0.306 mmol) of the compound from Example 145 were reacted to give 102 mg (83% of th.) of the title compound.

¹H-NMR (400 MHz, CDCl₃, δ/ppm): 8.10 (d, 2H), 8.03 (d, 1H), 7.39 (dd, 1H), 7.27 (d, 2H), 6.77 (s, 1H), 6.36 (d, 1H), 5.30 (s, 2H), 4.58 (broad, 1H), 2.91 (d, 3H), 2.53 (d, 2H), 2.31 (s, 3H), 1.92 (m, 1H), 0.92 (d, 6H).

LC/MS (method I, ESIpos): Rₜ=1.01 min, m/z=403 [M+H]⁺.

### Example 176

### 5-({3-[3-(4-Isopropylphenyl)-1,2,4-oxadiazol-5-yl]-5-methyl-1H-pyrazol-1-yl}methyl)-N-methyl-pyridin-2-amine

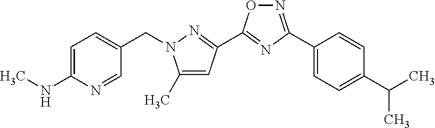

Analogously to the process described under Example 170, 125 mg (0.317 mmol) of the compound from Example 150 were reacted to give 96 mg (76% of th.) of the title compound.

¹H-NMR (400 MHz, CDCl₃, δ/ppm): 8.12 (d, 2H), 8.03 (d, 1H), 7.38 (dd, 1H), 7.34 (d, 2H), 6.77 (s, 1H), 6.36 (d, 1H), 5.30 (s, 2H), 4.59 (broad, 1H), 2.98 (sept, 1H), 2.91 (d, 3H), 2.31 (s, 3H), 1.30 (d, 6H).

LC/MS (method Q, ESIpos): Rₜ=1.89 min, m/z=389 [M+H]⁺.

### Example 177

### N-Ethyl-5-[(5-methyl-3-{3-[4-(trifluoromethoxy)phenyl]-1,2,4-oxadiazol-5-yl}-1H-pyrazol-1-yl)-methyl]pyridin-2-amine

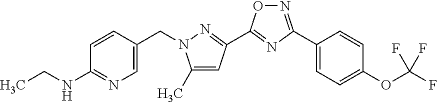

A mixture of 200 mg (0.459 mmol) of the compound from Example 2 and 4.6 ml and (9.2 mmol) of a 2 M solution of ethylamine in THF was heated at 170° C. in a microwave apparatus (CEM Discover, initial irradiation power 250 W) for 6 h. After cooling to RT, 1 ml (12.0 mmol) of a 70% strength ethylamine solution in water was added and the mixture was heated again at 170° C. in the microwave apparatus for 8 h. After cooling to RT, the mixture was concentrated and the residue was purified by means of preparative HPLC (method N). The combined product fractions were concentrated to a residual volume of aqueous phase and saturated aqueous sodium bicarbonate solution was added to the residue. The solid formed was filtered off, washed twice with water and dried in vacuo. 110 mg (54% of th.) of the title compound were obtained.

¹H-NMR (400 MHz, CDCl₃, δ/ppm): 8.25 (d, 2H), 8.02 (s, 1H), 7.38-7.31 (m, 3H), 6.77 (s, 1H), 6.33 (d, 1H), 5.30 (s, 2H), 4.51 (t, 1H), 3.32-3.25 (m, 2H), 2.31 (s, 3H), 1.26-1.22 (t, 3H).

LC/MS (method I, ESIpos): Rₜ=0.99 min, m/z=445 [M+H]⁺.

### Example 178

### N-Methyl-5-[(5-methyl-3-{3-[4-(trimethylsilyl)phenyl]-1,2,4-oxadiazol-5-yl}-1H-pyrazol-1-yl)-methyl]pyridin-2-amine

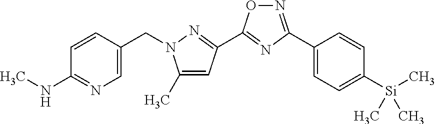

A mixture of 120 mg (0.283 mmol) of the compound from Example 9 and 3.5 ml (28.3 mmol) of a 33% strength methylamine solution in ethanol was heated at 140° C. in a microwave apparatus (CEM Discover, initial irradiation power 100 W) for 5 h. After cooling to RT, the mixture was purified directly by means of preparative HPLC (method N). The combined product fractions were concentrated to a residual volume of aqueous phase and saturated aqueous sodium bicarbonate solution was added to the residue and the mixture was extracted twice with ethyl acetate. The combined ethyl acetate phases were then dried over magnesium sulphate, filtered and concentrated. After the residue had been dried in vacuo, 99 mg (83% of th.) of the title compound were obtained.

¹H-NMR (400 MHz, CDCl₃, δ/ppm): 8.17 (d, 2H), 8.04 (d, 1H), 7.64 (d, 2H), 7.40-7.36 (dd, 1H), 6.78 (s, 1H), 6.36 (d, 1H), 5.30 (s, 2H), 4.63 (s, broad, 1H), 2.91 (d, 3H), 2.31 (s, 3H), 0.31 (s, 9H).

LC/MS (method I, ESIpos): Rₜ=1.01 min, m/z=419 [M+H]⁺.

### Example 179

### N-Ethyl-5-[(5-methyl-3-{3-[4-(1,1,1-trifluoro-2-methylpropan-2-yl)phenyl]-1,2,4-oxadiazol-5-yl}-1H-pyrazol-1-yl)methyl]pyridin-2-amine

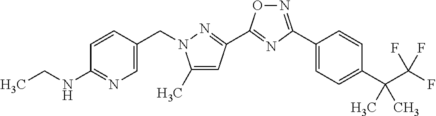

A mixture of 200 mg (0.433 mmol) of the compound from Example 68, 4.3 ml (8.66 mmol) of a 2 M solution of ethylamine in THF and 1.0 ml (12.4 mmol) of a 70% strength ethylamine solution in water was heated at 170° C. in a microwave apparatus (CEM Discover, initial irradiation power 250 W) for 6 h. After cooling to RT, a further 2.0 ml (24.8 mmol) of a 70% strength ethylamine solution in water was added and the mixture was heated again at 170° C. in the microwave apparatus for 18 h. After cooling to RT, the mixture was concentrated and the residue was purified by means of preparative HPLC (method N). The combined product fractions were concentrated to a residual volume of aqueous phase. Saturated sodium bicarbonate solution was added and the mixture was extracted twice with ethyl acetate. The ethyl acetate phases were combined, dried over magnesium sulphate, filtered and concentrated. After drying of the residue in vacuo, 161 mg (79% of th.) of the title compound were obtained.

¹H-NMR (400 MHz, CDCl₃, δ/ppm): 8.19 (d, 2H), 8.02 (s, 1H), 7.62 (d, 2H), 7.40-7.35 (dd, 1H), 6.77 (s, 1H), 6.34 (d, 1H), 5.29 (s, 2H), 4.55 (s, broad, 1H), 3.32-3.25 (m, 2H), 2.31 (s, 3H), 1.62 (s, 6H), 1.28-1.21 (t, 3H).

LC/MS (method I, ESIpos): Rₜ=1.01 min, m/z=471 [M+H]⁺.

### Example 180

### N-Ethyl-5-[(5-methyl-3-{3-[4-(trimethylsilyl)phenyl]-1,2,4-oxadiazol-5-yl}-1H-pyrazol-1-yl)-methyl]pyridin-2-amine

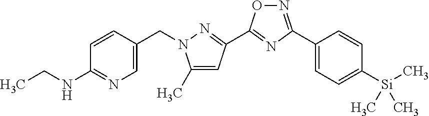

A mixture of 212 mg (0.50 mmol) of the compound from Example 9, 5.0 ml (10.0 mmol) of a 2 M solution of ethylamine in THF and 1.0 ml (12.4 mmol) of a 70% strength ethylamine solution in water was heated at 170° C. in a microwave apparatus (CEM Discover, initial irradiation power 250 W) for 6 h. After cooling to RT, a further 1.0 ml (12.4 mmol) of a 70% strength ethylamine solution in water was added and the mixture was heated again at 170° C. in the microwave apparatus for 10 h. After cooling to RT, the mixture was concentrated and the residue was purified by means of preparative HPLC (method N). The combined product fractions were concentrated to a residual volume of aqueous phase. Saturated sodium bicarbonate solution was added and the mixture was extracted twice with ethyl acetate. The ethyl acetate phases were combined, dried over magnesium sulphate, filtered and concentrated. After drying of the residue in vacuo, 76 mg (35% of th.) of the title compound were obtained.

¹H-NMR (400 MHz, CDCl₃, δ/ppm): 8.17 (d, 2H), 8.02 (s, 1H), 7.64 (d, 2H), 7.40-7.35 (dd, 1H), 6.78 (s, 1H), 6.35 (d, 1H), 5.29 (s, 2H), 4.62 (s, broad, 1H), 3.35-3.22 (m, 2H), 2.31 (s, 3H), 1.27-1.21 (t, 3H), 0.31 (s, 9H).

LC/MS (method F, ESIpos): Rₜ=1.23 min, m/z=433 [M+H]⁺.

### Example 181

### 5-[(3-{3-[3-Chloro-4-(trifluoromethoxy)phenyl]-1,2,4-oxadiazol-5-yl}-5-methyl-1H-pyrazol-1-yl)-methyl]-N-methylpyridin-2-amine

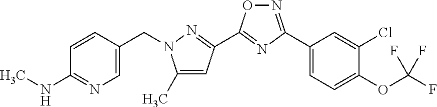

A mixture of 224 mg (0.410 mmol, purity of 86%) of the compound from Example 154 and 5.1 ml (41.0 mmol) of a 33% strength methylamine solution in ethanol was heated at 150° C. in a microwave apparatus (CEM Discover, initial irradiation power 100 W) for 3 h. After cooling to RT, water was added to the mixture. The solid formed was filtered off, washed with water and taken up in DMSO. This DMSO solution was then purified by means of preparative HPLC (method N). The combined product fractions were concentrated to a residual volume of aqueous phase, saturated aqueous sodium bicarbonate solution was added and the mixture was extracted twice with ethyl acetate. The combined ethyl acetate phases were dried over magnesium sulphate, filtered and concentrated. After the residue had been dried in vacuo, 118 mg (62% of th.) of the title compound were obtained.

¹H-NMR (400 MHz, CDCl₃, δ/ppm): 9.96 (s, broad, 1H), 8.36 (s, 1H), 8.13 (d, 1H), 7.82 (d, 1H), 7.63 (s, 1H), 7.45 (d, 1H), 6.83 (s, 1H), 6.72 (d, 1H), 5.29 (s, 2H), 2.98 (s, 3H), 2.36 (s, 3H).

LC/MS (method I, ESIpos): Rₜ=1.04 min, m/z=465/467 [M+H]⁺.

### Example 182

### 5-[(3-{3-[3-Fluoro-4-(trifluoromethoxy)phenyl]-1,2,4-oxadiazol-5-yl}-5-methyl-1H-pyrazol-1-yl)-methyl]-N-methylpyridin-2-amine

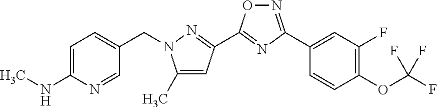

### Step 1: N-(3,4-Dimethoxybenzyl)-5-[(3-{3-[3-fluoro-4-(trifluoromethoxy)phenyl]-1,2,4-oxadiazol-5-yl}-5-methyl-1H-pyrazol-1-yl)methyl]-N-methylpyridin-2-amine

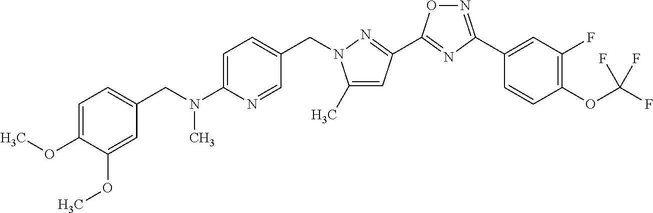

Analogously to the process described under Example 76A, 328 mg (1.00 mmol) of the compound from Example 85A and 418 mg (1.10 mmol) of the compound from Example 90A were reacted to give 154 mg (26% of th.) of the title compound.

¹H-NMR (400 MHz, CDCl₃, δ/ppm): 8.25 (d, 1H), 8.02-7.97 (m, 2H), 7.52-7.43 (m, 2H), 6.89 (s, 1H), 6.80-6.69 (m, 3H), 6.43 (d, 1H), 5.78 (s, 2H), 4.68 (s, 2H), 3.82 (s, 3H), 3.79 (s, 3H), 3.00 (s, 3H), 2.32 (s, 3H).

LC/MS (method F, ESIpos): Rₜ=1.41 min, m/z=599 [M+H]⁺.

### Step 2: 5-[(3-{3-[3-Fluoro-4-(trifluoromethoxy)phenyl]-1,2,4-oxadiazol-5-yl}-5-methyl-1H-pyrazol-1-yl)methyl]-N-methylpyridin-2-amine

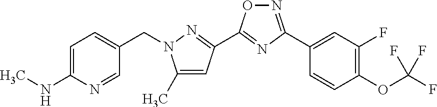

137 mg (0.228 mmol) of the compound from Example 182/step 1 were dissolved in 1 ml of methylene chloride, 1 ml (12.98 mmol) of TFA was added and the mixture was stirred at RT for 48 h. The mixture was then concentrated and the residue was purified by means of preparative HPLC (method N). The combined product fractions were concentrated to a residual volume of water and saturated aqueous sodium bicarbonate solution was added to the residue. The solid formed was filtered off, washed twice with water and dried in vacuo. 78 mg (76% of th.) of the title compound were obtained.

¹H-NMR (400 MHz, CDCl₃, δ/ppm): 8.10-8.00 (m, 2H), 7.89 (s, 1H), 7.52 (d, 1H), 7.48-7.40 (t, 1H), 6.80 (s, 1H), 6.60 (s, broad, 1H), 6.50 (d, 1H), 5.30 (s, 2H), 2.92 (s, 3H), 2.32 (s, 3H).

LC/MS (method I, ESIpos): Rₜ=0.95 min, m/z=449 [M+H]⁺.

### Example 183

### N-Methyl-5-{[5-methyl-3-(3-{4-[1-(trifluoromethyl)cyclopropyl]phenyl}-1,2,4-oxadiazol-5-yl)-1H-pyrazol-1-yl]methyl}pyridin-2-amine

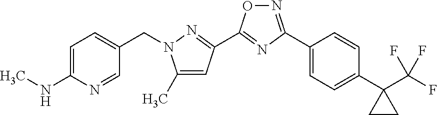

A mixture of 150 mg (0.294 mmol, purity of 90%) of the compound from Example 151 and 3.64 ml (29.4 mmol) of a 33% strength methylamine solution in ethanol was heated at 150° C. in a microwave apparatus (CEM Discover, initial irradiation power 100 W) for 3 h. After cooling to RT, the mixture was concentrated and the residue was purified by means of preparative HPLC (method N). The combined product fractions were concentrated to a residual volume of aqueous phase. Saturated aqueous sodium bicarbonate solution was added. The solid formed was filtered off and washed twice with water. After drying in vacuo, 64 mg (46% of th.) of the title compound were obtained.

¹H-NMR (400 MHz, CDCl₃, δ/ppm): 8.18 (d, 2H), 8.04 (d, 1H), 7.58 (d, 2H), 7.40-7.36 (dd, 1H), 6.77 (s, 1H), 6.36 (d, 1H), 5.30 (s, 2H), 4.65-4.57 (m, broad, 1H), 2.91 (d, 3H), 2.32 (s, 3H), 1.42-1.38 (m, 2H), 1.12-1.05 (m, 2H).

LC/MS (method F, ESIpos): Rₜ=1.10 min, m/z=455 [M+H]⁺.

### Example 184

### 5-{[3-(3-{4-[(Diisopropylamino)methyl]phenyl}-1,2,4-oxadiazol-5-yl)-5-methyl-1H-pyrazol-1-yl]-methyl}-N-methylpyridin-2-amine

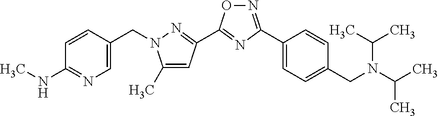

A mixture of 280 mg (0.542 mmol, purity of 90%) of the compound from Example 152 and 6.72 ml (54.193 mmol) of a 33% strength methylamine solution in ethanol was heated at 150° C. in a microwave apparatus (CEM Discover, initial irradiation power 100 W) for 3 h. After cooling to RT, the mixture was concentrated and the residue was purified by means of preparative HPLC (method N). The combined product fractions were concentrated to a residual volume of aqueous phase. Saturated aqueous sodium bicarbonate solution was added and the mixture was extracted twice with ethyl acetate. The combined ethyl acetate phases were dried over magnesium sulphate, filtered and concentrated. After the residue had been dried in vacuo, 136 mg (66% of th.) of the title compound were obtained.

¹H-NMR (400 MHz, CDCl₃, δ/ppm): 8.10 (d, 2H), 8.02 (d, 1H), 7.50 (d, 2H), 7.40-7.38 (dd, 1H), 6.76 (s, 1H), 6.35 (d, 1H), 5.30 (s, 2H), 4.65-4.49 (m, 1H), 3.70 (s, 2H), 3.10-3.00 (m, 2H), 2.90 (d, 3H), 2.30 (s, 3H), 1.02 (d, 12H).

LC/MS (method F, ESIpos): Rₜ=0.65 min, m/z=460 [M+H]⁺.

### Example 185

### N-Methyl-5-[(5-methyl-3-{3-[4-(pentafluoro-λ⁶-sulphanyl)phenyl]-1,2,4-oxadiazol-5-yl}-1H-pyrazol-1-yl)methyl]pyridin-2-amine

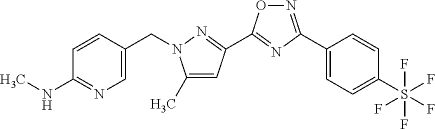

### Step 1: N-(3,4-Dimethoxybenzyl)-N-methyl-5-[(5-methyl-3-{3-[4-(pentafluoro-λ⁶-sulphanyl)phenyl]-1,2,4-oxadiazol-5-yl}-1H-pyrazol-1-yl)methyl]pyridin-2-amine

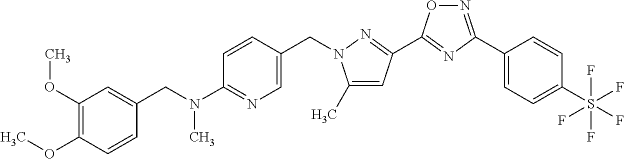

Analogously to the process described under Example 66, 400 mg (0.923 mmol, purity of 92%) of the compound from Example 92A and 242 mg (0.923 mmol) of the compound from Example 11A were reacted to give 222 mg (37% of th., purity of 95%) of the title compound. The crude product was purified by means of preparative HPLC (method N).

¹H-NMR (400 MHz, CDCl₃, δ/ppm): 8.31 (d, 2H), 8.12 (d, 1H), 7.88 (d, 2H), 7.42-7.38 (dd, 1H), 6.82-6.70 (m, 4H), 6.49 (d, 1H), 5.31 (s, 2H), 4.71 (s, 2H), 3.84 (s, 3H), 3.81 (s, 3H), 3.04 (s, 3H), 2.34 (s, 3H).

LC/MS (method I, ESIpos): Rₜ=1.25 min, m/z=623 [M+H]⁺.

### Step 2: N-Methyl-5-[(5-methyl-3-{3-[4-(pentafluoro-λ⁶-sulphanyl)phenyl]-1,2,4-oxadiazol-5-yl}-1H-pyrazol-1-yl)methyl]pyridin-2-amine

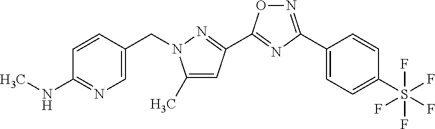

1 ml of trifluoroacetic acid was added to a solution of 180 mg (0.289 mmol) of the compound from Example 185/step 1 in 1 ml of methylene chloride and the mixture was stirred at RT for 3 days. The mixture was then concentrated and the residue was purified by means of preparative HPLC (method N). The combined product fractions were concentrated to a residual volume of aqueous phase and saturated aqueous sodium bicarbonate solution was added. The solid formed was filtered off, washed twice with water and dried in vacuo. 109 mg (80% of th.) of the title compound were obtained.

¹H-NMR (400 MHz, CDCl₃, δ/ppm): 8.31 (d, 2H), 8.03 (d, 1H), 7.89 (d, 2H), 7.40-7.37 (dd, 1H), 6.78 (s, 1H), 6.36 (d, 1H), 5.30 (s, 2H), 4.65-4.57 (m, broad, 1H), 2.91 (d, 3H), 2.33 (s, 3H).

LC/MS (method D, ESIpos): Rₜ=1.90 min, m/z=473 [M+H]⁺.

### Example 186

### N-Methyl-5-{[5-methyl-3-(3-{4-[(trifluoromethyl)sulphonyl]phenyl}-1,2,4-oxadiazol-5-yl)-1H-pyrazol-1-yl]methyl}pyridin-2-amine

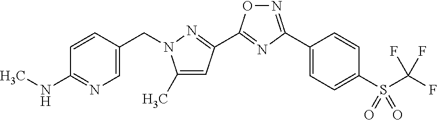

### Step 1: N-(3,4-Dimethoxybenzyl)-N-methyl-5-{[5-methyl-3-(3-{4-[(trifluoromethyl)-sulphonyl]phenyl}-1,2,4-oxadiazol-5-yl)-1H-pyrazol-1-yl]methyl}pyridin-2-amine

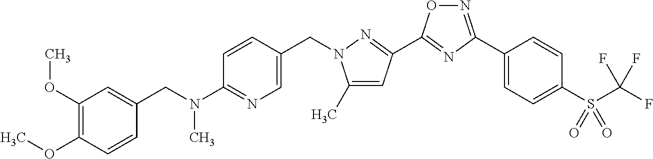

Analogously to the process described under Example 3, 200 mg (0.558 mmol) of the compound from Example 26A and 171 mg (0.558 mmol) of the compound from Example 90A were reacted to give 127 mg (36% of th.) of the title compound. In deviation from the instructions mentioned, after a reaction time of 18 h at RT a further 16 mg (0.140 mmol) of potassium tert-butylate were added here and the mixture was stirred again at RT for 4 h. The crude product was purified by means of preparative HPLC (method N).

¹H-NMR (400 MHz, CDCl₃, δ/ppm): 8.46 (d, 2H), 8.26 (d, 1H), 8.21 (d, 2H), 7.52-7.49 (dd, 1H), 6.92 (s, 1H), 6.79-6.70 (m, 2H), 6.45 (d, 1H), 5.79 (s, 2H), 4.67 (s, 2H), 3.82 (s, 3H), 3.79 (s, 3H), 3.01 (s, 3H), 2.36 (s, 3H).

LC/MS (method D, ESIpos): Rₜ=2.39 min, m/z=629 [M+H]⁺.

### Step 2: N-Methyl-5-{[5-methyl-3-(3-{4-[(trifluoromethyl)sulphonyl]phenyl}-1,2,4-oxadiazol-5-yl)-1H-pyrazol-1-yl]methyl}pyridin-2-amine

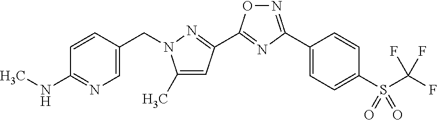

Analogously to the process described under Example 185/step 2, 100 mg (0.159 mmol) of the compound from Example 186/step 1 were reacted to give 76 mg (79% of th., purity of 95%) of the title compound. In deviation from the instructions mentioned, after addition of the sodium bicarbonate solution the mixture was extracted here three times with ethyl acetate. The combined ethyl acetate extracts were dried over magnesium sulphate, filtered and concentrated and the residue was dried in vacuo to give the title compound.

¹H-NMR (400 MHz, CDCl₃, δ/ppm): 8.46 (d, 2H), 8.22-8.18 (m, 3H), 7.52-7.49 (dd, 1H), 6.92 (s, 1H), 6.32 (d, 1H), 5.76 (s, 2H), 4.60 (s, broad, 1H), 2.88 (d, 3H), 2.35 (s, 3H).

LC/MS (method F, ESIpos): Rₜ=1.09 min, m/z=479 [M+H]⁺.

### Example 187

### N-Methyl-5-{[5-methyl-3-(3-{4-[N-methyl-S-(trifluoromethyl)sulphonimidoyl]phenyl}-1,2,4-oxa-diazol-5-yl)-1H-pyrazol-1-yl]methyl}pyridin-2-amine (racemate)

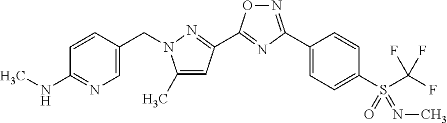

### Step 1: N-(3,4-Dimethoxybenzyl)-N-methyl-5-{[5-methyl-3-(3-{4-[N-methyl-S-(trifluoro-methyl)sulphonimidoyl]phenyl}-1,2,4-oxadiazol-5-yl)-1H-pyrazol-1-yl]methyl}-pyridin-2-amine (racemate)

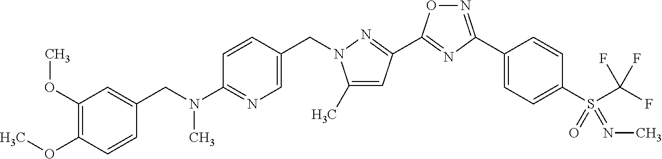

170 μl (1.95 mmol) of oxalyl chloride were added to a solution of 282 mg (0.650 mmol, purity of 92%) of the compound from Example 92A and one drop of DMF in 6.5 ml of methylene chloride at 0° C. and the mixture was stirred at RT for 1 h. The mixture was then concentrated and the residue was dried in vacuo and subsequently taken up in 4 ml of methylene chloride. This mixture was then added to a solution of 188 mg (0.650 mmol, purity of 97%) of the compound from Example 68A and 181 μl (1.30 mmol) of triethylamine in 2.5 ml of methylene chloride at 0° C. and the mixture was stirred at RT for 1 h. The mixture was then concentrated and the residue was dried in vacuo and subsequently taken up in 6.5 ml of DMSO. This mixture was then heated at 120° C. in a microwave apparatus (CEM Discover, initial irradiation power 100 W) for 30 min. After cooling to RT, the mixture was purified directly by means of preparative HPLC (method N). 89 mg (21% of th., purity of 96%) of the title compound were obtained in this way.

LC/MS (method D, ESIpos): Rₜ=2.36 min, m/z=642 [M+H]⁺.

### Step 2: N-Methyl-5-{[5-methyl-3-(3-{4-[N-methyl-S-(trifluoromethyl)sulphonimidoyl]phenyl}-1,2,4-oxadiazol-5-yl)-1H-pyrazol-1-yl]methyl}pyridin-2-amine (racemate)

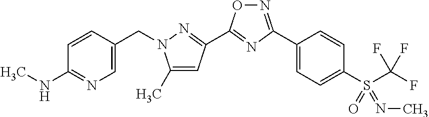

0.5 ml (6.49 mmol) of trifluoroacetic acid was added to a solution of 89 mg (0.133 mmol, purity of 96%) of the compound from Example 187/step 1 in 0.5 ml of methylene chloride and the mixture was stirred at RT overnight. It was subsequently concentrated and the residue was purified by means of preparative HPLC (method N). The combined product fractions were concentrated to a residual volume of aqueous phase, saturated aqueous sodium bicarbonate solution was added and the mixture was extracted twice with ethyl acetate. The combined ethyl acetate phases were dried over magnesium sulphate, filtered and concentrated. The residue was dried in vacuo and then purified again by means of preparative HPLC (method N). The combined product fractions were concentrated again to a residual volume of aqueous phase, saturated aqueous sodium bicarbonate solution was added and the mixture was extracted twice with ethyl acetate. The combined ethyl acetate phases were dried over magnesium sulphate, filtered and concentrated. After drying of the residue in vacuo, 38 mg (54% of th.) of the title compound were obtained.

¹H-NMR (400 MHz, CDCl₃, δ/ppm): 8.44 (d, 2H), 8.21 (d, 2H), 8.02 (d, 1H), 7.42-7.39 (dd, 1H), 6.79 (s, 1H), 6.38 (d, 1H), 5.30 (s, 2H), 4.92 (s, broad, 1H), 3.12 (d, 3H), 2.92 (d, 3H), 2.33 (s, 3H).

LC/MS (method I, ESIpos): Rₜ=0.96 min, m/z=492 [M+H]⁺.

### Example 188

### N-Methyl-5-{[5-methyl-3-(3-{4-[S-(trifluoromethyl)sulphonimidoyl]phenyl}-1,2,4-oxadiazol-5-yl)-1H-pyrazol-1-yl]methyl}pyridin-2-amine (racemate)

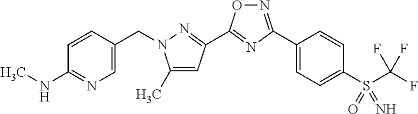

### Step 1: N-(3,4-Dimethoxybenzyl)-N-methyl-5-{[5-methyl-3-(3-{4-[S-(trifluoromethyl)-sulphonimidoyl]phenyl}-1,2,4-oxadiazol-5-yl)-1H-pyrazol-1-yl]methyl}pyridin-2-amine (racemate)

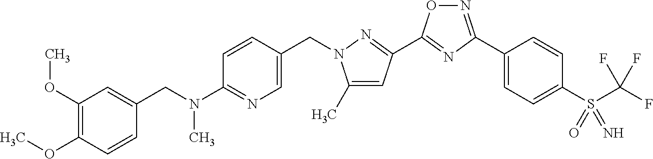

322 mg (1.677 mmol) of EDC and 227 mg (1.677 mmol) of HOBt were added to a solution of 700 mg (1.68 mmol, purity of 95%) of the compound from Example 92A in 8 ml of DMF, the mixture was stirred at RT for 30 min and 498 mg (1.68 mmol, purity of 90%) of the compound from Example 69A were then added. The mixture was stirred at RT for a further 30 min and then heated at 150° C. for 30 min, while stirring. After cooling to RT, the mixture was concentrated and the residue was taken up in ethyl acetate and water. After separation of the phases, the aqueous phase was extracted once with ethyl acetate. The combined ethyl acetate phases were washed once with saturated sodium chloride solution, dried over magnesium sulphate, filtered and concentrated. The residue was purified by means of preparative HPLC (method N). After the product had been dried in vacuo, 87 mg (8% of th., purity of 98%) of the title compound were obtained.

LC/MS (method I, ESIpos): Rₜ=1.08 min, m/z=628 [M+H]⁺.

### Step 2: N-Methyl-5-{[5-methyl-3-(3-{4-[S-(trifluoromethyl)sulphonimidoyl]phenyl}-1,2,4-oxadiazol-5-yl)-1H-pyrazol-1-yl]methyl}pyridin-2-amine (racemate)

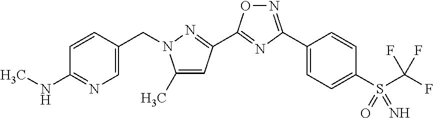

0.7 ml (9.086 mmol) of trifluoroacetic acid was added to a solution of 85 mg (0.135 mmol, purity of 96%) of the compound from Example 188/step 1 in 0.7 ml of methylene chloride and the mixture was stirred at RT for 28 h. It was subsequently concentrated and the residue was purified by means of preparative HPLC (method N). The combined product fractions were concentrated to a residual volume of aqueous phase and saturated aqueous sodium bicarbonate solution was added. The solid formed was filtered off, washed twice with water and dried in vacuo. 39 mg (60% of th.) of the title compound were obtained.

¹H-NMR (400 MHz, CDCl₃, δ/ppm): 8.49 (d, 2H), 8.28 (d, 2H), 8.03 (s, 1H), 7.40 (d, 1H), 6.80 (s, 1H), 6.36 (d, 1H), 5.30 (s, 2H), 4.61 (s, broad, 1H), 3.72 (s, 1H), 2.92 (d, 3H), 2.32 (s, 3H).

LC/MS (method F, ESIpos): Rₜ=0.94 min, m/z=478 [M+H]⁺.

### Example 189

### Ethyl 4-{4-[5-(5-methyl-1-{[6-(methylamino)pyridin-3-yl]methyl}-1H-pyrazol-3-yl)-1,2,4-oxa-diazol-3-yl]phenyl}tetrahydro-2H-pyran-4-carboxylate

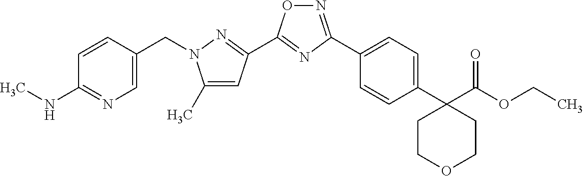

80 mg (0.16 mmol) of the compound from Example 153 were heated in 0.97 ml (7.87 mmol) of a 33% strength solution of methylamine in ethanol at 160° C. in a microwave oven for 9 h. After cooling to RT, the mixture was purified directly by means of preparative HPLC (method P). The combined product fractions were concentrated on a rotary evaporator. After the residue had been dried in vacuo, 36 mg (40% of th.) of the title compound were obtained.

¹H-NMR (400 MHz, DMSO-d₆, δ/ppm): 8.06 (d, 2H), 7.99 (d, 1H), 7.59 (d, 2H), 7.30 (dd, 1H), 6.87 (s, 1H), 6.54 (m, 1H), 6.42 (d, 1H), 5.27 (s, 2H), 4.12 (q, 2H), 3.84 (m, 2H), 3.46 (t, 2H), 2.74 (d, 3H), 2.42 (m, 2H), 2.38 (s, 3H), 1.94 (m, 2H), 1.12 (t, 3H).

LC/MS (method D, ESIpos): Rₜ=1.74 min, m/z=503 [M+H]⁺.

### Example 190

### (4-{4-[5-(5-Methyl-1-{[6-(methylamino)pyridin-3-yl]methyl}-1H-pyrazol-3-yl)-1,2,4-oxadiazol-3-yl]phenyl}tetrahydro-2H-pyran-4-yl)methanol

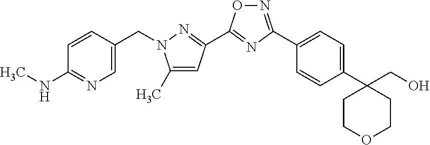

50 mg (0.11 mmol) of the compound from Example 155 were stirred in 505 mg (5.36 mmol) of a 33% strength solution of methylamine in ethanol at 150° C. in a microwave oven for 9 h. After cooling to RT, the mixture was purified directly by means of preparative HPLC (method P). The combined product fractions were concentrated on a rotary evaporator. After the residue had been dried in vacuo, 15 mg (29% of th.) of the title compound were obtained.

¹H-NMR (400 MHz, DMSO-d₆, δ/ppm): 8.02 (d, 2H), 7.99 (d, 1H), 7.57 (d, 2H), 7.30 (dd, 1H), 6.87 (s, 1H), 6.56 (q, 1H), 6.42 (d, 1H), 5.27 (s, 2H), 4.70 (t, 1H), 3.71 (m, 2H), 3.43 (d, 2H), 3.38 (m, 2H), 2.74 (d, 3H), 2.38 (s, 3H), 2.03 (m, 2H), 1.89 (m, 2H).

LC/MS (method D, ESIpos): Rₜ=1.43 min, m/z=461 [M+H]⁺.

### Example 191

### N,N-Dimethyl-4-{4-[5-(5-methyl-1-{[6-(methylamino)pyridin-3-yl]methyl}-1H-pyrazol-3-yl)-1,2,4-oxadiazol-3-yl]phenyl}tetrahydro-2H-pyran-4-carboxamide

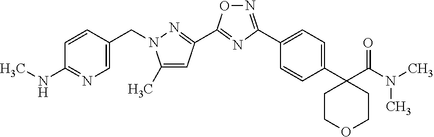

60 mg (0.12 mmol) of the compound from Example 156 were stirred in 1.2 ml of ethanol and 1.2 ml of an 8 M solution of methylamine in ethanol at 160° C. in a microwave oven for 10 h. After cooling to RT, the mixture was purified directly by means of preparative HPLC (method P). The combined product fractions were concentrated on a rotary evaporator. After the residue had been dried in vacuo, 25 mg (42% of th.) of the title compound were obtained.

¹H-NMR (400 MHz, DMSO-d₆, δ/ppm): 8.07 (d, 2H), 7.99 (d, 1H), 7.46 (d, 2H), 7.30 (dd, 1H), 6.86 (s, 1H), 6.56 (q, 1H), 6.41 (d, 1H), 5.27 (s, 2H), 3.78 (m, 2H), 3.61 (t, 2H), 2.73 (d, 3H), 2.38 (s, 3H), 2.21 (d, 2H), 1.95 (m, 2H).

LC/MS (method I, ESIpos): Rₜ=0.76 min, m/z=502 [M+H]⁺.

### Example 192

### N-Methyl-4-{4-[5-(5-methyl-1-{[6-(methylamino)pyridin-3-yl]methyl}-1H-pyrazol-3-yl)-1,2,4-oxadiazol-3-yl]phenyl}tetrahydro-2H-pyran-4-carboxamide

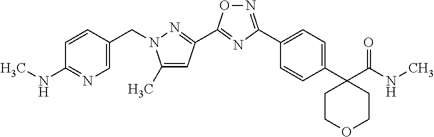

Analogously to the process described under Example 191, 34 mg (34% of th.) of the title compound were obtained from 100 mg (0.20 mmol) of the compound from Example 157.

¹H-NMR (400 MHz, DMSO-d₆, δ/ppm): 8.03 (d, 2H), 7.99 (d, 1H), 7.67 (q, 1H), 7.55 (d, 2H), 7.29 (dd, 1H), 6.86 (s, 1H), 6.54 (q, 1H), 6.41 (d, 1H), 5.27 (s, 2H), 3.75 (m, 2H), 3.47 (t, 2H), 2.73 (d, 3H), 2.56 (d, 3H), 2.46 (d, 2H), 2.38 (s, 3H), 1.89 (m, 2H).

LC/MS (method I, ESIpos): Rₜ=0.70 min, m/z=488 [M+H]⁺.

### Example 193

### 2,2-Dimethyl-3-({5-[(5-methyl-3-{3-[4-(1,1,1-trifluoro-2-methylpropan-2-yl)phenyl]-1,2,4-oxa-diazol-5-yl}-1H-pyrazol-1-yl)methyl]pyridin-2-yl}amino)propan-1-ol

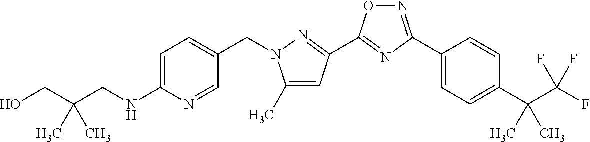

100 mg (0.22 mmol) of the compound from Example 68 and 111 mg (1.08 mmol) of 3-amino-2,2-dimethylpropan-1-ol were dissolved in 1 ml of DMSO and the solution was heated at 160° C. overnight. After cooling to RT, the mixture was purified directly by means of preparative HPLC (method P). The combined product fractions were concentrated on a rotary evaporator. After the residue had been dried in vacuo, 18 mg (15% of th.) of the title compound were obtained.

¹H-NMR (400 MHz, DMSO-d₆, δ/ppm): 8.09 (d, 2H), 7.92 (d, 1H), 7.77 (d, 2H), 7.29 (dd, 1H), 6.88 (s, 1H), 6.67 (broad, 1H), 6.54 (d, 1H), 5.26 (s, 2H), 4.97 (broad, 1H), 3.10 (d, 2H), 3.06 (d, 2H), 2.38 (s, 3H), 1.61 (s, 6H), 0.81 (s, 6H).

LC/MS (method C, ESIpos): Rₜ=2.00 min, m/z=529 [M+H]⁺.

### Example 194

### 3-(Methyl {5-[(5-methyl-3-{3-[4-(1,1,1-trifluoro-2-methylpropan-2-yl)phenyl]-1,2,4-oxadiazol-5-yl}-1H-pyrazol-1-yl)methyl]pyridin-2-yl}amino)propan-1-ol

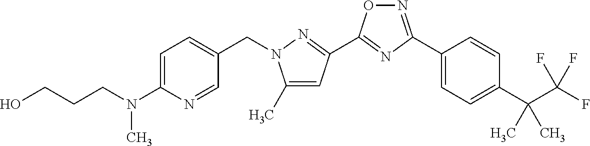

90 mg (0.19 mmol) of the compound from Example 68 and 87 mg (0.97 mmol) of 3-(methylamino)propan-1-ol were dissolved in 1 ml of N-methylpyrrolidin-2-one and the solution was heated at 160° C. in a microwave oven for 8 h. After cooling to RT, the mixture was purified directly by means of preparative HPLC (method P). The combined product fractions were concentrated on a rotary evaporator. After the residue had been dried in vacuo, 48 mg (48% of th.) of the title compound were obtained.

¹H-NMR (400 MHz, DMSO-d₆, δ/ppm): 8.09 (d, 2H), 8.07 (d, 1H), 7.77 (d, 2H), 7.41 (dd, 1H), 6.88 (s, 1H), 6.60 (d, 1H), 5.30 (s, 2H), 4.51 (t, 1H), 3.51 (d, 2H), 3.40 (q, 2H), 2.97 (s, 3H), 2.39 (s, 3H), 1.64 (m, 2H), 1.61 (s, 6H).

LC/MS (method I, ESIpos): Rₜ=1.03 min, m/z=515 [M+H]⁺.

### Example 195

### 2-Hydrazino-5-[(5-methyl-3-{3-[4-(trifluoromethoxy)phenyl]-1,2,4-oxadiazol-5-yl}-1H-pyrazol-1-yl)methyl]pyridine

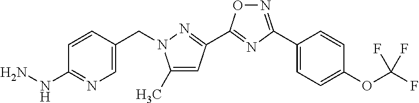

5.0 ml (103 mmol) of hydrazine hydrate were added to 1.00 g (2.29 mmol) of the compound from Example 2 under argon at RT. The mixture was heated under reflux for 16 h, while stirring, a further 5.0 ml of hydrazine hydrate were then added and the mixture was stirred under reflux again for 16 h. The solid thereby formed was dissolved again by addition of 10 ml of ethanol and the mixture was then heated under reflux for a further 24 h, while stirring. After cooling to RT, the solid formed was filtered off, washed once with a 1:1 mixture of water and ethanol and dried in vacuo. 788 mg (80% of th.) of the title compound were obtained.

¹H-NMR (400 MHz, CDCl₃, δ/ppm): 8.25 (d, 2H), 8.06 (d, 1H), 7.43-7.39 (dd, 1H), 7.32 (d, 2H), 6.79 (s, 1H), 6.69 (d, 1H), 5.88 (s, 1H), 5.31 (s, 2H), 3.80 (s, broad, 2H), 2.31 (s, 3H).

LC/MS (method I, ESIpos): Rₜ=0.90 min, m/z=432 [M+H]⁺.

### Example 196

### N-Methyl-5-[(2-methyl-4-{3-[4-(1,1,1-trifluoro-2-methylpropan-2-yl)phenyl]-1,2,4-oxadiazol-5-yl}-1H-pyrrol-1-yl)methyl]pyridin-2-amine

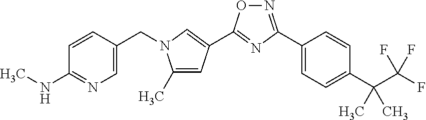

A mixture of 200 mg (0.434 mmol) of the compound from Example 75 and 5.4 ml (43.4 mmol) of a 33% strength methylamine solution in ethanol was heated at 160° C. in a microwave apparatus (CEM Discover, initial irradiation power 100 W) for 5 h. After cooling to RT, the mixture was purified by preparative HPLC (method N) twice. The combined product fractions were concentrated to a residual volume of aqueous phase, saturated aqueous sodium bicarbonate solution was added and the mixture was extracted twice with ethyl acetate. The combined ethyl acetate phases were then dried over magnesium sulphate, filtered and concentrated. After drying of the residue in vacuo, 62 mg (31% of th.) of the title compound were obtained.

¹H-NMR (400 MHz, CDCl₃, δ/ppm): 8.10 (d, 2H), 7.99 (d, 1H), 7.60 (d, 2H), 7.42 (d, 1H), 7.21-7.18 (dd, 1H), 6.53 (s, 1H), 6.38 (d, 1H), 4.92 (s, 2H), 4.63-4.55 (m, broad, 1H), 2.93 (d, 3H), 2.24 (s, 3H), 1.61 (s, 6H).

LC/MS (method F, ESIpos): Rₜ=1.14 min, m/z=456 [M+H]⁺.

### Example 197

### N-Ethyl-5-[(2-methyl-4-{3-[4-(1,1,1-trifluoro-2-methylpropan-2-yl)phenyl]-1,2,4-oxadiazol-5-yl}-1H-pyrrol-1-yl)methyl]pyridin-2-amine

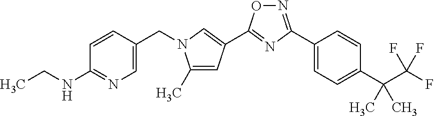

A mixture of 200 mg (0.434 mmol) of the compound from Example 75, 4.3 ml (8.68 mmol) of a 2 M solution of ethylamine in THF and 2.0 ml (24.8 mmol) of a 70% strength ethylamine solution in water was heated at 170° C. in a microwave apparatus (CEM Discover, initial irradiation power 250 W) for 6 h. After cooling to RT, a further 1.0 ml (12.4 mmol) of a 70% strength ethylamine solution in water was added and the mixture was heated again at 170° C. in the microwave apparatus for 6 h. After cooling to RT, the mixture was concentrated and the residue was purified by means of preparative HPLC (method N). The combined product fractions were concentrated to a residual volume of aqueous phase. Saturated aqueous sodium bicarbonate solution was added and the mixture was extracted twice with ethyl acetate. The ethyl acetate phases were combined, dried over magnesium sulphate, filtered and concentrated. After drying of the residue in vacuo, 83 mg (40% of th.) of the title compound were obtained.

¹H-NMR (400 MHz, CDCl₃, δ/ppm): 8.10 (d, 2H), 7.96 (s, 1H), 7.61 (s, 2H), 7.43 (s, 1H), 7.21-7.17 (dd, 1H), 6.54 (s, 1H), 6.37 (d, 1H), 4.92 (s, 2H), 4.65 (s, broad, 1H), 3.35-3.28 (m, 2H), 2.24 (s, 3H), 1.61 (s, 6H), 1.29-1.22 (t, 3H).

LC/MS (method F, ESIpos): Rₜ=1.20 min, m/z=470 [M+H]⁺.

### Example 198

### 2-(Methylsulphanyl)-5-[(5-methyl-3-{3-[4-(1,1,1-trifluoro-2-methylpropan-2-yl)phenyl]-1,2,4-oxa-diazol-5-yl}-1H-pyrazol-1-yl)methyl]pyridine

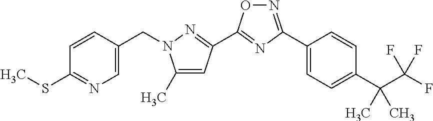

A mixture of 100 mg (0.217 mmol) of the compound from Example 68 and 46 mg (0.650 mmol) of a sodium methanethiolate in 1 ml of dioxane was heated under reflux for 5 h, while stirring. After cooling to RT, 20 ml of water and 20 ml of ethyl acetate were added to the mixture, the phases were separated and the aqueous phase was extracted twice more with 20 ml of ethyl acetate. The combined ethyl acetate phases were dried over sodium sulphate, filtered and concentrated. The residue was purified by means of flash chromatography (silica gel, mobile phase: cyclohexane/ethyl acetate 3:2). After drying in vacuo, 64 mg (62% of th.) of the title compound were obtained.

¹H-NMR (400 MHz, CDCl₃, δ/ppm): 8.35 (s, 1H), 8.19 (d, 2H), 7.62 (d, 2H), 7.37 (d, 1H), 7.15 (d, 1H), 6.81 (s, 1H), 5.39 (s, 2H), 2.55 (s, 3H), 2.32 (s, 3H), 1.63 (s, 6H).

LC/MS (method F, ESIpos): Rₜ=1.55 min, m/z=474 [M+H]⁺.

### Example 199

### 2-Methoxy-5-[(5-methyl-3-{3-[4-(trifluoromethoxy)phenyl]-1,2,4-oxadiazol-5-yl}-1H-pyrazol-1-yl)methyl]pyridine

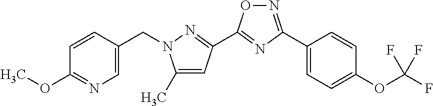

Analogously to the process described under Example 2, 155 mg (0.50 mmol) of the compound from Example 23A and 118 mg (0.750 mmol) of 5-(chloromethyl)-2-methoxypyridine [H. Harada et al., WO 2006/101081] were reacted to give 24 mg (11% of th., purity of 99%) of a first batch and 49 mg (19% of th., purity of 83%) of a second batch of the title compound. In deviation from the instructions mentioned, the reaction time in this case was 36 h at a temperature of 50° C. The purification of the crude product was carried out by means of preparative HPLC (method N). The title compound was isolated by concentrating each of the combined product fractions in the two separate batches to a residual volume of aqueous phase, adding saturated aqueous sodium bicarbonate solution to the residue and extracting the mixture twice with ethyl acetate. The combined ethyl acetate phases in the two batches were then dried over magnesium sulphate, filtered and concentrated and each of the particular residues were dried in vacuo.

¹H-NMR (400 MHz, CDCl₃, δ/ppm): 8.25 (d, 2H), 8.09 (d, 1H), 7.51-7.48 (dd, 1H), 7.33 (d, 2H), 6.80 (s, 1H), 6.72 (d, 1H), 5.38 (s, 2H), 3.92 (s, 3H), 2.32 (s, 3H).

LC/MS (method F, ESIpos): Rₜ=1.47 min, m/z=432 [M+H]⁺.

### Example 200

### 2-Methoxy-5-[(5-methyl-3-{3-[4-(1,1,1-trifluoro-2-methylpropan-2-yl)phenyl]-1,2,4-oxadiazol-5-yl}-1H-pyrazol-1-yl)methyl]pyridine

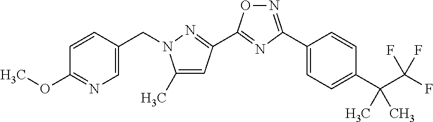

Analogously to the process described under Example 2, 168 mg (0.50 mmol) of the compound from Example 24A and 118 mg (0.750 mmol) of 5-(chloromethyl)-2-methoxypyridine [H. Harada et al., WO 2006/101081] were reacted to give 83 mg (36% of th.) of the title compound. In deviation from the instructions mentioned, the reaction time here was 36 h at a temperature of 50° C. The purification of the crude product was carried out by means of preparative HPLC (method N). The title compound was isolated by concentrating the combined product fractions to a residual volume of aqueous phase, adding saturated aqueous sodium bicarbonate solution and extracting the mixture twice with ethyl acetate. The combined ethyl acetate phases were then dried over magnesium sulphate, filtered and concentrated and the residue was dried in vacuo.

¹H-NMR (400 MHz, CDCl₃, δ/ppm): 8.19 (d, 2H), 8.08 (d, 1H), 7.62 (d, 2H), 7.51-7.48 (dd, 1H), 6.80 (d, 1H), 6.72 (d, 1H), 5.38 (s, 2H), 3.92 (s, 3H), 2.32 (d, 3H), 1.62 (s, 6H).

LC/MS (method F, ESIpos): Rₜ=1.50 min, m/z=458 [M+H]⁺.

### Example 201

### 2-Cyclopropyl-5-[(5-methyl-3-{3-[4-(trifluoromethoxy)phenyl]-1,2,4-oxadiazol-5-yl}-1H-pyrazol-1-yl)methyl]pyridine

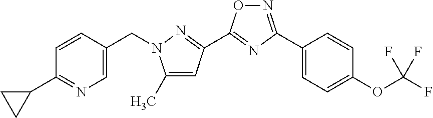

16 mg (0.014 mmol) of tetrakis(triphenylphosphine)palladium(0) and 1.14 ml (0.569 mmol) of a 0.5 M solution of bromo(cyclopropyl)zinc in THF were added to a solution of 150 mg (0.284 mmol) of the compound from Example 158 in 1.5 ml of DMF under argon at RT and the mixture was stirred at RT overnight. 20 ml of water were subsequently added and the solid formed was filtered off and this was washed twice with water and dried in vacuo. The solid was then stirred in a mixture of water, acetonitrile and DMSO under the influence of heat. The precipitate which remained was filtered off, washed twice with 2 ml of water and dried in vacuo. 92 mg (73% of th.) of the title compound were obtained.

¹H-NMR (400 MHz, CDCl₃, δ/ppm): 8.35 (s, 1H), 8.25 (d, 2H), 7.42-7.31 (m, 4H), 7.10 (d, 1H), 6.80 (s, 1H), 5.40 (s, 2H), 2.31 (s, 3H), 2.06-1.99 (m, 1H), 1.03-0.99 (m, 4H).

LC/MS (method I, ESIpos): Rₜ=1.28 min, m/z=442 [M+H]⁺.

### Example 202

### 2-Chloro-4-[(5-methyl-3-{3-[4-(tetrahydro-2H-pyran-4-yl)phenyl]-1,2,4-oxadiazol-5-yl}-1H-pyrazol-1-yl)methyl]pyridine

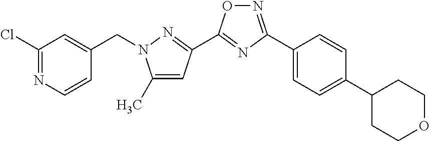

Analogously to the process described under Example 3, 204 mg (1.26 mmol) of the compound from Example 38A and 300 mg (0.967 mmol) of the compound from Example 74A were reacted to give 220 mg (52% of th.) of the title compound. The product was isolated by means of preparative HPLC (method M).

¹H-NMR (400 MHz, CDCl₃, δ/ppm): 8.37 (d, 1H), 8.14 (d, 2H), 7.35 (d, 2H), 7.05 (d, 1H), 6.96 (dd, 1H), 6.88 (s, 1H), 5.43 (s, 2H), 4.12-4.08 (m, 2H), 3.58-3.52 (m, 2H), 2.88-2.79 (m, 1H), 2.31 (s, 3H), 1.92-1.79 (m, 4H).

LC/MS (method I, ESIpos): Rₜ=1.16 min, m/z=436/438 [M+H]⁺.

### Example 203

### 2-Chloro-4-[(5-methyl-3-{3-[3-methyl-4-(tetrahydro-2H-pyran-4-yl)phenyl]-1,2,4-oxadiazol-5-yl}-1H-pyrazol-1-yl)methyl]pyridine

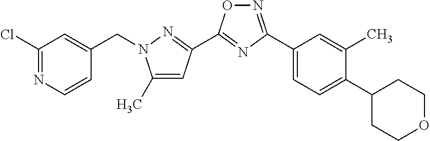

Analogously to the process described under Example 3, 121 mg (0.749 mmol) of the compound from Example 38A and 187 mg (0.576 mmol) of the compound from Example 75A were reacted to give 150 mg (58% of th.) of the title compound. In this case, the reaction mixture was heated under reflux for 8 h. The product was isolated by means of preparative HPLC (method M).

¹H-NMR (400 MHz, CDCl₃, δ/ppm): 8.37 (d, 1H), 8.02 (d, 1H), 8.01 (dd, 1H), 7.34 (d, 1H), 7.05 (d, 1H), 6.96 (dd, 1H), 6.88 (s, 1H), 5.43 (s, 2H), 4.13-4.09 (m, 2H), 3.61-3.53 (m, 2H), 3.07-2.99 (m, 1H), 2.43 (s, 3H), 2.31 (s, 3H), 1.92-1.81 (m, 2H), 1.74-1.69 (m, 2H).

LC/MS (method F, ESIpos): Rₜ=1.34 min, m/z=448/450 [M+H]⁺.

### Example 204

### 4-({3-[3-(4-tert-Butylphenyl)-1,2,4-oxadiazol-5-yl]-5-methyl-1H-pyrazol-1-yl}methyl)-2-chloro-pyridine

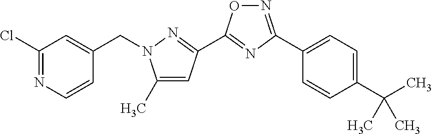

Analogously to the process described under Example 3, 1.15 g (7.08 mmol) of the compound from Example 38A and 1.0 g (3.54 mmol) of the compound from Example 82A were reacted to give 578 mg (40% of th.) of the title compound.

¹H-NMR (400 MHz, CDCl₃, δ/ppm): 8.37 (d, 1H), 8.13 (d, 2H), 7.51 (d, 2H), 7.06 (s, 1H), 6.97 (d, 1H), 6.89 (s, 1H), 5.44 (s, 2H), 2.30 (s, 3H), 1.36 (s, 9H).

LC/MS (method F, ESIpos): Rₜ=1.55 min, m/z=408/410 [M+H]⁺.

### Example 205

### 2-Chloro-4-{[3-(3-{4-[1-(methoxymethyl)cyclobutyl]phenyl}-1,2,4-oxadiazol-5-yl)-5-methyl-1H-pyrazol-1-yl]methyl}pyridine

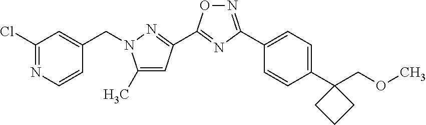

Analogously to the process described under Example 3, 749 mg (4.62 mmol) of the compound from Example 38A and 750 mg (2.31 mmol) of the compound from Example 77A were reacted to give 447 mg (43% of th.) of the title compound.

¹H-NMR (400 MHz, CDCl₃, δ/ppm): 8.37 (d, 1H), 8.13 (d, 2H), 7.30 (d, 2H), 7.06 (s, 1H), 6.97 (d, 1H), 6.88 (s, 1H), 5.43 (s, 2H), 3.56 (s, 2H), 3.29 (s, 3H), 2.41-2.29 (m, 4H), 2.31 (s, 3H), 2.16-2.04 (m, 1H), 1.93-1.83 (m, 1H).

LC/MS (method I, ESIpos): Rₜ=1.34 min, m/z=450/452 [M+H]⁺.

### Example 206

### 2-Chloro-4-[(3-{3-[3-chloro-4-(trifluoromethoxy)phenyl]-1,2,4-oxadiazol-5-yl}-5-methyl-1H-pyrazol-1-yl)methyl]pyridine

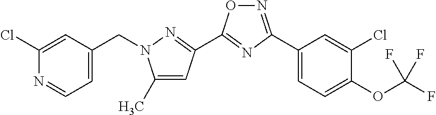

Analogously to the process described under Example 2, 500 mg (1.38 mmol, purity of 95%) of the compound from Example 84A and 290 mg (1.79 mmol) of the compound from Example 38A were reacted to give 386 mg (57% of th., purity of 96%) of the title compound. In this case, the reaction mixture was heated under reflux for 14 h.

¹H-NMR (400 MHz, CDCl₃, δ/ppm): 8.40-8.37 (m, 2H), 8.12 (d, 1H), 7.44 (d, 1H), 7.05 (s, 1H), 6.96 (d, 1H), 6.89 (s, 1H), 5.45 (s, 2H), 2.31 (s, 3H).

LC/MS (method I, ESIpos): Rₜ=1.36 min, m/z=469/471 [M+H]⁺.

### Example 207

### 2-Chloro-4-{[5-methyl-3-(3-{4-[1-(trifluoromethyl)cyclopropyl]phenyl}-1,2,4-oxadiazol-5-yl)-1H-pyrazol-1-yl]methyl}pyridine

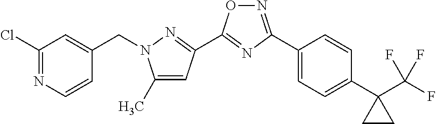

A mixture of 450 mg (1.35 mmol) of the compound from Example 86A, 284 mg (1.75 mmol) of 2-chloro-4-(chloromethyl)pyridine and 166 mg (1.48 mmol) of potassium tert-butylate in 12 ml of THF was heated under reflux overnight, while stirring. After cooling to RT, ethyl acetate and water were added to the mixture. The phases were separated and the aqueous phase was extracted twice more with ethyl acetate. The combined ethyl acetate phases were washed once with saturated sodium chloride solution, dried over magnesium sulphate, filtered and concentrated. The residue was purified by means of column chromatography (silica gel, mobile phase: cyclohexane/ethyl acetate 7:3). After drying in vacuo, 352 mg (57% of th.) of the title compound were obtained. ¹H-NMR (400 MHz, CDCl₃, δ/ppm): 8.37 (d, 1H), 8.19 (d, 2H), 7.60 (d, 2H), 7.05 (s, 1H), 6.96 (d, 1H), 6.88 (s, 1H), 5.44 (s, 2H), 2.30 (s, 3H), 1.42-1.39 (m, 2H), 1.09 (s, broad, 2H).

LC/MS (method F, ESIpos): Rₜ=1.48 min, m/z=460/462 [M+H]⁺.

### Example 208

### 2-Bromo-4-[(5-methyl-3-{3-[4-(trifluoromethoxy)phenyl]-1,2,4-oxadiazol-5-yl}-1H-pyrazol-1-yl)-methyl]pyridine

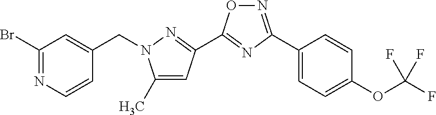

Analogously to the process described under Example 1, 1.05 g (4.19 mmol) of the compound from Example 87A were reacted with 1.0 g (3.22 mmol) of the compound from Example 23A to give 0.71 g (45% of th.) of the title compound. The duration of the reaction in this case was 16 h.

¹H-NMR (400 MHz, CDCl₃, δ/ppm): 8.35 (d, 1H), 8.24 (d, 2H), 7.33 (d, 2H), 7.22 (d, 1H), 6.99 (dd, 1H), 6.89 (s, 1H), 5.42 (s, 2H), 2.31 (s, 3H).

LC/MS (method I, ESIpos): Rₜ=1.32 min, m/z=480/482 [M+H]⁺.

### Example 209

### N-Methyl-4-[(5-methyl-3-{3-[4-(trifluoromethoxy)phenyl]-1,2,4-oxadiazol-5-yl}-1H-pyrazol-1-yl)methyl]pyridin-2-amine

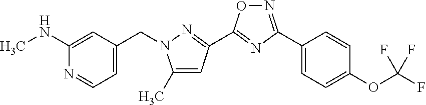

Analogously to the process described under Example 24, 150 mg (0.344 mmol) of the compound from Example 3 and 4.3 ml (34.4 mmol) of a 33% strength methylamine solution in ethanol were reacted to give 97 mg (66% of th.) of the title compound. The duration of the reaction in this case was 3 h at 140° C. in a microwave apparatus (initial irradiation power 100 W).

¹H-NMR (400 MHz, CDCl₃, δ/ppm): 8.31-8.20 (m, 2H), 8.05 (d, 1H), 7.34 (d, 2H), 6.84 (s, 1H), 6.33 (d, 1H), 6.03 (s, 1H), 5.34 (s, 2H), 4.54 (d, 1H), 2.88 (d, 3H), 2.29 (s, 3H).

LC/MS (method I, ESIpos): Rₜ=0.94 min, m/z=431 [M+H]⁺.

### Example 210

### 2-Cyclopropyl-4-[(5-methyl-3-{3-[4-(trifluoromethoxy)phenyl]-1,2,4-oxadiazol-5-yl}-1H-pyrazol-1-yl)methyl]pyridine

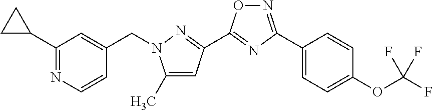

833 μl (0.416 mmol) of a 0.5 M solution of cyclopropylzinc bromide in THF was added to a solution of 100 mg (0.208 mmol) of the compound from Example 208 and 12 mg (0.010 mmol) of tetrakis(triphenylphosphine)palladium(0) in 2 ml of anhydrous DMF under inert conditions. After the reaction mixture had been stirred at RT for 16 h, hydrolysis was carried out with 3 drops of water and the mixture was diluted with approx. 2 ml of ethanol. The solution obtained in this way was separated directly into its components by means of preparative HPLC (method M). 68 mg (73% of th.) of the title compound were obtained.

¹H-NMR (400 MHz, CDCl₃, δ/ppm): 8.39 (d, 1H), 8.25 (d, 2H), 7.33 (d, 2H), 6.86 (dd, 1H und s, 1H), 6.75 (dd, 1H), 5.40 (s, 2H), 2.29 (s, 3H), 2.00-1.93 (m, 1H), 1.03-0.94 (m, 4H).

LC/MS (method I, ESIpos): Rₜ=1.22 min, m/z=442 [M+H]⁺.

### Example 211

### 4-{4-[(5-Methyl-3-{3-[4-(trifluoromethoxy)phenyl]-1,2,4-oxadiazol-5-yl}-1H-pyrazol-1-yl)-methyl]pyridin-2-yl}tetrahydro-2H-pyran-4-ol

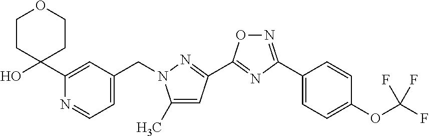

17 mg (0.15 mmol) of potassium tert-butylate were added to 39 mg (0.13 mmol) of the compound from Example 23A in 1.3 ml of THF at 0° C., a solution of 40 mg (max. 0.14 mmol) of the compound from Example 88A in 1 ml of THF was then added and the mixture was stirred overnight at RT. The batch was subsequently concentrated on a rotary evaporator and the residue was purified by means of preparative HPLC (method P). The product obtained in this way was subsequently purified by preparative HPLC (method R) again. 13 mg (20% of th.) of the title compound were obtained.

¹H-NMR (400 MHz, DMSO-d₆, δ/ppm): 8.53 (d, 1H), 8.20 (d, 2H), 7.60 (d, 2H), 7.54 (s, 1H), 7.04 (d, 1H), 7.00 (s, 1H), 5.63 (s, 2H), 3.72 (d, 4H), 2.34 (s, 3H), 2.15 (m, 2H), 1.43 (d, 2H).

LC/MS (method D, ESIpos): Rₜ=2.25 min, m/z=502 [M+H]⁺.

### Example 212

### 2-{4-[(5-Methyl-3-{3-[4-(trifluoromethoxy)phenyl]-1,2,4-oxadiazol-5-yl}-1H-pyrazol-1-yl)-methyl]pyridin-2-yl}propan-2-ol

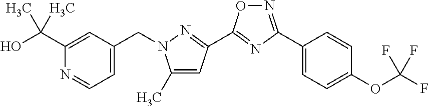

Analogously to the process described under Example 211, 103 mg (0.33 mmol) of the compound from Example 23A and 113 mg (0.37 mmol) of the compound from Example 89A were reacted to give 50 mg (33% of th.) of the title compound.

¹H-NMR (400 MHz, DMSO-d₆, δ/ppm): 8.45 (d, 1H), 8.20 (d, 2H), 7.59 (d, 2H), 7.46 (s, 1H), 6.98 (s, 1H), 6.92 (d, 2H), 5.58 (s, 2H), 5.20 (s, 1H), 2.34 (s, 3H), 1.41 (s, 6H).

LC/MS (method D, ESIpos): Rₜ=2.18 min, m/z=459 [M+H]⁺.

### B. EVALUATION OF THE PHARMACOLOGICAL ACTIVITY

The pharmacological activity of the compounds according to the invention can be demonstrated by in vitro and in vivo studies such as are known to the person skilled in the art. The usefulness of the substances according to the invention can be illustrated by way of example by in vitro (tumour) cell experiments and in vivo tumour models such as are described below. The connection between an inhibition of the HIF transcription activity and the inhibition of tumour growth is demonstrated by numerous studies described in the literature (cf. e.g. Warburg, 1956; Semenza, 2007).

### B-1. HIF-Luciferase Assay

HCT 116 cells were transfected in a stable manner with a plasmid which contained a luciferase reporter under the control of an HIF-responsive sequence. These cells were sown in microtitre plates [20,000 cells/cavity in RPMI 1640 medium with 10% foetal calf serum (FCS) and 100 μg/ml of hygromycin]. Incubation was carried out overnight under standard conditions (5% CO₂, 21% O₂, 37° C., moistened). The following morning the cells were incubated with various concentrations of the test substances (0-10 μmol/l) in a hypoxia chamber (1% O₂). After 24 h, Bright Glo reagent (Promega, Wisconsin, USA) was added in accordance with the manufacturer'/s instructions, and after 5 min the luminescence was measured. Cells which were incubated under normoxia served as background controls.

The IC₅₀ values from this assay for representative embodiment examples are listed in the following table:

| Example no. | IC₅₀ [nmol/l] |
|---|---|
| 24 | 1 |
| 25 | 8 |
| 29 | 10 |
| 33 | 30 |
| 68 | 10 |
| 72 | 10 |
| 79 | 4 |
| 81 | 10 |
| 94 | 20 |
| 100 | 9 |
| 121 | 0.4 |
| 133 | 1.5 |
| 134 | 2 |
| 135 | 4 |
| 166 | 2 |
| 170 | 0.3 |
| 171 | 0.4 |
| 178 | 0.6 |
| 180 | 1 |
| 181 | 2 |
| 182 | 2 |
| 183 | 0.6 |
| 184 | 2 |
| 187 | 0.6 |
| 188 | 1 |
| 196 | 0.5 |

### B-2. Suppression of HIF Target Genes In Vitro:

Human bronchial carcinoma cells (H460 and A549) were incubated for 16 h with variable concentrations of the test substances (1 nM to 10 μM) under normoxic conditions and under a 1% oxygen partial pressure (see HIF-luciferase assay). The total RNA was isolated from the cells and transcribed into cDNA and the mRNA expression of HIF target genes was analysed in real time PCR. Active test substances already lower the mRNA expression of the HIF target genes compared with untreated cells under normoxic conditions, but above all under hypoxic conditions.

### B-3. Human Xenograft and Syngenic Tumour Models:

Human tumour xenograft models in immunodeficient mice and syngenic tumour mouse models were used for evaluation of the substances. For this, tumour cells were cultured in vivo and implanted subcutaneously, or tumour xenotransplant pieces were transplanted further subcutaneously. The animals were treated by oral, subcutaneous or intraperitoneal therapy after the tumour was established. The activity of the test substances was analysed in monotherapy and in combination therapy with other pharmacological active substance. The tumour inhibitory potency of the test substance on tumours of advanced size (approx. 100 mm²) was moreover characterized. The state of health of the animals was checked daily, and the treatments were performed in accordance with animal protection regulations. The tumour area was measured with slide gauges (length L, breadth B=shorter dimension). The tumour volume was calculated from the formula (L×B²)/2. The inhibition in tumour growth was determined at the end of the study as the T/C ratio of the tumour areas and tumour weights and as the TGI value (tumour growth inhibition, calculated from the formula [1−(T/C)]×100) (T=tumour size in the treated group; C=tumour size in the untreated control group).

The influence of the test substances on the tumour vessel architecture and the blood flow within the tumour was identified with the aid of computer microtomography and ultrasound microstudies on treated and untreated tumour-carrying mice.

### B-4. Determination of Pharmacokinetic Parameters Following Intravenous and Peroral Administration:

The substance to investigated was administered to animals (e.g. mice or rats) intravenously as a solution (e.g. in corresponding plasma with a small addition of DMSO or in a PEG/ethanol/water mixture), and peroral administration took place as a solution (e.g. in a Solutol/ethanol/water or PEG/ethanol/water mixture) or as a suspension (e.g. in tylose), in each case via a stomach tube. After administration of the substance, blood was taken from the animals at specified points in time. This was heparinized, and plasma was then obtained therefrom by centrifugation. The substance was quantified analytically in the plasma via LC-MS/MS. From the plasma concentration/time plots determined in this way, the pharmacokinetic parameters, such as AUC (area under the concentration/time curve), Cₘₐₓ(maximum plasma concentration), T_{1/2} (half life), V_{SS} (distribution volume) and CL (clearance), and the absolute and the relative bioavailability (i.v./p.o. comparison or comparison of suspension to solution after p.o. administration), were calculated using an internal standard and with the aid of a validated computer program.

### C. EMBODIMENT EXAMPLES FOR PHARMACEUTICAL COMPOSITIONS

The compounds according to the invention can be converted into pharmaceutical formulations as follows.

### Tablet:

### Composition:

100 mg of the compound according to the invention, 50 mg of lactose (monohydrate), 50 mg of maize starch (native), 10 mg of polyvinylpyrrolidone (PVP 25) (BASF, Ludwigshafen, Germany) and 2 mg magnesium stearate.

Tablet weight 212 mg. Diameter 8 mm, radius of curvature 12 mm.

### Preparation:

The mixture of compound according to the invention, lactose and starch is granulated with a 5% strength solution (w/w) of the PVP in water. After drying, the granules are mixed with the magnesium stearate for 5 minutes. This mixture is pressed with a conventional tablet press (for tablet format see above). A pressing force of 15 kN is used as the recommended value for the pressing.

### Suspension for Oral Administration:

### Composition:

1,000 mg of the compound according to the invention, 1,000 mg of ethanol (96%), 400 mg of Rhodigel® (xanthan gum from FMC, Pennsylvania, USA) and 99 g of water.

10 ml of oral suspension correspond to an individual dose of 100 mg of the compound according to the invention.

### Preparation:

The Rhodigel is suspended in ethanol and the compound according to the invention is added to the suspension. The water is added with stirring. The mixture is stirred for approx. 6 h until swelling of the Rhodigel has ended.

### Solution for Oral Administration:

### Composition:

500 mg of the compound according to the invention, 2.5 g of polysorbate and 97 g of polyethylene glycol 400.20 g of oral solution correspond to an individual dose of 100 mg of the compound according to the invention.

### Preparation:

The compound according to the invention is suspended in the mixture of polyethylene glycol and polysorbate, while stirring. The stirring operation is continued until solution of the compound according to the invention is complete.

i.v. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and is transferred into sterile and pyrogen-free injection containers.

### D. LITERATURE REFERENCES

Globocan 2002 Report
IARC International Agency for Research on Cancer: Globocan 2002, http://www-dep.iarc.fr/globocan/downloads.htm
American Cancer Society, Cancer Facts and Figures 2005
American Cancer Society: Cancer Facts and Figures 2007, http://www.cancer.org/docroot/STT/content/STT_{—}1x_Cancer_Facts_Figures_{—}2007. asp
Gibbs J B, 2000
Gibbs J B: Mechanism-based target identification and drug discovery in cancer research, Science 2000, 287 (5460), 1969-1973.
Semenza and Wang, 1992
Semenza G L, Wang G L: A nuclear factor induced by hypoxia via de novo protein synthesis binds to the human erythropoietin gene enhancer at a site required for transcriptional activation, Mol. Cell. Biol. 1992, 12 (12), 5447-5454.
Wang and Semenza, 1995
Wang G L, Semenza G L: Purification and characterization of hypoxia-inducible factor 1, J. Biol. Chem. 1995, 270 (3), 1230-1237.
Wang, Jiang et al., 1995
Wang G L, Jiang B H, Rue E A, Semenza G L: Hypoxia-inducible factor 1 is a basic-helix-loop-helix-PAS heterodimer regulated by cellular O₂ tension, PNAS 1995, 92 (12), 5510-5514.
Jiang, Rue et al., 1996
Jiang B H, Rue E, Wang G L, Roe R, Semenza G L: Dimerization, DNA binding, and transactivation properties of hypoxia-inducible factor 1, J. Biol. Chem. 1996, 271 (30), 17771-17778.
Makino, Cao et al., 2001
Makino Y, Cao R, Svensson K, Bertilsson G, Asman M, Tanaka H, Cao Y, Poellinger L: Nature 2001, 414 (6863), 550-554.
Jiang, Semenza et al., 1996
Jiang B H, Semenza G L, Bauer C, Marti H H: Hypoxia-inducible factor 1 levels vary exponentially over a physiologically relevant range of O₂ tension, Am. J. Physiol. 1996, 271, 1172-1180.
Maxwell, Wiesener et al., 1999
Maxwell P H, Wiesener M S, Chang G W, Clifford S C, Vaux E C, Cockman M E, Wykoff C C, Ratcliffe P J: The tumour suppressor protein VHL targets hypoxia-inducible factors for oxygen-dependent proteolysis, Nature 1999, 399 (6733), 271-275.
Hirota and Semenza, 2006
Hirota K, Semenza G L: Regulation of angiogenesis by hypoxia-inducible factor 1, Crit. Rev. Oncol. Hematol. 2006, 59 (1), 15-26.
Chen, Zhao et al., 2003
Chen J, Zhao S, Nakada K, Kuge Y, Tamaki N, Okada F, Wang J, Shindo M, Higashino F, Takeda K, Asaka M, Katoh H, Sugiyama T, Hosokawa M, Kobayashi M: Dominant-negative hypoxia-inducible factor-1alpha reduces tumorigenicity of pancreatic cancer cells through the suppression of glucose metabolism, Am. J. Pathol. 2003, 162 (4), 1283-1291.
Stoeltzing, McCarty et al., 2004
Stoeltzing O, McCarty M F, Wey J S, Fan F, Liu W, Belcheva A, Bucana C D, Semenza G L, Ellis L M: Role of hypoxia-inducible factor-1alpha in gastric cancer cell growth, angiogenesis, and vessel maturation, J. Natl. Cancer Inst. 2004, 96 (12), 946-956.
Li, Lin et al., 2005
Li L, Lin X, Stayer M, Shoemaker A, Semizarov D, Fesik S W, Shen Y: Evaluating hypoxia-inducible factor-1alpha as a cancer therapeutic target via inducible RNA interference in vivo, Cancer Res. 2005, 65 (16), 7249-7258.
Mizukami, Jo et al., 2005
Mizukami Y, Jo W S, Duerr E M, Gala M, Li J, Zhang X, Zimmer M A, Iliopoulos O, Zukerberg L R, Kohgo Y, Lynch M P, Rueda B R, Chung D C: Induction of interleukin-8 preserves the angiogenic response in HIF-1alpha-deficient colon cancer cells, Nat. Med. 2005, 11 (9), 992-997.
Li, Shi et al., 2006
Li J, Shi M, Cao Y, Yuan W, Pang T, Li B, Sun Z, Chen L, Zhao R C: Knockdown of hypoxia-inducible factor-1alpha in breast carcinoma MCF-7 cells results in reduced tumor growth and increased sensitivity to methotrexate, Biochem. Biophys. Res. Commun. 2006, 342, 1341-1351.
Semenza, 2007
Semenza G L: Drug Discov. Today 2007, 12 (19-20), 853-859.
Weidemann and Johnson, 2008
Weidemann A, Johnson R S: Cell Death and Differentiation 2008, 15, 621-627.
Aiello et al., 1994
Aiello et al.: New Engl. J. Med. 1994, 331, 1480.
Peer et al., 1995
Peer et al.: Lab. Invest. 1995, 72, 638.
Lopez et al., 1996
Lopez et al.: Invest. Ophthalmol. Vis. Sci. 1996, 37, 855.
Warburg, 1956
Warburg O: Science 1956, 123 (3191), 309-314.

1. A compound of the formula (I) 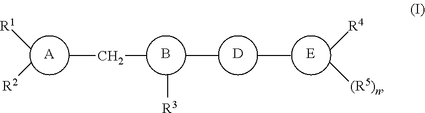
in which
either (a)
the ring 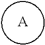
represents a pyridyl ring
and
the ring 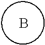
with the substituent R³ represents a heteroaryl ring of the formula 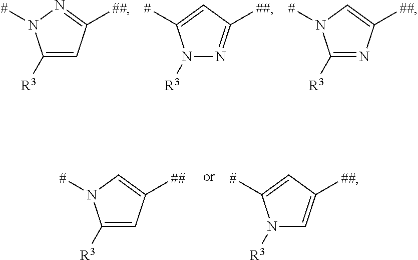
wherein
# designates the linkage point with the adjacent CH₂ group
and
## designates the linkage point with the ring 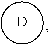
or (b)
the ring 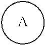
represents a phenyl ring
and
the ring 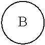
with the substituent R³ represents a heteroaryl ring of the formula 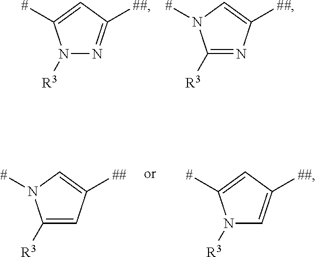
wherein
# designates the linkage point with the adjacent CH₂ group
and
## designates the linkage point with the ring 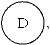
the ring 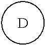
represents a heteroaryl ring of the formula 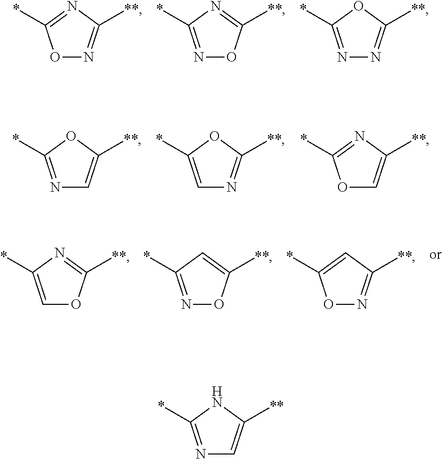
wherein
designates the linkage point with the ring 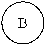
and
** designates the linkage point with the ring 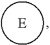
the ring 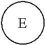
represents a phenyl or pyridyl ring,
R¹ represents hydrogen or a substituent chosen from the series halogen, cyano, (C₁-C₆)-alkyl, (C₂-C₆)-alkenyl, (C₂-C₆)-alkynyl, (C₃-C₆)-cycloalkyl, oxetanyl, tetrahydrofuranyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷, —NR⁶R⁸, —N(R⁶)—C(═O)—R⁷ and —N(R⁶)—S(═O)₂—R⁷,
wherein (C₁-C₆)-alkyl, (C₂-C₆)-alkenyl and (C₂-C₆)-alkynyl in their turn can be substituted up to three times by fluorine and up to two times in an identical or different manner by a radical chosen from the series hydroxyl, (C₁-C₄)-alkoxy, trifluoromethoxy, tri-(C₁-C₄)-alkylsilyl, (C₁-C₄)-alkoxycarbonyl and (C₃-C₆)-cycloalkyl
and
oxetanyl, tetrahydrofuranyl, tetrahydropyranyl and the cycloalkyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
and wherein
R⁶ and R⁷ independently of each other denote hydrogen, (C₁-C₆)-alkyl or (C₃-C₆)-cycloalkyl,
wherein (C₁-C₆)-alkyl can be substituted up to three times by fluorine and up to two times in an identical or different manner by a radical chosen from the series hydroxyl, (C₁-C₄)-alkoxy, trifluoromethoxy, (C₁-C₄)-alkoxycarbonyl and (C₃-C₆)-cycloalkyl
and
the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
and
R⁸ denotes hydrogen, amino, (C₁-C₆)-alkyl, (C₃-C₆)-cycloalkyl or 5- or 6-membered heteroaryl,
wherein (C₁-C₆)-alkyl can be substituted up to three times by fluorine and up to two times in an identical or different manner by a radical chosen from the series hydroxyl, (C₁-C₄)-alkoxy, trifluoromethoxy, (C₁-C₄)-alkoxycarbonyl, (C₃-C₆)-cycloalkyl, oxetanyl, tetrahydrofuranyl, tetrahydropyranyl and 5- or 6-membered heteroaryl
and wherein
oxetanyl, tetrahydrofuranyl, tetrahydropyranyl and the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and (C₁-C₄)-alkoxycarbonyl,
and
the heteroaryl groups mentioned can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
R² represents hydrogen or a substituent chosen from the series fluorine, chlorine, cyano, methyl, trifluoromethyl, hydroxyl, methoxy and trifluoromethoxy,
R³ represents methyl, ethyl or trifluoromethyl,
R⁴ represents hydrogen or a substituent chosen from the series halogen, cyano, pentafluorothio, (C₁-C₆)-alkyl, tri-(C₁-C₄)-alkylsilyl, —OR⁹, —NR⁹R¹⁰, —N(R⁹)—C(═O)—R¹⁰, —N(R⁹)—C(═O)—OR¹⁰, —N(R⁹)—S(═O)₂—R¹⁰, —C(═O)—OR⁹, —C(═O)—NR⁹R¹⁰, —SR⁹, —S(═O)—R⁹, —S(═O)₂—R⁹, —S(═O)₂—NR⁹R¹⁰, —S(═O)(═NH)—R⁹, —S(═O)(═NCH₃)—R⁹, (C₃-C₆)-cycloalkyl, 4- to 6-membered heterocyclyl and 5- or 6-membered heteroaryl,
wherein (C₁-C₆)-alkyl in its turn can be substituted up to three times by fluorine and up to two times in an identical or different manner by a radical chosen from the series —OR⁹, —NR⁹R¹⁰, —N(R⁹)—C(═O)—R¹⁰, —N(R⁹)—C(═O)—OR¹⁰, —C(═O)—OR⁹, —C(═O)—NR⁹R¹⁰, (C₃-C₆)-cycloalkyl, 4- to 6-membered heterocyclyl and 5- or 6-membered heteroaryl
and wherein
the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkylcarbonyl, (C₁-C₄)-alkoxycarbonyl, aminocarbonyl, mono-(C₁-C₄)-alkyl-aminocarbonyl and di-(C₁-C₄)-alkylaminocarbonyl
and
the heteroaryl groups mentioned in their turn can be substituted up to two times in an identical or different manner by a radical chosen from the series fluorine, chlorine, cyano, (C₁-C₄)-alkyl and (C₁-C₄)-alkoxy
wherein the (C₁-C₄)-alkyl substituents mentioned herein and the (C₁-C₄)-alkoxy substituents mentioned herein in their turn can be substituted by hydroxyl, (C₁-C₄)-alkoxy, trifluoromethoxy, (C₁-C₄)-alkylcarbonyloxy, (C₁-C₄)-alkoxycarbonyl, aminocarbonyl, mono-(C₁-C₄)-alkylaminocarbonyl or di-(C₁-C₄)-alkylaminocarbonyl or up to three times by fluorine,
and wherein
R⁹ and R¹⁰ independently of each other for each individual occurrence denote hydrogen, (C₁-C₆)-alkyl, (C₃-C₆)-cycloalkyl or 4- to 6-membered heterocyclyl,
wherein (C₁-C₆)-alkyl can be substituted up to three times by fluorine and up to two times in an identical or different manner by a radical chosen from the series hydroxyl, (C₁-C₄)-alkoxy, trifluoromethoxy, amino, mono-(C₁-C₄)-alkylamino, di-(C₁-C₄)-alkylamino, (C₁-C₄)-alkoxycarbonyl, (C₃-C₆)-cycloalkyl and 4- to 6-membered heterocyclyl
and
the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
or
R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
R⁵ represents a substituent chosen from the series fluorine, chlorine, cyano, methyl, trifluoromethyl and hydroxyl
and
n represents the number 0, 1 or 2,
wherein in the case where the substituent R⁵ occurs twice, its meanings can be identical or different,
or a salt thereof.

2. A compound according to claim 1, in which
the ring 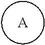
represents a phenyl or pyridyl ring and the adjacent groups R¹ and CH₂ are bonded to ring carbon atoms 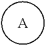
in 1, 3 or 1,4 relation to one another
and
the ring 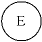
with the substituents R⁴ and R⁵ represents a phenyl ring of the formula 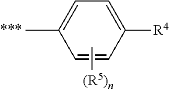
wherein
designates the linkage point with the ring 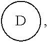
or a salt thereof.

3. A compound according to claim 1, in which
either (a)
the ring 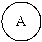
represents a pyridyl ring and the adjacent groups R¹ and CH₂ are bonded to ring carbon atoms of this pyridyl ring in 1, 3 or 1,4 relation to one another
and
the ring 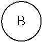
with the substituent R³ represents a heteroaryl ring of the formula 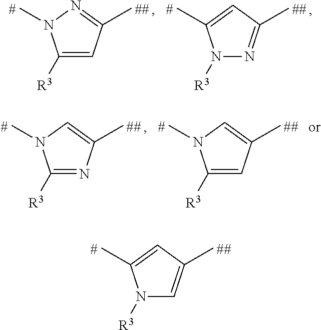
wherein
# designates the linkage point with the adjacent CH₂ group
and
## designates the linkage point with the ring 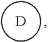
or (b)
the ring 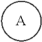
represents a phenyl ring and the adjacent groups R¹ and CH₂ are bonded to this phenyl ring in 1, 3 or 1,4 relation to one another,
and
the ring 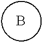
with the substituent R³ represents a heteroaryl ring of the formula 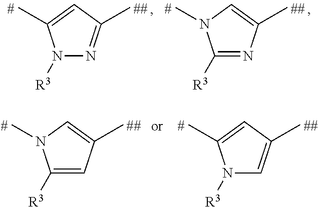
wherein
# designates the linkage point with the adjacent CH₂ group
and
## designates the linkage point with the ring 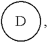
the ring 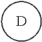
represents a heteroaryl ring of the formula 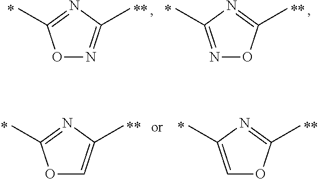
wherein
* designates the linkage point with the ring 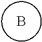
and
** designates the linkage point with the ring 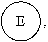
the ring 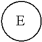
with the substituents R⁴ and R⁵ represents a phenyl ring of the formula 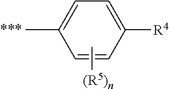
wherein
*** designates the linkage point with the ring 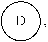
R¹ represents hydrogen or a substituent chosen from the series fluorine, chlorine, bromine, cyano, (C₁-C₄)-alkyl, (C₂-C₄)-alkynyl, (C₃-C₆)-cycloalkyl, oxetanyl, tetrahydrofuranyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
wherein (C₁-C₄)-alkyl and (C₂-C₄)-alkynyl in their turn can be substituted by a radical chosen from the series hydroxyl, (C₁-C₄)-alkoxy, trifluoromethoxy, trimethylsilyl, (C₁-C₄)-alkoxycarbonyl and (C₃-C₆)-cycloalkyl and up to three times by fluorine
and
oxetanyl, tetrahydrofuranyl, tetrahydropyranyl and the cycloalkyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy and (C₁-C₄)-alkoxycarbonyl,
and wherein
R⁶ and R⁷ independently of each other denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
and
R⁸ denotes hydrogen, amino, (C₁-C₆)-alkyl, (C₃-C₆)-cycloalkyl or 5- or 6-membered heteroaryl,
wherein (C₁-C₆)-alkyl can be substituted by a radical chosen from the series hydroxyl, (C₁-C₄)-alkoxy, trifluoromethoxy, (C₁-C₄)-alkoxycarbonyl, (C₃-C₆)-cycloalkyl, tetrahydrofuranyl, tetrahydropyranyl and 5- or 6-membered heteroaryl and up to three times by fluorine
and wherein
tetrahydrofuranyl, tetrahydropyranyl and the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and (C₁-C₄)-alkoxycarbonyl,
and
the heteroaryl groups mentioned can be substituted up to three times in an identical or different manner by a radical chosen from the series (C₁-C₄)-alkyl, trifluoromethyl, (C₁-C₄)-alkoxy and trifluoromethoxy
R² represents hydrogen or a substituent chosen from the series fluorine, chlorine, methyl, trifluoromethyl, methoxy and trifluoromethoxy,
R³ represents methyl, ethyl or trifluoromethyl,
R⁴ represents a substituent chosen from the series chlorine, cyano, pentafluorothio, (C₁-C₆)-alkyl, tri-(C₁-C₄-)alkylsilyl, —OR⁹, —NR⁹R¹⁰, —SR⁹, —S(═O)—R⁹, —S(═O)₂—R⁹, —S(═O)(═NH)—R⁹, —S(═O)(═NCH₃)—R⁹, (C₃-C₆)-cycloalkyl and 4- to 6-membered heterocyclyl,
wherein (C₁-C₆)-alkyl in its turn can be substituted by a radical chosen from the series —OR⁹, —NR⁹R¹⁰, —N(R⁹)—C(═O)—R¹⁰, —C(═O)—NR⁹R¹⁰, (C₃-C₆)-cycloalkyl, 4- to 6-membered heterocyclyl and 5- or 6-membered heteroaryl and up to three times by fluorine
and wherein
the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, oxo, (C₁-C₄)-alkylcarbonyl, mono-(C₁-C₄)-alkylaminocarbonyl and di-(C₁-C₄)-alkylaminocarbonyl
and
the heteroaryl groups mentioned in their turn can be substituted up to two times in an identical or different manner by a radical chosen from the series fluorine, chlorine, cyano, (C₁-C₄)-alkyl and (C₁-C₄)-alkoxy
wherein the (C₁-C₄)-alkyl substituents mentioned herein and the (C₁-C₄)-alkoxy substituents mentioned herein in their turn can be substituted by hydroxyl, (C₁-C₄)-alkoxy, trifluoromethoxy, (C₁-C₄)-alkoxycarbonyl, mono-(C₁-C₄)-alkyl-aminocarbonyl or di-(C₁-C₄)-alkylaminocarbonyl or up to three times by fluorine,
and wherein
R⁹ and R¹⁰ independently of each other for each individual occurrence denote hydrogen, (C₁-C₄)-alkyl, (C₃-C₆)-cycloalkyl or 4- to 6-membered heterocyclyl,
wherein (C₁-C₄)-alkyl can be substituted by a radical chosen from the series hydroxyl, (C₁-C₄)-alkoxy, trifluoromethoxy, (C₃-C₆)-cycloalkyl and 4- to 6-membered heterocyclyl and up to three times by fluorine
and
the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
and
n represents the number 0 or 1,
or a salt thereof.

4. A compound according to claim 1, in which
the ring 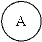
with the substituents R¹ and R² represents a pyridyl ring of the formula 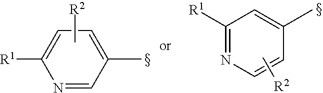
wherein
§ designates the linkage point with the adjacent CH₂ group,
the ring 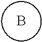
with the substituent R³ represents a heteroaryl ring of the formula 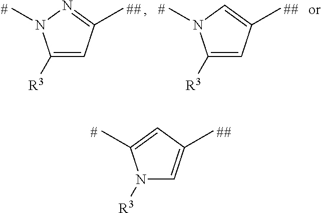
wherein
# designates the linkage point with the adjacent CH₂ group
and
## designates the linkage point with the ring 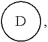
the ring 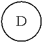
represents a heteroaryl ring of the formula 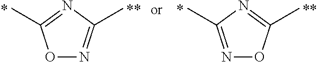
wherein
* designates the linkage point with the ring 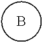
and
** designates the linkage point with the ring 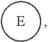
the ring 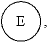
with the substituents R⁴ and R⁵ represents a phenyl ring of the formula 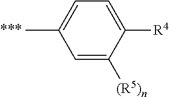
wherein
*** designates the linkage point with the ring 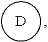
R¹ represents hydrogen or a substituent chosen from the series chlorine, cyano, (C₁-C₄)-alkyl, (C₂-C₄)-alkynyl, cyclopropyl, cyclobutyl, oxetanyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
wherein (C₁-C₄)-alkyl and (C₂-C₄)-alkynyl in their turn can be substituted by a radical chosen from the series hydroxyl, methoxy, ethoxy, trifluoromethoxy, cyclopropyl and cyclobutyl and up to three times by fluorine
and
oxetanyl and tetrahydropyranyl in their turn can be substituted by methyl, ethyl, hydroxyl, methoxy or ethoxy
and
the cyclopropyl and cyclobutyl groups mentioned in their turn can be substituted up to two times in an identical or different manner by a radical chosen from the series fluorine, methyl, ethyl and trifluoromethyl,
and wherein
R⁶ and R⁷ independently of each other denote hydrogen, (C₁-C₄)-alkyl or (C₃-C₆)-cycloalkyl,
wherein (C₁-C₄)-alkyl can be substituted by a radical chosen from the series hydroxyl, methoxy, ethoxy, trifluoromethoxy, cyclopropyl and cyclobutyl and up to three times by fluorine
and
R⁸ denotes hydrogen, (C₁-C₄)-alkyl, (C₃-C₆)-cycloalkyl or 5- or 6-membered heteroaryl,
wherein (C₁-C₄)-alkyl can be substituted by a radical chosen from the series hydroxyl, methoxy, ethoxy, trifluoromethoxy, (C₃-C₆)-cycloalkyl, tetrahydrofuranyl, tetrahydropyranyl and 5- or 6-membered heteroaryl and up to three times by fluorine
and wherein
tetrahydrofuranyl, tetrahydropyranyl and the cycloalkyl groups mentioned can be substituted up to two times in an identical or different manner by a radical chosen from the series fluorine, methyl, ethyl, trifluoromethyl, hydroxyl, methoxy and ethoxy
and
the heteroaryl groups mentioned can be substituted up to three times in an identical or different manner by a radical chosen from the series methyl, ethyl and trifluoromethyl
R² represents hydrogen or a substituent chosen from the series fluorine, chlorine, methyl and methoxy,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—R⁹, —S(═O)(═NCH₃)—R⁹, (C₃-C₆)-cycloalkyl and 4- to 6-membered heterocyclyl,
wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
and
the cycloalkyl and heterocyclyl groups mentioned in their turn can be substituted up to two times in an identical or different manner by a radical chosen from the series fluorine, (C₁-C₄)-alkyl, (C₁-C₄)-alkoxy and oxo,
wherein the (C₁-C₄)-alkyl substituent mentioned and the (C₁-C₄)-alkoxy substituent in their turn can be substituted by hydroxyl, methoxy, trifluoromethoxy, ethoxy, methoxycarbonyl, ethoxycarbonyl, tert-butoxycarbonyl, methylaminocarbonyl or dimethylaminocarbonyl or up to three times by fluorine,
and wherein
R⁹ and R¹⁰ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
or
R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
or a salt thereof.

5. A compound according to claim 1, in which
the ring 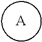
with the substituents R¹ and R² represents a phenyl ring of the formula 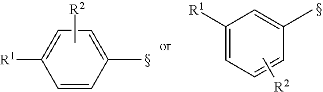
wherein
§ designates the linkage point with the adjacent CH₂ group,
the ring 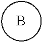
with the substituent R³ represents a heteroaryl ring of the formula 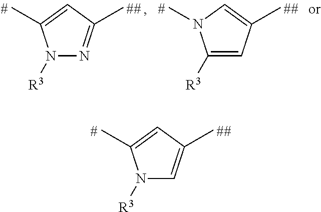
wherein
# designates the linkage point with the adjacent CH₂ group
and
## designates the linkage point with the ring 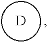
the ring 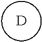
represents a heteroaryl ring of the formula 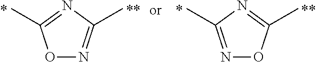
wherein
* designates the linkage point with the ring 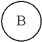
and
** designates the linkage point with the ring 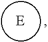
the ring 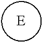
with the substituents R⁴ and R⁵ represents a phenyl ring of the formula 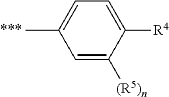
wherein
*** designates the linkage point with the ring 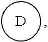
R¹ represents hydrogen or a substituent chosen from the series chlorine, cyano, (C₁-C₄)-alkyl, (C₂-C₄)-alkynyl, cyclopropyl, cyclobutyl, oxetanyl, tetrahydropyranyl, —OR⁶, —SR⁶, —S(═O)—R⁶, —S(═O)₂—R⁶, —C(═O)—OR⁶, —C(═O)—NR⁶R⁷, —S(═O)₂—NR⁶R⁷ and —NR⁶R⁸,
wherein (C₁-C₄)-alkyl and (C₂-C₄)-alkynyl in their turn can be substituted by a radical chosen from the series hydroxyl, methoxy, ethoxy, trifluoromethoxy, cyclopropyl and cyclobutyl and up to three times by fluorine
and
oxetanyl and tetrahydropyranyl in their turn can be substituted by methyl, ethyl, hydroxyl, methoxy or ethoxy
and
the cyclopropyl and cyclobutyl groups mentioned in their turn can be substituted up to two times in an identical or different manner by a radical chosen from the series fluorine, methyl, ethyl and trifluoromethyl,
and wherein
R⁶ and R⁷ independently of each other denote hydrogen, (C₁-C₄)-alkyl or (C₃-C₆)-cycloalkyl,
wherein (C₁-C₄)-alkyl can be substituted by a radical chosen from the series hydroxyl, methoxy, ethoxy, trifluoromethoxy, cyclopropyl and cyclobutyl and up to three times by fluorine
and
R⁸ denotes hydrogen, (C₁-C₄)-alkyl, (C₃-C₆)-cycloalkyl or 5- or 6-membered heteroaryl,
wherein (C₁-C₄)-alkyl can be substituted by a radical chosen from the series hydroxyl, methoxy, ethoxy, trifluoromethoxy, (C₃-C₆)-cycloalkyl, tetrahydrofuranyl, tetrahydropyranyl and 5- or 6-membered heteroaryl and up to three times by fluorine
and wherein
tetrahydrofuranyl, tetrahydropyranyl and the cycloalkyl groups mentioned can be substituted up to two times in an identical or different manner by a radical chosen from the series fluorine, methyl, ethyl, trifluoromethyl, hydroxyl, methoxy and ethoxy
and
the heteroaryl groups mentioned can be substituted up to three times in an identical or different manner by a radical chosen from the series methyl, ethyl and trifluoromethyl
R² represents hydrogen or a substituent chosen from the series fluorine, chlorine, methyl and methoxy,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—R⁹, —S(═O)(═NCH₃)—R⁹, (C₃-C₆)-cycloalkyl and 4- to 6-membered heterocyclyl,
wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
and
the cycloalkyl and heterocyclyl groups mentioned in their turn can be substituted up to two times in an identical or different manner by a radical chosen from the series fluorine, (C₁-C₄)-alkyl, (C₁-C₄)-alkoxy and oxo,
wherein the (C₁-C₄)-alkyl substituent mentioned and the (C₁-C₄)-alkoxy substituent in their turn can be substituted by hydroxyl, methoxy, trifluoromethoxy, ethoxy, methoxycarbonyl, ethoxycarbonyl, tert-butoxycarbonyl, methylaminocarbonyl or dimethylaminocarbonyl or up to three times by fluorine,
and wherein
R⁹ and R¹⁰ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
or
R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
or a salt thereof.

6. A compound according to claim 1, in which
the ring 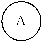
with the substituents R¹ and R² represents a pyridyl ring of the formula 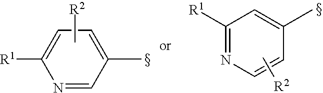
wherein
§ designates the linkage point with the adjacent CH₂ group,
the ring 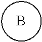
with the substituent R³ represents a heteroaryl ring of the formula 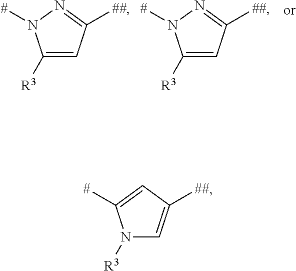
wherein
# designates the linkage point with the adjacent CH₂ group
and
## designates the linkage point with the ring 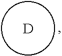
the ring 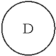
represents a heteroaryl ring of the formula 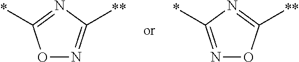
wherein
* designates the linkage point with the ring 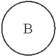
and
** designates the linkage point with the ring 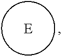
the ring 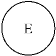
with the substituents R⁴ and R⁵ represents a phenyl ring of the formula 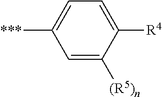
wherein
*** designates the linkage point with the ring 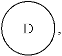
R¹ represents methyl or the group —NR⁶R⁸, wherein
R⁶ denotes hydrogen, methyl, ethyl or cyclopropyl,
and
R⁸ denotes (C₁-C₄)-alkyl or (C₃-C₆)-cycloalkyl,
wherein (C₁-C₄)-alkyl can be substituted by a radical chosen from the series hydroxyl, methoxy, ethoxy, (C₃-C₆)-cycloalkyl, tetrahydrofuranyl, tetrahydropyranyl and 5- or 6-membered heteroaryl and up to three times by fluorine
and wherein
tetrahydrofuranyl, tetrahydropyranyl and the cycloalkyl groups mentioned can be substituted up to two times in an identical or different manner by a radical chosen from the series fluorine, methyl, ethyl, trifluoromethyl, hydroxyl, methoxy and ethoxy
and
the heteroaryl group mentioned can be substituted up to three times in an identical or different manner by a radical chosen from the series methyl, ethyl and trifluoromethyl
R² represents hydrogen,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—CH₃, —S(═O)(═NH)—CF₃, —S(═O)(═NCH₃)—CH₃, —S(═O)(═NCH₃)—CF₃, (C₃-C₆)-cycloalkyl and 4- to 6-membered heterocyclyl,
wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
and
the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
wherein the (C₁-C₄)-alkyl substituent mentioned in its turn can be substituted by methoxy, trifluoromethoxy or ethoxy,
and wherein
R⁹ and R¹⁰ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
or
R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
or a salt thereof.

7. A compound according to claim 1, in which
the ring 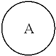
with the substituents R¹ and R² represents a phenyl ring of the formula 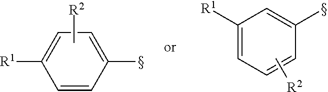
wherein
§ designates the linkage point with the adjacent CH₂ group,
the ring 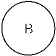
with the substituent R³ represents a heteroaryl ring of the formula 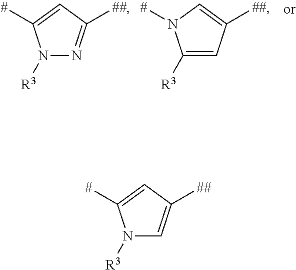
wherein
# designates the linkage point with the adjacent CH₂ group
and
## designates the linkage point with the ring 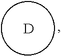
the ring 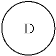
represents a heteroaryl ring of the formula 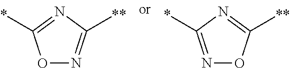
wherein
* designates the linkage point with the ring 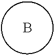
and
** designates the linkage point with the ring 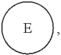
the ring 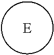
with the substituents R⁴ and R⁵ represents a phenyl ring of the formula 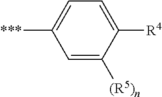
wherein
*** designates the linkage point with the ring 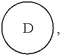
R¹ represents chlorine, cyano, methyl, ethyl, isopropyl, cyclopropyl, cyclobutyl, methoxy, ethoxy, methylsulphonyl, ethylsulphonyl, isopropylsulphonyl or the group —C(═O)—NR⁶R⁷, wherein
R⁶ and R⁷ independently of each other denote hydrogen, (C₁-C₄)-alkyl or (C₃-C₆)-cycloalkyl,
wherein (C₁-C₄)-alkyl can be substituted by a radical chosen from the series hydroxyl, methoxy, ethoxy, cyclopropyl and cyclobutyl and up to three times by fluorine
R² represents hydrogen,
R³ represents methyl,
R⁴ represents a substituent chosen from the series chlorine, pentafluorothio, (C₁-C₆)-alkyl, trimethylsilyl, —OR⁹, —SR⁹, —S(═O)—R⁹, —S(═O)₂—R⁹, —S(═O)(═NH)—CH₃, —S(═O)(═NH)—CF₃, —S(═O)(═NCH₃)—CH₃, —S(═O)(═NCH₃)—CF₃, (C₃-C₆)-cycloalkyl and 4- to 6-membered heterocyclyl,
wherein (C₁-C₆)-alkyl in its turn can be substituted by a radical chosen from the series —OR⁹, —NR⁹R¹⁰, —C(═O)—NR⁹R¹⁰, (C₃-C₆)-cycloalkyl and 4- to 6-membered heterocyclyl and up to three times by fluorine
and
the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
wherein the (C₁-C₄)-alkyl substituent mentioned in its turn can be substituted by methoxy, trifluoromethoxy or ethoxy,
and wherein
R⁹ and R¹⁰ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, (C₁-C₄)-alkoxy and trifluoromethoxy,
or
R⁹ and R¹⁰ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, hydroxyl, (C₁-C₄)-alkoxy, oxo, acetyl and propionyl,
R⁵ represents fluorine,
and
n represents the number 0 or 1,
or a salt thereof.

8. (canceled)

9. (canceled)

10. (canceled)

11. (canceled)

12. (canceled)

13. A pharmaceutical composition comprising a compound according to claim 1 and one or more inert, non-toxic, pharmaceutically suitable auxiliary substances.

14. A pharmaceutical composition comprising a compound according to claim 1 in combination with one or more further active compounds.

15. (canceled)

16. (canceled)

17. A method for the treatment and/or prevention of cancer diseases or tumour diseases comprising administering to a human or animal in need thereof a pharmaceutically effective amount of at least one compound according to claim 1.

18. A method for the treatment and/or prevention of ischaemic cardiovascular diseases, cardiac insufficiency, cardiac infarction, arrhythmia, stroke, pulmonary hypertension, fibrotic diseases of the kidney and lung, psoriasis, diabetic retinopathy, macular degeneration, rheumatic arthritis of Chugwash polycythaemia comprising administering to a human or animal in need thereof a pharmaceutically effective amount of at least one compound according to claim 1.

19. A process for the preparation of a compound of the formula (I-F) 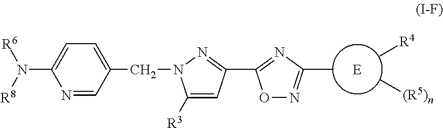
in which the ring E and R³, R⁴, R⁵, R⁶, R⁸ and n in each case have the meanings given in claim 1,
characterized in that an N′-hydroxyamidine of the formula (VIII) 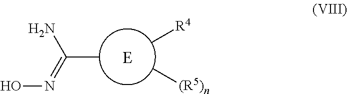
in which the ring E and R⁴, R⁵ and n have the meanings given above,
first can either be
[A] subjected to a condensation reaction with a pyrazolecarboxylic acid of the formula (XXVI) 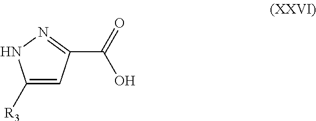
in which R³ has the meaning given above,
to give a 1,2,4-oxadiazole derivative of the formula (XXVII) 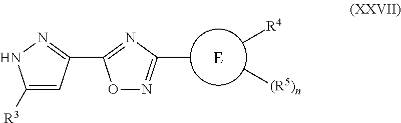
in which the ring E and R³, R⁴, R⁵ and n have the meanings given above,
and this is then alkylated in the presence of a base with a compound of the formula (XXVIII) 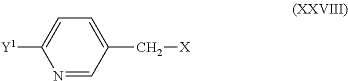
in which
Y¹ represents chlorine, bromine or iodine
and
X represents chlorine, bromine, iodine, mesylate, triflate or tosylate,
to give a compound of the formula (XXIX) 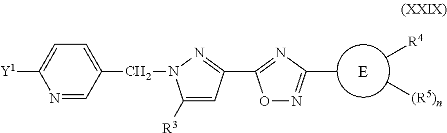
in which the ring E and R³, R⁴, R⁵, n and Y¹ have the meanings given above,
or
[B] subjected to a condensation reaction with a pyrazolecarboxylic acid of the formula (XXX) 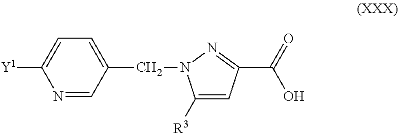
in which R³ has the meaning given above
and
Y¹ represents chlorine, bromine or iodine,
to give the compound of the formula (XXIX) 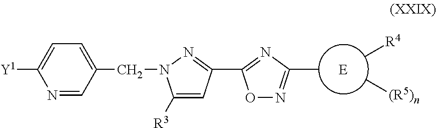
in which the ring E and R³, R⁴, R⁵, n and Y¹ have the meanings given above,
and the compound of the formula (XXIX) obtained in this way in then reacted, optionally in the presence of an auxiliary base, with a compound of the formula (XII) 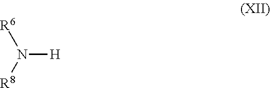
in which R⁶ and R⁸ have the meanings given above.
